(19) United States
(12) Patent Application Publication
(10) Pub. No.: US 2005/0260187 A1
(43) Pub. Date: Nov. 24, 2005

(54) Therapeutic and cosmetic uses of heparanases

(75) Inventors: Neta Ilan, Rehovot (IL); Israel Vlodavsky, Mevaseret Zion (IL); Oron Yacoby-Zeevi, Moshav Bizaron (IL); Iris Pecker, Rishon LeZion (IL); Elena Feinstein, Rehovot (IL)

Correspondence Address: Martin MOYNIHAN;c/o ANTHONY CASTORINA, SUITE 207, 2001 JEFFERSON DAVIS HIGHWAY, ARLINGTON, VA, 22202, US

(73) Assignees: Insight Strategy & Marketing Ltd.; Hadasit Medical Research Services and Development Ltd.

(21) Appl. No.: 11/106,672

(22) Filed: Apr. 15, 2005

Related U.S. Application Data
(63) Continuation of application No. 10/341,582, filed on Jan. 14, 2003.
Application No. 10/341,582 is a continuation-in-part of application No. 09/988,113, filed on Nov. 19, 2001, now Pat. No. 6,790,658.
Application No. 09/988,113 is a continuation of application No. 09/776,874, filed on Feb. 6, 2001.
Application No. 09/776,874 is a continuation of application No. 09/258,892, filed on Mar. 1, 1999, now abandoned.
Application No. 09/258,892 is a continuation-in-part of application No. PCT/US98/17954, filed on Aug. 31, 1998.
Application No. 10/341,582 is a continuation-in-part of application No. PCT/IL01/00830, filed on Sep. 5, 2001.
Application No. PCT/IL01/00830 is a continuation-in-part of application No. 09/727,479, filed on Dec. 4, 2000, now abandoned.
(60) Provisional application No. 60/231,551, filed on Sep. 11, 2000.
(60) Provisional application No. 60/244,593, filed on Nov. 1, 2000.

(51) Int. Cl. A61K 38/47; C12N 9/24

(52) U.S. Cl. 424/94.61; 435/200

(57) ABSTRACT

Methods and compositions for inducing and/or accelerating wound healing and/or angiogenesis via the catalytic activity of heparanase are disclosed.

This is a continuation of U.S. patent application Ser. No. 10/341,582, filed Jan. 14, 2003, which is a continuation-in-part of U.S. patent application Ser. No. 09/988,113, filed Nov. 19, 2001, now U.S. Pat. No. 6,790,658, which is a continuation of U.S. patent application Ser. No. 09/776,874, filed Feb. 6, 2001, which is a continuation of U.S. patent application Ser. No. 09/258,892, filed Mar. 1, 1999, now abandoned, which is a continuation-in-part of PCT/US98/17954, filed: Aug. 31, 1998, which claims priority from U.S. patent application Ser. No. 09/109,386, filed Jul. 2, 1998, now abandoned, which is a continuation-in-part of U.S. patent application Ser. No. 08/922,170, filed Sep. 2, 1997, now U.S. Pat. No. 5,968,822.

U.S. patent application Ser. No. 10/341,582 also was a continuation-in-part of PCT/IL01/00830, filed Sep. 5, 2001, whereby this application is also a continuation-in-part of PCT/IL01/00830, filed Sep. 5, 2001, which claims the benefit of priority from U.S. patent application Ser. No. 09/727,479, filed Dec. 4, 2000, now abandoned, which claims the benefit of priority from U.S. Provisional Patent Application Nos. 60/231,551, filed Sep. 11, 2000, and 60/244,593, filed Nov. 1, 2000.

### FIELD OF THE INVENTION

The present invention relates to a polynucleotide, referred to hereinbelow as hpa, encoding a polypeptide having heparanase activity, vectors (nucleic acid constructs) including same and genetically modified cells expressing heparanase. The invention further relates to a recombinant protein having heparanase activity and to antisense oligonucleotides, constructs and ribozymes for down regulating heparanase activity. In addition, the invention relates to heparanase promoter sequences and their uses.

### BACKGROUND OF THE INVENTION

Proteoglycans (PGs):

Proteoglycans (previously named mucopolysaccharides) are remarkably complex molecules and are found in every tissue of the body. They are associated with each other and also with other major structural components, such as collagen and elastin. Some PGs interact with certain adhesive proteins, such as fibronectin and laminin.

Glycosaminoglycans (GAGs):

Glycosaminoglycans (GAGs) proteoglycans are polyanions and hence bind polycations and cations, such as Na⁺ and K⁺. This latter ability attracts water by osmotic pressure into the extracellular matrix and contributes to its turgor. GAGs also gel at relatively low concentrations. The long extended nature of the polysaccharide chains of GAGs and their ability to gel, allow relatively free diffusion of small molecules, but restrict the passage of larger macromolecules. Because of their extended structures and the huge macromolecular aggregates they often form, they occupy a large volume of the extracellular matrix relative to proteins [Murry R K and Keeley F W; Harper's Biochemistry, 24th Ed. Ch. 57 pp. 667-85].

Heparan sulfate proteoglycans: Heparan sulfate proteoglycans (HSPG) are ubiquitous macromolecules associated with the cell surface and extra cellular matrix (ECM) of a wide range of cells of vertebrate and invertebrate tissues (1-4). The basic HSPG structure includes a protein core to which several linear heparan sulfate chains are covalently attached. These polysaccharide chains are typically composed of repeating hexuronic and D-glucosamine disaccharide units that are substituted to a varying extent with N- and O-linked sulfate moieties and N-linked acetyl groups (1-4) Studies on the involvement of ECM molecules in cell attachment, growth and differentiation revealed a central role of HSPG in embryonic morphogenesis, angiogenesis, neurite outgrowth and tissue repair (1-5). HSPG are prominent components of blood vessels (3). In large blood vessels they are concentrated mostly in the intima and inner media, whereas in capillaries they are found mainly in the subendothelial basement membrane where they support proliferating and migrating endothelial cells and stabilize the structure of the capillary wall. The ability of HSPG to interact with ECM macromolecules such as collagen, laminin and fibronectin, and with different attachment sites on plasma membranes suggests a key role for this proteoglycan in the self-assembly and insolubility of ECM components, as well as in cell adhesion and locomotion. HSPG maintains tissue integrity and endothelial cell function. It serves as an adhesion molecule and presents adhesion-inducing cytokines (especially chemokines), facilitating localization and activation of leukocytes. The adhesive effect of heparan sulfate-bound chemokines can be abrogated by exposing the extracellular matrices to heparanase before or after the addition of chemokines. Heparan sulfate modulates the activation and the action of enzymes secreted by inflammatory cells. The function of heparan sulfate changes during the course of the immune response are due to changes in the metabolism of heparan sulfate and to the differential expression of and competition between heparan sulfate-binding molecules [Selvan R S et al.; Ann. NY Acad. Sci. 1996; 797: 127-139]

Cleavage of the heparan sulfate (HS) chains may therefore result in degradation of the subendothelial ECM and hence may play a decisive role in extravasation of blood-borne cells. HS catabolism is observed in inflammation, wound repair, diabetes, and cancer metastasis, suggesting that enzymes which degrade HS play important roles in pathologic processes. Heparanase activity has been described in activated immune system cells and highly metastatic cancer cells (6-8), but research has been handicapped by the lack of biologic tools to explore potential causative roles of heparanase in disease conditions.

Other PGs and GAGs, such as hyaluronic acid, chondroitin sulfates, keratan sulfates I, II, dermatan sulfate and heparin have also important physiological functions.

GAG Degrading Enzymes:

Degradation of GAGs is carried out by a battery of lysosomal hydrolases. These include certain endoglycosidases, such as, but not limited to, mammal heparanase (U.S. Pat. No. 5,968,822 for recombinant and WO91/02977 for native human heparanase) and connective tissue activating peptide III (CTAP, WO95/04158, for native and U.S. Pat. No. 4,897,348 for recombinant CTAP) which degrade heparan sulfate and to a lesser extent heparin; heparinase I, II and III (U.S. Pat. No. 5,389,539 for the native form and WO95/34635 A1, U.S. Pat. No. 5,714,376 and U.S. Pat. No. 5,681,733 for the recombinant form), e.g., from Flavbacterium heparinum and Bacillus sp., which cleave heparin-like molecules; heparitinase T-I, T-II, T-III and T-VI from Bacillus circulans (U.S. Pat. No. 5,405,759, JO 4278087 and JP04-278087); glucuronidase; chondroitinase ABC (EC 4.2.2.4) from Proteus vulgaris, AC (EC 4.2.2.5) from Arthrobacter aurescens or Flavobacterium heparinum, B and C (EC -4.2.2) from Flavobacterium heparinum which degrade chondroitin sulfate; hyaluronidase from sheep of bovine testes which degrade hyaluronidase and chondroitin sulfate; various exoglycosidases (e.g., β-glucuronidase EC 3.2.1.31) from bovine liver, mollusks and various bacteria; and sulfatases (e.g., iduronate sulfatase) EC 3.1.6.1 from limpets (Patella vulgaris), Aerobacter aerogens, Abalone entrails and Helix pomatia, generally acting in sequence to degrade the Various GAGs.

Heparanase:

One important enzyme involved in the catabolism of certain GAGs is heparanase. It is an endo-β-glucuronidase that cleaves heparan sulfate at specific interchain sites. Interaction of T and B lymphocytes, platelets, granulocytes, macrophages and mast cells with the subendothelial extracellular matrix (ECM) is associated with degradation of heparan sulfate by heparanase activity. The enzyme is released from intracellular compartments (e.g., lysosomes or specific granules) in response to various activation; signals (e.g., thrombin, calcium ionophore, immune complexes, antigens and mitogens), suggesting its regulated involvement in inflammation and cellular immunity [Vlodavsky I et al.; Invasion Metas. 1992; 12(2):112-27].

Involvement of Heparanase in Tumor Cell Invasion and Metastasis: Circulating tumor cells arrested in the capillary beds of different organs must invade the endothelial cell lining and degrade its underlying basement membrane (BM) in order to invade into the extravascular tissue(s) where they establish metastasis (9, 10). Metastatic tumor cells often attach at or near the intercellular junctions between adjacent endothelial cells. Such attachment of the metastatic cells is followed by rupture of the junctions, retraction of the endothelial cell borders and migration through the breach in the endothelium toward the exposed underlying BM (9). Once located between endothelial cells and the BM, the invading cells must degrade the subendothelial glycoproteins and proteoglycans of the BM in order to migrate out of the vascular compartment. Several cellular enzymes (e.g., collagenase IV, plasminogen activator, cathepsin B elastase, etc.) are thought to be involved in degradation of BM (10). Among these enzymes is an endo-β-D-glucuronidase (heparanase) that cleaves HS at specific intrachain sites (6, 8, 11). Expression of a HS degrading heparanase was found to correlate with the metastatic potential of mouse lymphoma (11), fibrosarcoma and melanoma (8) cells. Moreover, elevated levels of heparanase were detected in sera from metastatic tumor bearing animals and melanoma patients (8) and in tumor biopsies of cancer patients (12).

The control of cell proliferation and tumor progression by the local microenvironment, focusing on the interaction of cells with the extracellular matrix (ECM) produced by cultured corneal and vascular endothelial cells, was investigated previously by the present inventors. This cultured ECM closely resembles the subendothelium in vivo in its morphological appearance and molecular composition. It contains collagens (mostly type III and IV, with smaller amounts of types I and V), proteoglycans (mostly heparan sulfate- and dermatan sulfate-proteoglycans, with smaller amounts of chondroitin sulfate proteoglycans), laminin, fibronectin, entactin and elastin (13, 14). The ability of cells to degrade HS in the cultured ECM was studied by allowing cells to interact with a metabolically sulfate labeled ECM, followed by gel filtration (Sepharose 6B) analysis of degradation products released into the culture medium (11). While intact HSPG are eluted next to the void volume of the column (Kav<0.2, Mr˜0.5×10⁶), labeled degradation fragments of HS side chains are eluted more toward the Vt of the column (0.5<kav<0.8, Mr=5-7×10³) (11).

The heparanase inhibitory effect of various non-anticoagulant species of heparin that might be of potential use in preventing extravasation of blood-borne cells was also investigated by the present inventors. Inhibition of heparanase was best achieved by heparin species containing 16 sugar units or more and having sulfate groups at both the N and O positions. While O-desulfation abolished the heparanase inhibiting effect of heparin, O-sulfated, N-acetylated heparin retained a high inhibitory activity, provided that the N-substituted molecules had a molecular size of about 4,000 daltons or more (7). Treatment of experimental animals with heparanase inhibitors (e.g., non-anticoagulant species of heparin) markedly reduced (>90%) the incidence of lung metastases induced by B16 melanoma, Lewis lung carcinoma and mammary adenocarcinoma cells (7, 8, 16). Heparin fractions with high and low affinity to anti-thrombin III exhibited a comparable high anti-metastatic activity, indicating that the heparanase inhibiting activity of heparin, rather than its anticoagulant activity, plays a role in the anti-metastatic properties of the polysaccharide (7).

Heparanase activity in the urine of cancer patients: In an attempt to further elucidate the involvement of heparanase in tumor progression and its relevance to human cancer, urine samples for heparanase activity were screened (16a). Heparanase activity was detected in the urine of some, but not all, cancer patients. High levels of heparanase activity were determined in the urine of patients with an aggressive metastatic disease and there was no detectable activity in the urine of healthy donors.

Heparanase activity was also found in the urine of 20% of normal and microalbuminuric insulin dependent diabetes mellitus (IDDM) patients, most likely due to diabetic nephropathy, the most important single disorder leading to renal failure in adults.

Involvement of Heparanase in Tumor Cell Invasion and Metastasis:

Circulating tumor cells arrested in the capillary beds of different organs must invade the endothelial cell lining and degrade its underlying basement membrane (BM) in order to escape into the extravascular tissue(s) where they establish metastasis [Liotta, L. A., Rao, C. N.; and Barsky, S. H. (1983). Tumor invasion and the extracellular matrix. Lab. Invest., 49, 639-649]. Several cellular enzymes (e.g., collagenase IV, plasminogen activator, cathepsin B, elastase) are thought to be involved in degradation of the BM [Liotta, L. A., Rao, C. N., and Barsky, S. H. (1983). Tumor invasion and the extracellular matrix. Lab. Invest., 49, 639-649]. Among these enzymes is an endo-β-D-glucuronidase (heparanase) that cleaves HS at specific intrachain sites [Vlodavsky, I., Eldor, A., Haimovitz-Friedman, A., Matzner, Y., Ishai-Michaeli, R., Levi, E., Bashkin, P., Lider, O., Naparstek, Y., Cohen, I. R., and Fuks, Z. (1992). Expression of heparanase by platelets and circulating cells of the immune system: Possible involvement in diapedesis and extravasation. Invasion & Metastasis, 12, 112-127; Nakajima, M., Irimura; T., and Nicolson, G. L. (1988). Heparanase and tumor metastasis. J. Cell. Biochem., 36, 157-167; Vlodavsky, I., Fuks, Z., Bar-Ner, M., Ariav, Y., and Schinmacher, V. (1983). Lymphoma cell mediated degradation of sulfated proteoglycans in the subendothelial extracellular matrix: Relationship to tumor cell metastasis. Cancer Res., 43, 2704-2711; Vlodavsky, I., Ishai-Michaeli, R., Bar-Ner, M., Fridman, R., Horowitz, A. T., Fuks, Z. and Biran, S. Involvement of heparanase in tumor metastasis and angiogenesis. Is. J. Med. 24:464-470, 1988]. HS degrading heparanase activity was found to correlate with the metastatic potential at mouse lymphoma cells [Vlodavsky, I., Fuks, Z., Bar-Ner, M., Ariav, Y., and Schirrmacher, V. (1983). Lymphoma cell mediated degradation of sulfated proteoglycans in the subendothelial extracellular matrix. Relationship to tumor cell metastasis. Cancer. Res., 43, 2704-2711], fibrosarcoma and melanoma [Nakajima M Irimura, T., and Nicolson, G. L. (1988). Heparanase and tumor metastasis. J. Cell. Biochem., 36, 157-167]. The same is true for human breast, bladder and prostate carcinoma cells [see U.S. patent application Ser. No. 09/109,386, which is incorporated by reference as if fully set forth herein]. Moreover, elevated; levels heparanase were detected in sera [Nakajima, M., Irimura, T., and Nicolson, G. L. (1988). Heparanase and tumor metastasis. J. Cell. Biochem., 36, 157-167] and urine (U.S. patent application Ser. No. 09/109,386) of metastatic tumor bearing animals and cancer patients and in tumor biopsies [Vlodavsky, I., Ishai-Michaeli, R., Bar-Ner, M., Fridman, R., Horowitz, A. T., Fuks, Z. and Biran, S. Involvement of heparanase in tumor metastasis and angiogenesis. Is. J. Med. 24:464-470, 1988]. Treatment of experimental animals with heparanase alternative substrates and inhibitor (e.g., non-anticoagulant species of low molecular weight heparin, laminarin sulfate) markedly reduced (>90%) the incidence of lung metastases induced by BA16 melanoma, Lewis lung carcinoma and mammary adenocarcinoma cells [Vlodavsky, I., Mohsen, M., Lider, O., Ishai-Michaeli, R., Ekre, H.-P., Svahn, C. M., Vigoda, M., and Peretz, T. (1995). Inhibition of tumor metastasis by heparanase inhibiting species of heparin Invasion & Metastasis 14: 290-302; Nakajima, M., Irimura, T, and Nicolson G. L. (1988). Heparanase and tumor metastasis. J. Cell. Biochem., 36, 157-167; Parish, C. R., Coombe, D. R., Jakobsen, K. B., and Underwood, P. A. (1987). Evidence that sulfated polysaccharides inhibit tumor metastasis by blocking tumor cell-derived heparanase. Int. J. Cancer, 40, 511-517], indicating that heparanase inhibitors may be applied to inhibit tumor cell invasion and metastasis.

The studies on the control of tumor progression by its local environment, focus on the interaction of cells with the extracellular matrix (ECM) produced by cultured corneal and vascular endothelial cells (EC) [Vlodavsky, I., Liu, G. M., and Gospodarowicz, D. (1980). Morphological appearance, growth behavior and migratory activity of human tumor cells maintained on extracellular matrix vs. plastic. Cell, 19, 607-616; Vlodavsky, I., Bar-Shavit, R., Ishai-Michaeli, R., Bashkin, P., and Fuks, Z. (1991). Extracellular sequestration and release of fibroblast growth factor: a regulatory mechanism? Trends Biochem. Sci., 16, 268-271]. This ECM closely resembles the subendothelium in vivo in its morphological appearance and molecular composition. It contains collagens (mostly type III and IV, with smaller amounts of types I and V), proteoglycans (mostly heparan: sulfate- and dermatan sulfate-proteoglycans, with smaller amounts of chondroitin sulfate proteoglycans), laminin, fibronectin, entactin and elastin [Parish, C. R., Coombe, D. R., Jakobsen K. B., and Underwood, P. A., (1987). Evidence that sulfated polysaccharides inhibit tumor metastasis by blocking tumor cell-derived heparanase. Int. J. Cancer, 40, 511-517; Vlodavsky, I., Liu, G. M., and Gospodarowiczi D. (1980). Morphological appearance, growth behavior and migratory activity of human tumor cells maintained on extracellular matrix vs. plastic. Cell, 19, 607-616]. The ability of cells to degrade HS in the ECM was studied by allowing cells to interact with a metabolically sulfate labeled ECM, followed by gel filtration (Sepharose 6B) analysis of degradation products released into the culture medium [Vlodavsky, I., Fuks, Z., Bar-Ner, M.; Ariav, Y., and Schirrmacher, V. (1983). Lymphoma cell mediated degradation of sulfated proteoglycans in the subendothelial extracellular matrix. Relationship to tumor cell metastasis. Cancer Res., 43, 2704-2711]. While intact HSPG are eluted next to the void volume of the column (Kav<0.2, Mr of about 0.5×10⁶), labeled degradation fragments of HS side chains are eluted more toward the Vt of the column; (0.5<kav<0.8, Mr of about 5.7×10³) [Vlodavsky, I., Fuks, Z., Bar-Ner, M., Ariav, Y., and Schirrmacher, V. (1983). Lymphoma cell mediated degradation of sulfated proteoglycans in the subendothelial extracellular matrix. Relationship to tumor cell metastasis. Cancer Res., 43, 2704-2711]. Compounds which efficiently inhibit the ability of heparanase to degrade the above-described naturally produced basement membrane-like substrate, were also found to inhibit experimental metastasis in mice and rats [Vlodavsky, I., Mohsen, M., Lider, O., Ishai-Michaeli, R., Ekre, H.-P., Svahn, C. M., Vigoda, M., and Peretz, T. (1.995). Inhibition of tumor metastasis by heparanase inhibiting species of heparin. Invasion & Metastasis, 14: 290-302; Nakajima, M., Irimura, T., and Nicolson, G. L. (1988). Heparanase and tumor metastasis. J. Cell. Biochem., 36, 157-167; Parish, C. R., Coombe, D. R., Jakobsen, K. B., and Underwood, P. A. (1987). Evidence that sulfated polysaccharides inhibit tumor metastasis by blocking tumor cell-derived heparanase. Int. J. Cancer, 40, 511-517; Coombe D R, Parish C R, Ramshaw I A, Snowden J M: Analysis of the inhibition of tumor metastasis by: sulfated polysaccharides. Int J Cancer 1987; 39:82-8].

Possible Involvement of Heparanase in Tumor Angiogenesis:

It was previously demonstrated that heparanase may not only function in cell migration and invasion, but may also elicit an indirect neovascular response [Vlodavsky, I., Bar-Shavit, R., Ishai-Michaeli, R., Bashkin, P., and Fuks, Z. (1991). Extracellular sequestration- and release of fibroblast growth factor: a regulatory mechanism? Trends Biochem. Sci., 16, 268-271]. The results suggest that the ECM HSPGs provide a natural storage depot for βFGF and possibly other heparin-binding growth promoting factors. Heparanase mediated release of active βFGF from its storage within ECM may therefore provide a novel mechanism for induction of neovascularization in normal and pathological situations [Vlodavsky, I., Bar-Shavit, R., Korner, G., and Fuks, Z. (1993). Extracellular matrix-bound growth factors, enzymes and plasma proteins. In Basement membranes: Cellular and molecular aspects (eds. D. H. Rohrbach and R. Timpl), pp 327-343. Academic press Inc., Orlando, Fl.; Thunberg L, Backstrom G, Grundberg H, Risenfield J, Lindahl U: The molecular size of the antithrombin-binding sequence in heparin. FEBS Lett 1980; 117:203-206]. However, these prior art references fail to demonstrate the involvement of heparanase in angiogenesis, which therefore still remains to be proved.

On the other hand, release of bFGF from storage in ECM as a complex with HS fragment, may elicit localized endothelial cell proliferation and neovascularization in processes such as wound healing, inflammation and tumor development (24, 25).

Expression of Heparanase by Cells of the Immune System:

Heparanase activity correlates with the ability of activated cells of the immune system to leave the circulation and elicit both inflammatory and autoimmune responses. Interaction of platelets, granulocytes, T and B lymphocytes, macrophages and mast cells with the subendothelial ECM is associated with degradation of HS by a specific heparanase activity (6). The enzyme is released from intracellular compartments (e.g., lysosomes, specific granules, etc.) in response to various activation signals (e.g., thrombin, calcium ionophore, immune complexes, antigens, mitogens, etc.), suggesting its regulated involvement in inflammation and cellular immunity.

Some of the Observations Regarding the Heparanase Enzyme were Reviewed in Reference No. 6 and are Listed Hereinbelow:

First, a proteolytic activity (plasminogen activator) and heparanase participate synergistically in sequential degradation of the ECM HSPG by inflammatory leukocytes and malignant cells.

Second, a large proportion of the platelet heparanase exists in a latent form, probably as a complex with chondroitin sulfate. The latent enzyme is activated by tumor cell-derived factor(s) and may then facilitate cell invasion through the vascular endothelium in the process of tumor metastasis.

Third, release of the platelet heparanase from α-granules is induced by a strong stimulant (i.e., thrombin), but not in response to platelet activation on ECM.

Fourth, the neutrophil heparanase is preferentially and readily released in response to a threshold activation and upon incubation of the cells on ECM.

Fifth, contact of neutrophils with ECM inhibited release of noxious enzymes (proteases, lysozyme) and oxygen radicals, but not of enzymes (heparanase, gelatinase) which may enable diapedesis. This protective role of the subendothelial ECM was observed when the cells were stimulated with soluble factors but not with phagocytosable stimulants.

Sixth, intracellular heparanase is secreted within minutes after exposure of T cell lines to specific antigens.

Seventh, mitogens (Con A, LPS) induce synthesis and secretion of heparanase by normal T and B lymphocytes maintained in vitro. T lymphocyte heparanase is also induced by immunization with antigen in vivo.

Eighth, heparanase activity is expressed by pre-B lymphomas and B-lymphomas, but not by plasmacytomas and resting normal B lymphocytes.

Ninth, heparanase activity is expressed by activated macrophages during incubation with ECM, but there was little or no release of the enzyme into the incubation medium. Similar results were obtained with human myeloid leukemia cells induced to differentiate to mature macrophages.

Tenth, T-cell mediated delayed type hypersensitivity and experimental autoimmunity are suppressed by low doses of heparanase inhibiting non-anticoagulant species of heparin (30).

Eleventh, heparanase activity expressed by platelets, neutrophils and metastatic tumor cells releases active bFGF from ECM and basement membranes. Release of bFGF; from storage in ECM may elicit a localized neovascular response in processes such as wound healing, inflammation and tumor development.

Twelfth, among the breakdown products of the ECM generated by heparanase is a tri-sulfated disaccharide that can inhibit T-cell mediated inflammation in vivo (31). This inhibition was associated with an inhibitory-effect of the disaccharide on the production of biologically active TNFα by activated T cells in vitro (31).

Other Potential Therapeutic Applications:

Apart from its involvement in tumor cell metastasis, inflammation and autoimmunity, mammalian heparanase may be applied to modulate: bioavailability of heparin-binding growth factors (15); cellular responses to heparin-binding growth factors (e.g., bFGF, VEGF) and cytokines (L-8) (31a, 29); cell interaction with plasma lipoproteins (32); cellular susceptibility to certain viral and some bacterial and protozoa infections (33, 33a, 33b); and disintegration of amyloid plaques (34). Heparanase may thus prove useful for conditions such as wound healing, angiogenesis, restenosis, atherosclerosis, inflammation, neurodegenerative diseases, and viral infections. Mammalian heparanase can be used to neutralize plasma heparin, as a potential replacement of protamine. Anti-heparanase antibodies may be applied for immunodetection and diagnosis of micrometastases, autoimmune lesions and renal failure in biopsy specimens, plasma samples, and body fluids. Common use in basic research is expected.

The identification of the hpa gene encoding for heparanase enzyme will enable the production of a recombinant enzyme in heterologous expression systems. Availability of the recombinant protein will pave the way for solving the protein structure function relationship and will provide a tool for developing new inhibitors.

Viral Infection:

The presence of heparan sulfate on cell surfaces have been shown to be the principal requirement for the binding of Herpes Simplex (33) and Dengue (33a), viruses to cells and for subsequent infection of the cells. Removal of the cell surface heparan sulfate by heparanase may therefore abolish virus infection. In fact, treatment of cells with bacterial heparitinase (degrading heparan sulfate) or heparinase (degrading heparan) reduced the binding of two related animal herpes viruses to cells and rendered the cells at least partially resistant to virus infection (33). There are some indications that the cell surface heparan sulfate is also involved in HIV infection (33b).

Neurodegenerative Diseases:

Heparan sulfate proteoglycans were identified in the prion protein amyloid plaques of Genstmann-Straussler Syndrome, Creutzfeldt-Jakob disease and Scrape (34). Heparanase may disintegrate these amyloid plaques which are also thought to play a role in the pathogenesis of Alzheimer's disease.

Restenosis and Atherosclerosis:

Proliferation of arterial smooth muscle cells (SMCs) in response to endothelial injury and accumulation of cholesterol rich lipoproteins are basic events in the pathogenesis of atherosclerosis and restenosis (35). Apart from its involvement in SMC proliferation (i.e., low affinity receptors for heparin-binding growth factors), HS is also involved in lipoprotein binding, retention and uptake (36). It was demonstrated that HSPG and lipoprotein lipase participate in a novel catabolic pathway that may allow substantial cellular and interstitial accumulation of cholesterol rich lipoproteins (32). The latter pathway is expected to be highly atherogenic by promoting accumulation of apoB and apoE rich lipoproteins (i.e. LDL, VLDL, chylomicrons), independent of feed back inhibition by the cellular sterol content. Removal of SMC HS by heparanase is therefore expected to inhibit both SMC proliferation and lipid accumulation and thus may halt the progression of restenosis and atherosclerosis.

Gene Therapy:

The ultimate goal in the management of inherited as well as acquired diseases is a rational therapy with the aim to eliminate the underlying biochemical defects associated with the disease rather then symptomatic treatment. Gene therapy is a promising candidate to meet these objectives. Initially it was developed for treatment of genetic disorders, however, the consensus view today is that it offers the prospect of providing therapy for a variety of acquired diseases, including cancer, viral infections, vascular diseases and neurodegenerative disorders.

The gene-based therapeutic can act either intracellularly, affecting only the cells to which it is delivered, or extracellularly, using the recipient cells as local endogenous factories for the therapeutic product(s). The application of gene therapy may follow any of the following strategies: (i) prophylactic gene therapy, such as using gene transfer to protect cells against viral infection; (ii) cytotoxic gene therapy, such as cancer therapy, where genes encode cytotoxic products to render the target cells vulnerable to attack by the normal immune response; (iii) biochemical correction, primarily for the treatment of single gene defects, where a normal copy of the gene is added to the affected or other cells.

To allow efficient transfer of the therapeutic genes, a variety of gene delivery techniques have been developed based on viral and non-viral vector systems. The most widely used and most efficient systems for delivering genetic material into target cells are viral vectors. So far, 329 clinical studies (phase I, I/II and II) with over 2,500 patients have been initiated Worldwide since 1989 (50).

The approach of gene addition pose serious barriers. The expression of many genes is tightly regulated and context dependent, so achieving the correct balance and function of expression is challenging. The gene itself is often quite large, containing many exons and introns. The delivery vector is usually a virus, which can infect with a high efficiency but may, on the other hand, induce immunological response and consequently decreases effectiveness, especially upon secondary administration. Most of the current expression vector-based gene therapy protocols fail to achieve clinically significant transgene expression required for treating genetic diseases. Apparently it is difficult to deliver enough virus to the right cell type to elicit an effective and therapeutic effect (51)

Homologous recombination, which was initially considered to be of limited use for gene therapy because of its low frequency in mammalian cells, has recently emerged as a potential strategy for developing gene therapy. Different approaches have been used to study homologous recombination in mammalian cells; some involve DNA repair mechanisms. These studies aimed at either gene disruption or gene correction and include RNA/DNA chimeric oligonucleotides, small or large homologous DNA fragments, or adeno-associated viral vectors. Most of these studies show a reasonable frequency of homologous recombination, which warrants further in vivo testing (52). Homologous recombination-based gene therapy has the potential to develop into a powerful therapeutic modality for genetic diseases. It can offer permanent expression and normal-regulation of corrected genes in appropriate cells or organs and probably can be used for treating dominantly inherited diseases such as polycystic kidney disease.

Genomic Sequences Function in: Regulation of Gene Expression:

The efficient expression of therapeutic genes in target cells or tissues is an important component of efficient and safe gene therapy. The expression of genes is driven by the promoter region upstream of the coding sequence, though regulation of expression may be supplemented by farther upstream or downstream DNA sequences or DNA in the introns of the gene. Since this important information is embedded in the DNA, the description of gene structure is crucial to the analysis of gene regulation. Characterization of cell specific or tissue specific promoters, as well as other tissue specific regulatory elements enables the use of such sequences to direct efficient cell specific, or developmental stage specific gene expression. This information provides the basis for targeting individual genes and for control of their expression by exogenous agents, such as drugs. Identification of transcription factors and other regulatory proteins required, for proper gene expression will point at new potential targets for modulating gene expression, when so desired or required

Efficient expression of many mammalian genes depends on the presence of at least one intron. The expression of mouse thymidylate synthase (TS) gene, for example, is greatly influenced by intron sequences. The addition of almost any of the introns from the mouse TS gene to an intronless TS minigene leads to a large increase in expression (42). The involvement of intron 1 in the regulation of expression was demonstrated for many other genes. In human factor IX (hFIX), intron 1 is able to increase the expression level about 3 fold mare as compared to that of the hFIX cDNA (43). The expression enhancing activity of intron 1 is due to efficient functional splicing sequences, present in the precursor mRNA. By being efficiently assembled into spliceosome complexes, transcripts with splicing sequences may be better protected in the nucleus from random degradations, than those without such sequences (44).

A forward-inserted intron1-carrying hFIX expression cassette suggested to be useful for directed gene transfer, while for retroviral-mediated gene transfer system, reversely-inserted intron 1-carrying hFIX expression cassette was considered (43).

A highly conserved cis-acting sequence element was identified in the first intron of the mouse and rat c-Ha-ras, and in the first exon of Ha- and Ki-ras genes of human, mouse and rat. This cis-acting regulatory sequence confers strong transcription enhancer activity that is differentially modulated by steroid hormones in metastatic and nonmetastatic sub populations. Perturbations in the regulatory activities of such cis-acting sequences: may play an important role in governing oncogenic potency of Ha-ras through transcriptional control mechanisms (45).

Intron sequences affect tissue specific, as well as inducible gene expression. A 182 bp intron 1 DNA segment of the mouse Col2a1 gene contains the necessary information to confer high-level, temporally correct, chondrocyte expression on a reporter gene in intact mouse embryos, while Col2a1 promoter sequences are dispensable for chondrocyte expression (46). In CoI1A1 gene the intron plays little or no role in constitutive expression of collagen in the skin, and in cultured cells derived from the skin, however, in the lungs of young mice, intron deletion results in decrease of expression to less than 50% (47).

A classical enhancer activity was shown in the 2 kb intron fragment in bovine beta-casein gene. The enhancer activity was largely dependent on the lactogenic hormones, especially prolactin. It was suggested that several elements in the intron-1 of the bovine beta-casein gene cooperatively interact not only with each other but also with its promoter for hormonal induction (48).

Identification and characterization of regulatory elements in genomic non-coding sequences, such as introns, provides a tool for designing and constructing novel vectors for tissue specific, hormone regulated or any other defined expression pattern, for gene therapy. Such an expression cassette was developed, utilizing regulatory elements from the human cytokeratin 18 (K18) gene, including 5′ genomic sequences and one of its introns. This cassette efficiently expresses reporter genes, as well as the human cystic fibrosis transmembrane conductance regulator (CFTR) gene, in cultured lung epithelial cells (49).

Alternative Splicing:

Alternative splicing of pre mRNA is a powerful and versatile regulatory mechanism that can effect quantitative control of gene expression and functional diversification of proteins. It contributes to major developmental decisions and also to a fine-tuning of gene function. Genetic and biochemical approaches have identified cis-acting regulatory elements and transacting factors that control: alternative splicing of specific mRNAs. This mechanism results in the generation of variant isoforms of various proteins from a single gene. These include cell surface molecules such as CD44, receptors, cytokines such as VEGF and enzymes. Products of alternatively spliced transcripts differ in their expression pattern, substrate specificity and other biological parameters.

The FGF receptor RNA undergoes alternative splicing which results in the production of several isoforms, which exhibit different ligand binding specificities. The alternative splicing is regulated in a cell specific manner (53).

Alternative spliced mRNAs are often correlated with malignancy. An increase in specific splice variant of tyrosinase was identified in murine melanomas (54). Multiple splicing variants of estrogen receptor are present in individual human breast tumors. CD44 has various isoform, some are characteristic of malignant tissues.

Identification of tumor specific alternative splice variants provide new tool for cancer diagnostics. CD44 variants have been used for detection of malignancy in urine samples from patients with urothelial cancer by competitive RT-PCR (55). CD44 exon 6 was suggested as prognostic indicator of metastasis in breast cancer (56).

Different enzymes or polypeptides generated by alternative splicing may have different function or catalytic specificity. The identification and characterization of the enzyme forms, which are involved in pathological processes, is crucial for the design of appropriate and efficient drugs.

Modulation of Gene Expression—Antisense Technology:

An antisense oligonucleotide (e.g., antisense oligodeoxyribonucleotide) may bind its target nucleic acid either by Watson-Crick base pairing or Hoogsteen and anti-Hoogsteen base pairing (64). According to the Watson-Crick base pairing, heterocyclic bases of the antisense oligonucleotide form hydrogen bonds with the heterocyclic bases of target single-stranded nucleic acids (RNA or single-stranded DNA), whereas according to the Hoogsteen base pairing, the heterocyclic bases of the target nucleic acid are double-stranded DNA, wherein a third strand is accommodated in the major groove of the B-form. DNA duplex by Hoogsteen and anti-Hoogsteen base pairing to form a triple helix structure.

According to both the Watson-Crick and the Hoogsteen base pairing models, antisense oligonucleotides have the potential to regulate gene expression and to disrupt the essential functions of the nucleic acids in cells., Therefore, antisense oligonucleotides have possible uses in modulating a wide range of diseases in which gene expression is altered.

Since the development of effective methods for chemically synthesizing oligonucleotides, these molecules have been extensively used in biochemistry and biological research and have the potential use in medicine, since carefully devised oligonucleotides can be used to control gene expression by regulating levels of transcription, transcripts and/or translation.

Oligodeoxyribonucleotides as long as 100 base pairs (bp) are routinely synthesized by solid phase methods using commercially available, fully automated synthesis machines. The chemical synthesis of oligoribonucleotides, however, is far less routine. Oligoribonucleotides are also much less stable than oligodeoxyribonucleotides, a fact which has contributed to the more prevalent use of oligodeoxyribonucleotides in medical and biological research, directed at, for example, the regulation of transcription or translation levels.

Gene expression involves few distinct and well regulated steps. The first major step of gene expression involves transcription of a messenger RNA (mRNA) which is an RNA sequence complementary to the antisense (i.e., −) DNA strand, or, in other words, identical in sequence to the DNA sense (i.e., +) strand, composing the gene. In eukaryotes, transcription occurs in the cell nucleus.

The second major step of gene expression involves translation of a protein (e.g., enzymes, structural proteins, secreted proteins, gene expression factors, etc.) in which the mRNA interacts with ribosomal RNA complexes (ribosomes) and amino acid activated transfer RNAs (tRNAs) to direct the synthesis of the protein coded for by the mRNA sequence.

Initiation of transcription requires specific recognition of a promoter DNA sequence located upstream to the coding sequence of a gene by an RNA-synthesizing enzyme—RNA polymerase. This recognition is preceded by sequence-specific binding of one or more transcription factors to the promoter sequence. Additional proteins which bind at or close to the promoter sequence may trans upregulate transcription via cis elements known as enhancer sequences. Other proteins which bind to or close to the promoter, but whose binding prohibits the action of RNA polymerase, are known as repressors.

There are also evidence that in some cases gene expression is down-regulated by endogenous antisense RNA repressors that bind a complementary mRNA transcript and thereby prevent its translation into a functional protein.

Thus, gene expression is typically upregulated by transcription factors and enhancers and downregulated by repressors.

However, in many disease situation gene expression is impaired. In many cases, such as different types of cancer, for various reasons the expression of a specific endogenous or exogenous (e.g., of a pathogen such as a virus) gene is upregulated. Furthermore, in infectious diseases caused by pathogens such as parasites, bacteria or viruses, the disease progression depends on expression of the pathogen genes, this phenomenon may also be considered as far as the patient is concerned as upregulation of exogenous genes.

Most conventional drugs function by interaction with and modulation of one or more targeted endogenous or exogenous proteins, e.g., enzymes. Such drugs, however, typically are not specific for targeted proteins but interact with other proteins as well. Thus, a relatively large dose of drug must be used to effectively modulate a targeted protein.

Typical daily doses of drugs are from 10⁻⁵-10⁻¹ millimoles per kilogram of body weight or 10⁻³-10 millimoles for a 100 kilogram person. If this modulation instead could be effected by interaction with and inactivation of mRNA, a dramatic reduction in the necessary amount of drug could likely be achieved, along with a corresponding reduction in side effects. Further reductions could be effected if such interaction could be rendered site specific. Given that a functioning gene continually produces mRNA, it would thus be even more advantageous if gene transcription could be arrested in its entirety.

Given these facts, it would be advantageous if gene expression could be arrested or downmodulated at the transcription level.

The ability of chemically, synthesizing oligonucleotides and analogs thereof having a selected predetermined sequence offers means for downmodulating gene expression. Three types of gene expression modulation strategies may be considered.

At the transcription level, antisense or sense oligonucleotides or analogs that bind to the genomic DNA by strand displacement or the formation of a triple helix, may prevent transcription (64).

At the transcript level, antisense oligonucleotides or analogs that bind target, mRNA molecules lead to the enzymatic cleavage of the hybrid by intracellular RNase H (65). In this case, by hybridizing to the targeted mRNA, the oligonucleotides or oligonucleotide analogs provide a duplex hybrid recognized and destroyed by the RNase H enzyme. Alternatively, such hybrid formation may lead to interference with correct splicing (66). As a result, in both cases, the number of the target mRNA intact transcripts ready for translation is reduced or eliminated.

At the translation level, antisense oligonucleotides or analogs that bind target mRNA molecules prevent, by steric hindrance, binding of essential translation factors (ribosomes), to the target mRNA, a phenomenon known in the art as hybridization arrest, disabling the translation of such mRNAs (67).

Thus, antisense sequences, which as described hereinabove may arrest the expression of any endogenous and/or exogenous gene depending on their specific sequence, attracted much attention by, scientists and pharmacologists who were devoted at developing the antisense approach into a new pharmacological tool (68).

For example, several antisense oligonucleotides have been shown to arrest hematopoietic cell proliferation (69), growth (70), entry into the S phase of the cell cycle (71), reduced survival (72) and prevent receptor mediated responses (73). For use of antisense oligonucleotides as antiviral agents the reader is referred to reference 74.

For efficient in vivo inhibition of gene expression using antisense oligonucleotides or analogs, the oligonucleotides or analogs must fulfill the following requirements (i) sufficient specificity in binding to the target sequence; (ii) solubility in water; (iii) stability against intra- and extracellular nucleases; (iv) capability of penetration through the cell membrane; and (v) when used to treat an organism, low toxicity.

Unmodified oligonucleotides are impractical for use as antisense sequences since they have short in vivo half-lives, during which they are degraded rapidly by nucleases. Furthermore, they are difficult to prepare in more than milligram quantities. In addition, such oligonucleotides are poor cell membrane penetraters (75).

Thus it is apparent that in order to meet all the above listed requirements, oligonucleotide analogs need to be devised in a suitable manner. Therefore, an extensive search for modified oligonucleotides has been initiated.

For example, problems arising in connection with double-stranded DNA (dsDNA) recognition through triple helix formations have been diminished by a clever “switch back” chemical linking, whereby a sequence of polypurine on one strand is recognized, and by “switching back”, a homopurine sequence on the other strand can be recognized. Also, good helix formation has been obtained by using artificial bases, thereby improving binding conditions with regard to ionic strength and pH.

In addition, in order to improve half-life as well as membrane penetration, a large number of variations in polynucleotide backbones have been done, nevertheless with little success.

Oligonucleotides can be modified either in the base, the sugar or the phosphate moiety. These modifications include, for example, the use of methylphosphonates, monothiophosphates, dithiophosphates, phosphoramidates, phosphate esters, bridged phosphorothioates, bridged phosphoramidates, bridged methylenephosphonates, dephospho internucleotide analogs with siloxane bridges, carbonate bridges, carboxymethyl ester bridges, carbonate bridges, carboxymethyl ester bridges, acetamide bridges, carbamate bridges, thioether bridges, sulfoxy bridges, sulfono bridges, various “plastic” DNAs, α-anomeric bridges and borane derivatives. For further details the reader is referred to reference 76.

International patent application WO 89/12060 discloses various building blocks for synthesizing oligonucleotide analogs, as well as oligonucleotide analogs formed by joining such building blocks in a defined sequence. The building blocks may be either “rigid” (i.e., containing a ring structure) or “flexible” (i.e., lacking a ring structure). In both cases, the building blocks contain a hydroxy group and a mercapto group, through which the building blocks are said to join to form oligonucleotide analogs. The linking moiety in the oligonucleotide analogs is selected from the group consisting of sulfide (—S—), sulfoxide (—SO—), and sulfone (—SO₂—). However, the application provides no data supporting the specific binding of an oligonucleotide analog to a target oligonucleotide.

International patent application WO 92/20702 describe an acyclic oligonucleotide which includes a peptide backbone on which any selected chemical nucleobases or analogs are stringed and serve as coding characters as they do in natural DNA or RNA. These new compounds, known as peptide nucleic acids (PNAs), are not only more stable in cells than their natural counterparts, but also bind natural DNA and RNA 50 to 100 times more tightly than the natural nucleic acids cling to each other (77). PNA oligomers can be synthesized from the four protected monomers containing thymine, cytosine, adenine and guanine by Merrifield solid-phase peptide synthesis. In order to increase solubility in water and to prevent aggregation, a lysine amide group is placed at the C-terminal.

Thus, antisense technology requires pairing of messenger RNA with an oligonucleotide to form a double helix that inhibits translation. The concept of antisense-mediated gene therapy was already introduced in 1978 for cancer therapy. This approach was based on certain genes that are crucial in cell division and growth of cancer cells. Synthetic fragments of genetic substance DNA can achieve this goal. Such molecules bind to the targeted gene molecules in RNA of tumor cells, thereby inhibiting the translation of the genes and resulting in dysfunctional growth of these cells. Other mechanisms has also been proposed. These strategies have been used, with some success in treatment of cancers, as well as other illnesses, including viral and other infectious diseases. Antisense oligonucleotides are typically synthesized in lengths of 13-30 nucleotides. The life span of oligonucleotide molecules in blood is rather short. Thus, they have to be chemically modified to prevent destruction by ubiquitous nucleases present in the body. Phosphorothioates are very widely used modification in antisense oligonucleotide ongoing clinical trials (57). A new generation of antisense molecules consist of hybrid antisense oligonucleotide with a central portion of synthetic DNA while four bases on each end have been modified with 2′O-methyl ribose to resemble RNA. In preclinical studies in laboratory animals, such compounds have demonstrated: greater stability to metabolism in body tissues and an improved safety profile when compared with the first-generation unmodified phosphorothioate (Hybridon Inc. news). Dosens of other nucleotide analogs have also been tested in antisense technology.

RNA oligonucleotides may also be used for antisense inhibition as they form a stable RNA-RNA duplex with the target, suggesting efficient inhibition. However, due to their low stability RNA oligonucleotides are typically expressed inside the cells using vectors designed for this purpose. This approach is favored when attempting to target a mRNA that encodes an abundant and long-lived-protein (57).

Recent scientific publications have validated the efficacy of antisense compounds in animal models of hepatitis, cancers, coronary artery restenosis and other diseases. The first antisense drug was recently approved by the FDA. This drug Fomivirsen, developed by Isis, is indicated for local treatment: of cytomegalovirus in patients with AIDS who are intolerant of or have a contraindication to other treatments for CMV retinitis or who were insufficiently responsive to previous treatments for CMV retinitis (Pharmacotherapy News Network).

Several antisense compounds are now in clinical trials in the United States. These include locally administered antivirals, systemic cancer therapeutics. Antisense therapeutics has the potential to treat many life-threatening diseases with a number of advantages over traditional drugs. Traditional drugs intervene after a disease-causing protein is formed. Antisense therapeutics, however, block mRNA transcription/translation and intervene before a protein is formed, and since antisense therapeutics target only one specific mRNA, they should be more effective with fewer side effects than current protein-inhibiting therapy.

A second option for disrupting gene expression at the level of transcription uses synthetic oligonucleotides capable of hybridizing with double stranded DNA. A triple helix is formed. Such oligonucleotides: may prevent binding of transcription factors to the gene's promoter and therefore inhibit transcription. Alternatively, they may prevent duplex unwinding and; therefore, transcription of genes within the triple helical structure.

Another approach is the use of specific nucleic acid sequences to act as decoys for transcription factors. Since transcription factors bind specific DNA sequences it is possible to synthesize oligonucleotides that will effectively compete with the native DNA sequences for available transcription factors in vivo. This approach requires the identification of gene specific transcription factor (57).

Indirect inhibition of gene expression was demonstrated for matrix metalloproteinase genes (MMP-1, -3, and -9), which are associated with invasive potential of human cancer cells. E1AF is a transcription activator of MMP genes. Expression of E1AF antisense RNA in HSC3AS cells showed decrease in mRNA and protein levels of MMP-1, -3, and -9. Moreover, 1HSC3AS showed lower invasive potential in vitro and in vivo. These results imply that transfection of antisense inhibits tumor invasion by down-regulating MMP genes (58).

Ribozymes:

Ribozymes are being increasingly used for the sequence-specific inhibition of gene expression by the cleavage of mRNAs encoding proteins of interest. The possibility of designing ribozymes to cleave any specific target RNA has rendered them valuable tools in both basic research and therapeutic applications. In the therapeutics area, ribozymes have been exploited to target viral RNAs in infectious diseases, dominant oncogenes in cancers and specific somatic mutations in genetic disorders. Most notably, several ribozyme gene therapy protocols for HIV patients are already in Phase 1 trials (62). More recently, ribozymes have been used for transgenic animal research, gene target validation and pathway elucidation. Several ribozymes are in various stages of clinical trials. ANGIOZYME was the first chemically synthesized ribozyme to be studied in human clinical trials. ANGIOZYME specifically inhibits formation of the VEGF-r (Vascular Endothelial Growth Factor receptor), a key component in the angiogenesis pathway. Ribozyme Pharmaceuticals, Inc., as well as other firms have demonstrated the importance of anti-angiogenesis therapeutics in animal models. HEPTAZYME, a ribozyme designed to selectively destroy. Hepatitis C Virus (HCV) RNA, was found effective in decreasing Hepatitis, C viral RNA in cell culture assays (Ribozyme Pharmaceuticals, Incorporated—WEB home page).

Gene Disruption in Animal Models:

The emergence of gene inactivation by homologous recombination methodology in embryonic stem cells has revolutionized the field of mouse genetics. The availability of a rapidly growing number of mouse null mutants has represented an invaluable source of knowledge on mammalian development, cellular biology and physiology, and has provided many models for human inherited diseases. Animal models are required for an effective drug delivery development program and evaluation of gene therapy approach. The improvement of the original knockout strategy, as well as exploitation of exogenous enzymatic systems that are active in the recombination process, has been considerably extended the range of, genetic manipulations that can be produced. Additional methods have been developed to provide versatile research tools: Double replacement method, sequential gene targeting, conditional cell type specific gene targeting, single copy integration method, inducible gene targeting, gene disruption by viral delivery, replacing one gene with another, the so called knock-in method: and the induction of specific balanced, chromosomal translocation. It is now possible to introduce a point mutation as a unique change in the entire genome, therefore allowing very fine dissection of gene function in vivo. Furthermore, the advent of methods allowing conditional gene targeting opens the way for analysis of consequence of a particular mutation in a defined organ and at a specific time during the life of the experimental animal (59).

DNA Vaccination:

Observations in the early 1990s that plasmid DNA could directly transfect animal cells in vivo sparked exploration of the use of DNA plasmids to induce immune response by direct injection into animal of DNA encoding antigenic protein. When a DNA vaccine plasmid enters the eukaryotic cell, the protein it encodes is transcribed and translated within the cell. In the case of pathogens, these proteins are presented to the immune system in their native form, mimicking the presentation of antigens during a natural infection. DNA vaccination is particularly useful for the induction of T cell activation. It was applied for viral and bacterial infectious diseases, as well as for allergy and for cancer. The central hypothesis behind active specific immunotherapy for cancer is that tumor cells express unique antigens that should stimulate the immune system. The first DNA vaccine against tumor was carcino-embrionic antigen (CEA). DNA vaccinated animals expressed immunoprotection and immunotherapy of human CEA-expressing syngeneic mouse colon and breast carcinoma (61). In a mouse: model of neuroblastoma, DNA immunization with HuD resulted in tumor growth inhibition with no neurological disease (60). Immunity to the brown locus protein, gp⁷⁵ tyrosinase-related protein-1, associated with melanoma, was investigated in a syngeneic mouse model. Priming with human gp75 DNA broke tolerance to mouse gp75. Immunity against mouse gp75 provided significant tumor protection (60).

Glycosyl Hydrolases:

Glycosyl hydrolases are a widespread group of enzymes that hydrolyze the o-glycosidic bond between two or more carbohydrates or between a carbohydrate and a noncarbohydrate moiety. The enzymatic hydrolysis of glycosidic bond occurs by using major one or two mechanisms leading to overall retention or inversion of the anomeric configuration. In both mechanisms catalysis involves two residues a proton donor and a nucleophile. Glycosyl hydrolyses have been classified into 58 families based on amino acid similarities. The glycosyl hydrolyses from families 1, 2, 5, 10, 17, 30, 35, 39 and 42 act on a large variety of substrates, however, they all hydrolyze the glycosidic bond in a general acid catalysis mechanism, with retention of the anomeric configuration. The mechanism involves two glutamic acid residues, which are the proton donors and the nucleophile, with an aspargine always preceding the proton donor. Analyses of a set of known 3D structures from this group revealed that their catalytic domains, despite the low level of sequence identity, adopt a similar (α/β) 8 fold with the proton donor and the nucleophile located at the C-terminal ends of strands β4 and β7, respectively. Mutations in the functional conserved amino acids of lysosomal glycosyl hydrolases were identified in lysosomal storage diseases.

Lysosomal glycosyl hydrolases including β-glucuronidase, β-manosidase, β-glucocerebrosidase, β-galactosidase and α-L iduronidase, are all exo-glycosyl hydrolases, belong to the GH-A clan and share a similar catalytic site. However, many endo-glucanases from various-organisms, such as bacterial and fungal xylenases and cellulases share this catalytic domain.

Genomic Sequence of hpa Gene; and its Implications:

It is well established that heparanase activity is correlated with cancer metastasis. This correlation was demonstrated at the level of enzymatic activity as well as the levels of protein and hpa cDNA expression in highly metastatic cancer cells as compared with non-metastatic cells. As such, inhibition of heparanase activity is desirable, and has been attempted by several means. The genomic region, encoding the hpa gene and the surrounding, provides a new powerful tool for regulation of heparanase activity at the level of gene expression. Regulatory sequences may reside in noncoding regions both upstream and downstream the transcribed region as well as in intron sequences. A DNA sequence upstream of the transcription start site contains the promoter region and potential regulatory elements. Regulatory factors, which interact with the promoter region may be identified and be used as potential drugs for inhibition of cancer, Metastasis and inflammation. The promoter region can be used to screen for inhibitors of heparanase gene, expression. Furthermore, the hpa promoter can be used to direct cell specific, particularly cancer cell specific, expression of foreign genes, such as cytotoxic or apoptotic genes, in order to specifically destroy cancer cells.

Cancer and yet unknown related genetic disorders may involve rearrangements and mutations in the heparanase gene, either in coding or non-coding regions. Such mutations may affect expression level or enzymatic activity. The genomic sequence of hpa enables the amplification of specific genomic DNA fragments, identification and diagnosis of mutations.

Possible Involvement of Heparanase in Wound Healing:

Repair of wounds is a chain of processes necessary for removal of damaged tissue or invaded pathogens from the body and for the recovery of the normal skin tissue. The healing process requires a sophisticated interaction between inflammatory cells, biochemical mediators including growth factors, extracellular matrix molecules, and microenvironment cell population. Inflammatory cells, keratinocytes and fibroblasts in the wound space and border produce and release a variety of growth factors such as platelet-derived growth factor (PDGF), epidermal growth factor (EGF), transforming growth factor (TGF) and fibroblast growth factor (FGF). These growth factors have biological activities which stimulate infiltration of inflammatory cells into the wound space and induce proliferation of keratinocytes and fibroblasts, leading to the formation of highly vascularized granulation tissue and extracellular matrix deposition. In deed, topical application of some growth factors (FGF, PDGF) accelerate healing of full-thickness wounds in normal mice and normalize a delayed healing response of diabetic mice [Tsuboi R. and D. B. Rifkin. 1991. Recombinant basic fibroblast growth factor stimulates wound healing-impaired db/db mice. J. Exp. Med. 172: 245-251; Brown R. E., M. P. Breeden and D. G. Greenhalgh. 1994. PDGF and TGF-alpha act synergistically to improve wound healing in the genetically diabetic mouse. J. Surg. Res. 56: 562-570].

Most skin lesions are healed rapidly and efficiently within a week or two. However, the end product is neither aesthetically nor functionally perfect. Moreover, under a number of pathological conditions wound healing is impaired. One such condition is the diabetic state, which result in a high degree of wound failure, often involved chronic complications including cutaneous infections, immunodisturbance and vascular and neuropathic dysfunction.

Repeated applications of bFGF accelerated closure of full-thickness excisional wounds in diabetic mice. Histological and: gross evaluation of wound tissues revealed enhanced angiogenesis in a dose-dependent manner: [Okumura M et al; Arzneimittelforschung 1996, 46(10):102-1-6]. The angiogenic effect of bFGF was also found to be effective for the treatment of ischemic heart disease and infracted myocardium. In acutely infracted myocardium, bFGF was found to increase the regional myocardial blood flow and salvage the myocardium (rabbit, dog, pig) [Hasegawa T et al; Angiology 1999 50(6):487-95; Scheinowitz M et al; Exp. Physiol. 1998, 83(5):585-93 Miyataka M et al; Angiology 1998, 49(5):381-90]. In addition, bFGF mediated new vessels formation and collateral growth: (human, pig, dog) [Watabane E et al; Basic Res. Cardiol. 1998, 93(1):30-7; Fleich M et al; Circulation 1999, 100(19):1945-50; Yang H T et: al; Am. J. Physiol. 1998, 274(6 Pt 2):H2053-61; Schumacher B et al; Circulation. 1998, 97(7):645-50; Arras M et al; J. Clin. Invest. 1998, 101(1):40-50]. bFGF plus heparin was the most effective method of enhancing angiogenesis (pig, dog)]Uchida Y et al; Ani. Heart J. 1995, 130(6):1182-8; Watabane E et al; Basic Res. Cardiol. 1998, 93(1):30-7].

As has already been mentioned above, by degrading HS, heparanase releases a repertoire of effectors such as growth factors from the BM. It may be speculated that the exact repertoire of effectors thus released to a very large extent depends on the specific BM being hydrolyzed.

Relevant Art:

U.S. patent application Nos. 08/922,170; 09/046,475; 09/071,739; 09/071,618; 09/109,386; 09/113,168; 09/140,888; 09/186,200; 09/260,037; 09/258,892; 09/260,038; 09/324,508; 09/322,977; 60/140,801; 09/435,739; 09/487,716; and PCT Application Nos. US98/17954; US99/06189; US99/09255; US99/09256; US99/15643; US99/25451; US00/03353; US00/03542 are incorporated herein by reference for the sake of providing information regarding the heparanase gene and protein, their alternatives, modifications, other GAG degrading genes and enzymes, their properties, their manufacture and their uses.

### SUMMARY OF THE INVENTION

The background art does not teach or suggest genomic, cDNA and composite polynucleotides encoding a polypeptide having heparanase activity, nor does the background art teach or suggest vectors including same. The background art also does not teach or suggest genetically modified cells expressing heparanase, nor does the background art teach or suggest a purified recombinant protein having heparanase activity free of contamination. The background art also does not teach or suggest antisense oligonucleotides, constructs and ribozymes which can be used for down regulation heparanase activity.

The present invention overcomes these disadvantages of the background art by providing genomic, cDNA and composite polynucleotides encoding a polypeptide having heparanase activity, vectors including same, genetically modified cells expressing heparanase and a purified recombinant protein having heparanase activity free of contamination, as well as antisense oligonucleotides, constructs and ribozymes which can be used for down regulation heparanase activity.

Cloning of the human hpa gene which encodes heparanase, and expression of recombinant heparanase by transfected host cells is reported herein, as well as downregulation of heparanase activity by antisense technology.

A purified preparation of heparanase isolated from human hepatoma cells was subjected to tryptic digestion. Peptides were separated by high pressure liquid chromatography and micro sequenced. The YGPDVGQPR (SEQ ID NO:8) sequence revealed was used to screen EST databases for homology to the corresponding back translated DNA sequence. Two closely related EST sequences were identified and were thereafter found to be identical. Both clones contained an insert of 1020 bp which included an open reading frame of 973 bp followed by a 27 bp of 3′ untranslated region and a Poly A tail. Translation start site was not identified.

Cloning of the missing 5′ end of hpa was performed by PCR amplification of DNA from placenta Marathon RACE cDNA composite using primers selected according to the EST clones sequence and the linkers of the composite. A 900 bp PCR fragment, partially overlapping with the identified 3′ encoding EST clones was obtained. The joined cDNA fragment (hpa), 1721 bp long (SEQ ID NO:9), contained an open reading frame which encodes a polypeptide of 543 amino acids (SEQ ID NO:10) with a calculated molecular weight of 61,192 daltons.

Cloning an extended 5′ sequence was enabled from the human SK-hep1 cell line by PCR amplification using the Marathon RACE. The 5′ extended sequence of the SK-hep1 hpa cDNA was assembled with the sequence of the hpa cDNA isolated from human placenta (SEQ ID NO:9). The assembled sequence contained an open reading frame, SEQ ID NOs: 13 and 15, which encodes, as shown in SEQ ID NOs:14 and 15, a polypeptide of 592 amino acids with a calculated molecular weight of 66,407 daltons.

The cloning procedures are described in length in U.S. Pat. No. 5,968,822; U.S. patent application Ser. Nos. 09/109,386, and 09/258,892; and PCT Application No. US98/17954, all of which are hereby incorporated by reference as if fully set forth herein.

The ability of the hpa gene product to catalyze degradation of heparan sulfate. (HS) in vitro was examined by expressing the entire open reading frame of hpa in High five and Sf21 insect cells, and the mammalian human 293 embryonic kidney cell line expression systems. Extracts of infected cells were assayed for heparanase catalytic activity. For this purpose, cell lysates were incubated with sulfate labeled, ECM-derived HSPG (peak I), followed by gel filtration analysis (Sepharose 6B) of the reaction mixture. While the substrate alone consisted of high molecular weight material, incubation of the HSPG substrate with lysates of cells infected with hpa containing virus resulted in a complete conversion of the high molecular weight substrate into low molecular weight labeled heparan sulfate degradation fragments (see, for example, U.S. patent application Ser. No. 09/260,038, hereby incorporated by reference as if fully set forth herein).

Extracts and conditioned media of cells infected with virus containing the hpa gene, demonstrated a high level of heparan sulfate degradation activity both towards soluble ECM-derived. HSPG and intact ECM. This degradation activity was inhibited by heparin, which is another substrate of heparanase. Cells infected with a similar construct containing no hpa gene had no such activity, nor did non-infected cells. The ability of heparanase expressed from the extended 5′ clone towards heparin was demonstrated in a mammalian expression system.

The expression pattern of hpa RNA in various tissues and cell lines was investigated using RT-PCR. It was found to be expressed only in tissues and cells previously known to have heparanase activity.

In subsequent experiments, the labeled HSPG substrate was incubated with the culture medium of infected High Five and Sf21 cells. Heparanase catalytic activity, reflected by the conversion of the high molecular weight HSPG substrate into low molecular weight HS degradation fragments, was found in the culture medium of cells infected with the pFhpa virus, but not the control pF1 virus.

Altogether, these results indicate that the heparanase enzyme is expressed in an active form by cells infected with Baculovirus or mammalian expression vectors containing the newly identified human hpa gene.

In other experiments, it was demonstrated that the heparanase enzyme expressed by cells infected with the pFhpa virus is capable of degrading HS complexed to other macromolecular constituents (e.g., fibronectin, laminin, collagen) present in a naturally produced intact ECM (09/260,038), in a manner similar to that reported for highly metastatic tumor cells or activated cells of the immune system [Vlodavsky, I., Eldor, A., Haimovitz-Friedman, A., Matzner, Y., Ishai-Michaeli, R., Levi, E., Bashkin, P., Lider, O., Naparstek, Y., Cohen, I. R., and Fuks, Z. (1992) Expression of heparanase by platelets and circulating cells of the immune system: Possible involvement in diapedesis and extravasation. Invasion & Metastasis, 12, 112-127; Vlodavsky, I., Mohsen, M., Lider, O., Ishai-Michaeli, R., Ekre, H.-P., Svahn, C. M., Vigoda, M., and Peretz, T. (1995). Inhibition of tumor metastasis by heparanase inhibiting species of heparin. Invasion & Metastasis, 14: 290-302].

As described above, the apparent molecular size of the recombinant enzyme produced in the baculovirus expression system was about 65 kDa. This heparanase polypeptide contains 6 potential N-glycosylation sites. Following deglycosylation by treatment with peptide N-glycosidase, the protein appeared as a 57 kDa band. This molecular weight corresponds to the deduced molecular mass (61,192 daltons) of the 543 amino acid polypeptide encoded by the full length hpa cDNA after cleavage of the predicted 3 kDa signal peptide. No further reduction in the apparent size of the N-deglycosylated protein was observed following concurrent O-glycosidase and neuramimidase treatment. Deglycosylation had no detectable effect on enzymatic activity.

Expression of the full length heparanase polypeptide in mammalian cells (e.g., 293 kidney cells, CHO) yielded a major protein of about 50 kDa and a minor of about 65 kDa in cell lysates. Comparison of the enzymatic activity of the two forms, revealed that the 50 kDa enzyme is at least 100-200 fold more active than the 65 kDa form. A similar difference was observed when the specific activity of the recombinant 65 kDa enzyme was compared to that of the 50 kDa heparanase preparations purified from human platelets, SK-hep-1 cells, or placenta. These results suggest that the 50 kDa protein is a mature processed form of a latent heparanase precursor. Amino terminal sequencing of the platelet heparanase indicated that cleavage occurs between amino acids Gln¹⁵⁷ and Lys¹⁵⁸. As indicated by the hydropathic plot of heparanase, this site is located within a hydrophillic peak, which is likely to be exposed and hence accessible to proteases.

According to Fairbank et al. (57) the precursor is cleaved at three sites to form a heterodimer of a 50 kDa polypeptide (the mature form) that is associated with a 8 kDa peptide.

In order to purify the recombinant heparanase enzyme, Sf21 insect cells were infected with pFhpa virus and the culture medium was applied onto a heparin-Sepharose column. Fractions were eluted with a salt gradient (0.35-2.0 M NaCl) and tested for heparanase catalytic activity and protein profile (SDS/PAGE followed by silver staining). Heparanase catalytic activity correlated with the appearance of a about 63 kDa protein band in fractions 19-24, consistent with the expected molecular weight of the hpa gene product. Active fractions eluted from heparin-Sepharose were pooled, concentrated and applied onto: a Superdex, 75 FPLC gel filtration column. Aliquots of each fraction were tested for heparanase catalytic activity and protein profile. A correlation was found between the appearance of a major protein (approximate molecular weight of 63 kDa) in fractions 4-7 and heparanase catalytic activity. This protein was not present in medium conditioned by control non-infected Sf21 cells subjected to the same purification protocol. Recently, an additional purification protocol was applied, using a single step chromatography with source-S ion exchange column.

Using this protocol P65 heparanase is purified from conditioned medium of CHO clones overexpressing and secreting recombinant human heparanase precursor, while the processed P50 heparanase is purified from cell extracts of similar CHO clones which overexpress and accumulate mature P50 heparanase. This purification resulted in a protein purified to a degree of 90%. Further details concerning heparanase production and purification procedures are disclosed in U.S. patent application Ser. No. 09/071,618, which is incorporated by reference as if fully set forth herein.

Recombinantly modified heparanases are also known. To this end, see U.S. patent application Ser. No. 09/260,038.

A panel of monochromosomal human/CHO and human/mouse somatic cell hybrids was used to localize the human heparanase gene to human chromosome 4. The newly isolated heparanase sequence can be used to identify a chromosome region harboring a human heparanase gene in a chromosome spread.

A human genomic library was screened and the human locus harboring the heparanase gene isolated, sequenced and characterized. Alternatively spliced heparanase mRNAs were identified and characterized. The human heparanase promoter has been isolated, identified and positively tested for activity. The mouse heparanase promoter has been isolated and identified as well. Antisense heparanase constructs were prepared and their influence on cells in vitro tested. A predicted heparanase active site was identified. And finally, the presence of sequences hybridizing with human heparanase sequences was demonstrated for a variety of mammalians and for an avian.

According to one aspect of the present invention there is provided an isolated nucleic acid comprising a genomic, complementary or composite polynucleotide sequence encoding a polypeptide having heparanase catalytic activity.

According to further features in preferred embodiments of the invention described below, the polynucleotide or a portion thereof is hybridizable with SEQ ID NOs: 9, 13, 42, 43 or a portion thereof at 68° C. in 6×SSC, 1% SDS, 5× Denharts, 10% dextran sulfate, 100 μg/ml salmon sperm DNA, and ³²P labeled probe and wash at 68° C. with 3×SSC and 0.1% SDS.

According to still further features in the described preferred embodiments the polynucleotide or a portion thereof is at least 60% identical with SEQ. ID NOs: 9, 13, 42, 43 or portions thereof as determined using the Bestfit procedure of the DNA sequence analysis software package developed by the Genetic Computer Group (GCG) at the university of Wisconsin (gap creation penalty −12, gap extension penalty −4).

According to still further features in the described preferred embodiments the polypeptide is as set forth in SEQ ID NOs:10, 14, 44 or portions thereof.

According to still further features in the described preferred embodiments the polypeptide is at least 60% homologous to SEQ ID NOs:10, 14, 44 or portions thereof as determined with the Smith-Waterman algorithm, using the Bioaccelerator platform developed by Compugene (gapop: 10.0, gapext: 0.5, matrix: blosum62).

According to, additional aspects of the present invention there are provided a nucleic acid construct (vector) comprising the isolated nucleic acid described herein and a host cell comprising the construct.

According to a further aspect of the present invention there is provided an antisense oligonucleotide a polynucleotide or a polynucleotide analog of at least 10 bases being hybridizable in vivo, under physiological conditions, with a portion of a polynucleotide strand encoding a polypeptide having heparanase catalytic activity.

According to an additional aspect of the present invention there is provided a method of in vivo downregulating heparanase-activity comprising the step of in vivo administering the antisense oligonucleotide herein described.

According to yet an additional aspect of the present invention there is provided a pharmaceutical composition comprising the antisense oligonucleotide herein described and a pharmaceutically acceptable carrier.

According to still an additional aspect of the present invention there is provided a ribozyme comprising the antisense oligonucleotide described herein and a ribozyme sequence.

According to a further aspect of the present invention there is provided an antisense nucleic acid construct comprising a promoter sequence and a polynucleotide sequence directing the synthesis of an antisense RNA sequence of at least 10 bases being hybridizable in vivo, under physiological conditions, with a portion of, a polynucleotide strand encoding a polypeptide having heparanase catalytic activity.

According to further features in preferred embodiments of the invention described below, the polynucleotide strand encoding the polypeptide having heparanase catalytic activity is as set forth in SEQ ID NOs: 9, 13, 42 or 43.

According to still further features in the described preferred embodiments the polypeptide having heparanase catalytic activity is as set forth in SEQ ID NOs: 10, 14 or 44.

According to still a further aspect of the present invention there is provided a method of in vivo downregulating heparanase activity comprising the step of in vivo administering the antisense nucleic acid construct herein described.

According to yet a further aspect of the present invention there is provided a pharmaceutical composition comprising the antisense nucleic acid construct herein described and a pharmaceutically acceptable carrier.

According to a further aspect of the present invention there is provided a nucleic acid construct comprising a polynucleotide sequence functioning as a promoter, the polynucleotide sequence is derived from SEQ ID NO:42 and includes at least nucleotides 2535-2635 thereof or from SEQ ID NO:43 and includes at least nucleotides 320-420.

According to a further aspect of the present invention there is provided a method of expressing a polynucleotide sequence comprising the step of ligating the polynucleotide sequence into the nucleic acid construct described above, downstream, of the polynucleotide sequence derived from SEQ ID NOs:42 or 43.

According to a further aspect of the present invention there is provided a recombinant protein comprising a polypeptide having heparanase catalytic activity.

According to further features in preferred embodiments of the invention described below, the polypeptide includes at least a portion of SEQ ID. NOs:10, 14 or 44.

According to still further features in the described preferred embodiments the protein is encoded by a polynucleotide hybridizable with SEQ ID NOs: 9, 13, 42, 43 or a portion thereof at 68° C. in 6×SSC, 1% SDS, 5× Denharts, 10% dextran sulfate, 100 μg/ml salmon sperm DNA, and ³²P labeled probe and wash at 68° C. with 3×SSC and 0.1% SDS.

According to still further features in the described preferred embodiments the protein is encoded by a polynucleotide at least 60% identical with SEQ ID NOs: 9, 13, 42, 43 or portions thereof as determined using the Bestfit procedure of the DNA sequence analysis software package developed by the Genetic Computer Group (GCG) at the University of Wisconsin (gap creation penalty −12, gap extension penalty −4).

According to a further aspect of the present invention there is provided a pharmaceutical composition comprising, as an active ingredient, the recombinant protein herein described.

According to a further aspect of the present invention there is provided a method of identifying a chromosome region harboring a heparanase gene in a chromosome spread comprising the steps of (a) hybridizing the chromosome, spread with a tagged polynucleotide probe encoding heparanase; (b) washing the chromosome spread thereby removing excess of non-hybridized probe, and (c) searching for signals associated with the hybridized tagged polynucleotide probe, wherein detected signals being indicative of a chromosome region harboring a heparanase gene.

According to a further aspect of the present invention there is provided a method of in vivo eliciting anti-heparanase antibodies comprising the steps of administering a nucleic acid construct including a polynucleotide segment corresponding to at least a portion of SEQ ID NOs:9, 13 or 43 and a promoter for directing the expression of said polynucleotide segment in vivo. Accordingly, there is provided also a DNA vaccine for in vivo eliciting anti-heparanase antibodies comprising a nucleic acid construct including a polynucleotide segment corresponding to at least a portion of SEQ ID NOs:9, 13 or 43 and a promoter for directing the expression of said polynucleotide segment in vivo.

The present invention can be used to develop new drugs to inhibit tumor cell metastasis, inflammation and autoimmunity. The identification of the hpa gene encoding for heparanase enzyme enables the production of a recombinant enzyme in heterologous expression systems. Additional features, advantages, uses and applications of the present invention in biological science and in diagnostic and therapeutic medicine are described hereinafter.

While reducing the present invention to practice, the ability of heparanase to induce angiogenesis and wound healing were put to test. As is further demonstrated below, the results were striking, rendering heparanase highly likely to become a medication for induction and/or acceleration of wound healing and/or angiogenesis. Cosmetic applications are also envisaged.

### BRIEF DESCRIPTION OF THE DRAWINGS

The invention herein described, by way of example only, with reference to the accompanying drawings, wherein:

FIG. 1 presents nucleotide sequence and deduced amino acid sequence of hpa cDNA. A single nucleotide difference at position 799 (A to T) between the EST (Expressed Sequence Tag) and the PCR amplified cDNA (reverse transcribed RNA) and the resulting amino acid substitution (Tyr to Phe) are indicated above and below the substituted unit, respectively. Cysteine residues and the poly adenylation consensus sequence are underlined. The asterisk denotes the stop codon TGA.

FIG. 2 demonstrates degradation of soluble sulfate labeled HSPG substrate by lysates of High Five cells infected with pFhpa2 virus. Lysates of High Five cells that were infected with pFhpa2 virus (●) or control pF2 virus, (□) Were incubated (18 h, 37° C.) with sulfate labeled ECM-derived soluble HSPG (peak I). The incubation medium was then subjected to gel filtration on Sepharose 6B. Low molecular weight HS degradation fragments (peak II) were produced only during incubation with the pFhpa2 infected cells, but there was no degradation of the HSPG substrate (⋄) by lysates of pF2 infected cells.

FIGS. 3a-b demonstrate degradation of soluble sulfate labeled HSPG substrate by the culture medium of pFhpa2 and pFhpa4 infected cells. Culture media of High Five cells infected with pFhpa2 (3a) or pFhpa4 (3b) viruses (●), or with control viruses (□) were incubated (18 h, 37° C.) with sulfate labeled ECM-derived soluble HSPG (Peak I, ⋄). The incubation media were then subjected to gel filtration on Sepharose 6B. Low molecular weight HS degradation fragments (peak II) were produced only during incubation with the hpa gene containing viruses. There was no degradation of the HSPG substrate by the culture medium of cells infected with control viruses.

FIG. 4 presents size fractionation of heparanase activity expressed by pFhpa2 infected cells. Culture medium of pFhpa2 infected High Five &ells was applied onto a 50 kDa cut-off membrane. Heparanase activity (conversion of the peak I substrate, (⋄) into peak II HS degradation fragments) was found in the high (>50 kDa) (●), but not low (<50 kDa) (∘) molecular weight compartment.

FIGS. 5a-b demonstrate the effect of heparin on heparanase activity expressed by pFhpa2 and pFhpa4 infected High Five cells. Culture media of pFhpa2 (5a) and pFhpa4 (5b) infected High Five cells were incubated (18 h, 37° C.) with sulfate labeled ECM-derived soluble HSPG (peak I, ⋄) in the absence (●) or presence (Δ) of 10 μg/ml heparin. Production of low molecular weight HS degradation fragments was completely abolished in the presence of heparin, a potent inhibitor of heparanase activity (6, 7).

FIGS. 6a-b demonstrate degradation of sulfate labeled intact ECM by virus infected High Five and Sf21 cells. High Five (6a) and Sf21 (6b) cells were plated on sulfate labeled ECM and infected (48 h, 28° C.) with pFhpa4 (●) or control pF1 (□) viruses. Control non-infected Sf2.1 cells (R) were plated on the labeled ECM as well. The pH of the cultured medium was adjusted to 6.0-6.2 followed by 24 h incubation at 37° C. Sulfate labeled material released into the incubation medium was analyzed by gel filtration on Sepharose 6B. HS degradation fragments were produced only by cells infected with the hpa containing virus.

FIG. 7a-b demonstrate degradation of §sulfate labeled intact ECM by virus infected cells. High Five (7a) and Sf21 (7b) cells were plated on sulfate labeled ECM and infected (48 h, 28° C.) with pFhpa4 (●) or control pF1 (□) viruses. Control non-infected Sf21 cells (R) were plate on labeled ECM as well. The pH of the cultured medium was adjusted to 6.0-6.2, followed by 48 h incubation at 28° C. Sulfate labeled degradation fragments released into the incubation medium was analyzed by gel filtration on. Sepharose 6B. HS degradation fragments were produced only by cells infected with the hpa containing virus.

FIGS. 8a-b demonstrate degradation of sulfate labeled intact ECM by the culture medium of pFhpa4 infected cells. Culture media of High Five (8a) and Sf21 (8b) cells that were infected with pFhpa4 (●) or control pF1 (□) viruses were incubated (48 h, 37° C., pH 6.0) with intact sulfate labeled ECM. The ECM was also incubated with the culture medium of control non-infected Sf21 cells (R). Sulfate labeled material released into the reaction mixture was subjected to gel filtration analysis. Heparanase activity was detected only in the culture medium of pFhpa4 infected cells.

FIGS. 9a-b demonstrate the effect of heparin on heparanase activity in the culture medium of pFhpa4 infected cells. Sulfate labeled ECM was incubated (24 h, 37° C., pH 6.0) with culture medium of pFhpa4 infected High Five (9a) and Sf21 (9b) cells in the absence (●) or presence (V) of 10 μg/mim heparin. Sulfate labeled material released into the incubation medium was subjected to gel filtration on-Sepharose 6B. Heparanase activity (production of peak II HS degradation fragments) was completely inhibited in the presence of heparin.

FIGS. 10a-b demonstrate purification of recombinant heparanase on heparin-Sepharose. Culture medium of Sf21 cells infected with pFhpa4 virus was subjected to heparin-Sepharose chromatography. Elution of fractions was performed with 0.35-2 M NaCl gradient (⋄). Heparanase activity in the eluted fractions is demonstrated in FIG. 10a (●). Fractions 15-28 were subjected to 15% SDS-polyacrylamide gel electrophoresis followed by silver nitrate staining. A correlation is demonstrated between a major protein band (MW ˜63,000) in fractions 19-24 and heparanase activity.

FIGS. 11a-b demonstrate purification of recombinant heparanase on a Superdex 75 gel filtration column. Active fractions eluted from heparin-Sepharose (FIG. 10a) were pooled, concentrated and applied onto Superdex 75 FPLC column. Fractions were collected and aliquots of each fraction were tested for heparanase activity (C, FIG. 11a) and analyzed by SDS-polyacrylamide gel electrophoresis followed by silver nitrate staining (FIG. 11b). A correlation is seen between the appearance of a major protein band (MW ˜63,000) in fractions 4-7 and heparanase activity.

FIGS. 12a-e demonstrate expression of the hpa gene by RT-PCR with total RNA from human embryonal tissues (12a), human extra-embryonal tissues (12b) and cell lines from different origins (12c-e). RT-PCR products using hpa specific primers (I), primers for GAPDH housekeeping gene (II), and control reactions without reverse transcriptase demonstrating absence of genomic DNA or other contamination in RNA samples (III). M-DNA molecular weight marker VI (Boehringer Mannheim). For 12a: lane 1—neutrophil cells (adult), lane 2—muscle, lane 3—thymus, lane 4—heart, lane 5—adrenal. For 12b: lane 1—kidney, lane 2—placenta. (8 weeks), lane 3—placenta (11 weeks), lanes 4-7—mole (complete hydatidiform mole), lane 8—cytotrophoblast cells (freshly isolated), lane 9—cytotrophoblast cells (1.5 h in vitro), lane 10—cytotrophoblast cells (6 h in vitro), lane 11—cytotrophoblast cells (18 h in vitro), lane 12—cytotrophoblast cells (48 h in vitro). For 12c. lane 1—JAR bladder cell line, lane 2—NCITT testicular tumor cell line, lane 3—SW-480 human hepatoma cell line, lane 4—HTR (cytotrophoblasts transformed by SV40), lane 5—HPTLP-I hepatocellular carcinoma cell line, lane 6—EJ-28 bladder carcinoma cell line. For 12d: lane 1—SK-hep-1 human hepatoma cell line, lane 2—DAMI human megakaryocytic cell line, lane 3—DAMI cell line+PMA, lane 4—CHRF cell line+PMA, lane 5—CHRF cell line. For 12e: lane 1—ABAE bovine aortic endothelial cells, lane 2-1063 human ovarian cell line, lane 0.3—human breast carcinoma MDA435 cell line, lane 4—human breast carcinoma MDA231 cell line.

FIG. 13 presents a comparison between nucleotide sequences of the human hpa and a mouse EST cDNA fragment (SEQ ID NO:12) which is 80% homologous to the 3′ end (starting at nucleotide 1066′ of SEQ ID NO:9) of the human hpa the aligned termination codons are underlined.

FIG. 14 demonstrates the chromosomal localization of the hpa gene. PCR products of DNA derived from somatic cell hybrids and of genomic DNA of hamster, mouse and human of were separated on: 0.7% agarose gel following amplification with hpa specific primers. Lane 1—Lambda DNA digested with BstEII, lane 2—no DNA control, lanes 3-29, PCR amplification products. Lanes 3-5—human, mouse and hamster genomic DNA, respectively. Lanes 6-29, human monochromosomal somatic cell hybrids representing chromosomes 1-22 and X and Y, respectively. Lane 30—Lambda DNA digested with BstEII. An amplification product of approximately 2.8 Kb is observed only in lanes 5 and 9, representing human genomic DNA and DNA derived from cell hybrid carrying human chromosome 4, respectively. These results demonstrate that the hpa gene is localized in human chromosome 4.

FIG. 15 demonstrates the genomic exon-intron structure of the human hpa locus (top) and the relative positions of the lambda clones used as sequencing templates to sequence the locus (below). The vertical rectangles represent exons (E) and the horizontal lines therebetween represent introns (I), upstream (U) and downstream (D) regions. Continuous lines represent DNA fragments, which were used for sequence analysis. The discontinuous line in lambda 6 represent a region, which overlaps with lambda 8 and hence was not analyzed. The plasmid contains a PCR product, which bridges the gap between L3 and L6.

FIG. 16 presents the nucleotide sequence of the genomic region of the hpa gene. Exon sequences appear in upper case and intron sequences in lower case. The deduced amino acid, sequence of the exons is printed below the nucleotide sequence. Two predicted transcription start sites are shown in, bold.

FIG. 17 presents an alignment of the amino acid sequences of human heparanase, mouse and partial sequences of rat homologues. The human and the mouse sequences were determined by sequence analysis of the isolated cDNAs. The rat sequence is derived from two different EST clones, which represent two different regions (5′ and 3′) of the rat hpa cDNA. The human sequence and the amino acids in the mouse and rat homologues, which are identical to the human sequence, appear in bold.

FIG. 18 presents a heparanase Zoo blot. Ten ricrograms of genomic DNA from various sources were digested with EcoRI and separated on 0.77% agarose—TBE gel. Following electrophoresis; the was gel treated with HCl and than with NaOH and the DNA fragments were downward transferred to a nylon membrane (Hybond N+, Amersham) with 0.4 N NaOH. The membrane was hybridized with a 1.6 Kb DNA probe that contained the entire hpa cDNA. Lane order: H—Human; M—Mouse; Rt—Rat; P—Pig; Cw—Cow; Hr—Horse; S—Sheep; Rb—Rabbit; D—Dog; Ch—Chicken; F—Fish. Size markers (Lambda BsteII) are shown on the left

FIG. 19 demonstrates the secondary structure prediction for heparanase performed using the PHD server—Profile network Prediction Heidelberg. H—helix, E—extended (beta strand), The glutamic acid predicted as the proton donor is marked by asterisk and the possible nucleophiles are underlined.

FIGS. 20a-b demonstrate the expression of heparanase by human endothelium. 20a—RT-PCR. Total RNA isolated from ECGF-stimulated proliferating human umbilical vein (HUVEC, lane 1) and bone marrow (TrHBMEC, lane 2) derived EC was analyzed by RT-PCR for expression of the heparanase mRNA, using human specific hpa primers amplifying a 564 bp cDNA [Vlodavsky, I. et al. Mammalian heparanase: gene cloning, expression and function, in tumor progression and metastasis. Nat Med 5, 793-802 (1999)] fragment. Lane 3, DNA molecular weight markers. 20b—Immunohistochemistry. Immunostaining of tissue specimens was performed as described in the Examples section that follows. Positive staining is reddish-brown. Preferential staining of the heparanase protein is seen in the endothelium of capillaries and small sprouting vessels (arrows, left & right panels) as compared to little or no staining of endothelial cells in mature quiescent blood vessels (concave arrows, left & middle panels). Enhanced expression of the heparanase protein is seen in the neoplastic colonic epithelium. Original magnification is 200× (left and right panels) and 100× (middle panel).

FIGS. 21a-c demonstrate release of ECM-bound bFGF by recombinant heparanase, and bFGF accessory activity of HS degradation fragments released from EC-vs. ECM. 21a-b—Release of ECM-bound bFGF. 21a—ECM-coated wells of four-well plates were incubated (3-hours, 24° C.) with ¹²⁵I-bFGF as described in the Examples section that follows. The ECM was washed 3, times and incubated (3 hours, 37° C.) with increasing concentrations; of recombinant heparanase. Released radioactivity is expressed as percent of the total ECM-bound ¹²⁵I-bFGF. About 10;% of the ECM-bound ¹²⁵I-bFGF was released in the absence of added heparanase. Each data point is the mean±SD of triplicate wells. Where error-bars cannot be seen, SD is smaller than the symbol. 21a (inset)—Release of sulfate labeled HS degradation fragments. Metabolically sulfate labeled ECM was incubated (3 hours, 37° C., pH 6.0) with 0.2 μg/ml recombinant heparanase. Sulfate labeled material released into the incubation medium was analyzed by gel filtration on Sepharose 6B. Labeled fragments eluted in fractions 15-35 (peak II) were 5-6 fold-smaller than intact HS side chains and were susceptible to deamination by nitrous acid [Vlodavsky, I. et al. Mammalian heparanase: gene cloning, expression and function in tumor progression and metastasis. Nat Med 5, 793-802 (1999)]. 21b—Release of endogenous ECM-resident bFGF by heparanase. Recombinant heparanase (0.5 g/ml) was incubated (4 hours, 37° C.) with ECM coated 35-mm dishes in 1 ml heparanase reaction mixture. Aliquots of the incubation media were taken for quantitation of bFGF by ELISA as described in the Examples section that follows. Each data point is the mean±S.D. of triplicate determinations. 21c—Stimulation of bFGF induced DNA synthesis in BaF3 lymphoid cells by HS degradation fragments. Confluent bovine aortic EC cultured in 35-mm plates and their underlying ECM [as described in Gospodarowicz D. Moran J Braun D and Birdwell C 1976 Clonal growth of bovine vascular endothelial cells fibroblast growth factor as a survival agent. Proc. Natl. Acad. Sci. 73: 4120-4124] were incubated (4 hours, 37° C., pH 6.5) with 0.1 μg/ml recombinant heparanase. Aliquots (5-200 μl) of the incubation media were then added to BaF3 cells seeded into 96 well plates in the presence of 5 ng/ml bFGF. ³H-thymidine (1 μCᵢ/well) was added 48 hours after seeding and 6‘hours ’ later the cells were harvested and measured for ³H-thymidine incorporation. Each data point represents the mean±S.D. of six culture wells. 21c (Inset)—Release of sulfate labeled material from EC (open circles) vs. ECM (closed circles). In control plates, both the EC and ECM were first metabolically labeled with Na₂[³⁵S]O₄. Sulfate labeled material released by heparanase (0.2 μg/ml, 4 hours, 37° C.) from EC and ECM was subjected to gel filtration.

FIGS. 22a-c demonstrate angiogenic response induced by Matrigel embedded with hpa vs. mock transfected Eb lymphoma cells. BALB/c mice (n=5) were injected subcutaneously with 0.4 ml cold Matrigel premixed with 2×10⁶ hpa- or mock-transfected Eb lymphoma cells. After 7 days, the mice were sacrified, and the Matrigel plugs were removed and photographed. Angiogenic response was then quantitated by measurement of the hemoglobin content as described in the Examples section that follows. 22a—Representative Matrigel plugs containing hpa transfected (left) and mock transfected (right) Eb cells photographed in situ, prior to their removal out of their subcutaneous location in the mice. 22b—Matrigel plugs containing heparanase producing (bottom) vs. control mock transfected (top) Eb cells. Shown are isolated Matrigel plugs removed from 10 different mice. 22c—Hemoglobin content of Matrigel plugs (shown in FIG. 22b) containing hpa transfected (dark bar) vs. control mock transfected (empty bar) Eb cells. Represented is the mean±SD (n#5, p=0.0089; unpaired t test).

FIGS. 23a-b demonstrate that topical administration of: active heparanase accelerate wound healing. 23a—Full-thickness wounds were created with a circular 8 mm punch at the back of the mouse skin. Wound areas were calculated after 7 days in control (1) or active heparanase-treated (2) mice and are shown as total area (23a) and percent (23b). Note the enhancement of wound healing upon exogenous application of heparanase. Data are statistically significant (P values equals 0.0023).

FIGS. 24a-d demonstrate an increase in granulation tissue cellularity upon heparanase treatment. Full-thickness wounds were created as described for FIGS. 24a-b. Wounds were left untreated (24a-b) or treated with heparanase for 7 days (24c-d). Wounds, including the underlying granulation tissue were formalin-fixed, paraffin-embedded and 5 micron sections were stained with hematoxilin-eosin. Note the increase in the granulation tissue cellularity upon heparanase treatment. Original magnifications: 24a and 24c×170; 24b and 24d×340.

FIGS. 25a-f demonstrate that heparanase treatment induces cellular proliferation and granulation tissue vascularization. Five micron sections from non-treated (25a, c and d) and heparanase-treated (25b, e and f) granulation tissues were stained for PCNA (25a-b and 25d-e) and for PECAM-1 (25c, f). Note the increase in PCNA-positive cells and PECAM-1 positive blood vessels structures upon heparanase treatment. Original magnifications: 25a-cX 170, 25d-fX 340.

FIGS. 26a-f demonstrates, that heparanase expression is restricted to differentiated keratinocytes in mouse skin tissue. Five micron skin tissue sections were stained for PCNA (26a, d) and heparanase (26b-c, e). Negative control (no primary antibody) is shown in 26f. Note intense PCNA staining at the basal epidermal cell layer (26a, d) while heparanase mainly stain the outer most, keratinocytes, cell layer (26b, e) and the cells composing the hair follicle (26c). In the latter case, nuclear staining is observed.

FIGS. 27a-d demonstrate expression of heparanase in human skin. 27a—cultures of HaCat keratinocytes cell line immunostained with anti-heparanase monoclonal antibody (HP-92). 27b—heparanase activity in intact cells and in extracts of HaCat cells, in an ECM-assay. 27c and d immuno-staining of normal skin tissue with HP-92.

FIG. 28 demonstrates stimulation of angiogenesis by heparanase in rat eye model. The central cornea of rats' eyes was scraped with a surgical knife. The right eye of each rat was then treated with heparanase, 50 μl drop (1 mg/ml) of purified recombinant human P50 heparanase, three times a day. The left eye served as a control and was treated with Lyeteers. Vascularization and epithelialization were evaluated following closure of the corneal lesion. Heparanase treated eyes exhibited vascularization of the cornea, as well as increased vascularization in the iris. Normal, minor vascularization of the iris and non vascular appearance of the cornea were observed in the controls

FIG. 29 demonstrates cornea sections of heparanase treated eye as compared to control, Lyeteers treated eyes. Control eyes demonstrate healing of the epithelia which is accompanied by a normal organized structure of the cornea. Heparanase treatment resulted in growth of blood vessels into the cornea (arrows), followed by a massive infiltration of lymphocytes. Vascularization associated inflammatory reaction interfered with corneal healing, as demonstrated by a disorganized structure of the cornea.

FIGS. 30a-e demonstrate that skin tissue morphology is impaired under diabetic conditions. Skin sections from normal (30a, 30d) and streptozotocin-induced diabetic (b, e) rats were hematoxilin-eosin stained (30a, 30b) or immunostained with anti-heparanase antibodies (30d, 30e). Measurements from 10 control or diabetic different rats are shown in (30c). Note a dramatic decrease in the skin tissue thickness and reduced heparanase expression under diabetic conditions.

FIGS. 31a-f demonstrate heparanase expression in the wound granulation tissue. Full-thickness wounds were generated by 8 mm punch at the back of rat skin. Seven days later the wounds, including the newly formed granulation tissue, were harvested, formalin-fixed and paraffin-embedded. Five microns sections-were stained for heparanase (31a-c), or double stained for heparanase (red) and SMA (green)(31d-f). Note heparanase expression in the granulation tissue (31a) and at the lumen-facing areas of endothelial cells lining blood vessels (31e, 31f). Original magnifications: a ×4, b ×10, c-f×40.

FIG. 32 demonstrates that heparanase accelerates wound healing in streptozotocin-induced rat diabetic. Four 8 mm full-thickness punches were created at the back of normal, non-diabetic (Nor), or diabetic rats. Wounds were treated with saline (Nor, Con), heparanase (Hep, 1; μg/wound) or PDGF (0.5 μg/wound) immediately following wounding, four hours later, and three additional times during the following day, at 4 hours intervals. Seven days after wounding, wounds were harvested, fixed and wound closure was evaluated under low power magnification of hematoxilin-eosin stained sections. Three animals were included min each group to yield 12 wounds for each treatment. Note improved wound healing upon heparanase treatment, similar to PDGF effect.

FIGS. 33a-b demonstrate that heparanase accelerates wound healing under ischemic conditions. FIG. 33a is a schematic representation of the flap/punch ischemic wound model. Two longitudinal incisions, each 6 cm in length, were connected at the caudal end with a third, 3 cm, incision across the midline. The flap was elevated to the base of the carnial pedicle, replaced in its: bed and secured with sutures. Two 8 mm punches were generated in the flap 3 cm from the carnial end. FIG. 33b—Wounds were treated with saline (Con), active heparanase (p45, 1 μg/wound), the heparanase precursor (p60, 5 μg/wound) and PDGF (0.5 μg/wound) immediately after wounding, 4 hours later and three more times, 4 hours apart, the next day (a total of 5 application, each at a volume of 50 μl). Longitude incisions were treated once just prior to clipping. Wounds closure was evaluated 10 days following wounding by histological examination. P45 as well: as, p60 heparanases significantly improved wound closure (p values are 0.03 and 0.016 for p45 and p60, respectively). Five rats were included in each group, and two wounds were created at each flap to yield a total of 10 wounds.

FIG. 34 demonstrates that heparanase induces reepithalialization of incisional wounds. Typical histological examination of control (left) and heparanase (p45)—treated incisional wounds from the flap described in FIG. 33a-b is shown Measurements of 10 incisions from control and V heparanase treated incisions are shown graphically. Note a robust increase in the epithelial layer thickness upon heparanase treatment.

FIG. 35 demonstrates % that heparanase treatment induces the recruitment of pericytes into blood vessels. Untreated (Con) and heparanase-treated (Hep) wound sections from the ischemic model were immunostained with anti-SMA antibodies. Representative photomicrographs are shown on the left and graphical evaluation of 10 different wounds, and at least 3 different fields in each wound, is shown on the right. Note the dramatic recruitment of SMA-positive pericytes into blood vessels upon heparanase treatment.

### DESCRIPTION OF THE PREFERRED EMBODIMENTS

The present invention is of a polynucleotide or nucleic acid, referred to hereinbelow interchangeably as hpa, hpa cDNA or hpa gene or identified by its SEQ ID NOs, encoding a polypeptide having heparanase activity, vectors or nucleic acid constructs including same and which are used for over-expression or antisense inhibition of heparanase, genetically modified cells expressing same, recombinant protein having heparanase activity, antisense oligonucleotides and ribozymes for heparanase modulation, and heparanase promoter sequences which can be used to direct the expression of desired genes. Specifically, the present invention describes methods and compositions comprising the recombinant heparanase which can be used for inducing and/or accelerating wound healing and/or angiogenesis, as well as for cosmetic treatment of hair and skin.

Before explaining at least one embodiment of the invention in detail, it is to be understood that the invention is not limited in its application to the details of construction and the arrangement of the components set forth in the following description or illustrated in the drawings. The invention is capable of other embodiments or of being practiced or carried out in various ways. Also, it is to be understood that the phraseology and terminology employed herein is for the purpose of description and should not be regarded as limiting.

Cloning of the human and mouse hpa genes, cDNAs and genomic sequence (for human), encoding heparanase and expressing recombinant heparanase by transfected cells is reported herein. These are the first mammalian heparanase genes to be cloned.

A purified preparation of heparanase isolated from human hepatoma cells was subjected to tryptic digestion and microsequencing.

The YGPDVGQPR (SEQ ID NO:8) sequence revealed was used to screen EST databases for homology to the corresponding back translated DNA sequences.

Two closely related EST sequences were identified and were thereafter found to be identical.

Both clones contained an insert of 1020 bp: which includes an open reading frame of 973 bp followed by a 3′ untranslated region of 27 bp and a Poly A tail, whereas a translation start site was not identified.

Cloning of the missing 5′ end was performed by PCR amplification of DNA from placenta Marathon RACE cDNA composite using primers, selected according to the EST clones sequence and the linkers of the composite.

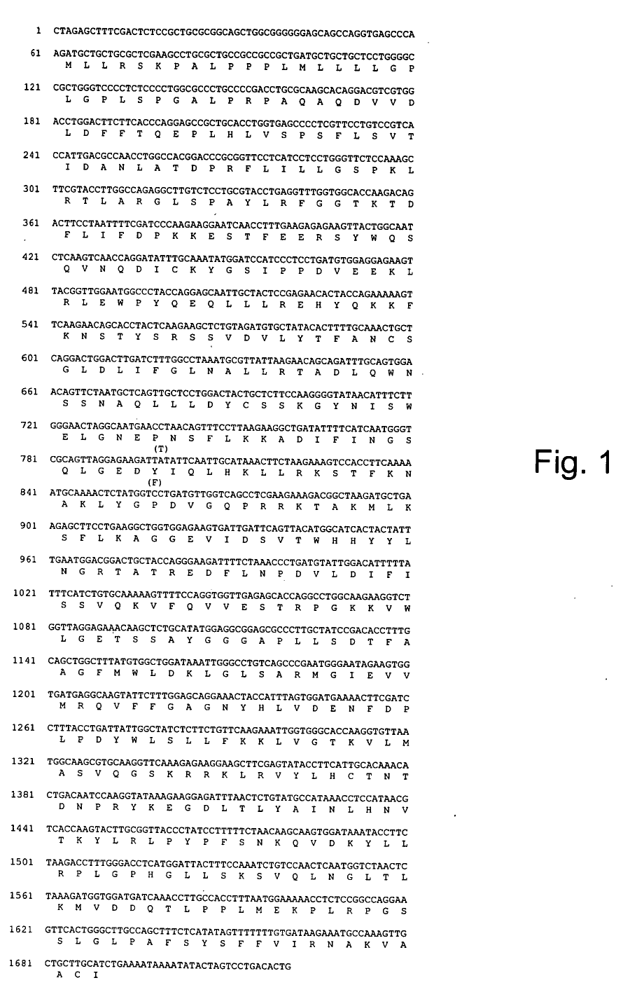

A 900 bp PCR: fragment, partially overlapping with the identified. 3′ encoding EST clones was obtained. The joined cDNA fragment (hpa), 1721 bp long (SEQ ID NO:9), contained an open reading frame which encodes, as shown in FIG. 1 and SEQ ID NO:11, a polypeptide of 543 amino acids (SEQ ID NO:10) with a calculated molecular weight of 61,192 daltons.

A single nucleotide difference at position 799 (A to T) between the EST clones and the PCR amplified cDNA was observed. This difference results in a single amino acid substitution (Tyr to Phe) (FIG. 1). Furthermore, the published EST sequences contained an unidentified nucleotide, which following DNA sequencing of both the EST clones was resolved into two nucleotides (G and C at positions 1630 and 1631 in SEQ ID NO:9, respectively).

The ability of the hpa gene product to catalyze degradation of heparan sulfate in an in vitro assay was examined by expressing the entire open reading frame in insect cells, using the Baculovirus expression system.

Extracts and conditioned media of cells infected with virus containing the hpa gene, demonstrated a high level of heparan sulfate degradation activity both towards soluble ECM-derived HSPG and intact ECM, which was inhibited by heparin, while cells infected with a similar construct containing no, hpa gene had no such activity, nor did non-infected cells.

The expression pattern of hpa RNA in various tissues and cell lines was investigated using RT-PCR. It was found to be expressed only in tissues and cells previously known to have heparanase activity.

Cloning an extended 5′ sequence was enabled from the human SK-hep1 cell line by PCR amplification using the Marathon RACE. The 5′ extended sequence of the SK-hep1 hpa cDNA was assembled with the sequence of the hpa cDNA isolated from human placenta (SEQ ID NO:9). The assembled sequence contained an open reading frame, SEQ ID NOs: 13 and 15, which encodes, as % shown in SEQ ID NOs:14 and 15, a polypeptide of 592 amino acids, with a calculated molecular weight of 66,407 daltons. This open reading frame was shown to direct the expression of catalytically active heparanase in a mammalian cell expression system. The expressed heparanase was detectable by anti heparanase antibodies in Western blot analysis.

A panel of monochromosomal human/CHO and human/mouse somatic cell hybrids was used to localize the human heparanase gene to human chromosome 4. The newly isolated heparanase sequence can therefore be used to identify a chromosome region harboring a human heparanase gene in a chromosome spread.

The hpa cDNA was then used as a probe to screen a a human genomic library. Several phages were positive. These phages were analyzed and were found to cover most of the hpa locus, except for a small portion which was recovered by bridging PCR. The hpa locus covers about 50,000 bp. The hpa gene includes 12 exons separated by 11 introns.

RT-PCR performed on a variety of cells revealed alternatively spliced hpa transcripts.

The amino acid sequence of human heparanase was used to search for homologous sequences in the DNA and protein databases. Several human EST's were identified, as well as mouse sequences highly homologous to human heparanase. The following mouse EST's' were identified AA177901, AA674378, AA67997, AA047943, AA690179, AI22034, all sharing an identical sequence and correspond to amino acids 336-543 of the human heparanase sequence. The entire mouse heparanase cDNA was cloned, based on the nucleotide sequence of the mouse EST's using Marathon cDNA libraries. The mouse and the human hpa genes share an average homology of 78% between the nucleotide sequences and 81% similarity between the deduced amino acid sequences hpa homologous sequences from rat were also uncovered (EST's AM060284 and AI237828).

Homology search of heparanase-amino acid sequence against the DNA and the protein databases and prediction of its protein secondary structure enabled to identify candidate amino acids that participate in the heparanase active site.

Expression of hpa antisense in mammalian cell lines resulted in about five fold decrease in the number of recoverable cells as compared to controls.

Human Hpa cDNA was shown to hybridize with genomic DNAs of a variety of mammalian species and with an avian.

The human and mouse hpa promoters were identified and the human promoter was tested positive in directing the expression of a reporter gene.

Thus, according to the present invention there is provided an isolated nucleic acid comprising a genomic, complementary or composite polynucleotide sequence encoding a polypeptide having heparanase catalytic activity.

The phrase “composite polynucleotide sequence” refers to a sequence which includes exonal sequences required to encode the polypeptide having heparanase activity, as well as any number of intronal sequences. The intronal sequences can be of any source and typically will include conserved splicing signal sequences. Such intronal sequences may further include cis acting expression regulatory elements.

The term “heparanase catalytic activity” or its equivalent term “heparanase activity” both refer to a mammalian endoglycosidase hydrolyzing activity which is specific for heparan or heparan sulfate proteoglycan substrates, as opposed to the activity of bacterial enzymes (heparinase I, II and III) which degrade heparin or heparan sulfate by means of β-elimination (37).

According to a preferred embodiment of the present invention the polynucleotide or a portion thereof is hybridizable with SEQ ID NOs: 9, 13, 42, 43 or a portion thereof at 68° C. in 6×SSC, 1% SDS, 5× Denharts, 10% dextran sulfate, 100 μg/ml salmon sperm DNA, and ³²P labeled probe and wash at 68° C. with 3, 2, 1, 0.5 or 0.1×SSC and 0.1% SDS.

According to another preferred embodiment of the present invention the polynucleotide or a portion thereof is at least 60%, preferably at least 65%, more preferably at least 70%, more preferably at least 75%, more preferably at least 80%, more preferably at least 85%, more preferably at least 90%, most preferably, 95-100% identical with SEQ ID NOs: 9, 13, 42, 43 or portions thereof as determined using the Bestfit procedure of the DNA sequence analysis software package developed by the Genetic Computer Group (GCG) at the university of Wisconsin (gap creation penalty −12, gap extension penalty −4—which are the default parameters).

According to another preferred embodiment of the present invention, the polypeptide encoded by the polynucleotide sequence is as set forth in SEQ ID NOs:10, 14, 44 or portions thereof having heparanase catalytic activity. Such portions are expected to include amino acids Asp-Glu 224-225 (SEQ ID NO:10), which can serve as proton donors and glutamic acid 343 or 396 which can serve as a nucleophile.

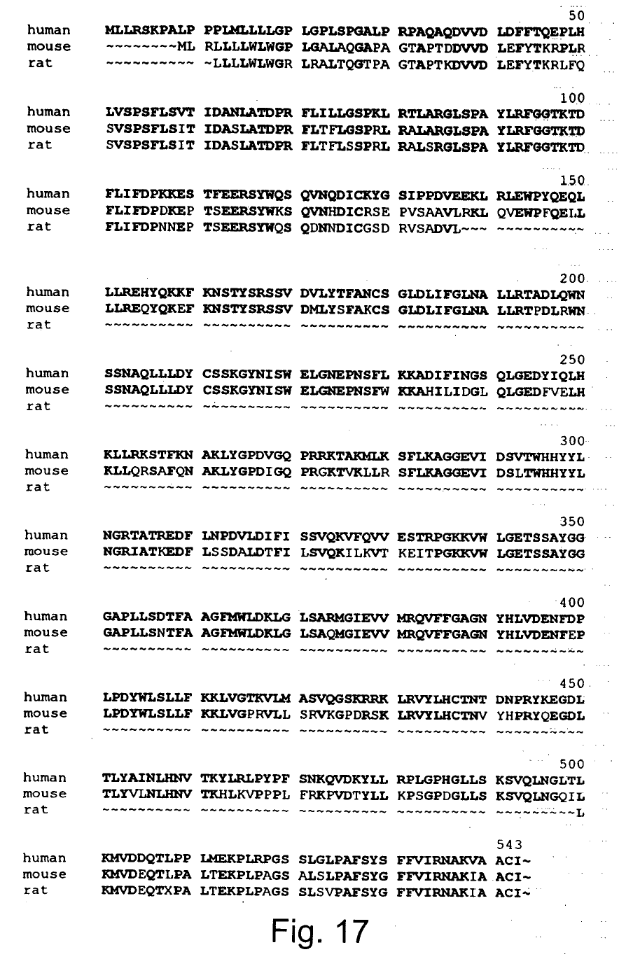

According to another preferred embodiment of the present invention the polypeptide encoded by the polynucleotide sequence is at least 60%, preferably at least 65%, more preferably at least 70%, more preferably at least 75%, more preferably at least 80%, more preferably at least 85%, more preferably at least 90%, most preferably, 95-100% homologous (both similar and identical acids) to SEQ ID NOs:10, 14, 44 or portions thereof as determined with the Smith-Waterman algorithm, using the Bioaccelerator platform developed by Compugene (gapop: 10.0, gapext: 0.5, matrix: blosum62, see also the description to FIG. 17).

Further according to the present invention there is provided a nucleic acid construct comprising the isolated nucleic acid described herein. The construct may, and preferably further include an origin of replication and trans regulatory elements, such as promoter and enhancer sequences.

The construct or vector can be of any type. It may be a phage which infects 7 bacteria or a virus which infects eukaryotic cells. It may also be a plasmid, phagemid, cosmid, bacmid or an artificial chromosome.

Further according to the present invention there is provided a host cell comprising the nucleic acid construct described herein. The host cell can be of any type. It may be a prokaryotic cell, an eukaryotic cell, a cell line, or a cell as a portion of an organism. The polynucleotide encoding heparanase can be permanently or transiently present in the cell. In other words, genetically modified cells obtained following stable or transient transfection, transformation or transduction are all within the scope of the present invention. The polynucleotide can be present in the cell in low copy (say 1-5 copies) or high copy number (say 5-50 copies or more). It may be integrated in one or more chromosomes at any location or be present as an extrachromosomal material.

The present invention is further directed at providing a heparanase over-expression system which includes a cell overexpressing heparanase catalytic activity. The cell may be a genetically modified host cell transiently or stably transfected or transformed with any suitable vector which includes a polynucleotide sequence encoding a polypeptide having heparanase activity and a suitable promoter and enhancer sequences to direct over-expression of heparanase. However, the overexpressing cell may also be a product of an insertion (e.g., via homologous recombination) of a promoter and/or enhancer sequence downstream to the endogenous heparanase gene of the expressing cell, which will direct over-expression from the endogenous gene.

The term “over-expression” as used herein in the specification and claims below refers to a level of expression which is higher than a basal level of expression typically characterizing a given cell under otherwise identical conditions.

According to, another aspect the present invention provides an antisense oligonucleotide comprising a polynucleotide or a polynucleotide analog of at least 10, preferably 11-15, more preferably 16-17, more preferably 18, more preferably 19-25, more preferably 26-35, most preferably 35-100 bases being hybridizable in vivo, under physiological conditions, with a portion of a polynucleotide strand encoding a polypeptide having heparanase catalytic activity. The antisense oligonucleotide can be used for downregulating heparanase activity by in vivo administration thereof to a patient. As such, the antisense oligonucleotide according to the present invention can be used to treat types of cancers which are characterized by impaired (over) expression of heparanase, and are dependent on the expression of heparanase for proliferating or forming metastases.

The antisense oligonucleotide can be DNA or RNA or even include nucleotide analogs, examples of which are provided in the Background section hereinabove. The antisense oligonucleotide according to the present invention can be synthetic and is preferably prepared by solid phase synthesis. In addition, it can be of any desired length which still provides specific base pairing. (e.g., 8 or 10, preferably more, nucleotides long) and it can include mismatches that do not hamper base pairing under physiological conditions.

Further according to the present invention there is provided a pharmaceutical composition comprising the antisense oligonucleotide herein described and a pharmaceutically acceptable carrier. The carrier can be, for example, a liposome loadable with the antisense oligonucleotide.

According to a preferred embodiment of the present invention the antisense oligonucleotide further includes a ribozyme sequence. The ribozyme sequence serves to cleave a heparanase RNA molecule to which the antisense oligonucleotide binds, to thereby downregulate heparanase expression.

Further according to the present invention there is provided an antisense nucleic acid construct comprising a promoter sequence and a polynucleotide sequence directing the synthesis of an antisense RNA sequence of at least 10 bases being hybridizable in vivo, under physiological conditions, with a portion of a polynucleotide strand encoding a polypeptide having heparanase catalytic activity. Like the antisense oligonucleotide, the antisense construct can, be used for downregulating heparanase activity by in vivo administration thereof to a patient. As such, the antisense construct, like the antisense oligonucleotide, according to the present invention can be used to treat types of cancers which are characterized impaired (over) expression of heparanase, and, are dependent on the expression of heparanase for proliferating or forming metastases.

Thus, further according to the present invention there is provided a pharmaceutical composition comprising the antisense construct herein described and a pharmaceutically acceptable carrier. The carrier can be, for example, a liposome loadable with the antisense construct.

Formulations for topical administration may include, but are not limited to, lotions, ointments, gels, creams, suppositories, drops, liquids, sprays and powders. Conventional pharmaceutical carriers, aqueous, powder or oily bases, thickeners and the like may be necessary or desirable. Coated condoms, stents, active pads, and other medical devices may also be useful. Compositions for oral administration include powders or granules, suspensions or solutions in water or non-aqueous media, sachets, capsules or tablets. Thickeners, diluents, flavorings, dispersing aids, emulsifiers or binders may be desirable. Formulations for parenteral administration may include, but are not limited to, sterile aqueous solutions which may also contain buffers, diluents and other suitable additives.

Dosing is dependent on severity and responsiveness of the condition to be treated, but will normally be one or more doses per day, week or month with course of treatment lasting from several days to several months or until a cure is effected or a diminution of disease state is achieved. Persons ordinarily skilled in the art can easily determine optimum dosages, dosing methodologies and repetition rates.

According to exemplary but preferred embodiments of the present invention, heparanase preferably has a concentration in a range of from about 0.005 microgram pet 1 cm² to about 50 microgram per 1 cm² of wound area. More preferably, heparanase has a concentration in a range of from about 0.5-microgram per. 1 cm² to about 5 microgram per 1 cm² of wound area. Optionally and preferably, heparanase may be present in a concentration in a range of from about 10 micrograms to about 150 micrograms per dose. It should be noted that the presence of one or more protein stabilizing agents, which are well known in the art and which could easily be selected by one of ordinary skill in the art, may increase the potential overall activity of heparanase during treatment by up to two orders of magnitude. Also, dosing may vary according to whether a single dose is administered or a plurality of doses is administered. The heparanase is preferably provided in a suitable therapeutic/pharmaceutical composition, preferably with a suitable carrier and more preferably with one or more stabilizing agents.

Further according to the present invention there is provided a nucleic acid construct comprising a polynucleotide sequence functioning as a promoter, the polynucleotide sequence is derived from SEQ ID NO:42 and includes at least nucleotides 2135-2635, preferably 2235-2635, more preferably 2335-2635, more preferably 2435-2635, most preferably 2535-2635 thereof, or SEQ ID NO:43 and, includes at least nucleotides 1-420, preferably 120-420, more preferably 220-420, most preferably 320-420, thereof. These nucleotides are shown in the example section that follows to direct the synthesis of a reporter gene in transformed cells. Thus, further according to the present invention there is provided a method of expressing a polynucleotide sequence comprising the step of ligating the polynucleotide sequence downstream to either of the promoter sequences described herein. Heparanase promoters can be isolated from a variety of mammalian an other species by cloning genomic regions present 5′ to the coding sequence thereof. This can be readily achievable by one ordinarily skilled in the art using the heparanase polynucleotides described herein, which are shown in the Examples section that follows to participate in efficient cross species hybridization.

Further according to the present invention there is provided a recombinant protein comprising a polypeptide having heparanase catalytic activity. The protein according to the present invention include modifications known as post translational modifications, including, but not limited to, proteolysis (e.g., removal of a signal peptide and of a pro- or preprotein sequence), ethionine modification, glycosylation, alkylation (e.g., methylation), acetylation, etc. According to preferred embodiments the polypeptide includes at least a portion of SEQ ID NOs:10, 14 or 44, the portion has heparanase catalytic activity. According to preferred embodiments of the present invention the protein is encoded by any of the above described isolated nucleic acids. Further according to the present invention there is provided a pharmaceutical composition comprising, as an active ingredient, the recombinant protein described herein.

The recombinant protein, may be isolated and purified by any conventional protein purification procedure close to homogeneity and/or be mixed with additives. The recombinant protein may be manufactured using any of the genetically modified cells described above, which include any of the expression nucleic acid constructs described herein. The recombinant protein may be in any form. It may be in a crystallized form, a dehydrated powder form or in solution. The recombinant protein may be useful in obtaining pure heparanase, which in turn may be useful in eliciting anti-heparanase antibodies, either poly or monoclonal antibodies, and as a screening active ingredient in an anti-heparanase inhibitors or drugs screening assay or system.

Further according to the present invention there is provided a method of identifying a chromosome region harboring a human heparanase gene in a chromosome spread the method is executed implementing the following method steps, in which in a first step the chromosome spread (either interphase or metaphase spread) is hybridized with a tagged polynucleotide probe encoding heparanase. The tag is preferably a fluorescent tag. In a second step according to the method the chromosome spread is washed, thereby excess of non-hybridized probe is removed. Finally, signals associated with the hybridized tagged polynucleotide probe are searched for, wherein detected signals being indicative of a chromosome region harboring the human heparanase gene. One ordinarily skilled in the art would know, how to use the sequences disclosed herein in suitable labeling reactions and how to use the tagged probes to detect, using in situ hybridization, a chromosome region harboring a human heparanase gene.

Further according to the present invention there is provided a method of in vivo eliciting anti-heparanase antibodies comprising the steps of administering a nucleic acid construct including a polynucleotide segment corresponding to at least a portion of SEQ ID NOs:9, 13 or 43 and a promoter for directing the expression of said polynucleotide segment in vivo. Accordingly, there is provided also a DNA vaccine for in vivo eliciting anti-heparanase antibodies comprising a nucleic acid construct including a polynucleotide segment corresponding to at least a portion of SEQ ID NOs:9, 13 or 43 and a promoter for directing the expression of said polynucleotide segment in vivo. The vaccine optionally further includes a pharmaceutically acceptable carrier, such as a virus, liposome or an antigen presenting cell. Alternatively, the vaccine is employed as a naked DNA vaccine

The present invention can be used to develop treatments for various diseases, to develop diagnostic assays for these diseases and to provide new tools for basic research especially in the fields of medicine and biology.

Specifically, the present invention can be used to develop new drugs to inhibit tumor cell metastasis, inflammation and autoimmunity. The identification of the hpa gene encoding for the heparanase enzyme enables, the production of a recombinant enzyme in heterologous expression systems.

Furthermore, the present invention can be used to modulate bioavailability of heparin-binding growth factors, cellular responses to heparin-binding growth factors (e.g., bFGF, VEGF) and cytokines (e.g., IL-8), cell interaction with plasma lipoproteins, cellular susceptibility to viral, protozoa and some bacterial infections, and disintegration of neurodegenerative plaques. Recombinant heparanase offers a potential treatment for wound healing, angiogenesis, restenosis, atherosclerosis, inflammation, neurodegenerative diseases (such as, for example, Genstmann-Straussler Syndrome, Creutzfeldt-Jakob disease, Scrape and Alzheimer's disease) and certain viral and some bacterial and protozoa infections. Recombinant heparanase can be used to neutralize plasma heparin, as a potential replacement of protamine.

In particular, the potential involvement of heparanase in neovascularization, both in vitro and in, vivo was investigated. In the present study, the availability of recombinant enzyme, specific antibodies and molecular probes enabled the demonstration of a causative involvement of the heparanase enzyme in tumor-associated angiogenesis and the elucidation its mode of action.

While reducing one aspect of the present invention to practice, the expression of heparanase by vascular EC in vitro and in angiogenic blood vessels was studied. Previously, it has been suggested that stimulated EC secrete heparanase-like activity [Godder, K. et al. Heparanase activity in cultured endothelial cells. J Cell Physiol 148, 274-280 (1991); Pillarisetti, S. et al. Endothelial cell heparanase modulation of lipoprotein lipase activity. Evidence that heparan sulfate oligosaccharide is an extracellular chaperone. J Biol Chem 272, 15753-15759 (1997)]. Using RT-PCR, is the present invention now unequivocally demonstrates, for the first time that the heparanase gene is expressed by proliferating human EC. Both cultured human umbilical vein EC (HUVEC) and human bone marrow EC (TrHBMEC) [Schweitzer, K. M. et al. Characterization of a newly established human bone marrow endothelial cell line: distinct adhesive properties for hematopoietic progenitors compared with human umbilical vein endothelial cells. Lab Invest 76, 25-36 (1997)] expressed the heparanase gene. Staining paraffin embedded sections from patients with primary colon adenocarcinoma with monoclonal anti-heparanase antibodies revealed that the heparanase protein is preferentially expressed in sprouting capillaries whereas the endothelium of mature quiescent vessels showed no detectable levels of heparanase. A similar expression pattern was observed in human mammary and pancreatic carcinomas, suggesting a significant role of endothelial heparanase in enabling EC to traverse BM and ECM barriers during sprouting angiogenesis.

As previously reported [Vlodavsky, I. et al. Mammalian heparanase: gene cloning, expression and function in tumor progression and metastasis. Nat Med 5, 793-802 (1999)] and also demonstrated herein, the neoplastic colonic mucosa exhibits an intense heparanase staining, as opposed to no expression of heparanase in normal colon epithelium [Vlodavsky, I. et al. Mammalian heparanase: gene cloning, expression and function in tumor progression and metastasis. Nat Med 5, 793-802 (1999)]. Carcinoma cells can therefore be regarded as the main source of heparanase in the tumor microenvironment. Moreover, at a later stage of tumor progression, heparanase was also found in the tumor stroma.

Without wishing to be limited by a single hypothesis, one possible explanation for the role of tumor- and stroma-derived heparanase in angiogenesis is release of ECM-resident bFGF and other heparin-binding angiogenic factors [Vlodavsky, I., Bar-Shavit, R., Korner, G. & Fuks, Z. Extracellular matrix-bound growth factors, enzymes and plasma proteins. In Basement membranes: Cellular and molecular aspects (eds. D. H. Rohrbach and R. Timpl), Academic Press Inc., Orlando, Fla., pp 327-343, (1993); Vlodavsky, I., Miao, H.Q., Medalion, B., Danagher, P. & Ron, D. 1996. Involvement of heparan sulfate and related molecules in sequestration and growth promoting activity of fibroblast growth factor. Cancer Metastasis Rev 15, 177-186 (1996)]. As is shown in the Examples section below, degradation of HS in the ECM resulted in release of as much as 70% of the ECM-bound bFGF. In another experiment it is shown that released bFGF stimulates 5-8 fold the proliferation of 3T3 fibroblasts and bovine aortic EC. These results clearly indicate that heparanase releases active bFGF sequestered as a complex with HS in the ECM. Both tumor and endothelial heparanase may hence elicit an indirect angiogenic response by means of releasing active HS-FGF complexes from storage in the ECM and tumor microenvironment.

The ability of heparanase cleaved HS degradation fragments to promote the mitogenic activity of bFGF was investigated using a cytokine-dependent lymphoid cell line (BaF3, clone 32): engineered to express FGF-receptor 1 (FGFR1) [Miao, H. Q., Ornitz, D. M., Aingorn, E., Ben-Sasson, S. A. & Vlodavsky, I. Modulation of fibroblast growth factor-2 receptor binding, dimerization, signaling, and angiogenic activity by a synthetic heparin-mimicking polyanionic compound. J Clin Invest 99, 1565-1575 (1997); Ornitz, D. M. et al. Heparin is required for cell-free binding of basic fibroblast growth factor to a soluble receptor and for mitogenesis in whole cells. Mol Cell Biol 12, 240-247 (1992)].

The results indicate that the heparanase enzyme potentiates the mitogenic activity of bFGF and possibly other heparin-binding angiogenic growth factors, through release of HS degradation fragments that promote bFGF-receptor binding and activation. The observed difference in biological activity between cell surface- and ECM-derived HS fragments indicates that the primary role of HS in the ECM is to sequester, protect and stabilize heparin-binding growth factors, while the cell surface HS plays a more active role in promoting the mitogenic and angiogenic activities of the growth factor by means of stimulating receptor binding, dimerization and activation. This concept is supported by the recently reported preferential ability of cell surface vs. ECM-HSPG to mediate the assembly of bFGF-receptor signaling complex [Chang, Z., Meyer, K., Rapraeger, A. C. & Friedl, A. Differential ability of heparan sulfate proteoglycans to assemble the fibroblast growth factor receptor complex in situ. FASEB J. 14, 137-144 (2000)].

The Matrigel plug assay [Passaniti, A. et al. A simple, quantitative method for assessing angiogenesis and antiangiogenic agents using reconstituted basement membrane, heparin, and fibroblast growth factor. Lab Invest 67, 519-528 (1992)] was applied to investigate whether the heparanase enzyme can elicit an angiogenic response in vivo. A pronounced angiogenic response was induced by Matrigel embedded Eb cells over expressing the heparanase enzyme, as compared to little or no neovascularization exerted by mock transfected Eb cells expressing no heparanase activity. The angiogenic response was reflected by a network of capillary blood vessels attracted toward the Matrigel plug containing heparanase transfected vs. control mock transfected Eb cells, and by a large amount of blood and vessels seen in the isolated Matrigel plugs excised from each of the mice. This difference was highly significant, as also demonstrated by measurements of the hemoglobin content of Matrigel plugs removed from each mouse of the respective groups.

These findings, together with previous results on the increased metastatic potential of heparanase transfected vs. mock transfected Eb cells [Vlodavsky, —I. et al. Mammalian heparanase: gene cloning, expression and function in tumor progression and metastasis. Nat Med 5, 793-802 (1999)]emphasize the significance of heparanase in the two critical events in tumor progression metastasis and angiogenesis.

Compounds that inhibit the heparanase enzyme are therefore anticipated to exert an anti-cancerous effect through inhibition of both tumor cell metastasis [Vlodavsky, I. et al. Mammalian heparanase: gene cloning, expression and function in tumor progression and metastasis. Nat Med 5, 793-802 (1999); Vlodavsky, I. et al. Inhibition of tumor metastasis by heparanase inhibiting species of heparin. Invasion Metastasis 14, 290-302 (1994)] and angiogenesis.

The primary goal in the treatment of wounds is to achieve wound closure. Open cutaneous wounds represent one major category of wounds and include burn wounds, neuropathic ulcers, pressure sores, venous stasis ulcers, and diabetic ulcers. Open cutaneous wounds routinely heal by a process which comprises six major components: (i) inflammation; (ii) fibroblast proliferation; (iii) blood vessel proliferation; (iv) connective tissue synthesis; (v) epithelialization; and (vi) wound contraction. Wound healing is impaired when these components, either individually or as a whole, do not function properly. Numerous factors can affect wound healing, including malnutrition, infection, pharmacological agents (e.g., actinomycin and steroids), advanced age immunodeficiency and diabetes [see Hunt and Goodson in. Current Surgical Diagnosis & Treatment (Way; Appleton & Lange), pp. 86-98 (1988)].

With respect to diabetes, diabetes mellitus is characterized by impaired insulin signaling, elevated plasma, glucose and a predisposition to develop chronic complications involving several distinctive tissues. Among all the chronic complications of diabetes mellitus, impaired wound healing leading to foot ulceration is among the least well studied. Yet skin ulceration in diabetic patients takes a staggering personal and financial cost [Knighton, D R. and Fiegel V. D. Growth factors and comprehensive surgical care of diabetic wounds. Curr. Opin. Gen. Surg.: 32-9: 32-39, 1993; Shaw, J. E. and Boulton, A. J. The pathogenesis of diabetic foot problems: an overview. Diabetes, 46 Suppl 2: S58-S61, 1997].

Moreover, foot ulcers and the subsequent amputation of a lower extremity are the most common causes of hospitalization among diabetic patients [Shaw, J. E. and Boulton, A. J. The pathogenesis of diabetic foot problems: an overview. Diabetes, 46 Suppl 2:S58-61: S58-S611997; Coghlan, M. P., Pillay, T. S., Tavare, J. M., and Siddle, K. Site-specific anti-phosphopeptide antibodies: use in assessing insulin receptor serine/threonine phosphorylation state and identification of serine-1327 as a novel site of phorbol ester-induced phosphorylation. Biochem. J., 303: 893-899, 1994; Grunfeld, C. Diabetic foot ulcers: etiology, treatment, and prevention. Adv. Intern. Med. 37:103-32: 103-132, 1992; Reiber, G. E., Lipsky, B. A., and Gibbons, G. W. The burden of diabetic foot ulcers. Am. J. Surg., 176: 5S-10S, 1998]. In diabetes, the wound healing process is impaired and healed wounds are characterized by diminished wound strength.

Skin is a stratified squamous epithelium in which cells undergoing growth and differentiation are strictly compartmentalized. In the physiologic state, proliferation is confined to the basal cells that adhere to the basement membrane. Differentiation is a spatial process where basal cells lose their adhesion to the basement membrane, cease DNA synthesis and undergo a series of morphological and biochemical changes. The ultimate maturation step is the production of the cornified layer forming the protective barrier of the skin [Hennings, H., Michael, D., Cheng, C., Steinert, P., Holbrook, K., and Yuspa, S. H. Calcium regulation of growth and differentiation of mouse epidermal cells in culture. Cell, 19: 245-254, 1980; Yuspa, S. H., Kilkenny, A. E., Steinert, P. M., and Roop, D. R. Expression of murine epidermal differentiation markers is tightly regulated by restricted extracellular calcium concentrations in vitro. J. Cell Biol., 109: 1207-1217, 1989].

The earliest changes observed when basal cells commit to differentiate is associated with the ability of the basal cells to detach and migrate away from the basement membrane [Fuchs, E. Epidermal differentiation: the bare essentials. J. Cell Biol., 111: 2807-2814, 1990.]. Similar changes are associated with the wound healing process where cells both migrate into the wound area and proliferative capacity is enhanced. These processes are mandatory for the restructing of the skin layers and induction of proper differentiation of the epidermal layers.

Adult skin includes two layers: a keratinized stratified epidermis and an underlying thick layer of collagen-rich dermal connective tissue providing support and nourishment. Skin serves as the protective barrier against the outside world. Therefore any injury or break in the skin must be rapidly and efficiently mended. As described hereinabove, the first stage of the repair is achieved by formation of the clot that plugs the initial wound. Thereafter, inflammatory cells, fibroblasts and capillaries invade the clot to form the granulation tissue. The following stages involve re-epithelization of the wound where basal keratinocytes have to lose their hemidesmosomal contacts, keratinocytes migrate upon the granulation tissue to cover the wound. Following keratinocyte migration, keratinocytes enter a proliferative boost, which allows replacement of cells lost during the injury. After the wound is covered by a monolayer of keratinocytes, new stratified epidermis is formed and the new basement membrane is reestablished [Weinstein, M. L. Update on wound healing: a review of the literature. Mil. Med., 163: 620-624, 1998; Singer, A. J. and Clark, R. A. Cutaneous wound healing. N. Engl. J. Med., 341: 738-746, 1999; Whitby, D. J. and Ferguson, M. W. Immunohistochemical localization of growth factors in fetal wound healing. Dev. Biol., 147: 207-215, 1991; Kiritsy, C. P., Lynch, B., and Lynch, S. E. Role of growth factors in cutaneous wound healing: a review. Crit. Rev. Oral Biol. Med., 4: 729-760, 1993].

Several growth factors have been shown to participate in this process including EGF family of growth factors, KGF, PDGF and TGFβ1 [Whitby, D. J. and Ferguson, M. W. Immunohistochemical localization of growth factors in fetal wound healing. Dev. Biol., 147: 207-215, 1991; Kiritsy, C. P., Lynch, B., and Lynch, S. E. Role of growth factors in cutaneous wound healing: a review. Crit. Rev. Oral Biol. Med., 4: 729-760, 1993; Andresen, J. L., Ledet, T., and Ehlers, N. Keratocyte migration and peptide growth factors: the effect of PDGF, bFGF, EGF, IGF-1, aFGF and TGF-beta on human keratocyte migration in a collagen gel. Curr. Eye Res., 16: 605-613, 1997]. Among these growth factors both EGF and KGF are thought to be intimately involved in the regulation of proliferation and migration of epidermal keratinocytes [Werner, S., Breeden, M., Hubner, G., Greenhalgh, D. G., and Longaker, M. T. Induction of keratinocyte growth factor expression is reduced and delayed during wound healing in the genetically diabetic mouse. J. Invest. Dermatol., 103: 469-473, 1994; Threadgill, D. W., Dlugosz, A. A., Hansen, L. A., Tennenbaum, T., Lichti, U., Yee, D., LaMantia C., Mourton, T., Herrup, K., Harris R. C., Barnard, J. A., Yuspa, S. H., Coffey, R. J, and Magnuson, T. Targeted disruption of mouse EGF receptor: effect of genetic background on mutant phenotype. Science, 269: 230-234, 1995].

As has already been mentioned hereinabove, heparan sulfate proteoglycan (HSPGs) are ubiquitous macromolecules associated with the cell surface and the extracellular matrix (ECM). The ability of heparan sulfate to interact with ECM molecules such as collagen, laminin and fibronectin indicates that this proteoglycan is essential for self-assembly, insolubility and function of the ECM. Initially envisioned as a physical tissue support, it is now clear that the ECM actively transmit biochemical signals, which affect a variety of cellular behaviors. These include cell adhesion, proliferation, migration, survival, locomotion and tissue integrity, function, morphology and architecture. Expression of HS-degrading endoglycosidases, commonly called heparanases, correlates with the metastatic potential of mouse and human lymphoma, fibrosarcoma, and melanoma cell lines, and with extravasation associated with inflammation and autoimmunity. In addition to being involved in the remodeling of ECM and egress of cells from the vasculature, heparanase may regulate angiogenesis, tissue repair and remodeling as well as wound healing by releasing HS-bound growth factors (e.g., bFGF, KGF, VEGF, HGF, HB-EGF), cytokines [interleukin (IL) 1, 8, 10] and chemokines (RANTES, MCP-1, MIP 1; [Vaday G. G. and O. Lider. 2000. Extracellular matrix moieties, cytokine, and enzymes: dynamic effect on immune cell behavior and inflammation. J. Leukoc. Biol. 67: 149-159]). The release of such proteins associated with low molecular weight HS can potentiate the interaction of soluble growth factors with their cell surface receptors, as has been shown for bFGF [Vlodavsky I., H.-Q. Miao, B. Medalion, P. Danagher and D. Ron. 1996. Involvement of heparan sulfate: and related molecules in sequestration and growth promoting activity of fibroblast growth factor. Cancer and Metastasis Reviews 15: 177-186], or can protect the bound protein from proteolytic cleavage.

Until recently, the nature of heparanase was a matter of dispute. For example, Fuks et al. discloses a partially purified polypeptide from Sk-Hep-1 cells having endoglycosidase activity inhibited by heparin (PCT No. WO 91/02977 to Fuks and Vlodavsky), characteristic of heparanase. However, the disclosed protein is clearly contaminated with the 50 kDa type 1 plaminogen activator PAI-1, and further purification is confounded by the cross-reactivity of anti-PAI-1 antibodies with the the purified heparanase (see page 28, lines 10-19). The PAI-1 contamination persisted throughout the various gel- and affinity purification steps, including cation exchange chromatography (page 28, lines 10-19), heparin-sepharose and gel filtration (page 29, lines 10-19) and concanavalin-A sepharose affinity column (page 30, lines 24-32). However, within the past two years, several laboratories have purified human heparanase and isolated the cDNA encoding this activity [Vlodavsky I., Y. Friedman, M. Elkin, H. Aingorn, R. Atzmon, R. Ishai-Michaeli, M. Bitan, O. Pappo, T. Peretz, I. Michal, L. Spector and I. Pecker. 1999. Mammalian heparanase: Gene cloning, expression and function in tumor progression and metastasis. Nature Med. 5: 793-802; Hulett M. D., C. Freeman, B. J. Hamdorf, R. T. Baker, M. J. Harris and C. R. Parish. 1999. Cloning of mammalian heparanase, an important enzyme in tumor invasion and metastasis. Nature Med. 5: 803-809; Toyoshima M. and M. Nakajima. 1999. Human heparanase: purification, characterization, cloning and expression. J. Biol. Chem. 274: 24153-24160]. Expression of the cloned cDNA in insect and mammalian cells yielded 65 and 50 kDa glycoproteins. The 50 kDa enzyme represent an N-terminal processed enzyme, which is at least 200-fold more active than the full-length 65 kDa protein [Vlodavsky I., Y. Friedman, M. Elkin, H. Aingorn, R. Atzmon, R. Ishai-Michaeli, M. Bitan, O. Pappo, T. Peretz, I. Michal, L. Spector and I. Pecker. 1999. Mammalian heparanase: Gene cloning, expression and function in tumor progression and metastasis. Nature Med. 5: 793-802]. Heparanase activities purified from different human and animal sources are related immunologically, share substrate specificities, yield similar oligosaccharide cleavage products and are inhibited by heparin substrate derivatives. This may suggest that the cloned enzyme represent the predominant heparanase in mammalian species. The availability of purified active enzyme made it possible to further explore the role of heparanase in a highly controlled manner and in a specific biological setting.

While reducing one aspect of the present invention to practice it was demonstrated that the active 50 kDa heparanase enzyme accelerates wound closure in a mouse skin model.

Indirect evidence correlated heparanase activity to angiogenesis and inflammation, which are both required for successful wound healing.

In order to directly study the effect of heparanase on the complex of events composing wound healing, active heparanase was applied topically onto full-thickness wounds. Careful evaluation of wounds areas revealed a significant improvement of wound closure upon heparanase treatment.

It is known that the inactive form of heparanase, P60, is activatable in vivo, via proteolysis into its active form P50 (see, for example, U.S. patent application Ser. No. 09/260,037), and may therefore also be used in accordance with the teachings of the present invention for wound healing, induction of angiogenesis and/or for cosmetic applications.

Having demonstrated, for the first time, a direct role for heparanase activity in the wound healing process, cellular and molecular mechanisms that are activated by heparanase in the course of wound healing were sought. Examination of hematoxilin-eosin stained wound sections revealed the expected granulation tissue morphology, composed of fibroblasts, blood vessels and inflammatory cells. Interestingly, the heparanase-treated granulation tissue was much more dense. Specifically, a significant increase in the number of inflammatory cells and blood vessels was observed. This was further confirmed by staining for PCNA, a marker for cell proliferation and for PECAM-1, a marker for endothelial cells. Indeed, an increase in PCNA and PECAM-1 staining was observed in the granulation tissue of heparanase-treated wounds. Thus, the acceleration of wound healing is, without limitation, due to the robust fibroblast and inflammatory cells-derived cytokine and chemokines and to increased vascularity. Heparanase was found to be expressed by all the major cell components of granulation tissue. Interestingly, heparanase expression was mainly detected in the differentiated, non-proliferating, cells composing the epidermis, while proliferating, PCNA-positive epidermal cells reconstituting the wound were poorly stained. In addition, heparanase staining was observed in non-proliferating hair follicle cells. Such staining pattern V suggests, without limitation, that heparanase plays a role in cellular terminal-differentiation which leads, as in the case of keratinocyes, to apoptosis and as an anti-infectant.

Heparan sulfates are prominent components of blood vessels. In capillaries they are found mainly in the subendothelial basement membrane, supporting and stabilizing the structure of blood vessels wall. Cleavage of the underlying ECM plays a decisive part not only in the extravasation of blood-born (immune) cells, but also in the sprouting of new capillaries from pre-existing blood vessels. This early step is believed to contribute significantly to the invasive ability of endothelial cells and their subsequent migration through the ECM toward the angiogenic stimulus. Heparanase expression was detected in proliferating endothelial cells in vitro and moreover, in sprouting capillaries in vivo. In contrast, the endothelium of mature, quiescent vessels showed no detectable heparanase expression, suggesting that heparanase activity may be involved in angiogenic sprout formation.

Wounded skin will cause leakage of blood from damaged blood vessels and the formation of fibrin clot. Importantly, the clot serves as a reservoir for cytokines and growth factors that are released as activated platelets degranulate [Martin P. 1997. Wound healing-Aiming for perfect skin regeneration. Science 276:75-81], and may be the target for the exogenous heparanase. This may also explain the increase of inflammatory cells recruited to granulation tissue observed after heparanase treatment.

Expression of heparanase gene and protein correlated with the metastatic potential of several human and mouse cell lines such as breast, bladder, prostate; melanoma and T-lymphoma [Vlodavsky I., Y. Friedman, M. Elkin, H. Aingorn, R. Atzmon, R. Ishai-Michaeli, M. Bitan; O. Pappo, T. Peretz, L Michal, L. Spector and I. Pecker. 1999. Mammalian heparanase: Gene cloning, expression and function in tumor progression and metastasis. Nature Med. 5: 793-802]. Similarly, heparanase activity was also correlated with extravasation of immune cells during normal and chronic inflammation and with angiogenesis. Here evidence is provided, for the first time, for a direct role for heparanase in the course of wound healing and, moreover, in the regulation of sprouting angiogenesis.

A few potential clinical benefits for heparanase come to mind.

1. Heparanase may be used as a therapeutic for a wide variety of wounds under pathological conditions. These include diabetic and pressure ulcers, burns and incisional wounds, and may expand further to tissue damage caused by ischemia, mainly in the context of heart and kidney diseases. Moreover, accelerated healing may contribute to the aesthetically appearance of the wounds, implicating a potential cosmetic benefit.

2. Heparanase may be considered as an infection-inhibiting reagent. This is based upon the observation that heparanase expression is restricted to the outer most layer of the skin (stratum corneum) and the ability of various pathogenic bacteria, viruses and protozoa to bind glycosaminoglycan-based receptors on host, cells, initiating infection. The combination of accelerated wound healing with inhibition of infection may provide for an even more potent reagent.

3. The intimate involvement in angiogenesis and the ability of heparanase to induce blood vessels formation, shown here directly for the first time, may have important clinical implication. Tumor growth is angiogenic-dependent and inhibition of blood vessel formation is sought as a cancer therapeutic. Other clinical situations critically suffer from severe tissue damage and induction of angiogenesis is believed to significantly improve tissue function. The most common and important example is ischemic heart damage, affecting millions of people every year.

4. Cutaneous wounds often cause anatomical and/or functional damage to peripheral sensory neurons widely distributed in the skin, and nerve growth factor (NGF) may be essential to regenerate the injured neurons. Neurotropic activity, of NGF has been shown to be potentiating by heparin. (Neufeld et al., 1987, Heparin modulation of the neurotropic effects of acidic and basic fibroblast growth factors and nerve growth factor on PC12 cells. J Cell Physiol. 1987 April; 131(1):131-40.) and heparan sulfate (Damon et al., 1988, Sulfated glycosaminoglycans modify growth factor-induced neurite outgrowth in PC12 cells. J Cell Physiol 1988 May; 135(2):293-300). Thus, heparanase activity may increase the availability of a variety of growth factors, including NGF and to support neuronal recovery.

5. As shown herein, the increase in granulation tissue cellularity is due, in part, to an increase in cell proliferation. However, a large cell population which is PCNA-negative also appears and is most likely composed of inflammatory cells. Thus, heparanase treatment may enhance the recruitment of inflammatory cells to specific sites. On the other hand, heparanase inhibitors may prevent or reduce inflammation under several pathological conditions, including chronic and acute inflammation.

6. Heparanase expression in the skin tissue correlated with terminal cellular differentiation and keratinocytes apoptosis, while proliferating epidermal cells, stained positively for PCNA, expressed only very low levels of heparanase. Interestingly, heparanase was found to be localized to the nucleus of hair follicle cells, while cytoplasmic staining was observed in keratinocytes. This may suggest a new potential function for heparanase, other than the traditional ones. More specifically, heparanase localization to the nucleus may be involve in the regulation of gene expression, most likely due to heparanase-associating factors, and cell fate.

Heparan sulfate is found throughout the epidermis [Tammi R H et al; Histochem. 1987, 87:243-50], but its function is unknown. The role of heparanase in normal, aging and pathological conditions of the skin is also not known, in part due to the lack of specific anti-heparanase antibodies and a purified enzyme. A few reports that describe altered HS metabolism, due to both quantitative and qualitative changes, may suggest a role for the heparanase enzyme, or its inhibitors, in the treatment of various skin conditions. It was found that cells which had aged in vivo, or in vitro, had an increased proportion of HSPG [Kent W M et al; Mech Aging Dev. 1986, 33:115-37]. It was also found that HS and blood vessels staining were increased in wounds of old animals at late time points, but the dermal organization was similar to that of normal skin. In contrast, young animals developed abnormal, dense scars. Intriguingly, some of the age-related changes in scar quality and inflammatory cell profile were similar to those seen in fetal wound healing [Ashcroft G S et al; J. Invest Dermatol. 1997, 108:430-7]. Another paper showed that under the influence of chronic UVB radiation animals exhibited a marked increase in the synthesis of HS [Margelin D et al; Photochem. PhotobioL 1993, 58:211-8]. HSPGs distribution changes during the differentiation stages of hair growth cycle, and they have an inductive effect on hair growth, both when injected and in diseases that result in accumulation of polysaccharides in the dermis [Westgate G et al; J Invet Dermatol. 1991, 96:191-5]. In addition to putative roles of HS in basement membrane assembly, and cell-matrix interactions, growth factor sequestration may be important for the hair follicle [Couchman J R et al; J Invest Dermatol. 1995, 104:40S]. Administration of exogenous bFGF has prolonged and marked effects on mouse hair follicle development and cycling [du Cros D L; Dev Biol. 1993, 156:444-53]. The heparin binding keratinocyte growth factors human-derived keratinocyte autocrine factor (KAF) and amphiregulin (AR) can be negatively regulated by heparin [Cook P W et al; Mol Cell Biol. 1991, 11:2547-57].

As described herein in the Examples section that follows, using an anti-heparanase monoclonal antibody (HP-92) cultures of HaCat keratinocytes cell line were immunostained. These cells exhibited significant heparanase staining in their cytoplasm. Moreover, intact cells, as well as an extract of these cells exhibited heparanase activity when assayed in an ECM-assay. Immuno-staining of normal skin tissues resulted in the intense staining of heparanase both in the dermis and epidermis.

The following describes potential applications of heparanase and/or heparanase inhibitors in skin and hair care:

Heparanase treatment may improve the appearance of the skin damaged by UV irradiation and aging. Removal of excess heparan sulfate following UV exposure may restore natural skin (a process termed “biochemical peeling”).

Heparanase treatment may aid in skin healing via its mitogenic and angiogenic properties.

Heparanase treatment may have regenerative properties for hair growth via mitogenesis and angiogenesis.

Heparanase inhibitors may prevent minor skin inflammations, irritations and allergies via inhibition of the inflammatory immune cell response.

Heparanase inhibitors may increase levels of heparan sulfate and thus affect hair growth, skin resiliency, etc.

To facilitate understanding of the invention set forth in this disclosure, a number of terms are defined below.

The term “wound” refers broadly to injuries to the skin and subcutaneous tissue initiated in any one of a variety of ways (e.g., pressure sores from extended bed rest, wounds induced by trauma, cuts, ulcers, burns and the like) and with varying characteristics. Wounds are typically classified into one of four grades depending on the depth of the wound: (i) Grade I: wounds limited to the epithelium; (ii) Grade II wounds extending into the dermis; (iii) Grade III: wounds extending into the subcutaneous tissue; and (iv) Grade IV (or full-thickness wounds): wounds wherein bones are exposed (e.g., a bony pressure point such as the greater trochanter or the sacrum). The term “partial thickness wound” refers to wounds that encompass Grades I-III; examples of partial thickness wounds include burn wounds, pressure sores; venous stasis ulcers, and diabetic ulcers. The term ““deep wound” is meant to included both Grade III and Grade IV wounds.

The term “healing” in respect to a wound refers to a process to repair a wound as by scar formation.

The phrase “inducing or accelerating a healing process of a wound” refers to either the induction of the formation of granulation tissue of wound contraction and/or the induction of epithelialization (i.e., the generation: of new cells in the epithelium). Wound healing is conveniently measured by decreasing wound area.

Hereinafter, the term “treating a wound” includes inducing or accelerating a healing process of a wound, as well as ameliorating a condition of the wound, and/or a complication (complicating condition) associated with the wound.

The present invention contemplates treating all wound types, including deep wounds and chronic-wounds.

The term “chronic wound” refers a wound that has not healed within 30 days.

The phrase “transforming cells” refers to a transient or permanent alteration of a cell's nucleic acid content by the incorporation of exogenous nucleic acid which either integrates into the cell genome and genetically modifies the cell or remains unintegrated.

The phrase “cis-acting element” is used herein to describe a genetic element that is located upstream of a coding sequence and controls the expression of a protein from the coding sequence. Such-elements include promoters and enhancers.

The term “angiogenesis” is used herein to described the process of blood vessels formation.

Wound healing and angiogenesis according to the present invention are induced and/or accelerated by the presence of heparanase. As is demonstrated herein, heparanase, by degrading HS releases and/or activates a plurality of factors which evidently induce and/or accelerate wound healing and angiogenesis, wherein wound healing is induced or accelerated by induced or accelerated angiogenesis and inflammation, whereas angiogenesis itself is induced by release of angiogenic factors from the ECM.

The phrase “heparanase coated cells” refers to cells to which natural or recombinant, active or activatable (proenzyme)-heparanase was externally adhered ex vivo. Such cells can form a part of a tissue soaked in a heparanase containing solution.

Hereinafter the term “heparanase being substantially free of contaminants” refers to heparanase having less than about 5%, preferably, less than about 2%, more preferably, less than about 1%, preferably, less than about 0.5%, still preferably, less than about 0.1% by weight of non-heparanase contaminants associated with the heparanase, wherein the contaminants may optionally include any one or more of contaminating proteins, contaminants capable of eliciting an antibody, contaminating human proteins, any type of protein exhibiting human glycosylation, contaminants in an amount sufficient for performing protein microsequencing or any combination thereof.

Thus, according to one aspect of the present invention there is provided a method of inducing or accelerating a healing process of a wound and/or angiogenesis. The method according to this aspect of the invention is effected by administering a therapeutically effective amount of heparanase, so as to induce or accelerate the healing process of the wound and/or angiogenesis.

According to another aspect of the present invention there is provided a pharmaceutical composition for inducing or accelerating a healing process of a wound and/or angiogenesis. The pharmaceutical composition comprising, as an active ingredient, heparanase and a pharmaceutically acceptable carrier.

According to yet another aspect of the present invention there is provided a method of inducing or accelerating a healing process of a wound and/or angiogenesis. The method according to this aspect of the invention is effected by implanting a therapeutically effective amount of heparanase expressing or secreting cells, or heparanase coated cells, so as to induce or accelerate the healing process of the wound and/or angiogenesis.

According to still another aspect of the present invention there is provided a pharmaceutical composition for inducing or accelerating a healing process of a wound and/or angiogenesis. The pharmaceutical composition according to this aspect of the invention comprising, as an active ingredient, heparanase expressing or secreting cells, or heparanase coated cells, and a pharmaceutically acceptable carrier.

According to an additional aspect of the present invention there is provided a method of inducing or accelerating a healing process of a wound and/or angiogenesis. The method according to this aspect of the invention is effected by transforming cells in vivo to produce and secrete heparanase, so as to induce or accelerate the healing process of the wound and/or angiogenesis.

According to yet an additional aspect of the present invention there is provided a pharmaceutical composition for inducing or accelerating a healing process of a wound and/or angiogenesis. The pharmaceutical composition according to this aspect of the invention comprising, as an active ingredient, a nucleic acid construct being designed for transforming cells in vivo to produce and secrete heparanase, and a pharmaceutically acceptable carrier.

Thus, wound healing and angiogenesis according to the present invention are induced and/or accelerated by heparanase.

One method is the direct administration of heparanase. Heparanase can be purified from natural sources or produced by recombinant technology.

In an alternative embodiment, cells expressing or secreting heparanase are implanted in vivo, so as to induce or accelerate the healing process of a wound or induce angiogenesis. Such heparanase producing cells may be cells naturally producing heparanase, or alternatively, such cells are transformed to produce and secrete heparanase. The cells can be transformed by a cis-acting element sequence, such as a strong and constitutive or inducible promoter integrated upstream to an endogenous heparanase gene of the cells, by way of gene knock-in, and produce and secrete natural heparanase. It will be appreciated that the still alternatively, the cells can be transformed by a recombinant heparanase gene to produce and secrete recombinant heparanase.

Advantageously, the heparanase expressing or secreting cells are capable of forming secretory granules, so as to secrete heparanase produced thereby. The heparanase expressing or secreting cells can be endocrine cells. They can be of a human source or of a histocompatibility humanized animal source. Most preferably, the heparanase expressing or secreting cells, either transformed or not, are of an autologous source. The heparanase produced by the heparanase expressing or secreting cells is preferably human heparanase or has the amino acid sequence of human heparanase. The heparanase expressing or secreting cells can be fibroblasts, epithelial cells, keratinocytes or cells present in a full thickness skin, provided that a transformation as described herein is employed so as to render such cells to produce and secrete heparanase. Cells or tissue such as full thickness skin implant or transplant can be coated with heparanase. Thus the cells of the present invention can be isolated cells or cells embedded in a tissue implant or transplant.

In still an alternative embodiment cells are transformed in vivo to produce and secrete heparanase, so as to induce or accelerate the healing process of a wound and/or” angiogenesis.

Any one of a plurality of transformation approaches described above e.g., transformation with a construct encoding heparanase, or transformation with a construct harboring a cis-acting element for activation of endogenous heparanase, production and secretion, can be employed in context of this embodiment of the present invention.

In some aspects the present invention utilizes in vivo and ex vivo (cellular) gene therapy techniques, which involve cell transformation and gene knock-in type transformation. Gene therapy as used herein refers to the transfer of genetic material (e.g., DNA or RNA) of interest into a host to treat or prevent a genetic or acquired disease or condition or phenotype. The genetic material of interest encodes a product (e.g., a protein, polypeptide, peptide, functional RNA, antisense: RNA) whose production in vivo is desired. For example, the genetic material of interest can encode a hormone, receptor, enzyme, polypeptide or peptide of therapeutic value. For review see, in general, the text “Gene Therapy” (Advanced in Pharmacology 40, Academic Press, 1997).

Two basic approaches to gene therapy have evolved (1) ex vivo; and (ii) in vivo gene therapy. In ex vivo gene therapy cells are removed from a patient or are derived from another source, and while being cultured are treated in vitro. Generally, a functional replacement gene is introduced into the cell via an appropriate gene delivery vehicle/method. (transfection, transduction, homologous recombination, etc.) and an expression system as needed and then the modified cells are expanded in culture and returned to the host/patient. These genetically reimplanted cells have been shown to express the transfected genetic material in situ.

In in vivo gene therapy, target cells are not removed from the subject rather the genetic material to be transferred is introduced into the cells of the recipient organism in situ, that is within the recipient. In an alternative embodiment, if the host gene is defective, the gene is repaired in situ [Culver, 1998. (Abstract); Antisense DNA & RNA based therapeutics, February 1998, Coronado, Calif.]. These genetically altered cells have been shown to express the transfected genetic material in situ.

The gene expression vehicle is capable of delivery/transfer of heterologous nucleic acid into a host cell. The expression vehicle may include elements to control targeting, expression and transcription of the nucleic acid in a cell selective manner as is known in the art. It should be noted that often the 5′UTR and/or 3′UTR of the gene may be replaced by the 5′UTR and/or 3′UTR of the expression vehicle. Therefore, as used herein the expression vehicle may, as needed, not include the 5′UTR and/or 3′UTR of the actual gene to be transferred and only include the specific amino acid coding region.

The expression vehicle can include a promoter for controlling transcription of the heterologous material and can be either a constitutive or inducible promoters to allow selective transcription. Enhancers that may be required to obtain necessary transcription levels can optionally be included. Enhancers are generally any nontranslated DNA sequence which works contiguously with the coding sequence (in cis) to change the basal transcription level dictated by the promoter. The expression vehicle can also include a selection gene as described herein below.

Vectors can be introduced into cells or tissues by any one of a variety of known methods within the art. Such methods can be found generally described in Sambrook et al., Molecular Cloning: A: Laboratory Manual, Cold Springs Harbor Laboratory, New York 0.1989, 1992, in Ausubel et al., Current Protocols in Molecular Biology, John Wiley and Sons, Baltimore, Md. 0.1989, Chang et al., Somatic Gene Therapy, CRC Press, Ann Arbor, MI 1995, Vega et al., Gene Targeting, CRC Press, Ann Arbor Mich. (995), Vectors: A Survey of Molecular Cloning Vectors and Their Uses, Butterworths, Boston Mass. 1988 and Gilboa et al., Biotechniques 4 (6): 504-512, 1986, and include, for example, stable or transient transfection, lipofection, electroporation and infection with recombinant viral vectors. In addition, see U.S. Pat. No. 4,866,042 for vectors involving the central nervous system and also U.S. Pat. Nos. 5,464,764 and 5,487,992 for positive-negative selection methods.

Introduction of nucleic acids by infection offers several advantages over the other listed methods. Higher efficiency can be obtained due to their infectious nature. Moreover, viruses are very specialized and typically infect and propagate in specific cell types. Thus, their natural specificity can be used to target the vectors to specific cell types in vivo or within a tissue or mixed culture of cells. Vital vectors can also be modified with specific receptors or ligands to alter target specificity through receptor mediated events.

A specific example of DNA viral vector introducing and expressing recombination sequences is the adenovirus-derived vector Adenop53TK. This vector expresses a herpes virus thymidine kinase (TK) gene for either positive or negative selection and an expression cassette for desired recombinant sequences. This vector can be used to infect cells that have an adenovirus receptor which includes most tissues of epithelial origin as well as others. This vector as well as others that exhibit similar desired functions can be used to treat a mixed population of cells and can include, for example, in vitro or ex vivo culture of cells, a tissue or a human subject.

Features that limit expression to particular cell types can also be included. Such features include, for example, promoter and regulatory elements that are specific for the desired cell type.

In addition, recombinant viral vectors are useful for in vivo expression of a desired nucleic acid because they offer advantages such as lateral infection and targeting specificity. Lateral infection is inherent in the life cycle of, for example, retrovirus and is the process by which a single infected cell produces many progeny virions that bud off and infect neighboring cells. The result is that, a large area becomes rapidly infected, most of which was not initially infected by the original viral particles. This is in contrast to vertical-type of infection in which the infectious agent spreads only through daughter progeny. Viral vectors can also be produced that are unable to spread laterally. This characteristic can be useful if the desired purpose is to introduce a specified gene into only a localized number of targeted cells.

As described above, viruses are very specialized infectious agents that have evolved, in many cases, to elude host defense mechanisms. Typically, viruses infect and propagate in specific cell types. The targeting specificity of viral vectors utilizes its natural specificity to specifically target predetermined cell types and thereby introduce a recombinant gene into the infected cell. The vector to be used in the methods and compositions of the invention will depend on desired cell type to be targeted and will be known to those skilled in the art.

Retroviral vectors can be constructed to function either as infectious particles or to undergo only a single initial round of infection. In the former case, the genome of the virus is modified so that it maintains all: the necessary genes, regulatory sequences and packaging signals to synthesize new viral proteins and RNA. Once these molecules are synthesized, the host cell packages the RNA into new viral particles which are capable of undergoing further rounds of infection. The vector's genome is also engineered to encode and express the desired recombinant gene. In the case of non-infectious viral vectors, the vector genome is usually mutated to destroy the viral packaging signal that is required to encapsulate the RNA into viral particles. Without such a signal, any particles that are formed will not contain a genome and therefore cannot proceed through subsequent rounds of infection. The specific type of vector will depend upon the intended application. The actual vectors are also known and readily available within the art or can be constructed by one skilled in the art using well-known methodology.

The recombinant vector can be administered in several ways. If viral vectors are used, for example, the procedure can take advantage of their target specificity and consequently, do not have to be administered locally at the diseased site. However, local administration can provide a quicker and more effective treatment.

Procedures for in vivo and ex vivo cell transformation including homologous recombination employed in knock-in procedures are set forth in, for example, U.S. Pat. Nos. 5,487,992, 5,464,764, 5,387,742, 5,360,735, 5,347,075, 5,298,422, 5,288,846, 5,221,778, 5,175,385, 5,175,384, 5,175,383, 4,736,866 as well as Burke and Olson, Methods in Enzymology, 194:251-270 1991); Capecchi, Science 244:1288-1292 1989); Davies et al., Nucleic Acids Research, 20 (11) 2693-2698′. 1992); Dickinson et al., Human Molecular Genetics, 2(8): 1299-1302 19993); Duff and Lincoln, “Insertion of a pathogenic mutation into a yeast artificial chromosome containing the human APP gene and expression in ES cells”; Research Advances in Alzheimer's Disease and Related Disorders, 1995; Huxley et al., Genomics, 9:742-750 1991); Jakobovits et al., Nature, 362:255-261 1993); Lamb et al., Nature-Genetics, 5:22-29 1993); Pearson and Choi, Proc. Natl. Acad. Sci. USA 1993). 90:10578-82; Rothstein, Methods in Enzymology, 194:281-3011991); Schedl et al., Nature, 362: 258-261 1993); Strauss et al., Science, 259:1904-1907 1993). Further, patent applications WO 94/23049, WO93/14200, WO 94/06908, WO 94/28123 also provide information.

Thus, transformations according to the present invention can employ naked DNA or viral vectors to introduce a sequence of interest into cells. Viral vectors are developed by modification of the viral genome in the form of replicative defective viruses. The most widely used viral vectors are the retroviruses and adenoviruses, which are used for experimental as well as gene therapy purposes. [Kuroki, T., Kashiwagi, M., Ishiino, K., Huh, N., and Ohba, M. Adenovirus-mediated gene transfer to keratinocytes—a review. J. Investig. Dermatol. Symp. Proc., 4: 153-157, 1999]. Specifically, the high efficiency of adenovirus infection in non replicating cells, the high titer of virus and the high expression of the transduced protein makes this system highly advantageous to primary, cultures compared to retroviral vectors. As adenoviruses do not integrate into the host genome and the stable viral titers can be rendered replication deficient, these viral constructs are associated with minimal risk for malignancies in human as well as animal models (Rosenfeld, M. A., Siegfried, W., Yoshimura, K., Yoneyama, K., Fukayama, M., Stier, L. E., Paakko, P. K., Gis, P., Stratford-Perricaudet, M., Jallet, J., Pavirani, A., Lecocq, J. P., and Crystal, R. Q. Adenovirus-mediated transfer of a recombinant al-antitrypsin gene to the lung epithelium in vivo. Science, 252: 431-434, 1991). To date, in skin, adenovirus constructs have also been used successfully with high efficiency of infection with ex vivo and in vivo approaches [Setoguchi, Y., Jaffe, H. A., Danel, C., and Crystal, R. G. Ex Vivo and in vivo gene transfer to the skin using replication-deficient recombinant adenovirus vectors. J. Invest. Dermatol., 102: 415-421, 1994; Greenhalgh, D. A., Rothnagel, J. A., and Roop, D. R. Epidermis: An attractive target tissue for gene therapy. J. Invest. Dermnatol., 103: 63S-69S, 1994). An adenovirus vector, which was developed by I. Saito and his associates [Miyake, S., Makimura, M., Kanegae, Y., Harada, S., Sato, Y., Takamori, K., Tokuda, C., and Saito, I. Efficient generation of recombinant adenoviruses using adenovirus DNA-terminal protein complex and a cosmid bearing the full-length virus genome. Proc. Natl. Acad. Sci. U.S.A., 93: 1320-1324, 1996] was used in the present study. The cosmid cassette (pAxCAwt) has nearly a full length adenovirus 5 genome but lacks E1A, E1B and E3 regions, rendering the virus replication defective. It contains a composite CAG promoter, consisting of the cytomegalovirus immediate-early enhancer, chicken β-actin promoter, and a rabbit β-globin polyadenylation signal, which strongly induces expression of inserted DNAs [Kuroki, T., Kashiwagi, M., Ishino, K., Huh, N., and Ohba, M. Adenovirus-mediated gene transfer to keratinocytes—a review. J. Investig. Dermatol. Symp. Proc., 4: 153-157, 1999; Miyake, s., Makimura, M., Kanegae, Y., Harada, S., Sato; Y., Takamori, K., Tokijda, C., and Saito, I. Efficient generation of recombinant adenoviruses using adenovirus DNA-terminal protein complex and a cosmid bearing the full-length virus genome. Proc. Natl. Acad. Sci. U.S.A., 93: 1320-1324, 1996]. A gene of interest is inserted into the cosmid cassette, which is then co-transfected into human embryonic kidney 293 cells together with adenovirus DNA terminal protein complex (TPC). In 293 cells that express E1A and E1B regions, recombination occurs between the cosmid cassette and adenovirus DNA-7′ TPC, yielding the desired recombinant virus at an efficiency 100-fold that of conventional methods. Such high efficiency is mainly due to, the use of the adenovirus DNA-TPC instead of proteinesed DNA. Furthermore, the presence of longer homologous regions increases the efficiency of the homologous recombination. Regeneration of replication competent viruses is avoided due to the presence of multiple EcoT221 sites.

The therapeutically/pharmaceutically active ingredients of the present invention can be administered per se, or in a pharmaceutical composition mixed with suitable carriers and/or excipients. Pharmaceutical compositions suitable for use in context of the present invention include those compositions in which the active ingredients are contained in an amount effective to achieve an intended therapeutic effect.

As used herein a “pharmaceutical composition” refers to a preparation of one or more of the active ingredients described herein, either protein, nucleic acids or cells, or physiologically acceptable salts or prodrugs thereof, with other chemical components such as traditional drugs, physiologically suitable carriers and excipients. The purpose of a pharmaceutical composition is to facilitate administration of a compound or cell to an organism. Pharmaceutical compositions of the present invention may be manufactured by processes well known in the art, e.g., by, means of conventional mixing, dissolving, granulating, dragee-making, levigating, emulsifying, encapsulating, entrapping or lyophilizing processes.

Hereinafter the phrases “physiologically suitable carrier” and “pharmaceutically acceptable carrier” are interchangeably used and refer to a carrier or a diluent that does not cause significant irritation to an organism and does not abrogate the biological activity and properties of the administered conjugate.

Herein the term “excipient” refers to an inert substance added to a pharmaceutical composition to further facilitate processes and administration of the active ingredients. Examples, without limitation, of excipients include calcium carbonate, calcium phosphate, various sugars and types of starch, cellulose derivatives, gelatin, vegetable oils and polyethylene glycols.

Techniques for formulation and administration of active ingredients may be found in “Remington's Pharmaceutical Sciences,” Mack Publishing Co., Easton, Pa., latest edition, which is incorporated herein by reference.

While various routes for the administration of active ingredients are possible, and were previously described; for the purpose of the present invention, the topical route is preferred, and is assisted by a topical carrier. The topical carrier is one, which is generally suited for topical active ingredients administration and includes any such materials known in the art. The topical carrier is selected so as to provide the composition in the desired form e.g., as a liquid or non-liquid carrier; lotion, cream, paste, gel, powder, ointment, solvent, liquid diluent, drops and the like, and may be comprised of a material of either naturally occurring or synthetic origin. It is essential, clearly, that the selected carrier does not adversely affect the active agent or other components of the topical formulation, and which is stable with respect to all components of the topical formulation. Examples of suitable topical carriers for use herein include water, alcohols and other nontoxic organic solvents, glycerin, mineral oil, silicone, petroleum jelly, lanolin, fatty acids, vegetable oils, parabens, waxes, and the like. Preferred formulations herein are colorless, odorless ointments, liquids, lotions, creams and gels.

Ointments are semisolid preparations, which are typically based on petrolatum or other petroleum derivatives. The specific ointment base to be used, as will be appreciated by those skilled in the art, is one that will provide for optimum active ingredients delivery, and, preferably, will provide for other, desired characteristics as well, e.g., emolliency or the like. As with other carriers or vehicles, an ointment base should be inert, stable, nonirritating and nonsesitizing. As explained in Remington: The Science and Practice of Pharmacy, 19th Ed. (Easton, Pa.:. Mack Publishing Co. 1995), at pages 1399-1404, ointment bases may be grouped in four classes: oleaginous bases; emulsifiable bases; emulsion bases; and water-soluble bases. Oleaginous ointment bases include, for example, vegetable oils, fats obtained from animals, and semisolid hydrocarbons obtained from petroleum. Emulsifiable ointment bases, also known as absorbent ointment bases, contain little or no water and include, for example, hydroxystearin sulfate, anhydrous lanolin and hydrophilic petrolatum. Emulsion ointment bases are either water-in-oil (W/O) emulsions or oil-in-water (O/W) emulsions, and include, for example, cetyl alcohol, glyceryl monostearate, lanolin and stearic acid. Preferred water-soluble ointment bases are prepared from polyethylene glycols of varying molecular weight; again, reference may be made to Remington: The Science and Practice of Pharmacy for further information.

Lotions are preparations to be applied to the skin surface without friction, and are typically liquid or semiliquid preparations, in which solid, particles, including the active agent, are present in a water or alcohol base. Lotions are usually suspensions, of solids, and may comprise a liquid oily emulsion of the oil-in-water type. Lotions are preferred formulations herein for treating large body areas, because of the ease of applying a more fluid composition. It is generally necessary that the insoluble matter in a lotion be finely divided. Lotions will typically contain suspending agents to produce better dispersions as well as active ingredients useful for localizing and holding the active agent in contact with the skin, e.g., methylcellulose, sodium carboxymethylcellulose, or the like.

Creams containing the selected active ingredients are, as known in the art, viscous liquid or semisolid emulsions, either oil-in-water or water-in-oil. Cream bases are water-washable, and contain an oil phase, an emulsifier and an aqueous phase. The oil phase, also sometimes called the “internal” phase, is generally comprised of petrolatum and a fatty alcohol such as cetyl or stearyl alcohol; the aqueous phase usually, although not necessarily, exceeds the oil phase in volume, and generally contains a humectant. The emulsifier in a cream formulation, as explained in Remington, supra is generally a nonionic, anionic, cationic or amphoteric surfactant.

Gel formulations are preferred for application to the scalp. As will be appreciated by those working in the field of topical active ingredients formulation, gels are semisolid, suspension-type systems. Single-phase gels contain organic macromolecules distributed substantially uniformly throughout the carrier liquid, which is typically aqueous, but also, preferably, contain an alcohol and, optionally, an oil.

Carriers for nucleic acids include but are not limited to, liposomes including targeted liposomes, nucleic acid complexing agents, viral coats and the like. However, transformation with naked nucleic acids may also be employed.

Various additives, known to those skilled in the art, may be included in the topical formulations of the invention. For example, solvents may be used to solubilize certain active ingredients substances. Other optional additives include skin permeation enhancers, opacifiers, anti-oxidants, gelling agents, thickening agents, stabilizers, and the like.

As has already been mentioned hereinabove, topical preparations for the treatment of wounds according to the present invention may contain other pharmaceutically active agents or ingredients; those traditionally used for the treatment of such wounds. These include immunosuppressants, such as cyclosporine, antimetabolites, such as methotrexate, corticosteroids, vitamin D and vitamin D analogs, vitamin A or its analogs, such etretinate, tar, coal tar, anti pruritic and keratoplastic agents, such as cade oil, keratolytic agents, such as salicylic acid, emollients, lubricants, antiseptic and disinfectants, such as the germicide dithranol (also known as anthralin) photosensitizers, such as psoralen and methoxsalen and UV irradiation. Other agents may also be added, such as antimicrobial agents, antifingal agents, antibiotics and anti-inflammatory agents. Treatment by oxygenation (high oxygen pressure) may also be co-employed.

The topical compositions of the present invention may also be delivered to the skin using conventional dermal-type patches or articles, wherein the active ingredients composition is contained within a laminated structure, that serves as a drug delivery device to be affixed to the skin. In such a structure, the active ingredients composition is contained in a layer, or “reservoir”, underlying an upper backing layer. The laminated structure may contain a single reservoir, or it may contain multiple reservoirs. In one embodiment, the reservoir comprises a polymeric matrix of a pharmaceutically acceptable contact adhesive material that serves to affix the system to the skin during active ingredients delivery. Examples of suitable skin contact adhesive materials include, but are not limited to, polyethylenes, polysiloxanes, polyisobutylenes, polyacrylates, polyurethanes; and the like. The particular polymeric adhesive selected will depend on the particular active ingredients vehicle, etc., i.e., the adhesive must be compatible with all components of the active ingredients-containing composition. Alternatively, the active ingredients-containing reservoir and skin contact adhesive are present as separate and distinct layers, with the adhesive underlying the reservoir which, in this case, may be either a polymeric matrix as described above, or it may be a liquid or hydrogel reservoir, or may take some other form.

The backing layer in these laminates, which serves as the upper surface of the device, functions as the primary structural element of the laminated structure and provides the device with much of its flexibility. The material selected for the backing material should be selected so that it is substantially impermeable to the active ingredients and to any other components of the active ingredients-containing composition, thus preventing loss of any components through the upper surface of the device. The backing layer may be either occlusive or nonocclusive, depending on whether it is desired that the skin become hydrated during active ingredients delivery the backing is preferably made of a sheet or film of a preferably flexible elastomeric material. Examples of polymers that are suitable for the backing layer include polyethylene, polypropylene, and polyesters.

During storage and prior to use, the laminated structure includes a release-liner. Immediately prior to use, this layer is removed from the device to expose the basal surface thereof, either the active ingredients reservoir or a separate contact adhesive layer, so that the system may be affixed to the skin. The release liner should be made from an active ingredients/vehicle impermeable material.

Such devices may be fabricated using conventional techniques, known in the art, for example by casting a fluid admixture of adhesive, active ingredients and vehicle onto the backing layer, followed by lamination of the release liner. Similarly, the adhesive mixture may be cast onto the release liner, followed by lamination of the backing layer. Alternatively, the active ingredients reservoir may be prepared in the absence of active ingredients or excipient, and then loaded by “soaking” in an active ingredients/vehicle mixture.

As with the topical formulations of the invention, the active ingredients composition contained within the active ingredients reservoirs of these laminated system may contain a number of components. In some cases, the active ingredients may be delivered “neat,” i.e., in the absence of additional liquid. In most cases, however, the active ingredients will be dissolved, dispersed or suspended in a suitable pharmaceutically acceptable vehicle, typically a solvent or gel. Other components, which may be present, include preservatives, stabilizers, surfactants, and the like.

The pharmaceutical compositions herein described may also comprise suitable solid or gel phase carriers or excipients. Examples of such carriers or excipients include, but are not limited to, calcium carbonate calcium phosphate, various sugars, starches, cellulose derivatives, gelatin and polymers such as polyethylene glycols.

Other suitable routes of administration may, for example, include oral, rectal, transmucosal, transdermal, intestinal or parenteral delivery, including intramuscular, subcutaneous and intramedullary injections as well as intrathecal, direct intraventricular, intravenous, intaperitoneal, intranasal, or intraocular injections.

Pharmaceutical compositions for use in accordance with the present invention thus may be formulated in conventional manner using one or more pharmaceutically acceptable carriers comprising excipients and auxiliaries, which facilitate processing of the active ingredients into preparations which, can be used pharmaceutically. Proper formulation is dependent upon the route of administration chosen.

For injection, the active ingredients of the invention may be formulated in aqueous solutions, preferably in physiologically compatible buffers such as Hank's solution, Ringer's solution, or physiological saline buffer. For transmucosal administration, penetrants are used in the formulation. Such penetrants are generally known in the art.

For oral administration, the active ingredients can be formulated readily by combining the active ingredients with pharmaceutically acceptable carriers well known in the art. Such carriers enable the active ingredients of the invention to be formulated as tablets, pills, dragees, capsules, liquids gels, syrups, slurries, suspensions, and the like, for oral ingestion by a patient. Pharmacological preparations for oral use can be made using a solid excipient optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carbomethylcellulose; and/or physiologically acceptable polymers such as polyvinylpyrrolidone (PVP). If desired, disintegrating agents may be added, such as cross-linked polyvinyl pyrrolidone, agar, or alginic acid or a salt thereof such as sodium alginate.

Dragee cores are provided with suitable coatings. For this purpose, concentrated sugar solutions may be used which may optionally contain gum arabic; talc, polyvinyl pyrrolidone, carbopol gel, polyethylene glycol, titanium dioxide, lacquer solutions and suitable organic solvents or solvent mixtures. Dyestuffs or pigments may be added to the tablets or dragee coatings for identification or to characterize different combinations of active ingredient doses.

Pharmaceutical compositions, which can be used orally, include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the active ingredients may be dissolved or suspended in suitable liquids, such as fatty oils, liquid paraffin, or liquid polyethylene glycols. In addition, stabilizers may be added. All formulations for oral administration should be in dosages suitable for the chosen route of administration.

For buccal administration, the compositions may take the form of tablets or lozenges formulated in conventional manner.

For administration by inhalation, the active ingredients for use according to the present invention are conveniently delivered in the form of an aerosol spray presentation from a pressurized pack or a nebulizer with the use of a suitable propellant, e.g., dichlorodifluoromethane, trichlorofluoromethane, dichlorotetrafluoroethane or carbon dioxide. In the case of a pressurized aerosol, the dosage unit may be determined by providing a valve to deliver a metered amount. Capsules and cartridges of, e.g.; gelatin for use in an inhaler or insufflator may be formulated containing a powder mix of the active ingredient and a suitable powder base such, as lactose or starch.

The active ingredients described herein may be formulated for parenteral, administration, e.g., by bolus injection or continues infusion. Formulations for injection may be presented in unit dosage form, e.g., in ampoules or in multidose containers with optionally, an added preservative. The compositions may be suspensions, solutions or emulsions in oily or aqueous vehicles, and may contain formulatory agents such as suspending, stabilizing and/or dispersing agents.

Pharmaceutical compositions for parenteral administration include aqueous solutions of the active preparation in water-soluble form. Additionally, suspensions of the active ingredients; may be prepared as appropriate oily injection suspensions. Suitable lipophilic solvents or vehicles include fatty oils such as sesame oil, or synthetic fatty acids esters such as ethyl oleate, triglycerides or liposomes. Aqueous injection suspensions may contain substances, which increase the viscosity of the suspension, such as sodium carboxymethyl cellulose, sorbitol or dextran. Optionally, the suspension may also contain suitable stabilizers or agents which increase the solubility of the active ingredients to allow for the preparation of highly concentrated solutions.

Alternatively, the active ingredient may be in powder form for constitution with a suitable vehicle, e.g., sterile, pyrogen-free water, before use

The active ingredients of the present invention may also be formulated in rectal compositions such as suppositories or retention enemas, using, e.g., conventional suppository bases such as cocoa butter or other glycerides, The pharmaceutical compositions herein described may also comprise suitable solid of gel phase carriers or excipients. Examples of such carriers or excipients include, but are not limited to, calcium carbonate, calcium phosphate, various sugars, starches, cellulose derivatives, gelatin and polymers such as polyethylene glycols.

Pharmaceutical compositions suitable for use in context of the present invention include compositions wherein the active ingredients are contained in an amount effective to achieve the intended purpose. More specifically, a therapeutically effective amount means an amount of active ingredient effective to prevent, alleviate or ameliorate symptoms of disease or prolong the survival of the subject being treated.

Determination of a therapeutically effective amount is well within the capability of those skilled in the art, especially in light of the detailed disclosure provided herein.

For any active ingredient used in the methods of the invention, the therapeutically effective amount or dose can be estimated initially from activity assays in animals. For example, a dose can be formulated in animal models to achieve a circulating concentration range that includes the IC₅₀ as determined by activity assays. Such information can be used to more accurately determine useful doses in humans.

Toxicity and therapeutic efficacy of the active ingredients described herein can be determined by standard pharmaceutical-procedures in experimental animals, e.g., by determining the IC₅₀ and the LD₅₀ (lethal dose causing death in 50% of the tested animals) for a subject active ingredient. The data obtained from these activity assays and animal studies can be used in formulating a range of dosage for use in human.

The dosage may vary depending upon the dosage form employed and the route of administration utilized. The exact formulation; route of administration and dosage can be chosen by the individual physician in view of the patient's condition. (See e.g., Fingl, et al., 1975, in “The Pharmacological Basis of Therapeutics”, Ch. 1 p. 1).

Dosage amount and interval may be adjusted individually to provide plasma levels of the active moiety which are sufficient to maintain the modulating effects, termed the minimal effective concentration (MEC). The MEC will vary for each preparation, but can be estimated from in vitro data; e.g., the concentration necessary to achieve 50-90% inhibition of a kinase may be ascertained using the assays described herein. Dosages necessary to achieve the MEC will depend on individual characteristics and route of administration. HPLC assays or bioassays can be used to determine plasma concentrations.

Dosage intervals can also be determined using; the MEC value. Preparations should be administered using a regimen, which maintains plasma levels above the MEC for 10-90% of the time, preferable between 30-90% and most preferably 50-90%.

Depending on the severity and responsiveness of the condition to be treated, dosing can also be a single administration of a slow release composition described hereinabove, with course of treatment lasting from several days to several weeks or until cure is effected or diminution of the disease state is achieved.

The amount of a composition to be administered will, of course, be dependent on the subject being treated, the severity of the affliction, the manner of administration, the judgment of the prescribing physician, etc.

Compositions of the present invention may, if desired, be presented in a pack or dispenser device, such as an FDA approved kit, which may contain one or more unit dosage forms containing the active ingredient. The pack may, for example, comprise metal or plastic foil, such as a blister pack. The pack or dispenser device may be accompanied by, instructions for administration. The pack or dispenser may also be accompanied by a notice associated with the containers in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert. Compositions comprising an active ingredient of the invention formulated in a compatible pharmaceutical carrier may also be prepared, placed in an appropriate container, and labeled for treatment of an indicated condition.

Preferably, the invention encompasses a pharmaceutical carrier adapted for application to a wound. Such carriers are well known in the art and may optionally include, but are not limited to, one or more of an ointment, a gels a liquid, a cream, a paste, a lotion, a spray, a suspension, a powder, a dispersion, a salve, or any other pharmaceutical composition adapted for topical application, as well as a bandage or other wound covering and/or solid support that is adapted for administration of heparanase to the wound, or a combination thereof.

As used herein, the term “modulate” includes substantially inhibition slowing or reversing the progression of a disease, substantially ameliorating clinical symptoms of a disease or condition, or substantially preventing the appearance of clinical symptoms of a disease or condition. A “modulator” therefore includes an agent which may modulate a disease or condition. Modulation of viral, protozoa and bacterial infections includes any effect which substantially interrupts, prevents or reduces any viral, bacterial or protozoa activity and/or stage of the virus, bacterium or protozoon life cycle, or which reduces or prevents infection by the virus, bacterium or protozoon in a subject, such as a human or lower animal.

Anti-heparanase antibodies, raised against the recombinant enzyme, would be useful for immunodetection and diagnosis of micrometastases, autoimmune lesions and renal failure in biopsy specimens, plasma samples, and body fluids. Such antibodies may also serve as neutralizing agents for heparanase activity.

The genomic heparanase sequences described herein can be used to construct knock-in and knock-out constructs. Such constructs include a fragment of 10-20 Kb of a heparanase locus and a negative and a positive selection markers and can be used to provide heparanase knock-in and knock-out animal models by methods known to the skilled artisan. Such animal models can be used for studying the function of heparanase in developmental processes, and in normal as well as pathological processes. They can also serve as an experimental model for testing drugs and gene therapy protocols. The complementary heparanase sequence (cDNA) can be used to derive transgenic animals, overexpressing heparanase for same. Alternatively, if cloned in the antisense orientation, the complementary heparanase sequence (cDNA) can be used to derive transgenic animals under-expressing heparanase for same.

The heparanase promoter sequences described herein and other cis regulatory elements linked to the hepatanase locus can be used to regulated the expression of genes. For example, these promoters can be used to direct the expression of a cytotoxic protein, such as TNF, in tumor cells. It will be appreciated that heparanase itself is abnormally expressed under the control of its own promoter and other cis acting elements in a variety of tumors, and its, expression is correlated with metastasis. It is also abnormally highly expressed inflammatory cells. The introns of the heparanase gene can be used for the same purpose, as it is known that introns, especially upstream introns include cis acting element which affect, expression. A heparanase promoter fused to a reporter protein can be used to study/monitor its activity.

The polynucleotide sequences described herein can also be used to provide. DNA vaccines which will elicit in vivo anti heparanase antibodies. Such vaccines can therefore be used to combat inflammatory and cancer.

Antisense oligonucleotides derived according to the heparanase sequences described herein, especially such oligonucleotides supplemented with ribozyme activity, can be used to modulate heparanase expression. Such oligonucleotides can be from the coding region, from the introns or promoter specific. Antisense heparanase nucleic acid constructs can similarly function, as well known in the art.

The heparanase sequences described herein can be used to study the catalytic mechanism of heparanase. Carefully selected site directed mutagenesis can be employed to provide modified heparanase proteins having modified characteristics in terms of, for example, substrate specificity, sensitivity to inhibitors, etc.

While studying heparanase expression in a variety of cell types alternatively spliced transcripts were identified. Such transcripts if found characteristic of certain pathological conditions can be used as markers for such conditions. Such transcripts are expected to direct the synthesis of heparanases with altered functions.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

Generally, the nomenclature used herein and the laboratory procedures in recombinant DNA technology described below are those well known and commonly employed in the art. Standard techniques are used for cloning, DNA and RNA isolation, amplification and purification. Generally enzymatic reactions involving DNA ligase, DNA polymerase, restriction endonucleases and the like are performed according to the manufacturers specifications. These techniques and various other techniques are generally performed according to Sambrook et al., Molecular Cloning—A Laboratory Manual, Cold Spring Harbor Laboratory, Cold Spring Harbor, N.Y. (1989), which is incorporated herein by reference. Other general references are provided throughout this document. The procedures therein are believed to be well known in the art and are provided for the convenience of the reader. All the information contained therein is incorporated herein by reference.

The following protocols and experimental details are referenced in the Examples that follow:

Purification and characterization of heparanase from a human hepatoma cell line and human placenta: A human hepatoma cell line (Sk-hep-1) was chosen as a source for purification of a human tumor-derived heparanase. Purification was essentially as described in U.S. Pat. No. 5,362,641 to Fuks, which is incorporated by reference as if fully set forth herein. Briefly, (500 liter)?, 5×10¹¹ cells were grown in suspension and the heparanase enzyme was purified about 240,000 fold by applying the following steps: (i) cation exchange (CM-Sephadex) chromatography performed at pH 6.0, 0.3-1.4 M NaCl gradient; (ii) cation exchange (CM-Sephadex) chromatography performed at pH 7.4 in the presence of 0.1% CHAPS, 0.3-1.1 M NaCl gradient; (iii) heparin-Sepharose chromatography performed at pH 7.4 in the presence of 0.1% CHAPS, 0.35-1.1 M NaCl gradient; (iv) ConA-Sepharose chromatography performed at pH 6.0 in buffer containing 0.1% CHAPS and 1 M NaCl, elution with 0.25 M α-methyl mannoside; and (v) HPLC cation exchange (Mono-S) chromatography performed at pH 7.4 in the presence of 0.1% CHAPS, 0.25-1 M NaCl gradient.

Active fractions were pooled, precipitated with TCA and the precipitate subjected to SDS polyacrylamide gel electrophoresis and/or tryptic digestion and reverse phase HPLC. Tryptic peptides of the purified protein were separated by reverse phase HPLC (C8-column) and homogeneous peaks were subjected to amino acid sequence analysis.

The purified enzyme was applied to reverse phase HPLC and subjected to N-terminal amino acid sequencing using the amino acid sequencer (Applied Biosystems).

Cells: Cultures of bovine corneal endothelial cells (BCECs) were established from steer eyes as previously described (19, 38). Stock cultures were maintained in DMEM (1 g glucose/liter) supplemented with 10% newborn calf serum and 5% FCS. bFGF (1 ng/ml) was added every other day during the phase of active cell growth (13, 14).

Cells for Wound Healing and Angiogenesis Experiments were prepared as follows: The methylcholanthrene: induced non-metastatic Eb T-lymphoma cells were grown in RPMI 1640 supplemented with 10% FCS [Vlodavsky, I. et al. Mammalian heparanase: gene cloning, expression and function in tumor progression and metastasis. Nat Med 5, 793-802 (1999)]. Bovine aortic EC were cultured in DMEM (1 gram glucose/liter) supplemented with 10% calf serum [Vlodavsky, I. in Current protocols in Cell Biology, Vol. I, Suppl. 1, Eds. J. S. Bonifacino, M. Dasso, J. B. Harford, J. Lippincott-Schwartz & K. M. Yamada, John Wiley & Sons, New York, N.Y., pp. 10.4.1-10.4.1⁴ (1999)] (Life Technologies). Bovine corneal EC were established and maintained as described [Vlodavsky, I. in Current protocols in Cell Biology, Vol. 1, Suppl. I, Eds. J. S. Bonifacino, M. Dasso, J. B. Harford J. Lippincott-Schwartz & K. M. Yamada, John Wiley & Sons, New York, N.Y., pp. 10.4.1-10.4.14 (1999)]. Cells were cultured at 37° C. in 10% CO₂ humidified incubators [Vlodavsky, I. in Current protocols in Cell Biology, Vol. I, Suppl. I, Eds. J. S. Bonifacino, M. Dasso, J. B. Harford, J. Lippincott-Schwartz & K. M. Yamada, John Wiley & Sons, New York, N.Y., pp. 10.4.1-10.4.14 (1999)]. Clone F32 of BaF3F lymphoid cells kindly provided by Dr. D. Ornitz (Department of Molecular Biology, Washington University in St. Louis), were grown in RPMl 1640 medium supplemented with 10% FCS, 10% interleukin-3 conditioned medium produced by X63-1L3 WHEI cells, L-glutamine and antibiotics [Ornitz, D. M. et al. Heparin is required for cell-free binding of basic fibroblast growth factor to a soluble receptor and for mitogenesis in whole cells. Mol Cell Biol 12,240-247 (1992)].

Preparation of dishes coated with ECM: BCECs (second to fifth passage) were plated into 4-well plates at an initial density of 2×10⁵ cells/ml, and cultured in sulfate-free Fisher medium plus 5% dextran T-40 for 12 days. Na₂³⁵SO₄ (25 μCi/ml) was added on day 1 and 5 after seeding and the cultures were incubated with the label without medium change. The subendothelial ECM was exposed by dissolving (5 min., room temperature) the cell layer with PBS containing 0.5% Triton X-100 and 20 mM NH₄OH, followed by four washes with PBS. The ECM remained, intact, free of cellular debris and firmly attached to the entire area of the tissue culture dish (19, 22).

To prepare soluble sulfate labeled proteoglycans (peak I material), the ECM was digested with trypsin (25 μg/ml, 6 h, 37° C.), the digest was concentrated by reverse dialysis and the concentrated material was applied onto a Sepharose 6B gel filtration column. The resulting high molecular weight material (Kav<0.2, peak D) was collected. More than 80% of the labeled material was shown to be composed of heparan sulfate proteoglycans (11, 39).

For Wound Healing and Angiogenesis Experiments, bovine corneal EC were cultured as described above except that 5% dextran T-40 was included in the growth medium and the cells were maintained without addition of bFGF for 12 days. The subendothelial ECM was exposed by dissolving the cell layer with PBS containing 0.5% Triton X-100 and 20 mM NH₄OH, followed by four washed in PBS [Vlodavsky, I. in Current protocols in Cell Biology, Vol. I, Suppl. I, Eds. J. S. Bonifacino, M. Dasso, J. B. Harford, J. Lippincott-Schwartz & K. M. Yamada, John Wiley & Sons, New York, N.Y., pp. 10.4.1-10.4.14 (1999)]. The ECM remained intact, free of cellular debris and firmly attached to the entire area of the tissue culture, dish [Vlodavsky, I. in Current protocols in Cell Biology, Vol. I, Suppl. I, Eds. J. S. Bonifacino, M. Dasso, J. B. Harford, J. Lippincott-Schwartz & K. M. Yamada, John Wiley & Sons, New York, N.Y., pp. 10.4.1-10.4.14 (1999)]. For preparation of sulfate-labeled ECM, corneal endothelial cells were cultured in the presence of Na₂[³⁵S]O₄ (Amersham) added (25 μCi/ml) one day and 5 days after seeding and the cultures were incubated with the label without medium change [Vlodavsky, I. in Current protocols in Cell Biology, Vol. I, Suppl. I, Eds. J. S. Bonifacino, M. Dasso, J. B. Harford, J. Lippincott-Schwartz & K. M. Yamada, John Wiley & Sons, New York, N.Y., pp. 10.4.1-10.4.14 (1999)]. Ten to twelve days after seeding, the cell monolayer was dissolved and the ECM exposed, as described above.

Heparanase activity: Cells: (1×10⁶/35-mm dish), cell lysates or conditioned media were incubated on top of ³⁵S-labeled ECM (18 h, 37° C.) in the presence of 20 mM phosphate buffer (pH 6.2). Cell lysates and conditioned media were also incubated with sulfate labeled peak I material (110-20 μl). The incubation medium was collected, centrifuged (18,000×g, 4° C.-3 min.), and sulfate labeled material analyzed by gel filtration on a Sepharose CL-6B column (0.9×30 cm). Fractions (0.2 ml) were eluted with PBS at a flow rate of 5′ ml/h and counted for radioactivity using Bio-fluor scintillation fluid. The excluded volume (VO) was marked by blue dextran and the total included volume (Vt) by phenol red. The latter was shown to comigrate with free sulfate (7, 11, 23). Degradation fragments of HS side chains were eluted from Sepharose 6B at 0.5<Kav<0.8 (peak, II) (7, 11, 23). A nearly intact HSPG released from ECM by trypsin—and, to a lower extent, during incubation with PBS alone—was, eluted next to V_{O} (Kav<0.2, peak 1). Recoveries of labeled material applied on the columns ranged from 85 to 95% in different experiments (11). Each experiment was performed at least three times and the variation of elution positions (Kav values) did not exceed +/−15%.

For Wound Healing and Angiogenesis experiments, degradation of sulfate is labeled ECM by heparanase was determined as described [Vlodavsky, I. et al. Mammalian heparanase: gene cloning, expression and function in tumor progression and metastasis. Nat Med 5, 793-802 (1999); Vlodavsky, I. in Current protocols in Cell Biology, Vol. I, Suppl. I, Eds. J. S. Bonifacino, M. Dasso, J. B. Harford, J. Lippincott-Schwartz & K. M. Yamada, John Wiley & Sons, New York, N.Y., pp. 10.4.1-10.4.14 (1999)]. Briefly, ECM was incubated (24 hours, 37° C., pH 6.2) with recombinant heparanase or hpa-transfected cells and sulfate labeled material released into the incubation medium was analyzed by gel filtration on a Sepharose 6B column [Vlodavsky, I. et al Mammalian heparanase: gene cloning, expression and function in tumor progression and metastasis. Nat Med 5, 793-802 (1999); Vlodavsky, I. in Current protocols in Cell Biology, Vol. I, Suppl. I, Eds. J. S. Bonifacino, M. Dasso, J. B. Harford, J. Lippincott-Schwartz & K. M. Yamada, John Wiley & Sons, New York, N.Y., pp. 10.4.1-10.4.14 (1999)]. Intact HSPGs were eluted just after the void volume (Kav<0.2, peak I) and HS degradation fragments eluted with 0.5<Kav<0.8 (peak II) [Vlodavsky, I. et al. Mammalian heparanase: gene cloning, expression and function in tumor progression and metastasis. Nat Med 5, 793-802 (1999); Vlodavsky, I. in Current protocols in Cell Biology, Vol. I, Suppl. I, Eds. J. S. Bonifacino, M. Dasso, J. B. Harford, J. Lippincott-Schwartz & K. M. Yamada, John Wiley & Sons, New York, New York, pp. 10.4.1-10.4.14 (1999)]. Release of ECM-bound bFGF: Recombinant bFGF was iodinated using chloramine T and bound to ECM as described [Vlodavsky, I. et al Inhibition of tumor metastasis by heparanase inhibiting species of heparin. Invasion Metastasis 14, 290-302 (1994)]. Briefly, tissue culture plates coated with ECM were incubated (3 hours, 24° C.) with 0.1 ng/ml ¹²⁵]-bFGF in PBS containing 0.02% gelatin. Unbound bFGF was removed by three washes with PBS containing 0.02% gelatin. The ECM was then incubated with increasing concentrations of recombinant heparanase at 37° C. for 3 hours. The incubation media were collected and counted in a γ-counter to determine the amount of released ¹²⁵I-bFGF. The remaining ECM was incubated (3 hours, 37 C) with 1N NaOH and the solubilized radioactivity counted in a γ-counter. The percentage of released ¹²⁵I-bFGF was calculated from the total ECM-associated radioactivity [Vlodavsky, I. et al. Inhibition of tumor metastasis by heparanase inhibiting species of heparin. Invasion Metastasis 14, 290-302 (1994)].

Release of endogenous bFGF from ECM: ECM coated 35 mm dishes were incubated (24° C., 4 hours) with either 1 ml heparanase reaction mixture (150 mM NaCl, 50 mM buffer phosphate-citrate, pH 6.2, 0.2% bovine serum albumin) or reaction buffer containing 0.5 μg/ml recombinant heparanase. ELISA (Quantikine HS human FGF basic, R&D systems) tested aliquots of the incubation medium for bFGF content. Each sample was tested in triplicates and the variation between different determinations did not exceed ±7% of the mean.

Effect of HS fragments released by heparanase from cell surfaces and ECM on BaF3 cell proliferation: Vascular EC and intact subendothelial ECM were incubated (4 hours, 37° C.) with 1 μg/ml heparanase (P50). Increasing amounts of the incubation medium containing the released HS degradation fragments were then added to BaF3 cells (2×10⁴ cells/well; 96 well plate) in the presence of 5 ng/ml bFGF. Forty-eight hours later, ³H-thymidine (1 μCi/well) (Amersham Pharmacia Biotech) was added for 6 hours, followed by cell-harvesting and measurement of ³H-thymidine incorporation [Miao, H. Q., Ornitz, D. M., Aingorn, E., Ben-Sasson, S. A. & Vlodavsky, I. Modulation of fibroblast growth factor-2 receptor binding, dimerization, signaling, and angiogenic activity by a synthetic heparin-mimicking polyanionic compound. J Clin Invest 99, 1565-1575 (1997); Ornitz, D. M. et al. Heparin is required for cell-free binding of basic fibroblast growth factor to a soluble receptor and for mitogenesis in whole cells. Mol Cell Biol 12, 240-247 (1992)].

Immunohistochemistry: Immunohistochemistry was performed as described before with minor modifications [Vlodavsky, I. et al. Mammalian heparanase: gene cloning, expression and function in tumor progression and metastasis. Nat Med 5, 793-802 (1999)]. Briefly, 5 m sections were deparaffinized and rehydrated. Tissue was then denatured for 3 minutes in a microwave oven in citrate buffer (0.01 M, PH 6.0). Blocking steps included successive incubations in 0.2% glycine, 3% H₂O₂ in methanol and 5% goat serum. Sections were incubated with a monoclonal (mAb 92.4) anti-human heparanase antibody diluted 1:3 in PBS, or with DMEM supplemented with 10% horse serum as control, diluted as above, followed by incubation with HRP conjugated goat anti-mouse IgG+IgM antibody (Jackson). mAb 92.4 is directed against the N-terminus region of the 50 kDa enzyme. The preparation and specificity of this mAb were previously described and demonstrated [Vlodavsky, I. et al. Mammalian heparanase: gene cloning, expression and function in tumor progression and metastasis. Nat Med 5, 793-802 (1999)]. Color was developed using Zymed AEC substrate kit (Zymed) for 10 minutes, followed by counter stain with Mayer's hematoxylin [Vlodavsky, I. et al. Mammalian heparanase: gene cloning, expression and function in tumor progression and metastasis. Nat Med 5, 793-802 (1999)].

Matrigel plug assay: Matrigel plug assay was performed as previously; described [Passaniti, A. et al. A simple, quantitative method for assessing angiogenesis and antiangiogenic agents using reconstituted basement membrane, heparin, and fibroblast growth factor. Lab Invest 67, 519-528 (1992)]. Six week, old male BALB/c mice (n=5) were injected subcutaneously at the lateral abdominal area with 0.4 ml of Matrigel (kindly provided by Dr. H. Kleinmann, NIDR, NIH, Bethesda Md.) premixed on ice with 2×10⁶ hpa transfected Eb murine lymphoma cells highly expressing and secreting a recombinant heparanase [Vlodavsky, I. et al. Mammalian heparanase: gene cloning, expression and function in tumor progression and metastasis. Nat Med 5, 793-802 (1999)]. Control mice were injected with Matrigel mixed with mock-transfected Eb cells, lacking hepatanase. Matrigel plugs were removed 7 days post implantation, photographed and transferred to tubes containing 0.4 ml DDW. Plugs were homogenized with a Politron homogenizer; until complete disintegration. The debris was centrifuged and the hemoglobin containing supernatant was collected. Hemoglobin content was determined using Drabkin reagent (Sigma) and quantitated against a standard curve of plasma hemoglobin.

Wound formation and treatment: Full-thickness wound were created with a 8 mm punch at the back of 10 anesthetized Balb C male mice skin. Purified 50 kDa active heparanase enzyme was applied topically twice a day at I [g/wound (about 2 ng/mm²) for 4 days, and once a day for the next 0.3 days. Wound closure was monitored after seven days with a fine digital caliber. Average, Wound areas were statistically analyzed by the two-sample t-test assuming equal variances.

3 sided ischemic wound healing: Acute, 3-sided, ischemic full thickness wounds were created by connecting two longitudinal incisions, each 7 cm in length, at the caudal end with a third, 3 cm, incision across the midline. The flap was elevated to the base of the cranial pedicle, replaced in its bed and secured with sutures. One application of purified recombinant human heparanase enzyme (10 μg or 50 μg per wound) was administered to the wound surface before suturing. Controls received no heparanase, and were treated with once daily saline irrigations of the sutured incisions for 3 days after wound closure. Histological examination of multiple samples from the healed incisions was performed 14 days post suturing (see below), and wound healing scored as one of three categories: Scar no epithelialization; Small Scar-partial epithelialization; No Scar-full epithelialization (Cure). Two preparations of the purified heparanase were tested, in two different series of experiments. Sample size was 8 rats per treatment in each series.

Histological examination of heparanase treated wounds: For histological examination, wound areas including the underlying granulation tissue, were removed and formalin-fixed paraffin-embedded sections were stained With hematoxylin-eosin. Immunohistochemistry was performed as previously described [Ilan N., S. Mahooti D. L. Rimm and Joseph A. Madri. 1999. PECAM-1 (CD31) functions as a reservoir for and a modulator of tyrosine-phosphorylated beta-catenin. J. Cell Sci. 112: 3005-3014]. Briefly, sections were subjected to antigen retrieval, blocked with 10% normal horse serum and incubated with anti-PECAM-1, anti-PCNA (Santa Cruz) and affinity purified anti-heparanase polyclonal antibodies over night at 4° C. Sections were then washed three times with PBS and staining was visualized by the Vectastain ABC kit and DAB substrate (Vector).

Cloning of hpa cDNA: cDNA clones 257548 and 260138 were obtained from the I.M.A.G.E Consortium (2130 Memorial Parkway SW, Hunstville, Ala. 35801). The cDNAs were originally cloned in EcoRI and NotI cloning sites in the plasmid vector pT3T7D-Pac. Although these clones are reported to be somewhat different, DNA sequencing demonstrated that these clones are identical to one another. Marathon RACE (rapid amplification of cDNA ends) human placenta (poly-A) cDNA composite was a gift of Prof. Yossi Shiloh of Tel Aviv University. This composite is vector free, as it includes reverse transcribed cDNA fragments to which double, partially single stranded adapters are attached on both sides. The construction of the specific composite employed is described in reference 39a.

Amplification of hp3 PCR fragment was performed according to the protocol provided by Clontech laboratories. The template used for amplification was a sample taken from the above composite. The primers used for amplification were:

First step:

SEQ ID NO:1

5′-primer: AP1:
5′-CCATCCTAATACGACTCACT ATAGGGC-3′,;

SEQ ID NO:2

3′-primer: HPL229:
5′-GTAGTGATGCCA TGTAACTGAATC-3′,.

Second step: nested 5′-primer: AP2: 5′-ACTCACTATAGGGCTCG AGCGGC-3′, SEQ ID NO:3; nested 3′-primer: HPL171: 5′-GCATCTTAGCCGTCTTTCTTCG-3′, SEQ ID NO:4. The HPL229 and HPL171 were selected according to the sequence of the EST clones. They include nucleotides 933-956 and 876-897 of SEQ ID NO:9, respectively.

PCR program was 94° C.-4 min., followed by 30 cycles of 94° C.-40 sec., 62° C.-1 min., 72° C.-2.5 min. Amplification was performed with Expand High Fidelity (Boehringer Mannheim). The resulting, ca. 900 bp hp3 PCR product was digested with BfrI and PvuII. Clone 257548 (phpa1) was digested with EcoRI, followed by end filling and was then further digested with BfrI. Thereafter the PvuII—BfrI fragment of the hp3 PCR product was cloned into the blunt end—BfrI end of clone phpa1 which resulted in having the entire cDNA cloned in pT3T7-pac vector, designated phpa2.

RT-PCR: RNA was prepared using TRI-Reagent (Molecular research center Inc.) according to the manufacturer instructions. 1.25 μg were taken for reverse transcription reaction using MuMLV Reverse: transcriptase (Gibco BRL) and Oligo (dT)₁₅ primer, SEQ ID NO:5, (Promega). Amplification of the resultant first strand cDNA was performed with Taq polymerase (Promega). The following primers were used:

SEQ ID NO:6

HPU-355: 5′-TTCGATCCCAAGAAGGAATCAAC-3′,,
nucleotides 372-394 in SEQ ID NOs:9 or 11.

SEQ ID NO:7

HPL-229: 5′-GTAGTGATGCCATGTAACTGAATC-3′,,
nucleotides 933-956 in SEQ ID NOs:9 or 11.

PCR program: 94° C.-4 min., followed by 30 cycles of 94° C.-40 sec., 62° C.-1 min., 72° C.-1 min.

Alternatively, total RNA was prepared from cell cultures using Tri-reagent (Molecular Research Center, Inc.) according to the manufacturer recommendation. Poly A+ RNA was isolated from total RNA using mRNA separator (Clontech). Reverse transcription was performed with total RNA using Superscript II (GibcoBRL). PCR was performed with Expand high fidelity (Boehringer Mannheim). Primers used for amplification were as follows:

SEQ ID NO:24

Hpu-685, 5′-GAGCAGCCAGGTGAGCCCAAGAT-3′,

SEQ ID NO:25

Hpu-355, 5′-TTCGATCCCAAGAAGGAATCAAC-3′,

SEQ ID NO:26

Hpu 565, 5′-AGCTCTGTAGATGTGCTATACAC-3′,

SEQ ID NO:27

Hpl 967, 5′-TCAGATGCAAGCAGCAACTTTGGC-3′,

SEQ ID NO:28

Hpl 171, 5′-GCATCTTAGCCGTCTTTCTTCG-3′,

SEQ ID NO:29

Hpl 229, 5′-GTAGTGATGCCATGTAACTGAATC-3′,

PCR reaction was performed as follows: 94° C. 3 minutes, followed by 32 cycles of 94° C. 40 seconds, 64° C. 1 minute, 72° C. 3 minutes, and one cycle 72° C., 7 minutes.

RNA isolation from Endothelial Cells and RT-PCR reaction: RNA from human endothelial cells was isolated and 500 ng total RNA was subjected to reverse transcription. The resulting single stranded cDNA was amplified by PCR using human specific oligonucleotide primers as described [Vlodavsky, I. et al. Mammalian heparanase: gene cloning, expression and function in tumor progression and metastasis. Nat Med 5, 793-802 (1999)]. Ten μl aliquots of the amplification-products were separated on a 1.5% agarose gel and visualized by ethidium bromide staining [Vlodavsky, I. et al. Mammalian heparanase: gene cloning, expression and function in tumor progression and metastasis. Nat Med 5, 793-802 (1999)].

Expression of recombinant heparanase in insect cells: Cells, High Five and Sf21 insect cell lines were maintained as monolayer cultures in SF9001]-SFM medium (GibcoBRL).

Expression of Recombinant heparanase in Chinese Hamster Ovary cells: Recombinant heparanase was produced in stable transfected Chinese hamster ovary (CHO) cells. The entire open reading frame of heparanase was subcloned into the EcoRI-NotI sites of the mammalian expression vector pSI (Promega), which was modified to harbor a dihydrofolate reductase expression cassette. The pSIhpa expression vector was transfected into CHO cells [Vlodavsky, I. et al. Mammalian heparanase: gene cloning, expression and function in tumor progression and metastasis. Nat Med 5, 793-802 (1999)]. Recombinant heparanase was purified from CHO cell extracts using a cation exchange CM-Sepharose column (Amersham Pharmacia Biotech).

Recombinant. Baculovirus: Recombinant virus containing the hpa gene was constructed using the Bac to Bac system (GibcoBRL). The transfer vector pFastBac was digested with SalI and NotI and ligated with a 1.7 kb fragment of phpa2 digested with XhoI and NotI. The resulting plasmid was designated pFasthpa2. An identical plasmid designated pFasthpa4 was prepared as a duplicate and both independently served for further experimentations. Recombinant bacmid was generated according to the instructions of the manufacturer with pFasthpa2, pFasthpa4 and with pFastBdac. The latter served as a negative control. Recombinant bacmid DNAs were transfected into Sf21 insect cells. Five days after transfection recombinant viruses were harvested and used to infect High Five insect cells, 3×10⁶ cells in T-25 flasks. Cells were harvested 2-3 days after infection. 4×10⁶ cells were centrifuged and resuspended in a reaction buffer containing 20 mM phosphate citrate buffer, 50 mM NaCl. Cells underwent three cycles of freeze and thaw and lysates were stored at −80° C. Conditioned medium was stored at 4° C.

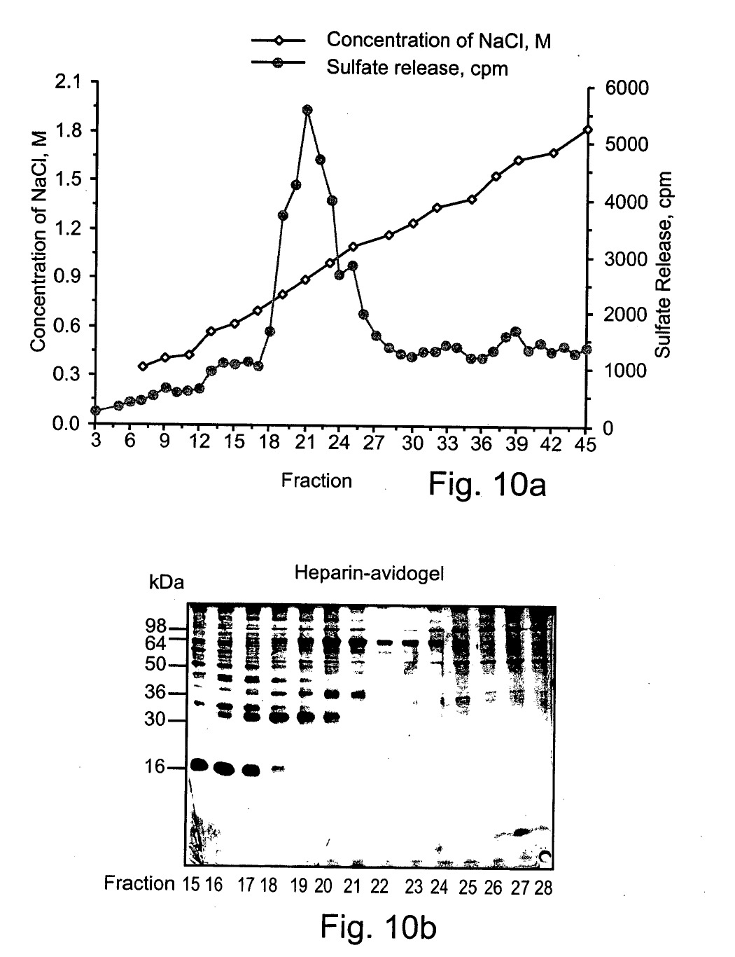

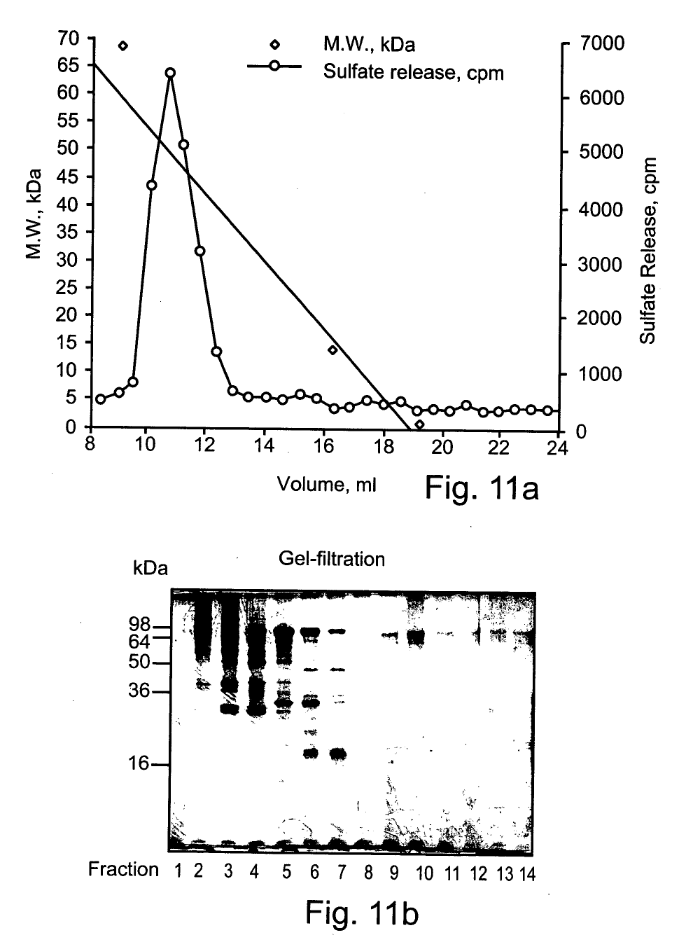

Partial purification of recombinant heparanase: Partial purification of recombinant heparanase was performed by heparin-Sepharose column chromatography followed by Superdex 75 column gel filtration. Culture medium (150 ml) of Sf21 cells infected with pFhpa4 virus was subjected to heparin-Sepharose chromatography. Elution of 1 ml fractions was performed with 0.35-2 M NaCl gradient in presence of 0.1% CHAPS and 1 mM DTT in 10 mM sodium acetate buffer, pH 5.0. A 25 μl sample of each fraction was tested for heparanase activity. Heparanase activity was eluted at the range of 0.65-1.1 M NaCl (fractions 18-26, FIG. 10a). 5 μl of each fraction was subjected to 15% SDS-polyacrylamide gel electrophoresis followed by silver nitrate staining. Active fractions eluted from heparin-Sepharose (FIG. 10a) were pooled and concentrated (×6) on YM3 cut-off membrane. 0.5 ml of the concentrated material was applied onto 30 ml Superdex 75 FPLC column equilibrated with 10 mM sodium acetate buffer, pH 5.0, containing 0.8 M NaCl, 1 mM DTT and 0.1% CHAPS. Fractions (0.56 ml) were collected at a flow rate of 0.75 ml/min. Aliquots of each fraction were tested for heparanase activity and 0.5 were subjected to SDS-polyacrylamide gel electrophoresis followed by silver nitrate staining (FIG. 11b).

PCR amplification of genomic DNA: 94° C. 3 minutes, followed by 32 cycles of 94° C. 45 seconds, 64° C. 1 minute, 68° C. 5 minutes, and one cycle at 72° C.; 7 minutes. Primers used for amplification of genomic DNA included:

SEQ ID NO:30

GHpu-L3 5′-AGGCACCCTAGAGATGTTCCAG-3′,

SEQ ID NO:31

GHp1-L6 5′-GAAGATTTCTGTTTCCATGACGTG-3′,.

Screening of genomic libraries: A human genomic library in Lambda phage EMBLE3 SP6/T7 (Clontech, Paulo. Alto, Calif.) was screened. 5×10⁵ plaques were plated at 5×10⁴ pfu/plate on NZCYM agar/top agarose plates. Phages were absorbed on nylon membranes in duplicates (Qiagen). Hybridization was performed at 65° C. in 5×SSC, 5× Denhart's, 10% dextran sulfate, 100 μg/ml Salmon sperm, ³²P labeled probe (10⁶ cpm/ml). A 1.6 kb fragment, containing the entire hpa cDNA was labeled by random priming (Boehringer Mannheim). Following hybridization membranes were washed once with 2×SSC, 0.11% SDS at 65° C. for 20 minutes, and twice with 0.2×SSC, 0.1% SDS at 65° C. for 15 minutes. Hybridizing plaques were picked, and plated at 100 pfu/plate. Hybridization was performed as above and single isolated positive plaques were picked.

Phage DNA was extracted using a Lambda DNA extraction kit (Qiagen).

DNA was digested with XhoI and EcoRI, separated on 0.7% agarose gel and transferred to nylon membrane Hybond N+ (Amersham). Hybridization and washes were performed as above.

cDNA Sequence analysis: Sequence determinations were performed with vector specific and gene specific primers, using an automated DNA sequencer (Applied Biosystems, model 373A). Each nucleotide was read from at least two independent primers.

Genomic sequence analysis: Large-scale sequencing was performed by Commonwealth Biotechnology Incorporation.

Isolation of mouse hpa: Mouse hpa cDNA was amplified from either Marathon ready cDNA library of mouse embryo or from mRNA isolated from mouse melanoma cell line BL6, using the Marathon RACE kit from Clontech. Both procedures were performed according to the manufacturer's recommendation.

Primers used for PCR amplification of mouse hpa:

SEQ ID NO:32

Mhpl773 5′-CCACACTGAATGTAATACTGAAGTG-3′,

SEQ ID NO:33

MHpl736 5′-CGAAGCTCTGGAACTCGGCAAG-3′,

SEQ ID NO:34

MHpl83 5′-GCCAGCTGCAAAGGTGTTGGAC-3′,

SEQ ID NO:35

Mhpl152 5′-AACACCTGCCTCATCACGACTTC-3′,

SEQ ID NO:36

Mhpl114 5′-GCCAGGCTGGCGTCGATGGTGA-3′,

SEQ ID NO:37

MHpl103 5′-GTCGATGGTGATGGACAGGAAC-3′,

SEQ ID NO:38

Ap1 5′-GTAATACGACTCACTATAGGGC-3′,
(Genome walker)

SEQ ID NO:39

Ap2 5′-ACTATAGGGCAdGCGTGGT-3′,
(Genome walker)

SEQ ID NO:40

Ap1 5′-CCATCCTAATACGACTCACTATAGGGC-3′,
(Marathon RACE)

SEQ ID NO:41

Ap2 5′-ACTCACTATAGGGCTCGAGCGGC-3′,
(Marathon RACE)

Southern analysis of genomic DNA: Genomic DNA was extracted from animal or from human blood using-Blood and cell culture DNA maxi kit (Qiagene). DNA was digested with EcoRI separated by gel electrophoresis and transferred to a nylon membrane Hybond N+ (Amersham). Hybridization was performed at 68° C. in 6×SSC, 1% SDS, 5× Denharts, 10% dextran sulfate, 100 μg/ml salmon sperm DNA, and ³²P labeled probe. A 1.6 kb fragment, containing the entire hpa cDNA was used as a probe. Following hybridization, the membrane Was washed with 3×SSC, 0.1% SDS, at 68° C. and exposed to X-ray film for 3 days. Membranes were, then washed with 1×SSC, 0.1% SDS, at 68° C. and were reexposed for 5 days.

Construction of hpa promoter-GFP expression vector: Lambda DNA of phage L3, was digested with SacI and BglII, resulting in a 1712 bp fragment which contained the hpa promoter (877-2688 of SEQ ID NO:42). The pEGFP-1 plasmid (Clontech) was digested with BglII and SacI and ligated with the 1712 bp fragment of the hpa promoter sequence. The resulting plasmid was designated phpEGL. A second hpa promoter-GFP plasmid was constructed containing a shorter fragment of the hpa promoter region: phpEGL was digested with: HindIII, and the resulting 1095 bp fragment (nucleotides 1593-2688 of SEQ ID NO:42) was ligated with HindIII digested pEGFP-1. The resulting plasmid was designated phpEGS.

Computer analysis of sequences: Homology searches were performed using, several computer servers, and various databases. Blast 2.0 service, at the NCBI server was used to screen the protein database swplus and DNA databases such as GenBank, EMBL, and the EST databases. Blast 2.0 search was performed using the basic search option of the NCBI server. Sequence analysis and alignments were done using the DNA sequence analysis software package developed by the Genetic Computer Group (GCG) at the university of Wisconsin. Alignments of two sequences were performed using Bestfit (gap creation penalty −12, gap extension penalty −4). Protein homology search was performed with the Smith-Waterman algorithm, using the Bioaccelerator platform developed by Compugene. The protein database swplus was searched using the following parameters: gapop: 10.0, gapext: 0.5, matrix: blosum62. Blocks homology was performed using the Blocks www server developed at Fred Hutchinson Cancer Research Center in Seattle, Wash., USA. Secondary structure prediction was performed using the PHD server—Profile network Prediction Heidelberg. Fold recognition (threading) was performed using the UCLA-DOE structure prediction server. The method used for prediction was gonnet+predss. Alignment of three sequences was performed using the pileup application (gap creation penalty −5, gap extension penalty −0.1). Promoter analysis was performed using TSSW and TSSG programs (BCM Search Launcher Human Genome Center, Baylor College of Medicine, Houston Tex.).

### EXAMPLE 1

### Cloning of human hpa cDNA

Purified fraction of heparanase isolated from human hepatoma cells (SK-hep-1) was subjected to tryptic digestion and microsequencing. EST (Expressed Sequence Tag) databases were screened for homology to the back translated DNA sequences corresponding to the obtained peptides. Two EST sequences (accession Nos. N41349 and N45367) contained a DNA sequence encoding the peptide YGPDVGQPR (SEQ ID NO:8). These two sequences were derived from clones 257548 and 260138 (I.M.A.G.E Consortium) prepared from 8 to 9 weeks placenta cDNA library (Soares). Both clones which were found to be: identical contained an insert of 1.020 bp which included an open reading frame (ORF) of 973 bp followed by a 3′ untranslated region of 27 bp and a Poly A tail. No translation start site (AUG) was identified at the 5′ end of these clones.

Cloning of the missing 5′ end was performed by PCR amplification of DNA from a placenta Marathon RACE cDNA composite. A 900 bp fragment (designated hp3), partially overlapping with the identified 3′-encoding EST clones was obtained.

The joined cDNA fragment, 1 721 bp long (SEQ ID NO:9), contained an open reading frame which encodes, as shown in FIG. 1 and SEQ ID NO:11, a polypeptide of 543 amino acids (SEQ ID NO:10) with a calculated molecular weight of 61,192 daltons. The 3′ end of the partial cDNA inserts contained in: clones 257548 and 260138 started at nucleotide G⁷²¹ of SEQ ID NO:9 and FIG. 1.

As further shown in FIG. 1, there was a single sequence discrepancy between the EST clones and the PCR amplified sequence, which led to an amino acid substitution from Tyr²⁴⁶ in the EST to Phe²⁴⁶ in the amplified cDNA. The nucleotide sequence of the PCR amplified cDNA fragment was verified from two independent amplification products. The new gene was designated hpa.

As stated above, the 3′ end of the partial, cDNA inserts contained in EST clones 257548 and 260138 started at nucleotide 721 of hpa (SEQ ID NO:9). The ability of the hpa cDNA to form stable secondary structures, such as stem and loop structures involving nucleotide stretches in the vicinity of position 721 was investigated using computer modeling. It was found that stable stem and loop structures are likely to be formed involving nucleotides 698-724 (SEQ ID NO:9). In addition, a high GC content, up to 70%, characterizes the 5′ end region of the hpa gene, as compared to about only 40% in the 3′ region. These findings may explain the immature termination and therefore lack of 5′ ends in the EST clones.

To examine the ability of the hpa gene product to catalyze degradation of heparan sulfate in an in vitro assay the entire open reading frame was expressed in insect cells, using the Baculovirus expression system. Extracts of cells, infected with virus containing the hpa gene, demonstrated a high level of heparan sulfate degradation activity, while cells infected with a similar construct containing no hpa gene had no such activity, nor did non-infected cells. These results are further demonstrated in the following Examples.

### EXAMPLE 2

### Degradation of Soluble ECM-Derived HSPG

Monolayer cultures of High Five cells were infected (72 h, 28° C.) with recombinant Bacoluvirus containing the pFasthpa plasmid or with control virus containing an insert free plasmid. The cells were harvested and lysed in heparanase reaction buffer by three cycles of freezing and thawing. The cell lysates were then incubated (18 h, 37° C.) with sulfate labeled, ECM-derived HSPG (peak I), followed by gel filtration analysis (Sepharose 6B) of the reaction mixture.

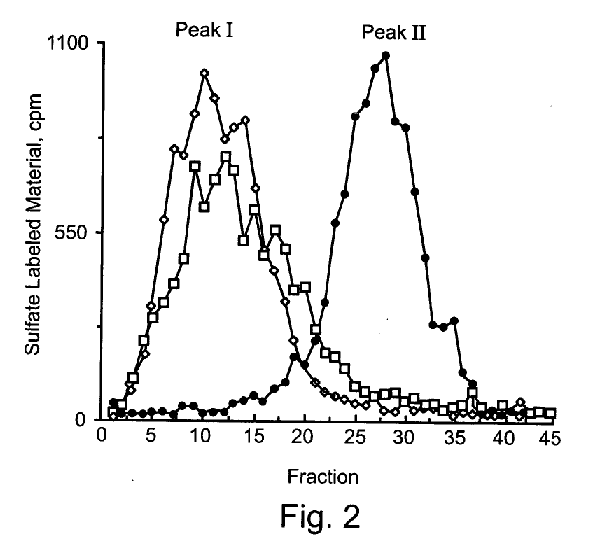

As shown in FIG. 2, the substrate alone included almost entirely high molecular weight (Mr) material eluted next to V_{O} (peak I, fractions 5-20, Kav<0.35). A similar elution pattern was obtained when the HSPG substrate was incubated with lysates of cells that were infected with control virus. In contrast, incubation of the HSPG substrate with lysates of cells infected with the hpa containing virus resulted in a complete conversion of the high Mr substrate into low Mr labeled degradation fragments (peak II, fractions 22-35, 0.5<Kav<0.75).

Fragments eluted in peak II were shown to be degradation products of heparan sulfate, as they were (i) 5- to 6-fold smaller than intact heparan sulfate side chains (Kav approx. 0.33) released from ECM by treatment with either alkaline borohydride or papain; and (ii) resistant to further digestion with papain or chondroitinase ABC, and susceptible to deamination by nitrous acid (6, 11). Similar results (not shown) were obtained with Sf21 cells. Again, heparanase activity was detected in cells infected with the hpa containing virus (pFhpa), but not with control virus (pF). This result was obtained with two independently generated recombinant viruses. Lysates of control not infected High Five cells failed to degrade the HSPG substrate.

In subsequent experiments, the labeled HSPG substrate was incubated with medium conditioned by infected-High Five or Sf21 cells.

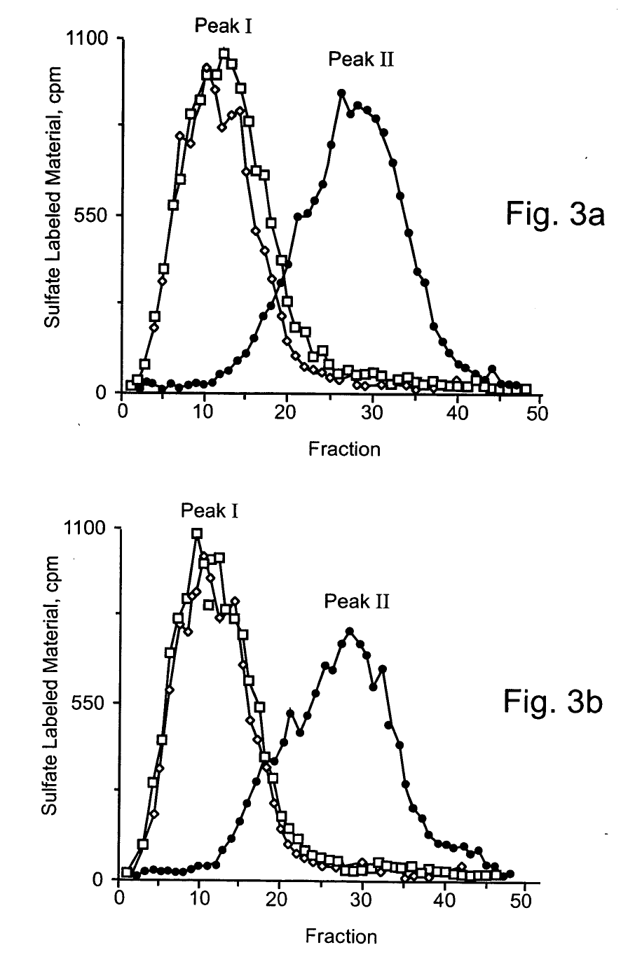

As shown in FIGS. 3a-b, heparanase activity, reflected by the conversion of the high Mr peak I substrate into the low Mr peak II which represents HS degradation fragments, was found in the culture medium of cells infected with the pFhpa2 or pFhpa4 viruses, but not with the control pF I or pF2 viruses. No heparanase activity was detected in the culture medium of control non-infected High-Five or Sf21 cells.

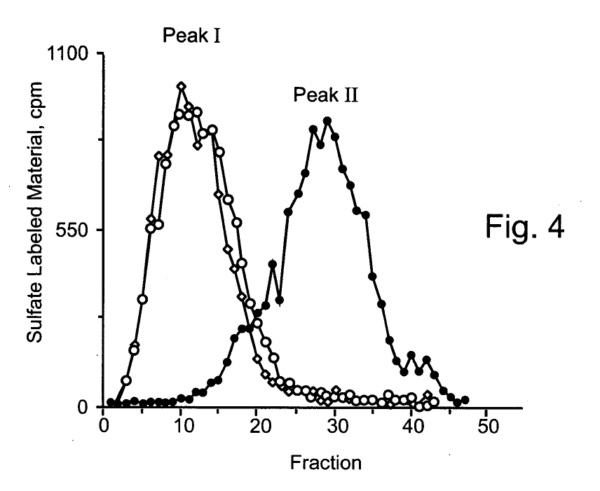

The medium of cells infected with the pFhpa4 virus was passed through a 50 kDa cut off membrane to obtain a crude estimation of the molecular weight of the recombinant heparanase enzyme. As demonstrated in FIG. 4, all the enzymatic activity was retained in the upper compartment and there was no activity in the flow through (<50 kDa) material. This result is consistent with the expected molecular weight of the hpa gene product.

In order to further characterize the hpa product the inhibitory effect of heparin, a potent inhibitor of heparanase mediated HS degradation (40) was examined.

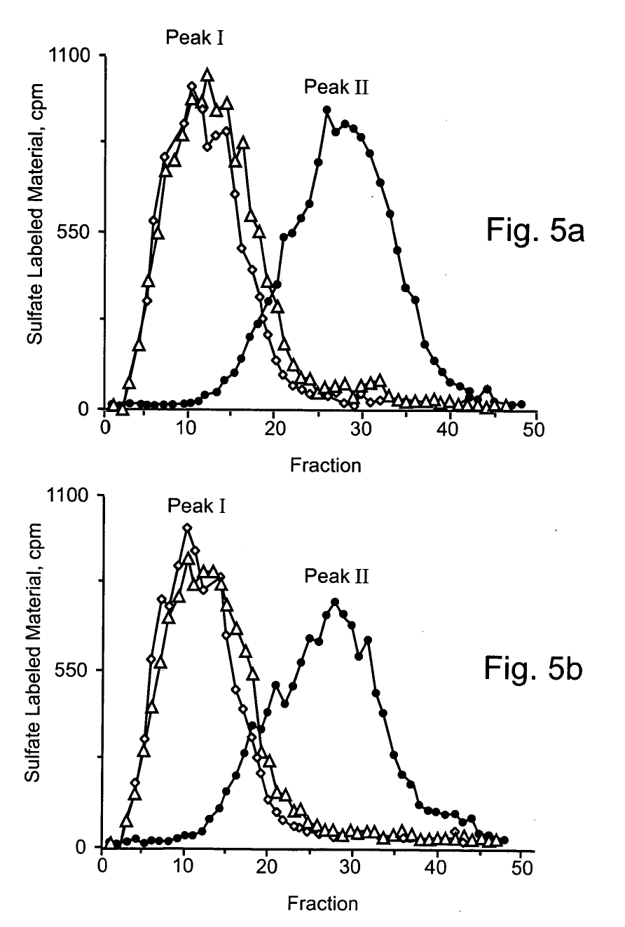

As demonstrated in FIGS. 5a-b, conversion of the peak I substrate into peak II HS degradation fragments was completely abolished in the presence of heparin.

Altogether, these results indicate that the heparanase enzyme is expressed in an active form by insect cells infected with Baculovirus containing the newly identified human hpa gene.

### EXAMPLE 3

### Degradation of HSPG in Intact ECM

Next, the ability of intact infected insect cells to degrade HS in intact, naturally produced ECM was investigated. For this purpose, High Five or Sf21 cells were seeded on metabolically sulfate labeled ECM followed by infection (48 h, 28° C.) with either the pFhpa4 or control pF2 viruses. The pH of the medium was then adjusted to pH 6.2-6.4 and the cells further incubated with the labeled ECM for another 48 h at 28° C. or 24 h at 37° C. Sulfate labeled material released into the incubation medium was analyzed by gel filtration on Sepharose 6B.

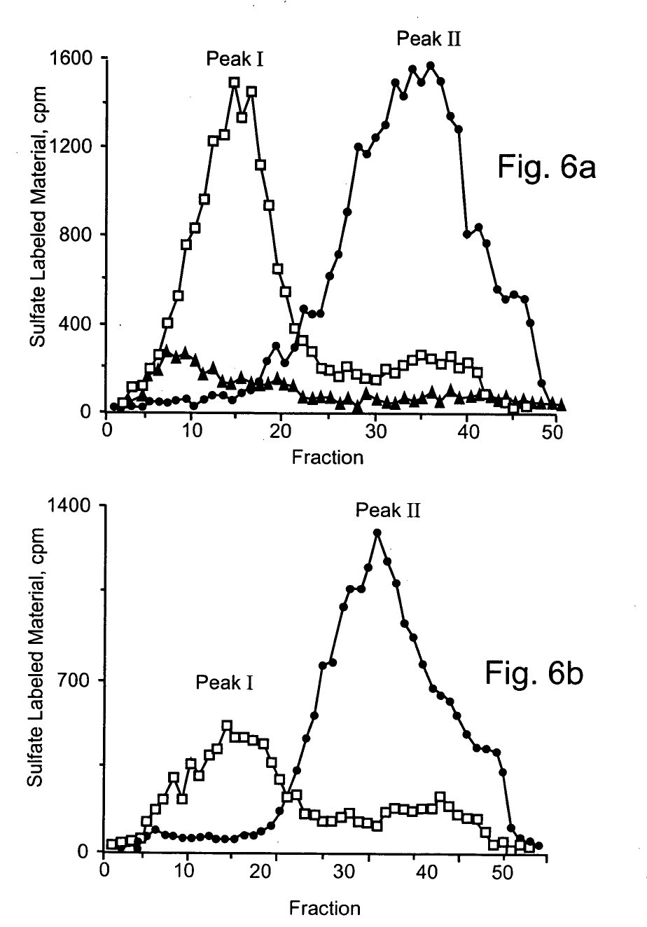

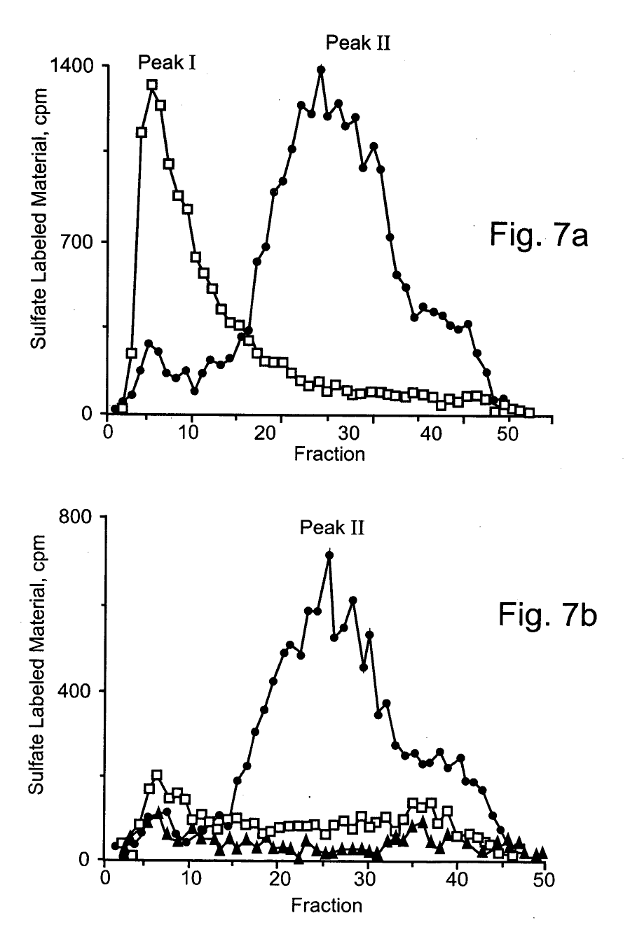

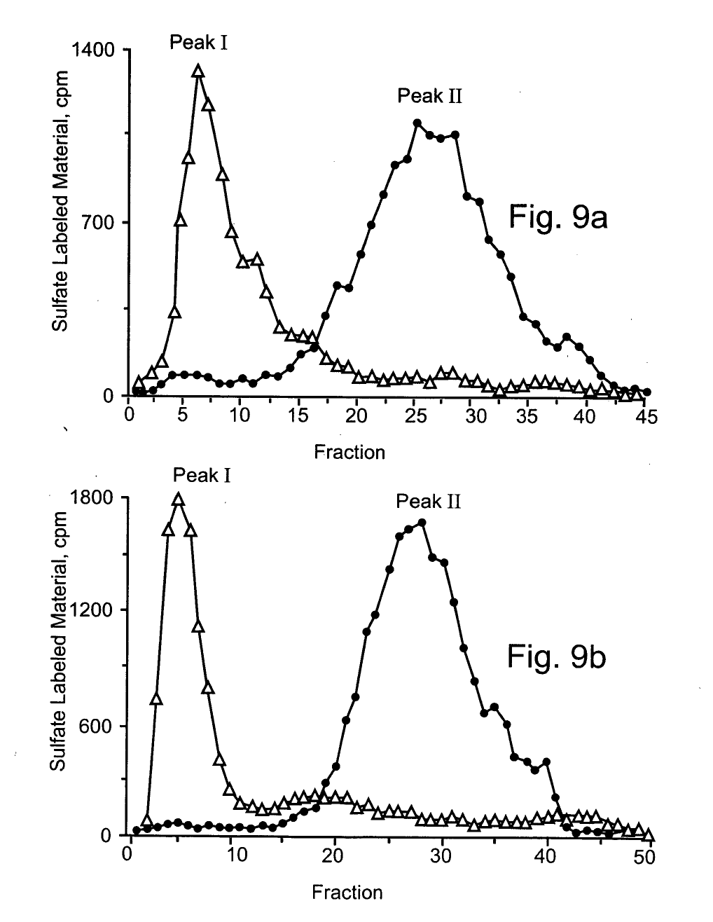

As shown in FIGS. 6a-b and 7a-b, incubation of the ECM with cells infected with the control pF2 virus resulted in a constant release of labeled material that consisted almost entirely (>90%) of high Mr fragments (peak I) eluted with or next to V_{O}. It was previously shown that a proteolytic activity residing in the ECM itself and/or expressed by cells is responsible for release of the high Mr material (6). This nearly intact HSPG provides a soluble substrate for subsequent degradation by heparanase, as also indicated by the relatively large amount of peak I material accumulating when the heparanase enzyme is inhibited by heparin (6, 7, 12, FIG. 9). On the other hand, incubation of the labeled ECM with cells infected with the pFhpa4 virus resulted in release of 60-70% of the ECM-associated radioactivity in the form of low Mr sulfate-labeled fragments (peakII, 0.5<Kav<0.75), regardless of whether the infected cells were incubated with the ECM at 28° C. or 37° C. Control intact non-infected Sf21 or High Five cells failed to degrade the ECM HS side chains.

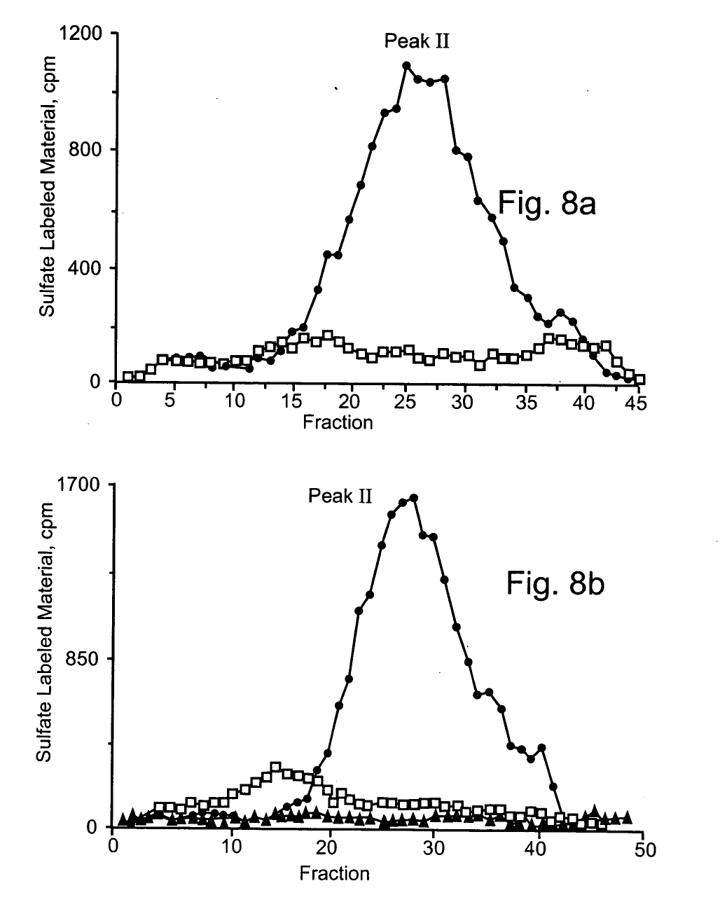

In subsequent experiments as demonstrated in FIGS. 8a-b, High Five and Sf21 cells were infected (96 h, 28° C.) with pFhpa4 or control pF1 viruses and the culture medium incubated with sulfate-labeled ECM. Low Mr HS degradation fragments were released from the ECM only upon incubation with medium conditioned by pFhpa4 infected cells. As shown in FIG. 9, production of these fragments was abolished in the presence of heparin. No heparanase activity was detected in the culture medium of control, non-infected cells. These results indicate that the heparanase enzyme expressed by cells infected with the pFhpa4 virus is capable of degrading HS when complexed to other macromolecular constituents (i.e. fibronectin, laminin, collagen) of a naturally produced intact ECM, in a manner similar to that reported for highly metastatic tumor cells or activated cells of the immune system (6, 7).

### EXAMPLE 4

### Purification of Recombinant Human Heparanase

The recombinant heparanase was partially purified from medium of pFhpa4 infected Sf21 cells by Heparin-Sepharose chromatography (FIG. 10a) followed by gel filtration of the pooled active fractions over an FPLC Superdex 75 column (FIG. 11a). A ˜63 kDa protein was observed, whose quantity, as was detected by silver stained SDS-polyacrylamide gel electrophoresis, correlated with heparanase activity in the relevant column fractions (FIGS. 10b and 11b, respectively). This protein was not detected in the culture medium of cells infected with the control pF1 virus and was subjected to a similar fractionation on heparin-Sepharose (not shown).

### EXAMPLE 5

### Expression of the Human hpa cDNA in Various Cell Types, Organs and Tissues

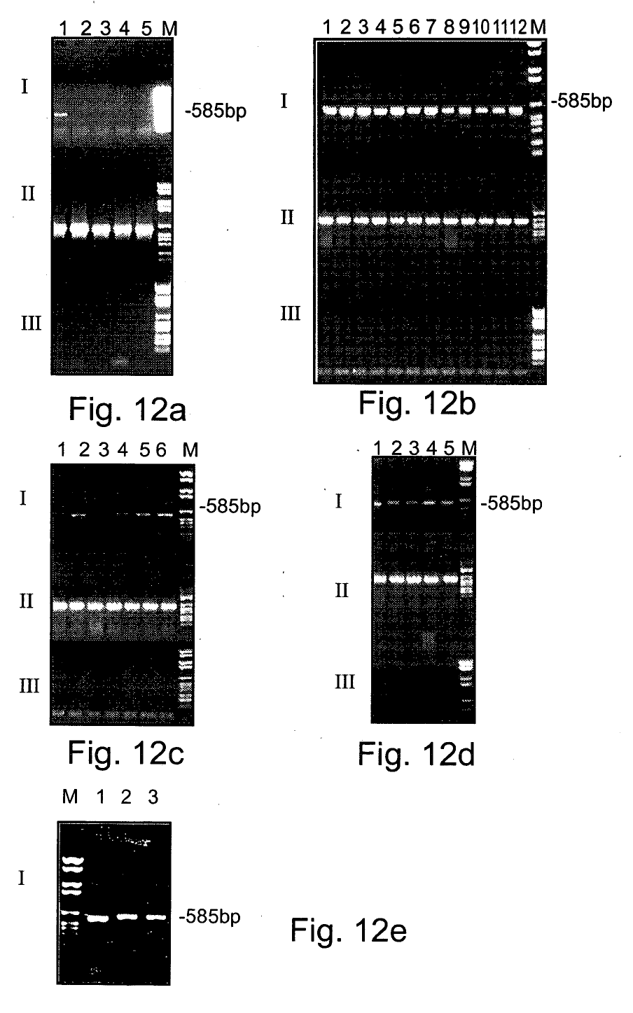

Referring now to FIGS. 12a-e, RT-PCR was applied to evaluate the expression of the hpa gene by various cell types and tissues. For this purpose, total RNA was reverse transcribed and amplified. The expected 585 bp long cDNA was clearly demonstrated in human kidney, placenta (8 and 11 weeks) and mole tissues, as well as in freshly isolated and short termed (1.5-48 h) cultured human placental cytotrophoblastic cells (FIG. 12a), all known to express a high heparanase activity (41). The hpa transcript was also expressed by normal human neutrophils (FIG. 12b). In contrast, there was no detectable expression of the hpa mRNA in embryonic human muscle tissue, thymus, heart and adrenal (FIG. 12b). The hpa gene was expressed by several, but not all, human bladder carcinoma cell lines (FIG. 12c), SK hepatoma (SK-hep-1), ovarian carcinoma (OV 1063), breast carcinoma (435, 231), melanoma and megakaryocytic (DAMI, CHRF) human cell lines (FIGS. 12d-e).

The above described expression pattern of the hpa transcript was determined to be in a very good correlation with heparanase activity levels determined in various tissues and cell types (not shown).

### EXAMPLE 6

### Isolation of an Extended 5′ end of hpa cDNA from Human SK-hep1 Cell Line

The 5′ end of hpa cDNA was isolated from human SK-hep1 cell line by PCR amplification using the Marathon RACE (rapid amplification of cDNA ends) kit (Clontech). Total RNA was prepared from SK-hep1 cells using the TR1-Reagent (Molecular research center Inc.) according to the manufacturer instructions. Poly A+ RNA was isolated using the mRNA separator kit (Clonetech).

The Marahton RACE SK-hep1 cDNA composite was constructed according to the manufacturer recommendations. First round of amplification was performed using an adaptor specific primer AP1: 5′-CCATCCTAATACG ACTCACTATAGGGC-3′, SEQ ID NO:1, and a hpa specific antisense primer hpl-629: 5′-CCCCAGGAGCAGCAGCATCAQ-3′, SEQ ID NO:17, corresponding to nucleotides 119-99 of SEQ ID NO:9. The resulting PCR product was subjected to a second round of amplification using an adaptor specific nested primer AP2: 5′-ACTCACTATAGGGCTCGAGCGGC-3′, SEQ ID NO:3, and a hpa specific antisense nested primer hpl-666 5′-AGGCTTCGAGCGCAGCAGCAT-3′, SEQ ID. NO: 18, corresponding to nucleotides 83-63 of SEQ ID NO:9. The PCR program was as follows: a hot start of 94° C. for 1 minute, followed by 30 cycles of 90° C.-30 seconds, 68° C.-4 minutes. The resulting 300 bp. DNA fragment was extracted from an agarose gel and cloned into the vector pGEM-T Easy (Promega). The resulting recombinant plasmid was designated pHPSK1.

The nucleotide sequence of the pHPSK1 insert was determined and it was found to contain 62 nucleotides of the 5′ end of the placenta; hpa cDNA (SEQ ID NO:9) and additional 178 nucleotides upstream, the first 178 nucleotides of SEQ ID NOs:13 and 15.

A single nucleotide discrepancy was identified between the SK-hep1 cDNA and the placenta cDNA. The “T” derivative at position 9 of the placenta cDNA (SEQ ID NO:9), is replaced by a “C” derivative at the corresponding position-187 of the SK-hep1 cDNA (SEQ ID NO:13).

The discrepancy is likely to be due to a mutation at the 5′ end of the placenta cDNA clone as confirmed by sequence analysis of several additional cDNA clones isolated from placenta, which like the SK-hep1 cDNA contained C at position 9 of SEQ ID NO:9.

The 5′ extended sequence of the SK-hep1 hpa cDNA was assembled with the sequence of the hpa cDNA isolated from human placenta (SEQ ID NO:9). The assembled sequence contained an open reading frame which encodes, as shown in SEQ ID NOs:14 and 15, a polypeptide of 592 amino acids with a calculated molecular weight of 66,407 daltons. The open reading frame is flanked by 93 bp 5′ untranslated region (UTR).

### EXAMPLE 7

### Isolation of the Upstream Genomic Region of the hpa Gene

The upstream region of the hpa gene was isolated using the Genome Walker kit (Clontech) according to the manufacturer recommendations. The kit includes five human genomic DNA samples each digested with a different restriction endonuclease creating blunt ends: EcoRV, ScaI, PraI, PvuII and SspI.

The blunt ended DNA fragments are ligated to partially single stranded adaptors. The Genomic DNA samples were subjected to PCR amplification using the adaptor specific primer and a gene specific primer. Amplification was performed with Expand High Fidelity (Boehringer Mannheim).

A first round of amplification was performed using the ap1 primer: 5′-G TAATACGACTCACTATAGGGC-3′, SEQ ID NO:19, and the hpa specific antisense primer hp1-666: 5′-AGGCTTCGAGCGCAGCAGCAT-3′, SEQ ID NO:18, corresponding to nucleotides 0.83-63 of SEQ ID NO:9. The PCR program was as follows: a hot start of 94° C.-3 minutes, followed by 36 cycles of 94° C.-40 seconds, 67° C.-4 minutes.

The PCR products of the first amplification were diluted 1:50. One μl of the diluted sample was used as a template for a second amplification using a nested adaptor specific primer ap2: 5′-ACTATAGGGCACGCGTGGT-31, SEQ ID NO:20, and a hpa specific antisense primer hp1-690, 5′-CTTGGGCTCACC TGGCTGCTC-3′, SEQ ID NO:21, corresponding to nucleotides 62-42 of SEQ ID:NO:9. The resulting amplification products were analyzed using agarose gel electrophoresis. Five different PCR products were obtained from the five amplification reactions. A DNA fragment of approximately 750 bp which was obtained from the SspI digested DNA sample was gel extracted. The purified fragment was ligated into the plasmid vector PGEM-T Easy (Promega). The resulting recombinant plasmid was designated pGHP6905 and the nucleotide sequence of the hpa insert was determined.

A partial sequence of 594 nucleotides is shown in SEQ ID NO:16. The last nucleotide in SEQ ID NO:13 corresponds to nucleotide 93 in SEQ ID:13. The DNA sequence in SEQ ID NO:16 contains the 5′ region of the hpa cDNA and 501 nucleotides of the genomic upstream region which are predicted to contain the promoter region of the hpa gene.

### EXAMPLE 8

### Expression of the 592 Amino Acids HPA Polypeptide in a Human 293 Cell Line

The 592 amino acids open reading frame (SEQ ID NOs:13 and 15) was constructed by ligation of the 110 bp corresponding to the 5′ end of the SK-hep1 hpa cDNA with the placenta cDNA. More specifically the Marathon RACE—PCR amplification product of the placenta hpa DNA was digested with SacI and an approximately 1 kb fragment was ligated into a SacI-digested pGHP6905 plasmid. The resulting plasmid was digested with Earl and AatII. The Earl sticky ends were blunted and an approximately 280 bp Earl/blunt-AatII fragment was isolated. This fragment was ligated with pFasthpa digested with EcoRI which was blunt ended-using Klenow fragment and further digested with AatII. The resulting plasmid contained a 1827 bp insert which includes an open reading frame of 1776 bp, 31 bp of 3′ UTR and 21 bp of 5′ UTR. This plasmid was designated pFastLhpa.

A mammalian expression vector was constructed to drive the expression of the 592 amino acids heparanase polypeptide in human-cells. The hpa cDNA was excised prom pFastLhpa with BssHII and NotI. The resulting 1850 bp BssHII-NotI fragment was ligated to a mammalian expression vector pSI (Promega) digested with MluI and NotI. The resulting recombinant plasmid, pSIhpaMet2 was transfected into a human 293 embryonic kidney cell line.

Transient expression of the 592 amino-acids heparanase was examined by western blot analysis and the enzymatic activity was tested using the gel shift assay. Both these procedures are described in length in U.S. patent application Ser. No. 09/071,739, filed May 1, 1998, which is incorporated by reference as if fully set forth herein. Cells were harvested 3 days following transfection. Harvested cells were re-suspended in lysis buffer containing 150 mM NaCl, 50 mM Tris-pH 7.5, 1% Triton X-100, 1 mM PMSF and protease inhibitor cocktail (Boehringer Mannheim). 40 μg protein extract samples were used for separation on a SDS-PAGE. Proteins were transferred onto a PVDF Hybond-P membrane (Amersham). The membrane was incubated with an affinity purified polyclonal anti heparanase antibody, as described in U.S. patent application Ser. No. 09/071,739. A major band of approximately 50 kDa was observed in the transfected cells as well as a minor band of approximately 65 kDa. A similar pattern was observed in extracts of cells transfected with the pShpa as demonstrated in U.S. patent application Ser. No. 09/071,739. These two bands probably represent two forms of the recombinant heparanase protein produced by the transfected cells. The 65 kDa protein probably represents a heparanase precursor, while the 50 kDa protein is suggested herein to be the processed or mature form.

The catalytic activity of the recombinant protein expressed in the pShpaMet2 transfected cells was tested by gel shift assay. Cell extracts of transfected and of mock transfected cells were incubated overnight with heparin (6 μg in each reaction) at 37° C., in the presence of 20 mM phosphate citrate buffer pH 5.4, 1 mM CaCl₂, 1 mM DTT and 50 mM NaCl. Reaction mixtures were then separated on a 10% polyacrylamide gel. The catalytic activity of the recombinant heparanase was clearly demonstrated by a faster migration of the heparin molecules incubated with the transfected cell extract as compared to the control. Faster migration indicates the disappearance of high molecular weight heparin molecules and the generation of low molecular weight degradation products.

### EXAMPLE 9

### Chromosomal Localization of the hpa Gene

Chromosomal mapping of the hpa gene was performed utilizing a panel of monochromosomal human/CHO and human/mouse somatic cell hybrids, obtained from the UK HGMP Resource Center (Cambridge, England).

40 ng of each of the somatic cell hybrid DNA samples were subjected to PCR amplification using the hpa primers: hpu565 5′-AGCTCTGTAGATGTGC TATACAC-3′, SEQ ID NO:22, corresponding to nucleotides 564-586 of SEQ ID NO:9 and an antisense primer hpll71 5′-GCATCTTAGCCGTCTTTCTTCG-3′; SEQ ID NO:23, corresponding to nucleotides 897-876 of SEQ ID NO:9.

The PCR program was as follows: a hot start of 94° C.-3 minutes, followed by 7 cycles of 94° C.-45 seconds, 66° C.-1 minute, 68° C.-5 minutes, followed by 30 cycles of 94° C.-45 seconds, 62° C. —1 minute; 68° C.-5 minutes, and a 1:0 minutes final extension at 72° C.

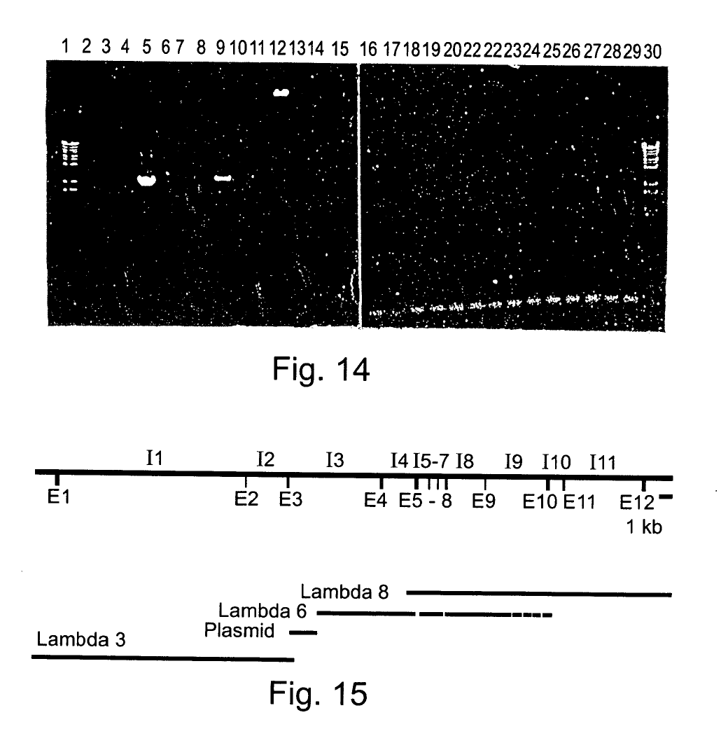

The reactions were performed with Expand long PCR (Boehringer Mannheim). The resulting amplification products were analyzed using agarose gel electrophoresis. As demonstrated in FIG. 14, a single band of approximately 2.8 Kb was obtained from chromosome 4, as well as from the control human genomic DNA. A 2.8 kb amplification product is expected based on amplification of the genomic hpa clone (data not shown). No amplification products were obtained neither in the control DNA samples of hamster and mouse nor in somatic hybrids of other human chromosome.

### EXAMPLE 10

### Human Genomic Clone Encoding Heparanase

Five plaques were isolated following screening of a human genomic library and were designated L3-1, L5-1, L8-1, L10-1 and L6-1. The phage DNAs were analyzed by Southern hybridization and by PCR with hpa specific and vector specific primers. Southern analysis was performed with three fragments of hpa cDNA: a PvuII-BamHI fragment (nucleotides 32-450, SEQ. ID NO:9), a BamHI-NdeI fragment (nucleotides 451-1102, SEQ ID NO:9) and an NdeI-XhoI fragment. (nucleotides. 1103-1721, SEQ ID NO:9).

Following Southern analysis, phages L3, L6, L8 were selected for further analysis. A scheme of the genomic region and the relative position of the three phage clones is depicted in FIG. 15. A 2 kb DNA fragment containing the gap between phages L6 and L3 was PCR amplified from human genomic DNA with two gene specific primers GHpuL3 and GHplL6. The PCR product was cloned into the plasmid vector pGEM-T-easy (Promega).

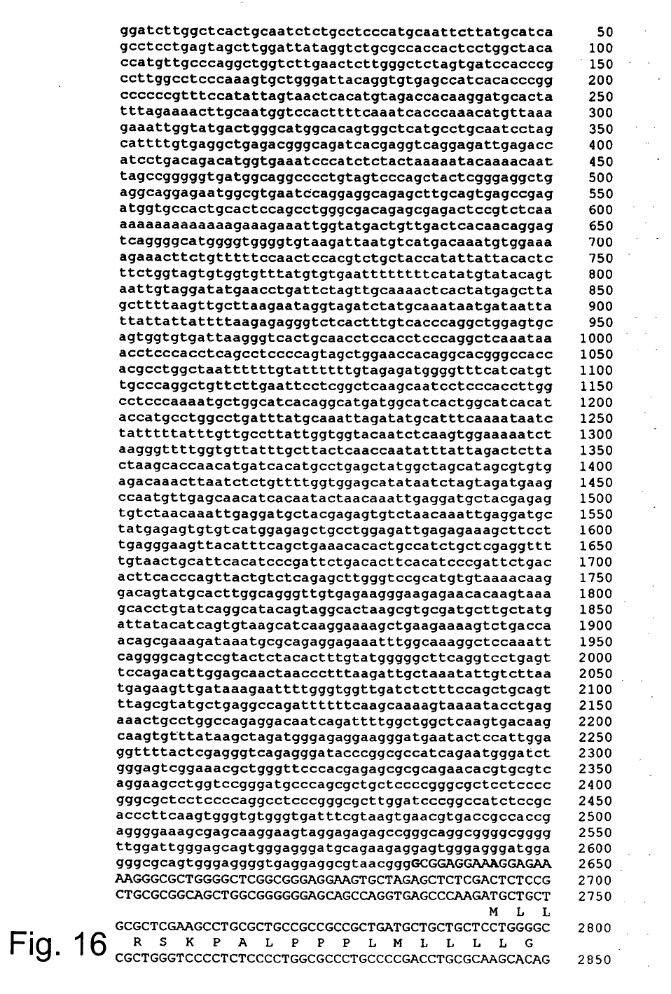

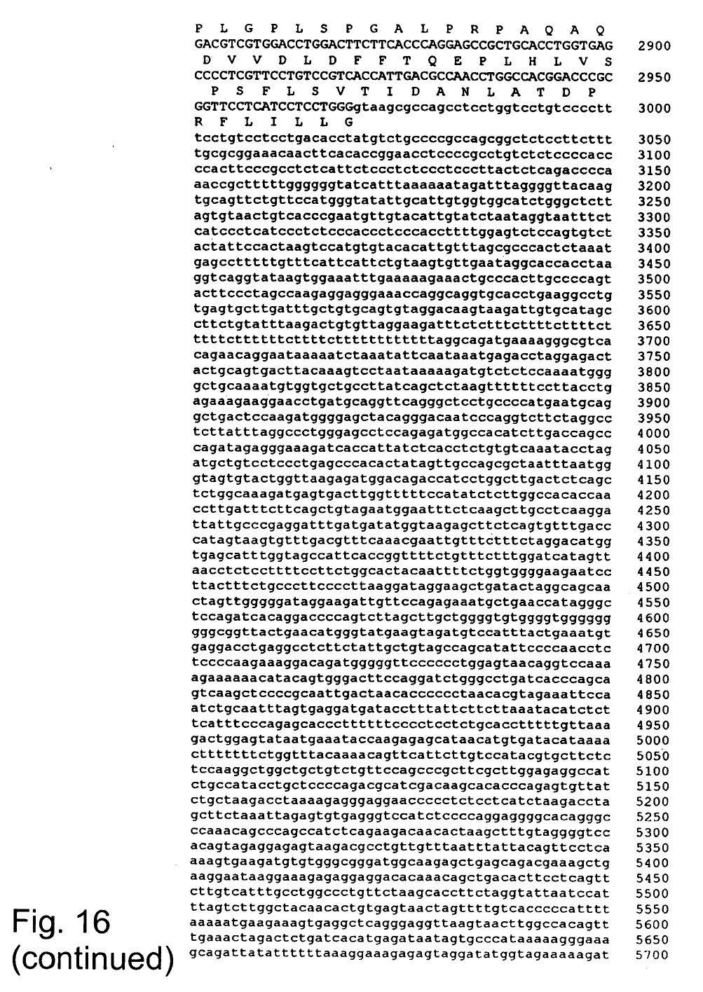

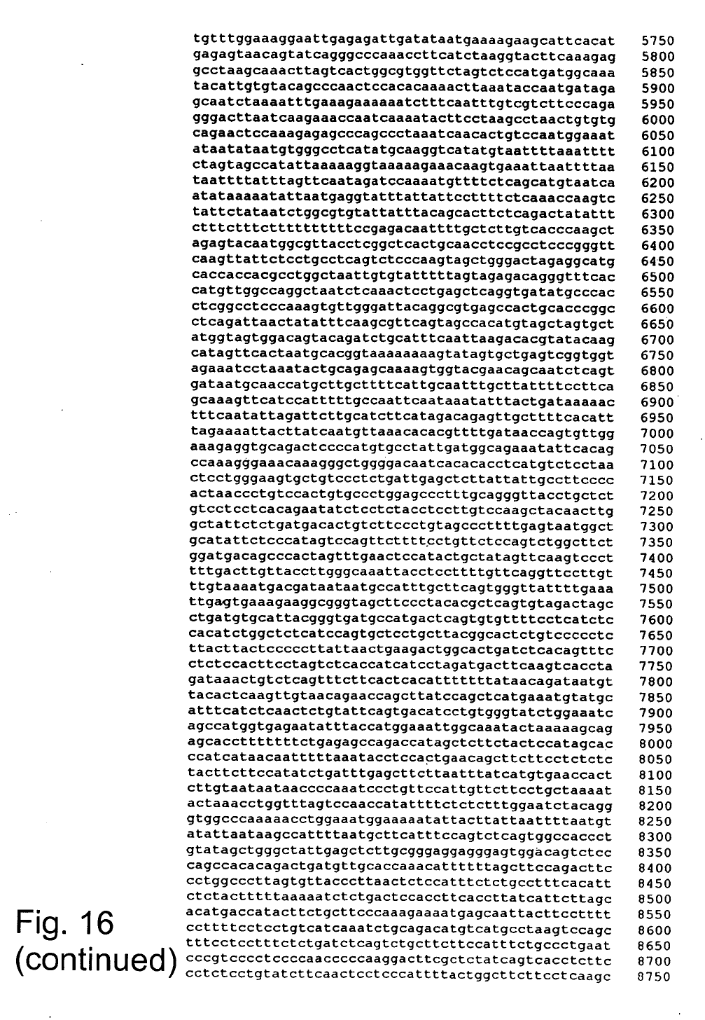

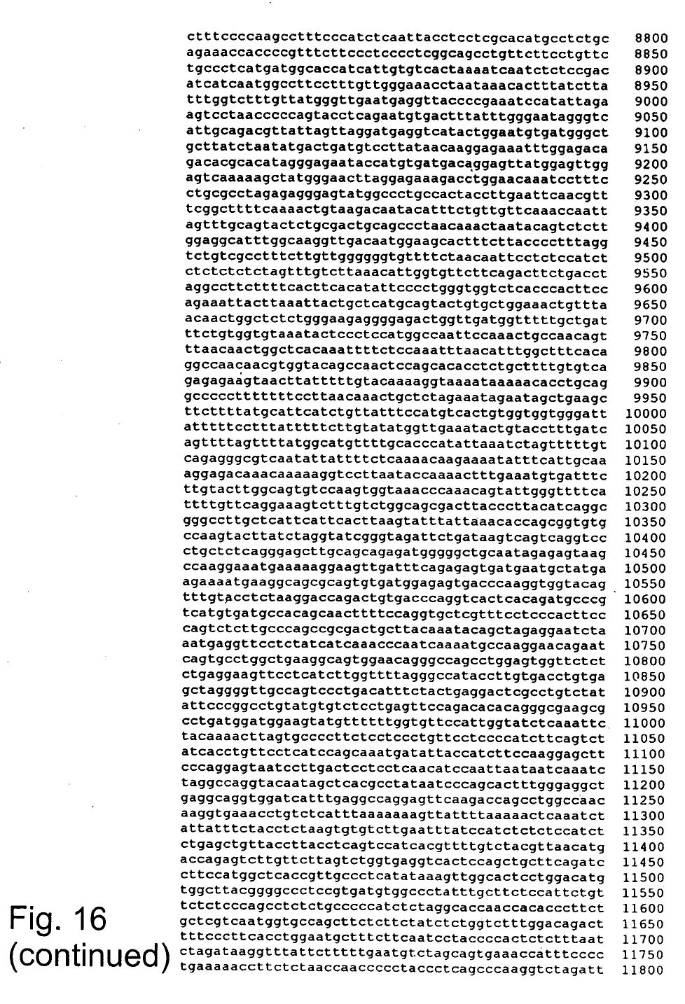

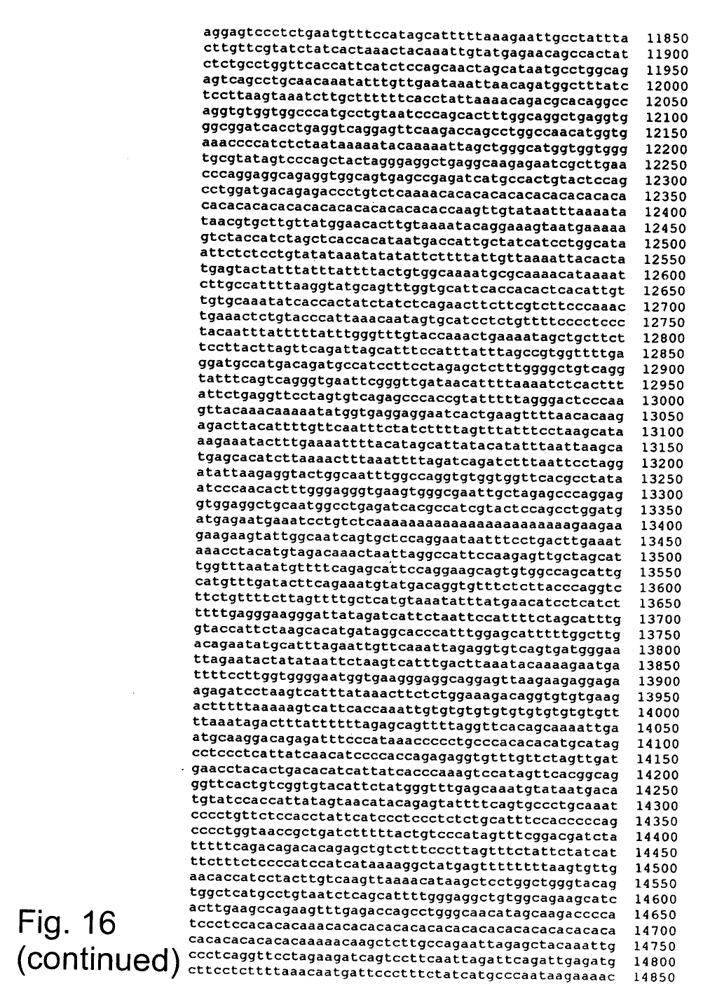

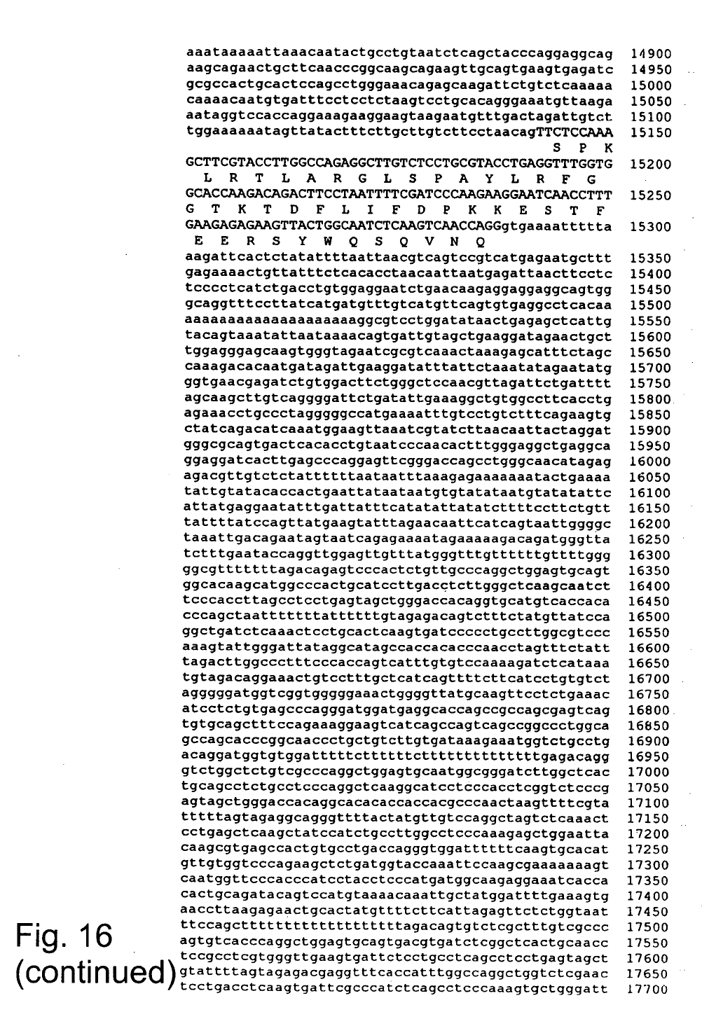

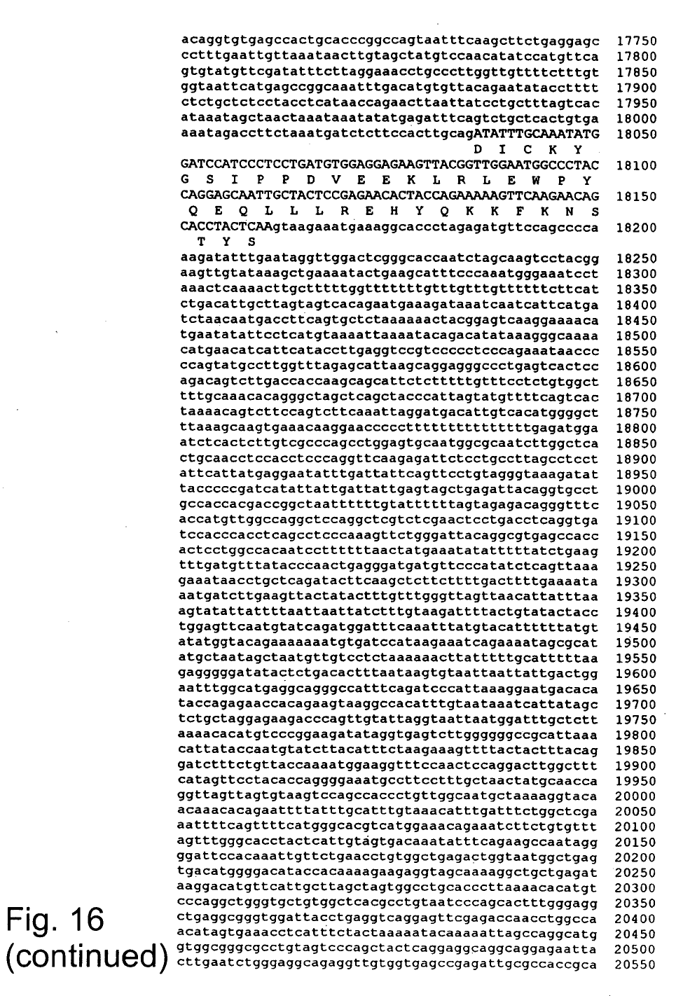

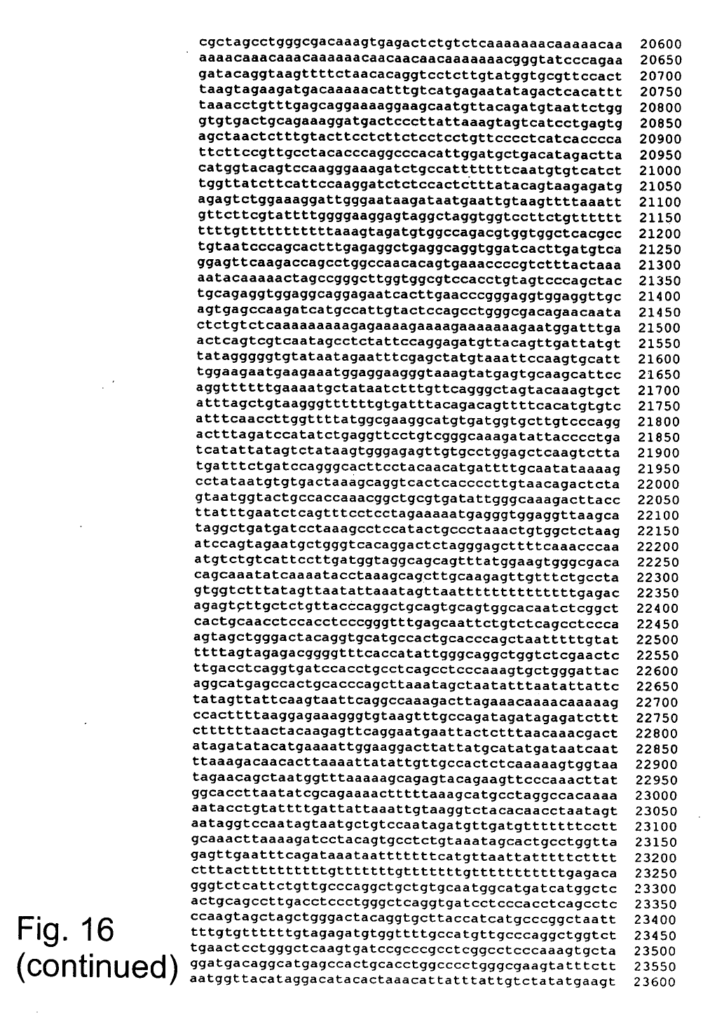

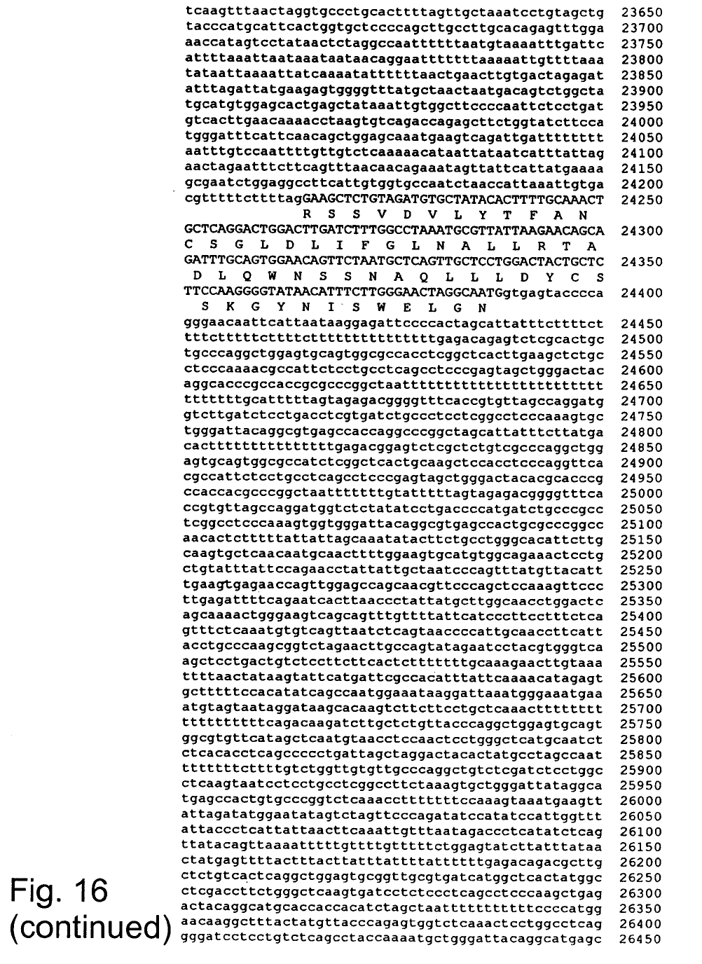

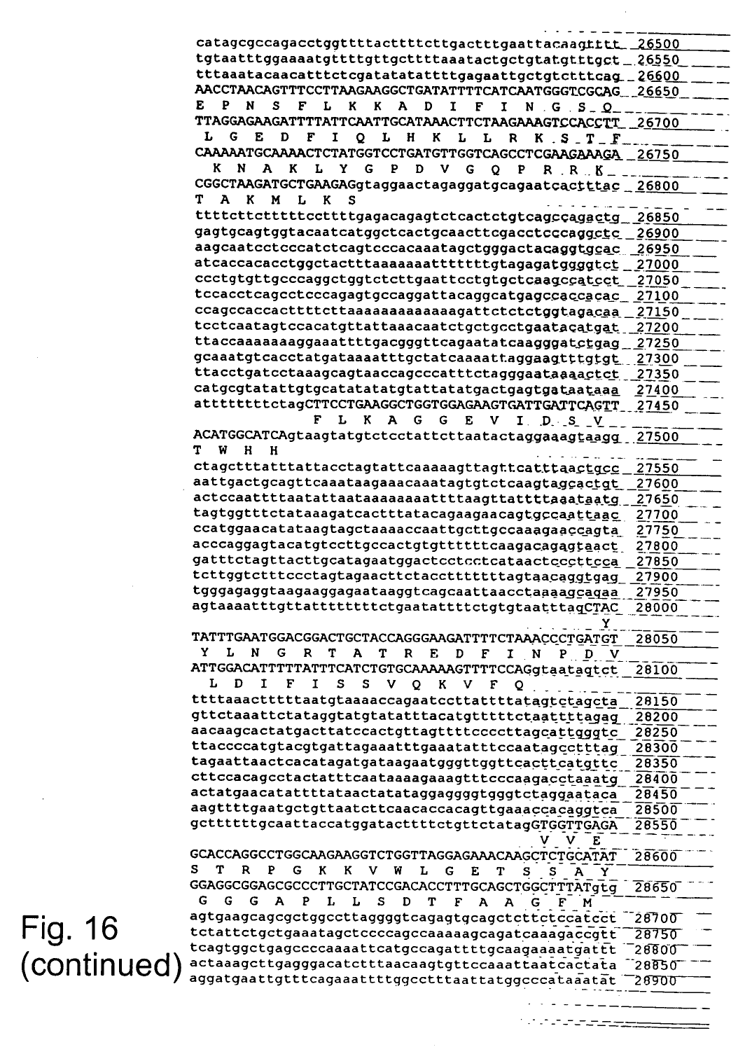

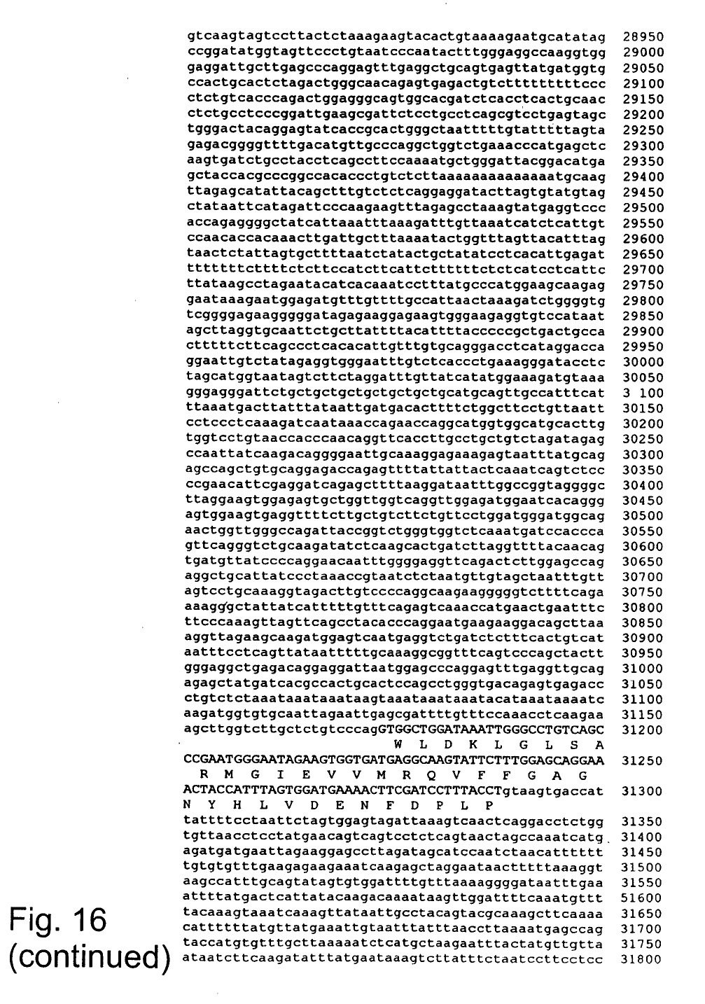

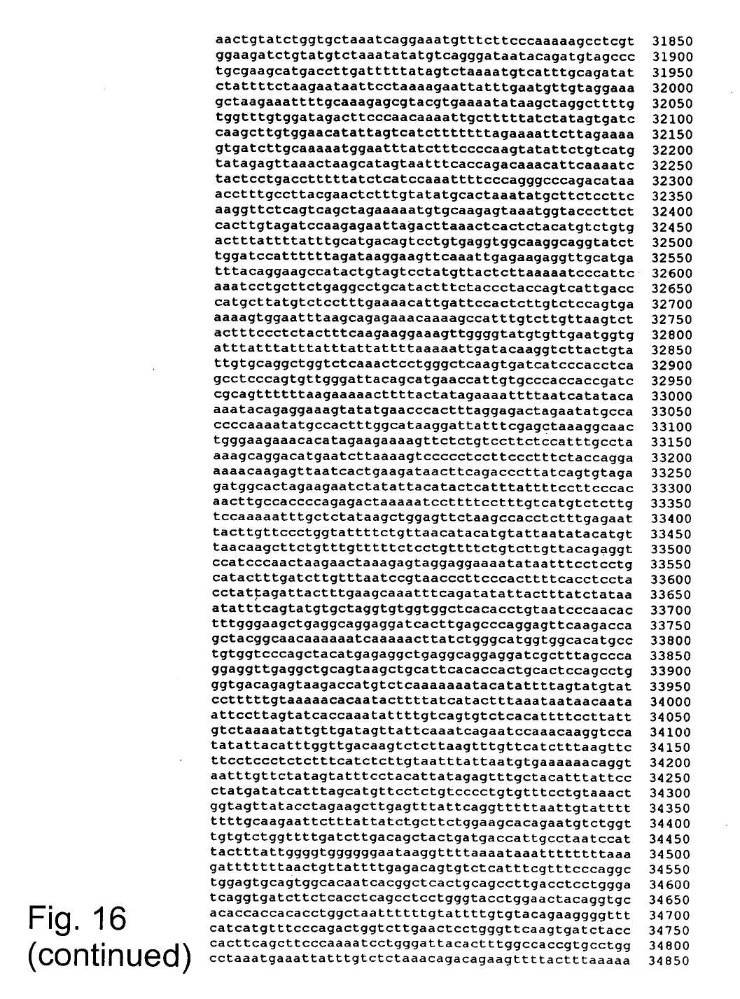

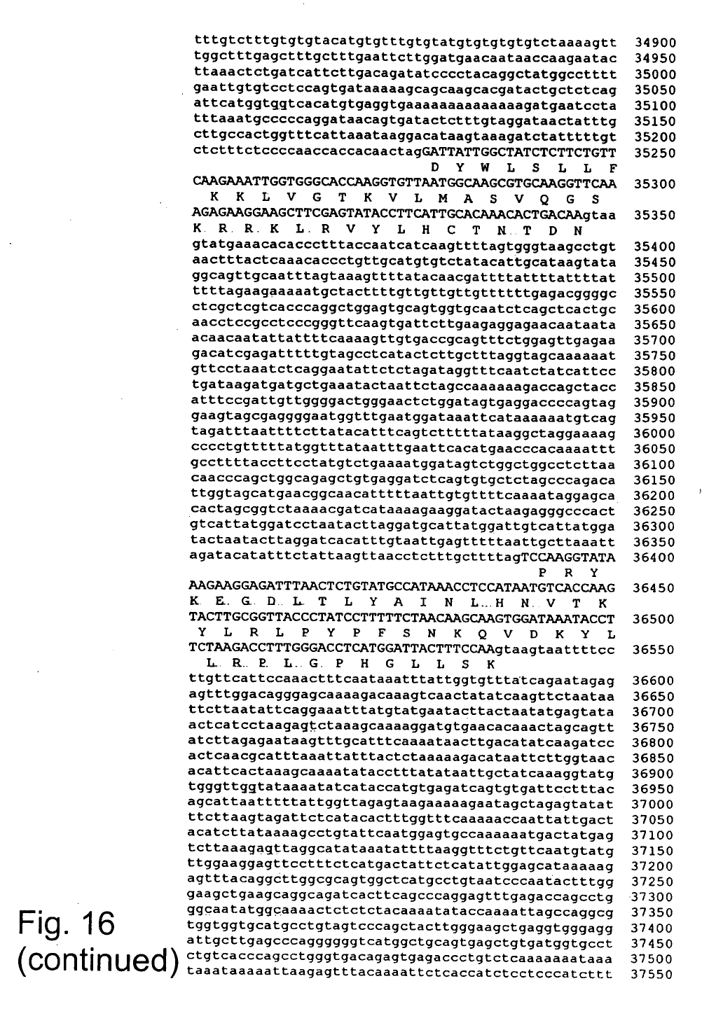

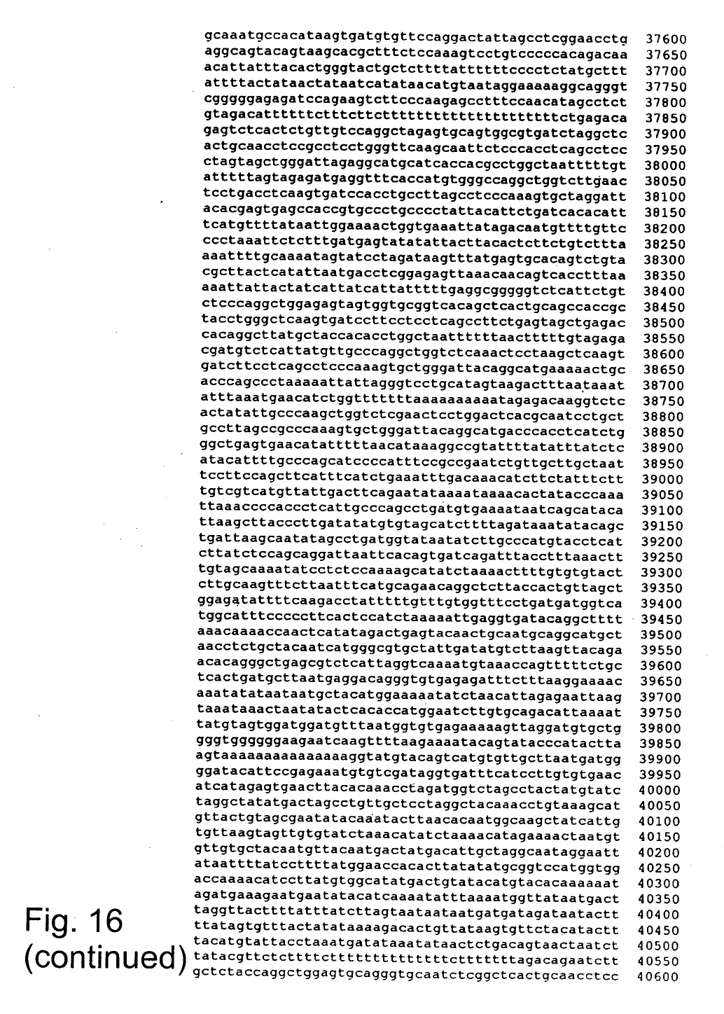

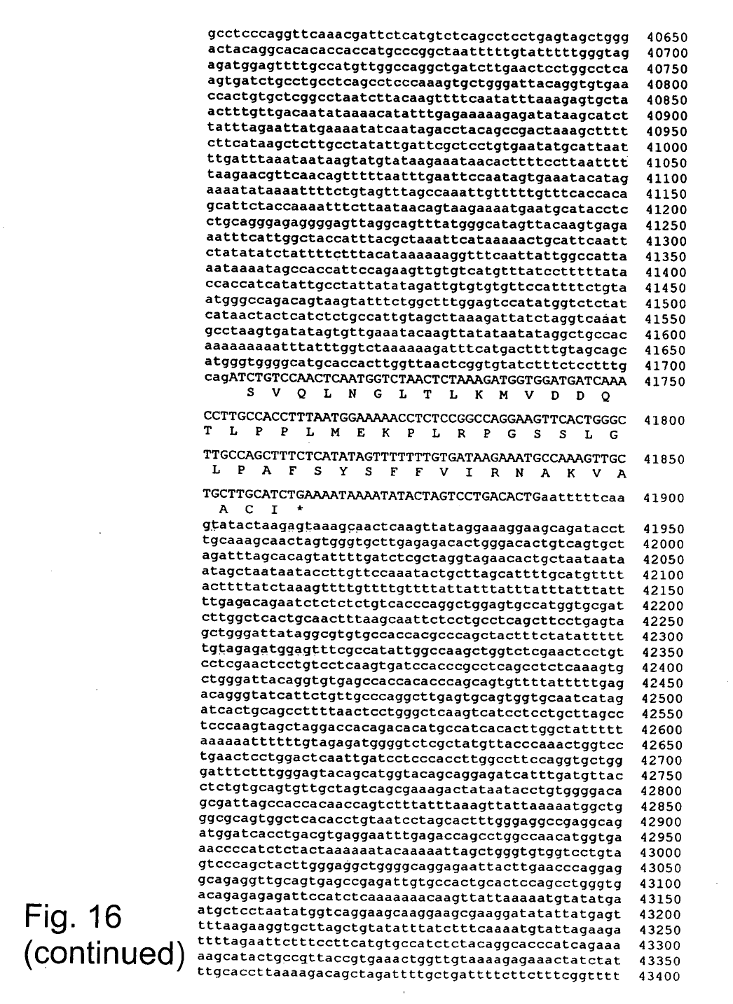

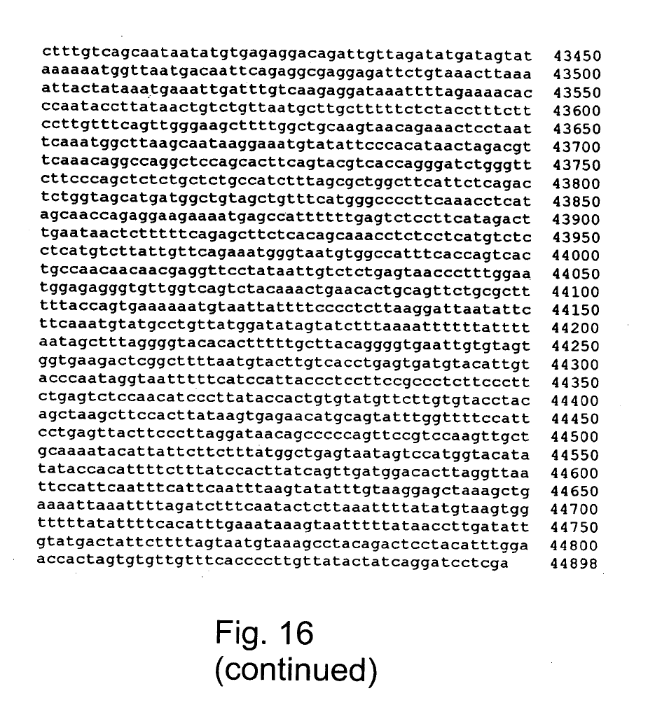

Large scale DNA sequencing of the three Lambda clones and the amplified fragment was performed with Lambda purified DNA by primer walking. A nucleotide sequence of 44,898 bp was analyzed (FIG. 16, SEQ ID NO:42). Comparison of the genomic sequence with that of hpa cDNA revealed 12 exons separated by 11 introns (FIGS. 15 an 16). The genomic organization of the hpa gene is depicted in FIG. 15 (top). The sequence include the coding region from the first ATG to the stop codon which spans 39,113 nucleotides, 2742 nucleotides upstream of the first ATG and 3043 nucleotides downstream of the stop codon. Splice site, consensus sequences were identified at exon/intron junctions.

### EXAMPLE 11

### Alternative Splicing

Several minor RT-PCR products were obtained from various cell types, following amplification with hpa specific primers. Each one found to contain a deletion of one or two exons. Some of these PCR products contain ORFs, which encode potential shorter proteins.

Table 1 below summarizes the alternative spliced products isolated from various cell lines.

Fragments of similar sizes were obtained following amplification with two cell lines, placenta and platelets.

| Cell type | Nucleotides deleted | Exons deleted | ORF |
|---|---|---|---|
| Platelets | 1047-1267 | 8, 9 | + |
| Platelets | 1154-1267 | 9 | − |
| Platelets | 289-435, 562-735 | 2, 4 | − |
| Sk-hep1, platelets, Zr75 | 562-735 | 4 | + |
| Sk-hep1 (hepatoma) | 561-904 | 4, 5 | − |
| Zr75 (breast carcinoma) | 96-203 | 1 (partial) | + |

### EXAMPLE 12

### Mouse and Rat hpa

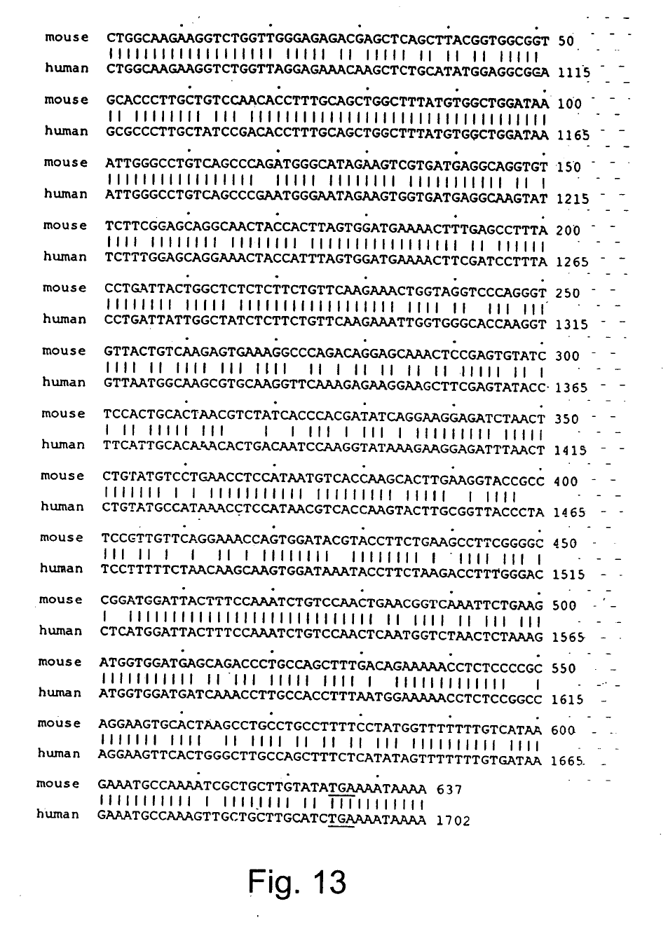

EST databases were screened for sequences homologous to the hpa gene. Three mouse ESTs were identified (accession No., Aa177901, from mouse spleen-Aa067997 from mouse skin, Aa47943 from mouse embryo), assembled into a 824 bp: cDNA fragment which contains a partial open reading frame (lacking a 5′ end) of 629 bp and a 3′ untranslated region of 195 bp (SEQ ID NO:12). As shown in FIG. 13, the coding region is 80% similar to the 3′ end of the hpa cDNA sequence. These EST's are probably cDNA fragments of the mouse hpa homolog that encodes for the mouse heparanase.

Searching for consensus protein domains revealed an amino terminal homology between the heparanase and several precursor proteins such as Procollagen Alpha 1 precursor, Tyrosine-protein kinase-RYK, Fibulin-1, Insulin-like growth factor binding protein and several others. The amino terminus is highly hydrophobic and contains a potential trans-membrane domain. The homology to known signal peptide sequences suggests that it could function as a signal peptide for protein localization.

The amino acid sequence of human heparanase was used to search for homologous sequences in the DNA and protein databases. Several human EST's were identified, as well as mouse sequences highly homologous to human heparanase. The following mouse EST's were identified AA177901, AA674378, AA67997, AA047943, AA690179, AI122034, all sharing an identical sequence and correspond to amino acids. 336-543 of the human heparanase sequence. The entire mouse heparanase cDNA was cloned, based on the nucleotide sequence of the mouse EST's. PCR primers were designed and a Marathon RACE was performed using a Marathon cDNA library from 15 days mouse embryo (Clontech) and from BL6 mouse melanoma cell line. The mouse hpa homologous cDNA was isolated following several amplification steps. A 1.1 kb fragment was amplified from mouse embryo Marathon cDNA library. The first cycle of amplification was performed with primers mhpl773 and Ap1 and the second cycle with primers mhpl736 and AP2. A 1.1 kb fragment was then amplified from BL6 Marathon cDNA library. The first cycle of amplification was performed with the primers mhp1152 and Ap1, and the second with mhpl83 and AP2. The combined sequence was homologous to nucleotides 157-1702 of the human hpa cDNA, which encode amino acids 33-543. The 5′ end of the mouse hpa gene was isolated from a mouse genomic DNA library using the Genome Walker kit (Clontech). An 0.9 kb fragment was amplified from a DraI digested Genome walker DNA library. The first cycle of amplification was performed with primers mhpl114 and Ap1 and the second with primers mhpl103 and AP2. The assembled sequence (SEQ ID NOs:43, 45) is 2396 nucleotides long. It contains an open reading frame of 1605 nucleotides, which encode a polypeptide of 535 amino acids (SEQ ID NOs:44, 45), 196 nucleotides of 3′ untranslated region (UTR), and an upstream sequence which includes the promoter region and the 5′-UTR of the mouse hpa cDNA. According to two promoter predicting programs TSSW and TSSG, the transcription start site is localized to nucleotide 431 of SEQ ID NOs:43, 45, 163 nucleotides upstream of the first ATG codon., The 431 upstream genomic sequence contains the promoter region. A TATA box is, predicted at position 394 of SEQ ID NOs:43, 45. The mouse and the human hpa genes share an average homology of 78% between the nucleotide sequences and 81% similarity between the deduce amino acid sequences.

Search for hpa homologous sequences, using the Blast 2.0 server revealed two EST's from rat: A1060284 (385 nucleotides, SEQ ID NO:46) which is homologous to the amino terminus (68% similarity to amino acids 12-136) of human heparanase and A1237828 (541 nucleotides, SEQ ID NO:47) which is homologous to the carboxyl terminus (81% similarity to amino acids 500-543) of human heparanase, and contains a 3′-UTR. A comparison between the human heparanase and the mouse and rat homologous sequences is demonstrated in FIG. 17.

### EXAMPLE 13

### Prediction of Heparanase Active Site

Homology search of heparanase amino acid sequence against the DNA and the protein databases revealed no significant homologies. The protein secondary structure as predicted by the PHD program consists of alternating alpha helices and beta sheets. The fold recognition server of UCLA predicted alpha/beta barrel structure, with under-threshold confidence.

Five of 15 proteins, which were predicted to have most similar folds, were glycosyl hydrolases from various organisms: 1xyza—xylanase from Clostridium Thermocellum, 1pbga—6-phospho-beta-δ-galactosidase from Lactococcus Lactis, 1amy—alpha-amylase from Barley, 1 ecea—endocellulase from Acidothermus Cellulolyticus and 1qbc—hexosaminidase alpha chain, glycosyl hydrolase.

Protein homology search using the bioaccelerator pulled out several, proteins, including glycosyl hydrolyses such as beta-fructofuranosidase from Vicia faba (broad bean) and from potato, lactase phlorizin hydrolase from human, xylanases from Clostridium thermocellum and from Streptomyces halstedii and cellulase from Clostridium thermocellum. Blocks 9.3 database pulled out the active site of glycosyl hydrolases family five, which includes cellulases from various bacteria and fungi. Similar active site motif is shared by several lysosomal acid hydrolases (63) and other glycosyl hydrolases. The common mechanism shared by these enzymes involves two glutamic acid residues, a proton donor and a nucleophile.

Despite the lack of an overall homology between the heparanase and other glycosyl hydolases, the amino acid couple Asp-Glu (NE), which is characteristic of the proton donor of glycosyl hydrolyses of the GH-A clan, was found at positions 224-225 of the human heparanase protein sequence. As in other clan members, this NE couple is located at the end of a sheet.

Considering the relative location of the proton donor and the predicted secondary structure, the glutamic acid that functions as nucleophile is most likely located at position 343, or at position 396. Identification of the active site and the amino acids directly involved in hydrolysis opens the way for expression of the defined catalytic domain. In addition, it will provide the tools for rational design of enzyme activity either, by modification of the microenvironment or catalytic site itself.

### EXAMPLE 14

### Expression of hpa Antisense in Mammalian Cell Lines

A mammalian expression vector Hpa2Kepcdna3 was constructed in order to express hpa antisense in mammalian cells hpa cDNA (1.7 kb EcoRI fragment) was cloned into the plasmid pcDNA3 in 3′>5′ (antisense) orientation. The construct was used to transfect MBT2-T50 and T24P cell lines. 2×10⁵ cells in 35 mm plates were transfected using the Fugene protocol (Boehringer Mannheim). 48 hours after transfection cells were trypsinized and seeded in six well plates. 24 hours later G418′ was added to initiate selection. The number of Colonies per 35 mm plate following-3 weeks:

| | Antisense | No insert |
|---|---|---|
| T24P | 15 | 60 |
| MBT-T50 | 1 | 6 |

The lower number of colonies obtained after transfection with hpa antisense, as compared with the control plasmid suggests that the introduction, of hpa antisense interfere with cell growth. This experiment demonstrates the use of complementary antisense hpa DNA sequence to control heparanase expression in cells. This approach may be used to inhibit expression of heparanase in vivo, in, for example, cancer cells and in other pathological processes in which heparanase is involved.

### EXAMPLE 15

### Zoo Blot

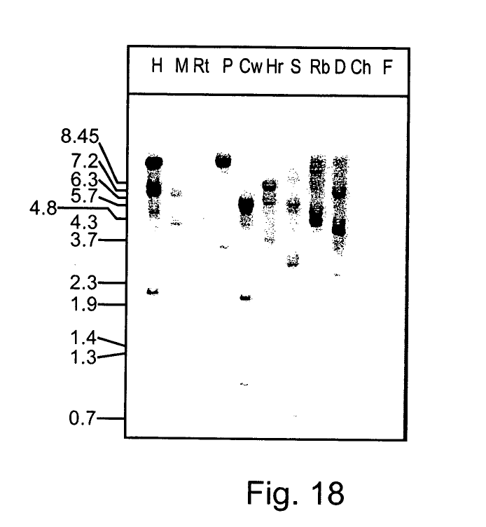

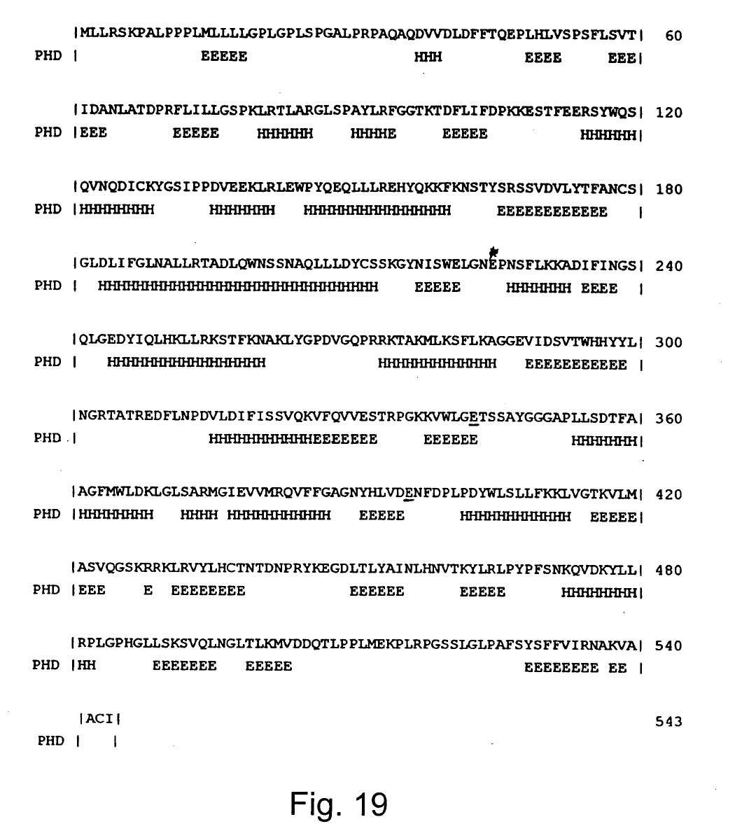

Hpa cDNA was used as a probe to detect homologous sequences in human DNA and in DNA of various animals. The autoradiogram of th Southern analysis is presented in FIG. 18. Several bands were detected in human DNA, which correlated with the accepted pattern according to the genomic hpa sequence. Several intense bands were detected in all mammals, while faint bands were detected in chicken. This correlates with the phylogenetic relation between human and the tested animals. The intense bands indicate that hpa is conserved among mammals as well as in more genetically distant organisms. The multiple bands patterns suggest that in all animals, like in human, the hpa locus occupy large genomic region. Alternatively the various bands could represent homologous sequences and suggest the existence of a gene family, which can be isolated based on their homology to the human hpa reported herein. This conservation was actually found, between the isolated human hpa cDNA and the mouse homologue.

### EXAMPLE 16

### Characterization of the hpa Promoter

The DNA sequence upstream of the hpa first ATG was subjected, to computational analysis in order to localize the predicted transcription start site and to identify potential transcription factors binding sites. Recognition of human. PolII promoter region and start of transcription were predicted using the TSSW and TSSG programs. Both programs identified a promoter region upstream of the coding region. TSSW pointed at nucleotide 2644 and TSSG at 2635 of SEQ ED NO:42. These two predicted transcription start sites are located 4 and 13 nucleotides upstream of the longest hpa cDNA isolated by RACE.

A hpa promoter-GFP reporter-vector was constructed in order to investigate the regulation of hpa transcription. Two constructs were made, containing 1.8 kb and 1.1 kb of the hpa promoter region. The reporter vector was transfected into T50-mouse bladder carcinoma cells. Cells transfected with both constructs exhibited green: fluorescence, which indicated the promoter activity of the genomic sequence upstream of the hpa-coding region. This reporter vector, enables the monitoring of hpa promoter activity, at various conditions and in different cell types and to characterize the factors involved regulation of hpa expression.

### EXAMPLE 17

### Expression of Heparanase by Vascular EC

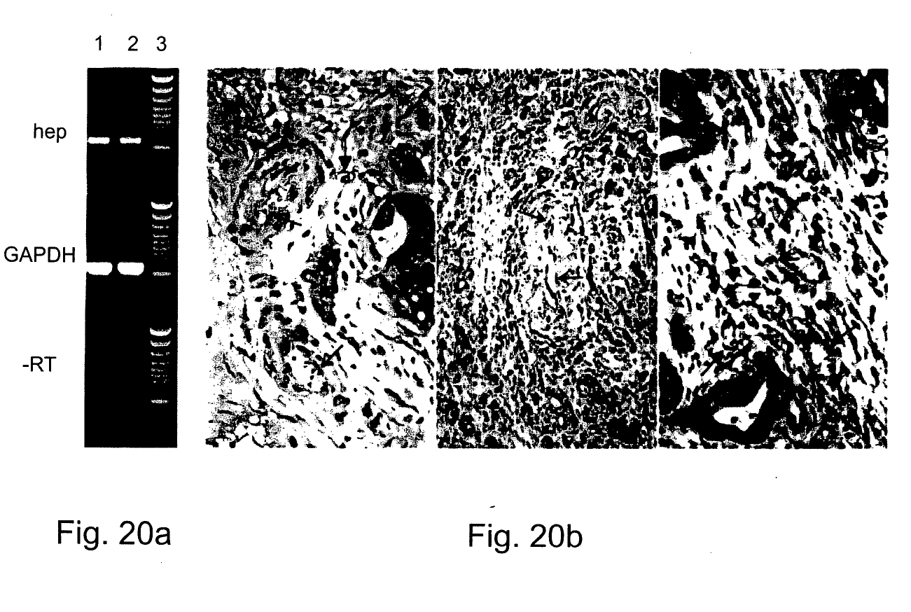

Previously, it has been suggested that stimulated EC secrete heparanase-like activity [Godder, K. et al. Heparanase activity in cultured endothelial cells. J Cell Physiol 148, 274-280 (1991); Pillarisetti, S. et al. Endothelial cell heparanase modulation of lipoprotein lipase activity. Evidence that heparan sulfate., oligosaccharide is an extracellular chaperone. J Biol Chem 272, 15753-15759 (1997)]. Using RT-PCR, it was unequivocally demonstrated, for the first time, that the heparanase gene is expressed by proliferating human ECs. Both cultured human umbilical vein EC (HUVEC) and human bone marrow EC (TrHBMEC) [Schweitzer, K. M. et al. Characterization of a newly established human bone marrow endothelial cell line: distinct adhesive properties for hematopoietic progenitors compared with human umbilical vein endothelial cells. Lab Invest 76, 25-36 (1997)] expressed the heparanase gene, as reflected by the 564-bp PCR product (FIG. 20a).

### EXAMPLE 18

### Expression of Heparanase in ECs in Human Blood Vessels

Paraffin embedded sections from patients with primary colon adenocarcinoma were subjected to immunohistochemical staining with monoclonal anti-heparanase antibodies. An interesting pattern of staining was noted in EC in blood vessels of different maturation stages. The heparanase protein is preferentially expressed in sprouting capillaries (FIG. 20b, left and right, arrows) whereas the endothelium of mature quiescent vessels showed no detectable levels of heparanase (FIG. 20b, left and middle, concave arrows). A similar expression pattern was observed in human mammary and pancreatic carcinomas. This result suggests a significant role of endothelial heparanase in enabling EC to traverse BM and ECM barriers during sprouting angiogenesis. As previously reported [Vlodavsky, I. et al. Mammalian heparanase: gene cloning, expression and function in tumor progression and metastasis. Nat Med 5, 793-802 (1999)] and also demonstrated in FIG. 20b, the neoplastic colonic mucosa exhibited an intense heparanase staining, as opposed to no expression of heparanase in normal colon epithelium [Vlodavsky, I. et al. Mammalian heparanase: gene cloning, expression and function in tumor progression and metastasis. Nat Med 5, 793-802 (1999)]. Carcinoma cells can therefore be regarded as the main source of heparanase in the tumor microenvironment. Moreover, at a later stage of tumor progression, heparanase was also found in the tumor stroma.

### EXAMPLE 19

### Release of ECM Bound ¹²⁵I-bFGF by Heparanase

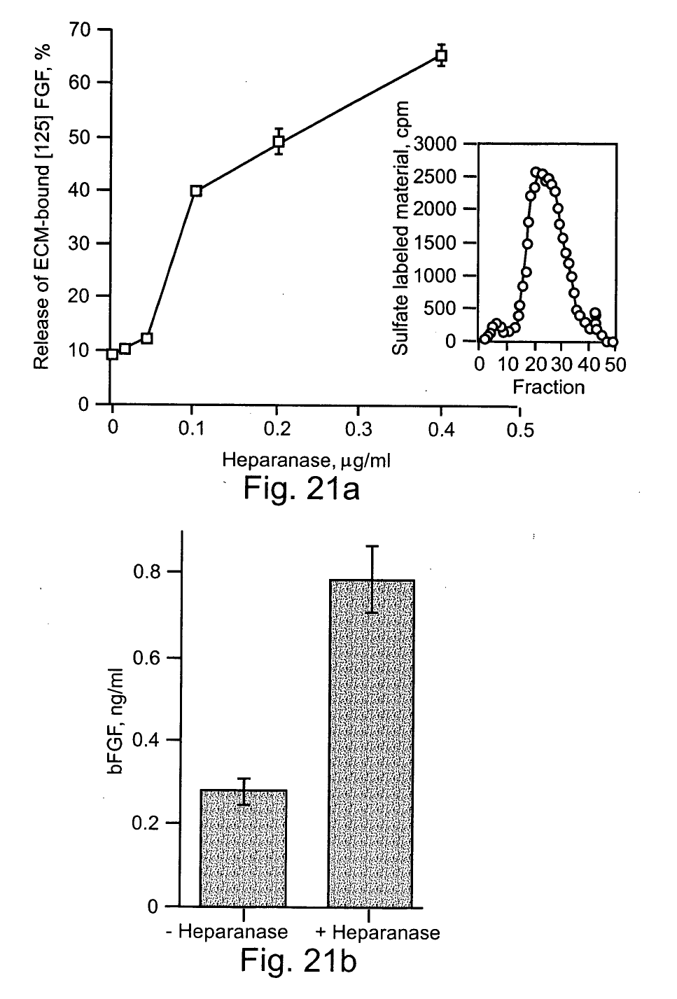

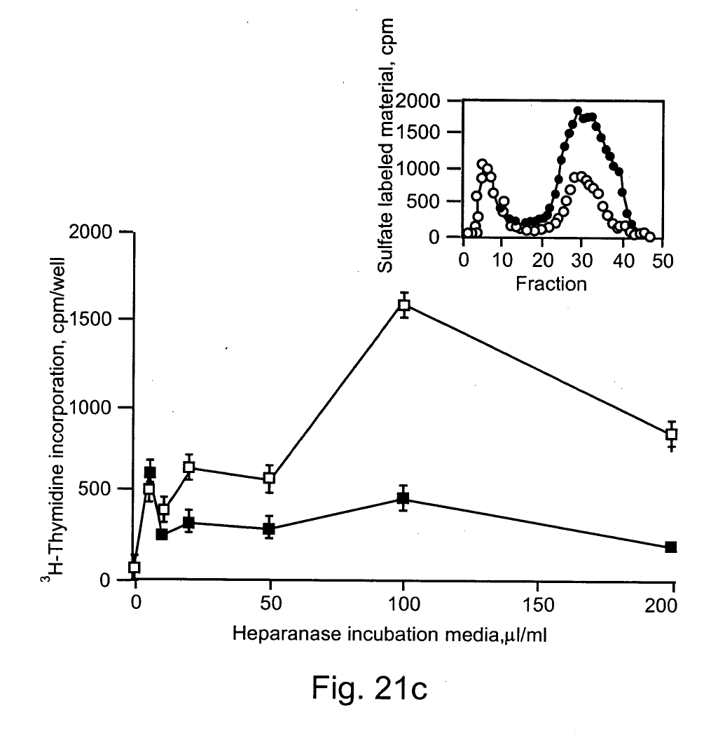

Naturally produced subendothelial ECM was preincubated with ¹²⁵I-bFGF, washed free of the unbound bFGF and incubated (3 hours, 37° C.) with the 50 kDa active form of the recombinant heparanase enzyme. As demonstrated in FIG. 21a, degradation of HS in the ECM, reflected by release of sulfate labeled HS degradation fragments (inset), resulted in release of as much as 70% of the ECM-bound ¹²⁵I bFGF. Alternatively, the enzyme was added to native ECM that was not preincubated with ¹²⁵I-bFGF. Aliquots of the incubation medium were then tested for the presence of bFGF, using a quantitative ELISA for bFGF. Nearly 0.8 ng endogenous bFGF were released from ECM coating the surface area of a 35 mm culture dish (FIG. 21b). The released bFGF stimulated 5-8 fold the proliferation of 3T3 fibroblasts and bovine aortic EC. These results clearly indicate that heparanase releases active bFGF sequestered as a complex with HS in the ECM. Both tumor and endothelial heparanase may hence elicit an indirect angiogenic response by means of releasing active HS-FGF complexes from storage in the ECM and tumor microenvironment.

### EXAMPLE 20

### Release of ECM Bound bFGF by Heparanase—bFGF Cellular Response Assay

The ability of heparanase cleaved HS degradation fragments to promote the mitogenic activity of bFGF was investigated using a cytokine-dependent lymphoid cell line (BaF3, clone 32) engineered to express FGF-receptor 1 (FGFR1) [Miao, H. Q., Ornitz, D. M., Aingom, E., Ben-Sasson, S. A. & Vlodavsky, I. Modulation of fibroblast growth factor-2 receptor binding, dimerization, signaling, and angiogenic activity by a synthetic heparin-mimicking polyanionic compound. J Clin Invest 99, 1565-1575 (1997); Ornitz, D. M. et al. Heparin is required for cell-free binding of basic fibroblast growth factor to a soluble receptor and for mitogenesis in whole cells. Mol Cell Biol 12, 240-247 (1992)]. These cells lack cell surface HS and respond to bFGF only in the presence of exogenously added species of heparin or HS [Miao, H. Q., Ornitz, D. M., Aingom, E., Ben-Sasson, S. A. & Vlodavskly, I. Modulation of fibroblast growth factor-2 receptor binding, dimerization, signaling, and angiogenic activity by a synthetic heparin mimicking polyanionic compound. J Clin Invest 99, 1565-1575 (1997); Ornitz, D. M. et al. Heparin is required for cell-free binding of basic fibroblast growth factor to a soluble receptor and for mitogenesis in whole cells. Mol Cell Biol 12, 240-2-47 (1992)]. Both native ECM and confluent vascular EC monolayer were first treated with the recombinant 50 kDa heparanase enzyme. Aliquots of the incubation media were-then added to BaF3 cells and tested for their ability to promote ³H-thymidine incorporation in response to bFGF. As expected, BaF3 cells exposed to either bFGF or heparanase alone exhibited almost no incorporation of ³H-thymidine. A marked stimulation (about 40 fold) of DNA synthesis was obtained in the presence of HS degradation fragments released by heparanase from EC surfaces (FIG. 21c). Interestingly, HS fragments released by heparanase from the subendothelial ECM exerted a much smaller effect (FIG. 21c). These results indicate that the heparanase enzyme potentiates the mitogenic activity of bFGF and possibly other heparin-binding angiogenic growth factors, through release of HS degradation fragments that promote bFGF-receptor binding and activation. The observed difference in biological activity between cell surface- and ECM-derived HS fragments indicates that the primary role of HS in the ECM is to sequester, protect and stabilize heparin-binding growth factors, while the ell surface H4S plays a more active role in promoting the mitogenic and angiogenic activities of the growth factor by means of stimulating receptor-binding, dimerization and activation. This concept is supported by the recently reported preferential ability of cell surface-vs. ECM-HSPG to mediate the assembly of bFGF-receptor signaling complex [Chang, Z., Meyer, K., Rapraeger, A. C. & Friedl, A. Differential ability of heparan sulfate proteoglycans to assemble the fibroblast growth factor receptor complex in situ. FASEB J. 14, 137-144 (2000)]. The biochemical nature of (e.g., size, sequence) of oligosaccharides released by heparanase from cells vs. ECM is being characterized.

### EXAMPLE 21

### Induction of Angiogenesis into a Matrigel Plug In Vivo

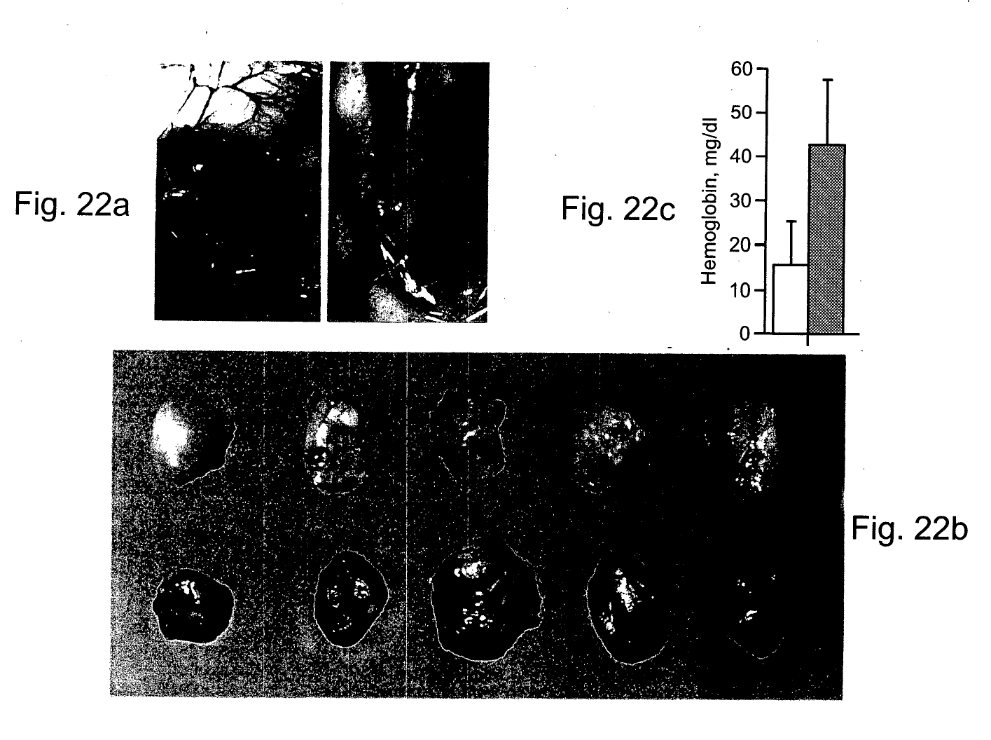

The Matrigel plug assay [Passaniti, A. et al. A simple, quantitative method for assessing angiogenesis and antiangiogenic agents using reconstituted basement membrane, heparin, and fibroblast growth factor. Lab Invest 67; 519-528 (1992)] was applied to investigate whether the heparanase enzyme can elicit an angiogenic response in vivo. For this purpose, stable heparanase transfected Eb lymphoma cells [Vlodavsky, I. et al. Mammalian heparanase: gene cloning, expression and function in tumor progression and metastasis. Nat Med 5, 793-802 (1999)] were mixed at 4° C. with Matrigel (reconstituted BM preparation extracted from EHS mouse sarcoma) and injected subcutaneously into BALB/c mice. Similarly treated mock-transfected Eb cells expressing no heparanase activity served as a control [Vlodavsky, I. et al. Mammalian heparanase: gene cloning, expression and function in tumor progression and metastasis. Nat Med 5, 793-802 (1999)]. Upon injection, the liquid Matrigel rapidly forms a solid gel plug that serves as a supporting medium for the lymphoma cells. Its major components, similar to intact BM, are laminin, collagen type IV and HSPGs. Matrigel also contains bFGF and other growth factors that are naturally found in BM and ECM [Vukicevic, S. et al. Identification of multiple active growth factors in basement membrane Matrigel suggests caution in inhibition of cellular activity related to extracellular matrix components. Exp Cell Res 202, 1-8 (1-992)]. Hence, the Matrigel in this experimental system serves not merely as an inert vehicle for the enzyme producing cells, but rather maintains the natural interactions existing between tumor cells and the surrounding ECM, providing, among other effects, a source of ECM-sequestered bFGF. As shown in FIG. 22, a pronounced angiogenic response was induced by Matrigel embedded Eb cells over expressing the heparanase enzyme, as compared to little or no neovascularization exerted by mock transfected Eb cells expressing no heparanase activity. The angiogenic response was reflected by a network of capillary blood vessels attracted toward the Matrigel plug containing heparanase transfected (FIG. 22a, left) vs. control mock transfected (FIG. 22a, right) Eb cells, and by a large amount of blood and vessels seen in the isolated Matrigel plugs excised from each of the mice. (FIG. 22b, bottom, vs. top, respectively). This difference was highly significant, as also demonstrated by measurements of the hemoglobin content of Matrigel plugs removed from each mouse of the respective groups (FIG. 22c).

### EXAMPLE 22

### Wound Closure

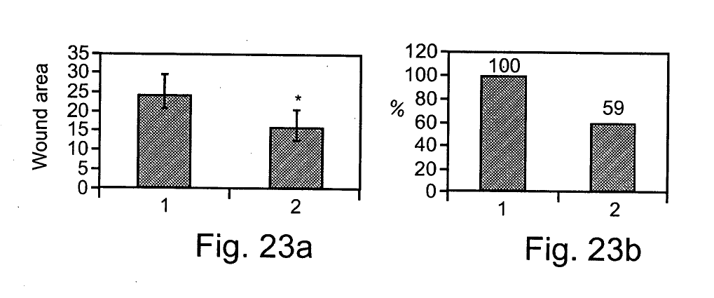

In order to directly study the effect of heparanase on the complex of events resulting in wound healing, 1 μg (in 20 μl saline) active heparanase was applied topically onto full-thickness wounds. This reflects a ten-fold lower protein concentration as compared with a previous study focusing on the role of nerve growth factor (NGF) in wound healing [Hiroshi M., H. Koyama, H. Sato, J. Sawada, A. Itakura, A. Tanaka, M. Matsumoto, K. Konno, H. Ushio and K. Matsuda. 1998. Role of nerve growth factor in cutaneous wound healing. Accelerating effect in normal and healing-impaired diabetic mice. J. Exp. Med. 187: 297-303]. Careful evaluation of wound areas revealed a significant improvement of wound closure upon heparanase treatment (FIGS. 23a-b). Thus, while average wound area was 24.3 mm² (+/−5.1) for saline-treated control wounds, heparanase-treated wounds area was 15.5 mm² (+/−3.1) (FIG. 23a), which represent a 40% decrease in wound area (FIG. 23b). Differences were found to be statistically significant (P=0.00238).

### EXAMPLE 23

### Microscopic Analysis of Heparanase Treated Wounds

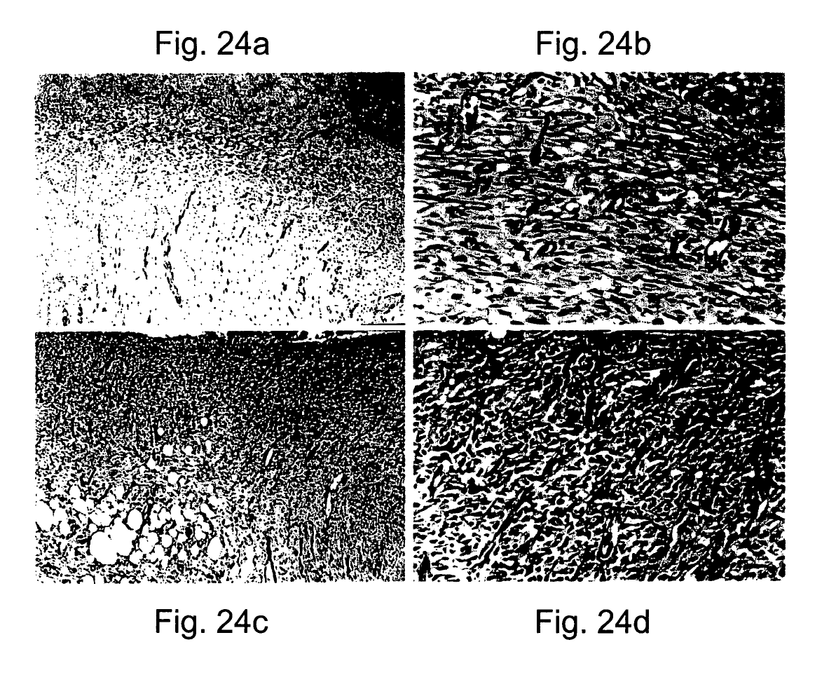

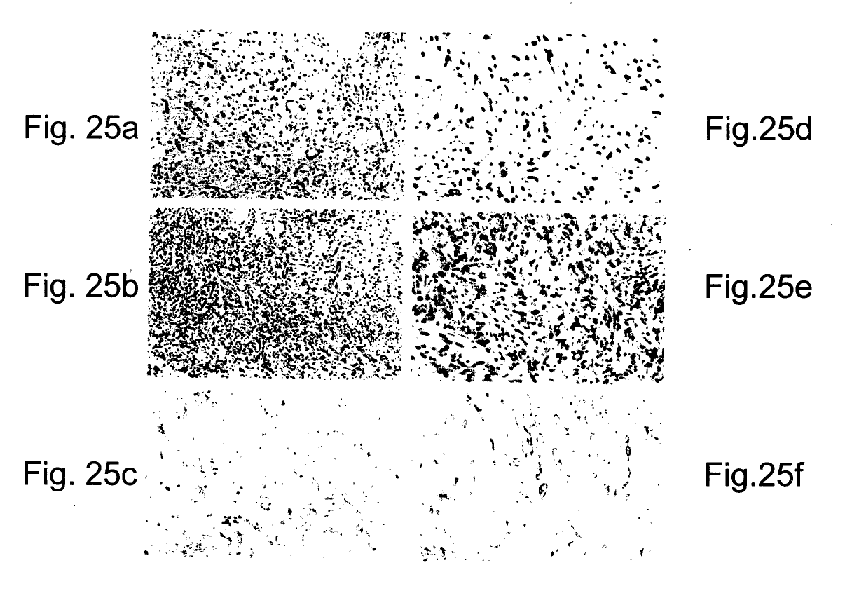

Having demonstrated, for the first time, a direct role for heparanase activity in the wound healing process, cellular and molecular mechanisms that are activated by heparanase in the course of wound healing were sought. Examination of hematoxilin-eosin stained wound sections revealed the expected granulation tissue morphology, composed of fibroblasts, blood vessels and inflammatory cells (FIGS. 24a-b). Interestingly, the heparanase-treated granulation tissue was much more dense. Specifically, a significant increase in the number of inflammatory cells and blood vessels was observed (FIGS. 24c-d). This was further confirmed by staining for PCNA, a marker for cell proliferation (FIGS. 25a-b and 25d-e) and for PECAM-1, a marker for endothelial cells (FIGS. 25c-f). Indeed, an increase in PCNA (FIGS. 25d-e) and PECAM-1 (FIGS. 25c and 25f) staining was observed in the granulation tissue of heparanase-treated wounds. Thus, the acceleration of wound healing may be due, without limitation, to the robust fibroblast and inflammatory cells-derived cytokine and chemokines and to increased vascularity.

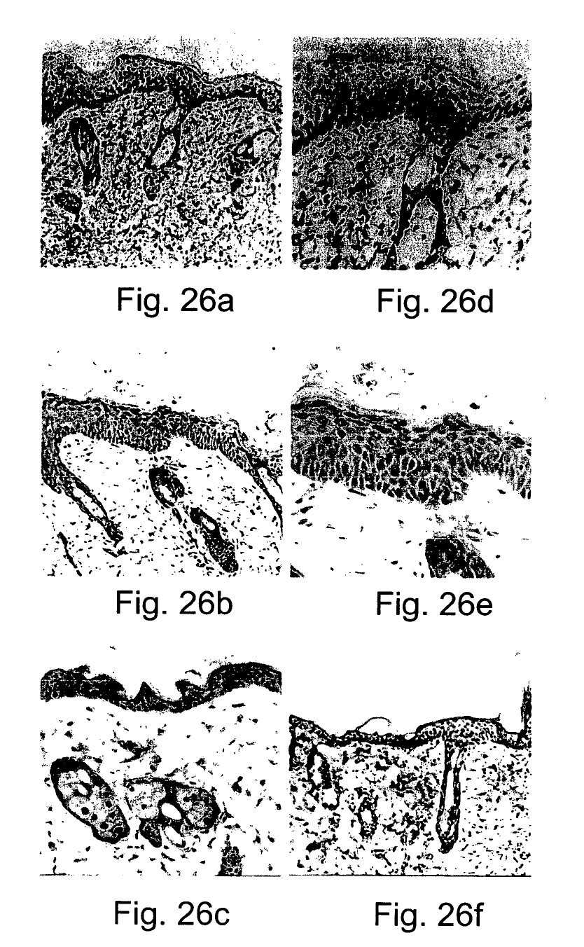

Heparanase was found to be expressed by all the major cell components of granulation tissue. Interestingly, heparanase expression was mainly detected in the differentiated, non-proliferating, cells composing the epidermis (FIGS. 26b and 26e-f), while proliferating, PCNA-positive epidermal cells (FIGS. 26a and 26d) reconstituting the wound were poorly stained. In addition, heparanase staining was observed in non-proliferating sebaceous glands (compare FIGS. 26a and 26d with FIG. 26c) cells. Such staining pattern suggests, without limitation, that heparanase plays a role in cellular terminal differentiation which leads, as in the case of keratinocyes, to apoptosis and further as an anti-infectant.

### EXAMPLE 24

### Stimulation of Angiogenesis by Heparanase in Wounded Rat Eye Model

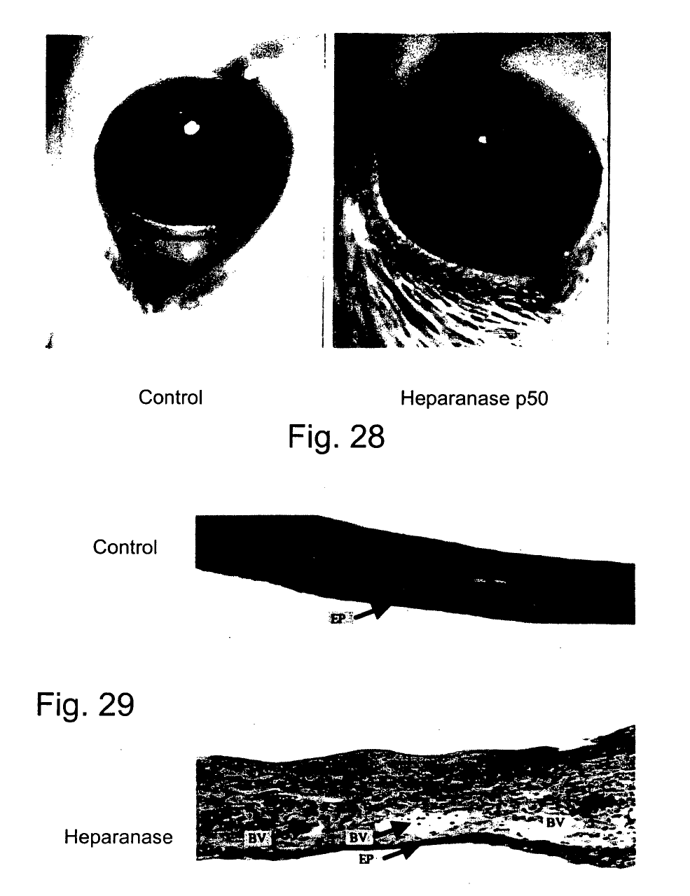

The central cornea of rats eyes was scraped with a surgical knife. The right eye of each rat was then treated with heparanase, 50 μl drop (1 mg/ml) of purified recombinant human P50 heparanase, three times a day. The left eye served as a control and was treated with Lyeteers. Vascularization and epithelialization were evaluated following closure of the corneal-lesion. As shown in FIG. 28a heparanase treated eyes exhibited vascularization of the cornea, as well as increased vascularization in the iris. Normal, minor vascularization of the iris and non vascular appearance of the cornea were observed in the controls (FIG. 28). Histological examination of cornea from control eyes (FIG. 29) showed healing of the epithelia which is accompanied by a normal organized structure of the cornea while heparanase treatment (FIG. 29) resulted in growth of blood vessels into the cornea (arrows), followed by a massive infiltration of lymphocytes. Vascularization associated inflammatory reaction interfered with corneal healing, as demonstrated by a disorganized structure of the cornea.

### EXAMPLE 25

### Treatment of Induced Diabetic Ulceration and Ischemic Wounds with Heparanase

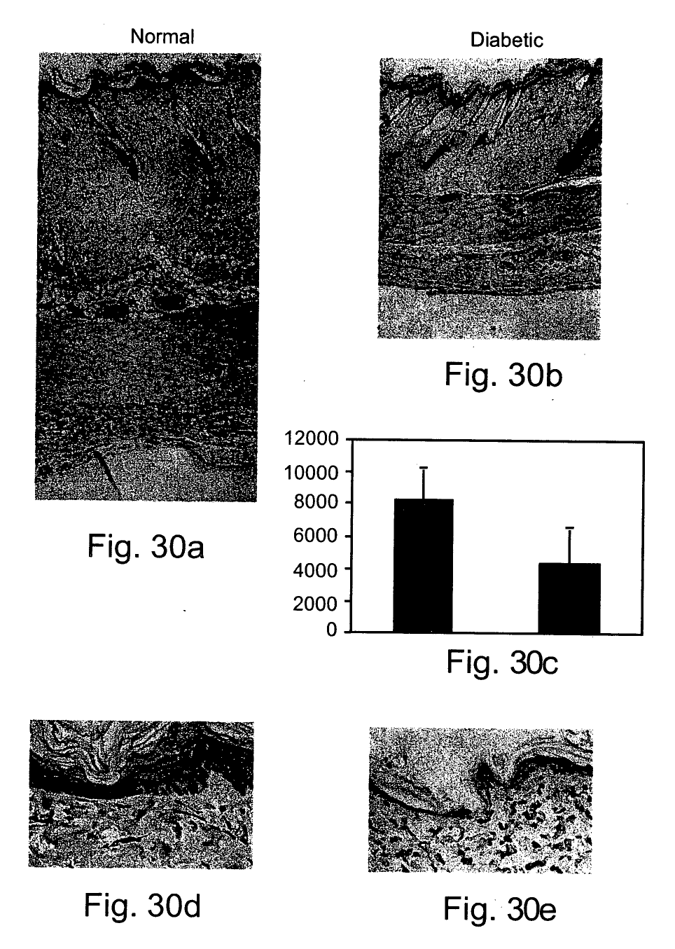

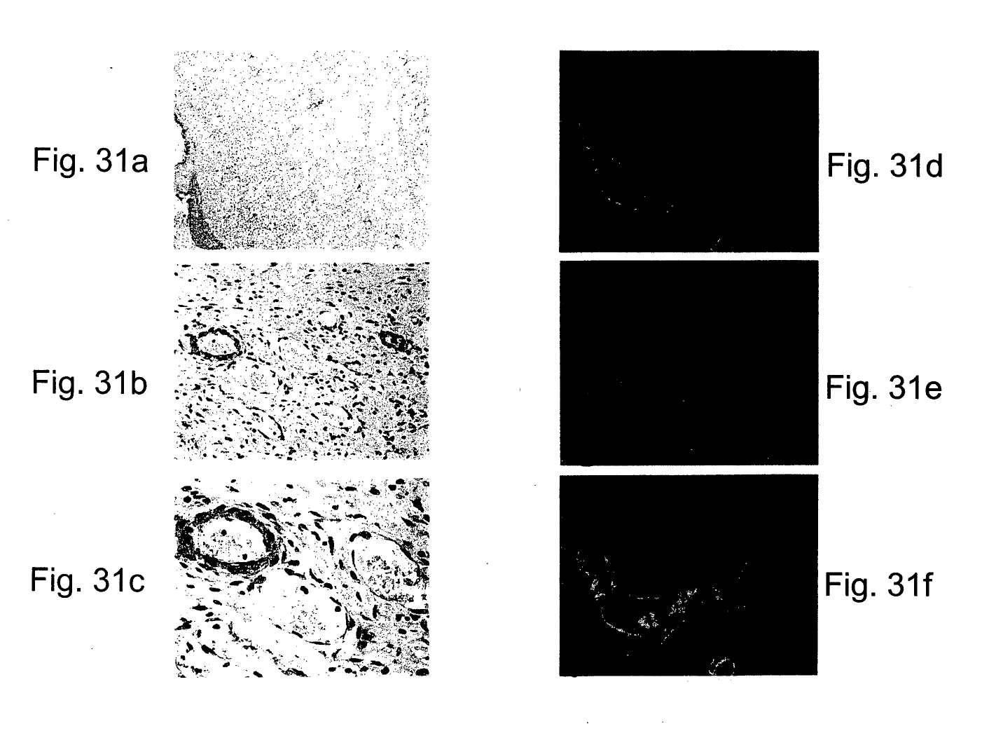

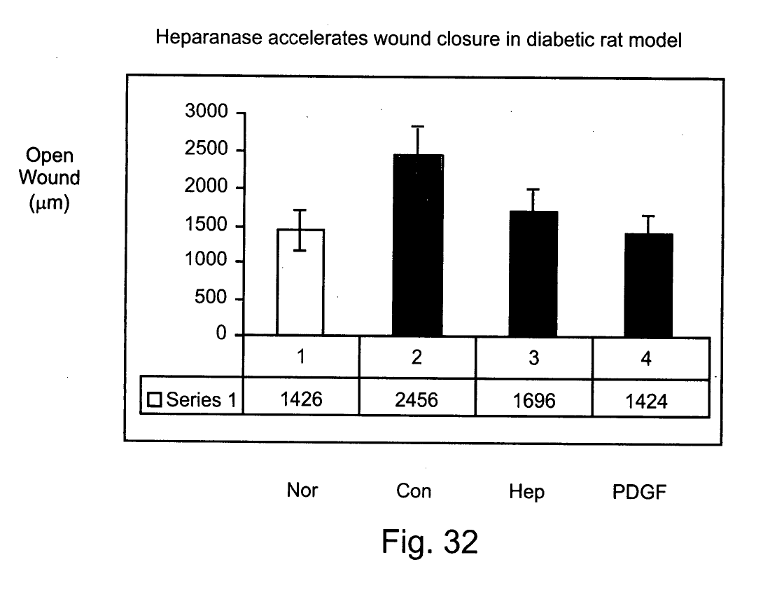

Wound healing is an efficient and rapid process under normal conditions and usually requires only minimal interventions. In contrast, wound healing is significantly impaired in diabetic patients and under ischemic conditions. The ability of heparanase to accelerate wound healing in animal models (streptozotocin-induced diabetic rats) that mimic such pathological conditions was hence tested. Interestingly, in model animals the whole skin tissue is dramatically altered under diabetic conditions, and the overall tissue thickness is reduced to about half (FIGS. 30a-c). This is due to a loss of tissue mass, mainly of the dermis and the sub-epidermal fat layers. Moreover, heparanase staining revealed a drastic reduction in the keratinocytes epidermis thickness and hence reduced heparanase expression under diabetic conditions (FIGS. 30d-e). In order to further explore the possible involvement of heparanase in normal wound healing, full-thickness wounds were immunostained with anti heparanase antibodies (FIGS. 31a-f). Heparanase expression was clearly detected in the newly formed wound granulation tissue (FIG. 31a). More specifically, blood vessels were noted to highly express heparanase (FIGS. 31b-c). In order to better define heparanase localization sections were double stained with anti-heparanase and anti-smooth muscle actin (SMA), a specific marker for blood vessels pericytes, antibodies (FIGS. 31d-f). Heparanase staining was only detected in the endothelial cells lining blood vessels, and mainly at lumen facing areas (FIGS. 31d-f). This suggests that at this stage of vessel maturation, heparanase may be secreted or acting on cell surface HSPG. Heparanase was not detected at the sub endothelial pericytes cell layer, which was specifically labeled with anti-SMA antibodies (FIGS. 31d and 31f). In additions, heparanase was also detected in non-endothelial cells, presumably fibroblasts (FIG. 31c). The presence of endogenous heparanase in the healing wound may suggest that heparanase forms a part of the complex healing mechanism. If this is indeed the case, then, the addition of exogenous heparanase may be beneficial and accelerate wound closure. Hence, wound closure in normal, non-diabetic rats (Nor) was compared with streptozotocin-induced diabetic rats (Con, FIG. 32 Full-thickness wounds were created with a circular 8 mm punch at the back of the rat. At 7 days post wounding wound diameter in normal rats was 1426 μm, which reflects 83% closure. In contrast, wound diameter in the diabetic animals was measured to be: 2456 μm, reflecting only 70% closure. Treating diabetic wounds with heparanase: (Hep, 1 μg/wound applied., topically in saline) resulted in some 30% improvement in wounds closure, while PDGF, the most potent wound healing treatment at the clinic, gave some 58% improvement only. Thus, heparanase seems to be a promising therapeutic agent already at this preliminary stage.

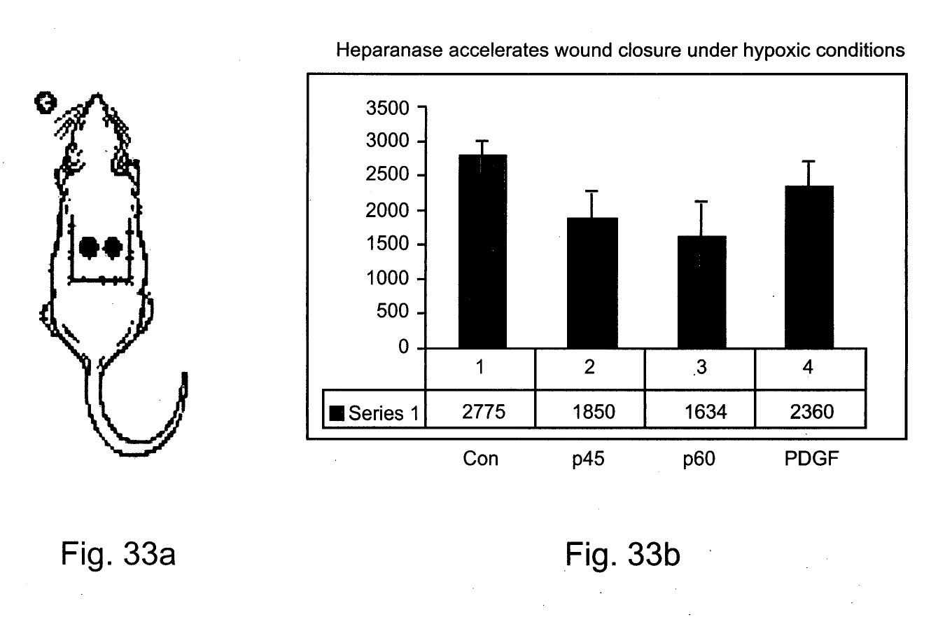

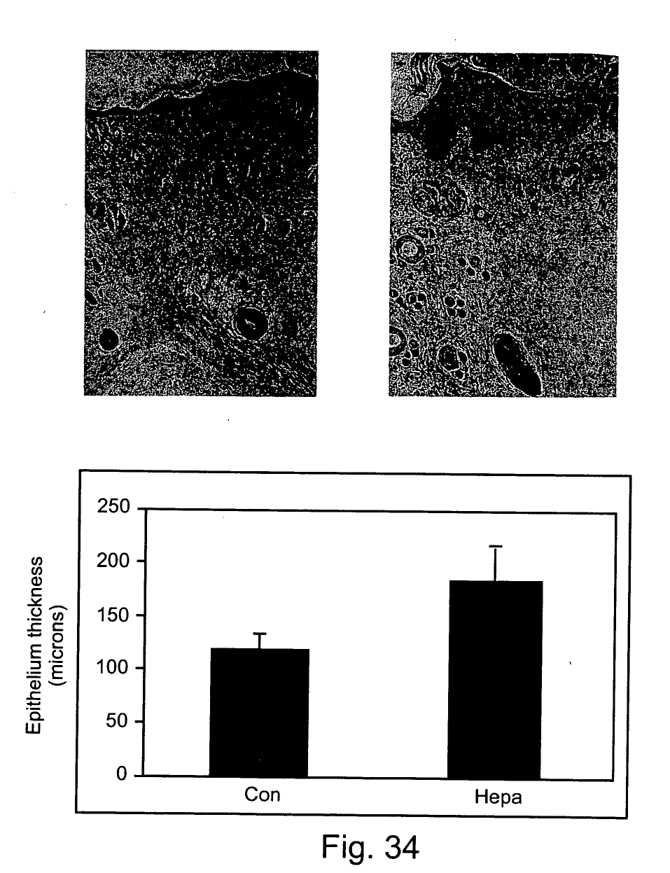

The diabetic state often involves ischemic conditions, which play a critical role in impaired wound healing. Ischemic conditions were generated by three incisions at the rat back skin, followed by punch wounds in the flap area, as describe in FIG. 33A (Norfleet A. M., Y. Huang, L. E. Sower, W. R. Redin, R. R. Fritz and D. H. Carney. 2000). Thrombin peptide TP508 accelerates closure of dermal excision in animal tissue with surgically induced ischemia (Wound Rep. Reg. 8: 517-529). Ischemic conditions significantly delay wound healing (compare Nor in FIG. 32 with Con in FIG. 33b), resulting in wounds twice as big. Interestingly, the active heparanase enzyme (p45), as well as the precursor version of the protein (p60), were both able to accelerate wound healing in this ischmic wound animal model to the level of wounds under non-ischemic conditions. These differences are statistically significant (p=0.032 and 0.016, for p45 and p60, respectively). Thus, heparanase was able to accelerate wound healing under both diabetic (FIG. 32) and ischemic (FIG. 33b) conditions. Moreover, a single p45 heparanase application at the incision made to create the flap resulted in significant increase in the epithelial cell layer thickness (FIG. 34). This observation not only supports the notion that heparanase may improve wound healing, but suggests that this ability also involves re-epithelialization which is the major mechanism that is responsible for human wound healing.

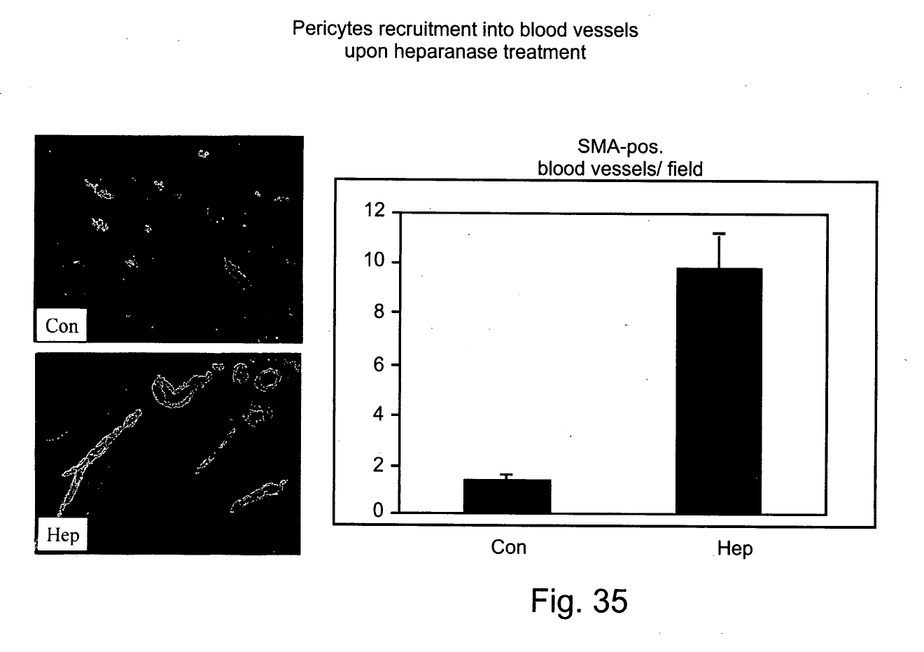

Heparanase (p45) induces granulation tissue vascularity, thus acts as an angiogenic factor (Elkin M, N. Ilan, R. Ishai-Michali, Y. Friedman, O. Papo, I. Pecker and I. Vlodavsky. 2001. Heparanase as a mediator of angiogenesis: mode of action. FASEB J. 15:1661). In order to further explore heparanase's angiogenic activity, wound sections were stained for smooth muscle actin (SMA), a specific marker for pericytes. Interestingly, the newly formed blood vessels in the wound granulation tissue were largely devoid of pericytes (FIG. 35a). In contrast, most blood vessels in the heparanase treated wound granulation tissues were stained positively for SMA (FIG. 35b). Careful counting revealed a 6 folds increase in SMA-positive blood vessels upon heparanase treatment (FIG. 35c). Thus, heparanase does not only increase vessels density, but also affects the recruitment of pericytes, which are believed to play a critical role in proper vascular development and vascular integrity (Benjamin L. E., I. Hemo and E. Keshet. 1998. A plasticity window for blood vessel remodeling is defined by pericytes coverage of the performed endothelial network and is regulated by PDGF-B and VEGF. Development 125: 1591-1598). A role for pericytes in maintaining vascular function was suggested by a number of gene knockout studies, including the disruption of the PDGF gene (Laveen P., M. Pekny, S. Gebre Medhin, B. Swolin, E. Larsson and C. Betsholtz. 1994. Mice deficient for PDGF B show renal, cardiovascular, and hematological abnormalities. Genes Dev. 8: 1875-1887; Lindahl P., B. R. Johansson and C. Betsholtz. 1997. Pericytes loss and microaneurysm formation in PDGF-B-deficient mice. Science 277: 242-245). PDGF, however, is not ECM-bound and heparanase effects on wound healing in general, and pericytes recruitment in particular, cannot be directly mediated by PDGF release. In addition to PDGF, the Tie-2 receptor and its ligand, angiopoetin −1 (Ang1), were implicated in blood vessels maturation. Mice lacking Tie-2 or Ang1 are embryonic lethal due to impaired development of the myocardium, defective remodeling of the primitive vascular plexus into small and large vessels, as well as complete lack of perivascular cells (Suri S., P. F. Jones, S. Patan, S. Bartukova, P. C. Maisonpierre, S. Davis, T. N. Sato and G. D. Yancopoulos. 1996. Requisite role of angiopoietin; 1, a ligand for the Tie2 receptor, during embryonic angiogenesis. Cell 87: 1171-1180; Sato T. N., Y. Tozawa, U. Deutsch, K. Wolburg-Buchholz, Y. Fujiwara, M. Gendron-Maguire, T. Gridley, H. Wolburg, W. Risau and Y. Qin. 1995. Distinct roles of the receptor tyrosine kinases Tie-1 and Tie-2 in blood vessels formation. Nature 376: 70-74). Interestingly, Ang1, but not Ang2, have recently been found to be incorporated into the ECM (Yin Xu and Qin Yu. 2001. Angiopoietin-1, unlike angiopoeitin-2, is incorporated into the extracellular matrix via its linker peptide region. J. Biol. Chem. In Press), suggesting that pericytes recruitment into blood vessels may be mediated by heparanase-mediated Ang1 release.

Taken together, heparanase is shown here to accelerate wound healing in two different animal models for diabetic and ischemic conditions. Moreover, the data further support the notion that heparanase may function as an angiogenic factor, inducing blood vessels formation and maturation (pericytes recruitment) and suggest a novel mechanism that may involved Ang1. These data considerably contribute to the field of vascular biology.

### EXAMPLE 26

### Recombinant Human Heparanase Enhances Epithelialization of 3 Sided Full Thickness Wounds

In order to test the effect of a single heparanase administration Ton epithelialization of wounds, and thus the degree of inhibition of scar formation, 3 sided full thickness ischemic wounds were treated with either 10 or 50 μg purified recombinant human heparanase, or with saline irrigation, and subjected to histological examination 14 days later.

Evaluation of the tissue from a number of locations along the healed incisions revealed that a single application of the recombinant heparanase at the time of suturing reduced the incidence of scarring, and improved epithelialization of the wounded tissue. After discounting the results of one experimental group in which no response to treatment was observed (likely due to prior loss of activity of the heparanase), epithelialization of wounds with the lower dose of heparanase (10 μg) was enhance only mildly, compared to the saline irrigated controls (70% Small Scar or No Scar, compared to 61% Small Scar or No Scar in irrigated controls). Most significantly, however, a single administration of the higher dose of heparanase (50 μg) conferred a greater advantage over the saline-irrigated controls (73% Small Scar or No Scar), and was clearly effective in complete epithelialization (curing) of the wounds (60% healing with No Scar compared with 31% No Scar in irrigated controls). Without wishing to be limited by a single hypothesis, the difference between the effects of the lower- and higher doses of heparanase on wound healing may be due to inconsistent dispersion and/or instability of the recombinant protein in the saline carrier, an effect which could be compounded by the single administration required in this series of trials. According to preferred embodiments of the present invention, various suitable pharmaceutical carriers could optionally and preferably be employed for administering the heparanase such as hydrogel or emulsion based compositions for example, for more effective dispersion, and repeated application, at least during initial phases of wound healing. Such changes can provide for a more prolonged and cumulative effect of the heparanase, and likely reduce effective dosage requirements.

Overall, these results clearly demonstrate the beneficial effects of direct: administration of heparanase on epithelialization of healing wounds and prevention of scar formation.

### EXAMPLE 27

### Cosmetic Use of Heparanase

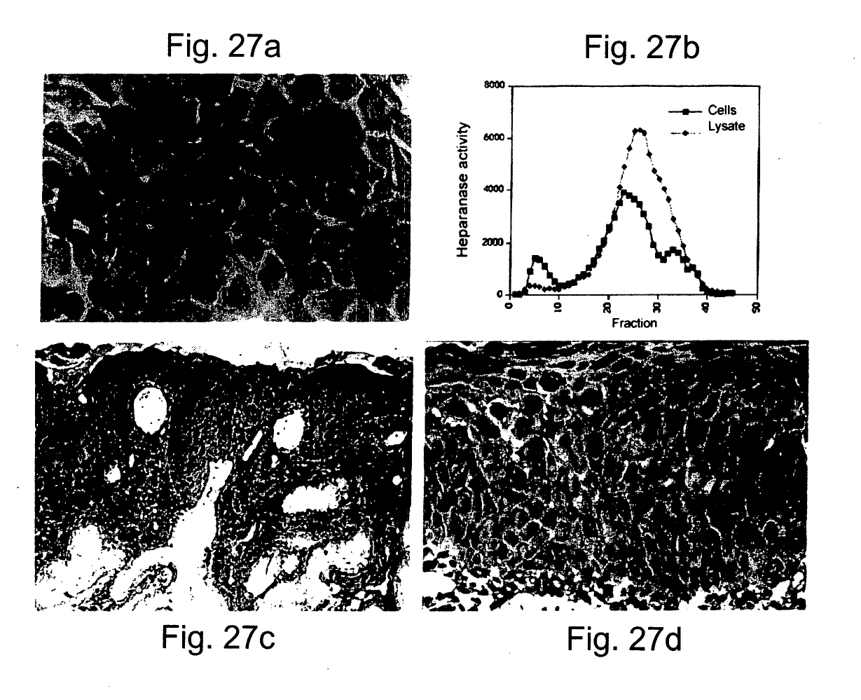

Using anti-heparanase monoclonal antibody (HP-92) cultures of HaCat keratinocytes cell line were immunostained. These cells exhibited significant heparanase staining in their cytoplasm (FIG. 27a). Moreover, intact cells, as well as an extract of these cells, exhibited heparanase activity when assayed in an ECM-assay (FIG. 27b). Immuno-staining of normal skin tissues resulted in the intense staining of heparanase both in the dermis and epidermis (FIGS. 27c-d).

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

### LIST OF REFERENCES

1. Wight, T. N., Kinsella, M. G., and Qwarnstromn, E. E. (1992). The role of proteoglycans in cell adhesion, migration and proliferation. Curr. Opin. Cell Biol., 4, 793′-801.
2. Jackson, R. L., Busch, S. J., and Cardin, A. L. (1991). Glycosaminoglycans: Molecular properties, protein interactions and role in physiological processes. Physiol. Rev., 71, 481-539.
3. Wight, T. N. (1989). Cell biology of arterial, proteoglycans. Arteriosclerosis, 9, 1-20.
4. Kjellen, L., and Lindahl, U. (1991). Proteoglycans: structures and interactions. Annu. Rev. Biochem., 60, 443-475.
5. Ruoslahti, E., and Yamaguchi, Y. (1991). Proteoglycans as modulators of growth factor activities. Cell, 64, 867-869.
6. Vlodavsky, I., Eldor, A., Haimovitz-Friedman, A., Matzner, Y., Ishai-Michaeli, R., Levi, E., Bashkin, P., Lider, O., Naparstek, Y., Cohen, I. R., and Fuks, Z. (1992). Expression of heparanase by platelets and circulating cells of the immune system: Possible involvement in diapedesis and extravasation. Invasion & Metastasis, 12, 112-127.
7. Vlodavsky, I., Mohsen, M., Lider, O., Ishai-Michaeli, R., Ekre, H. P., Svahn, C. M., Vigoda, M., and Peretz, T. (1995). Inhibition of tumor metastasis by heparanase inhibiting species of heparin. Invasion & Metastasis; 14, 290-302.
8. Nakajima, M., Irimura, T., and Nicolson, G. L. (1988). Heparanase and tumor metastasis. J. Cell. Biochem., 36, 157-167.
9. Nicolson, G. L. (1988). Organ specificity of tumor metastasis: Role of preferential adhesion, invasion and growth of malignant cells at specific secondary sites. Cancer Met. Rev., 7, 143-188.
10. Liotta, L. A., Rao, C. N., and Barsky, S. H. (1983). Tumor invasion and the extracellular matrix. Lab. Invest., 49, 639-649.
11. Vlodavsky, I., Fuks, Z., Bar-Ner, M., Ariav, Y., and Schirrmacher, V. (1983). Lymphoma cell mediated degradation of sulfated proteoglycans in the subendothelial extracellular matrix: Relationship to tumor cell metastasis. Cancer Res., 43, 2704-2711.
12. Vlodavsky, I., Ishai-Michaeli, R., Bar-Ner, M., Fridman, R., Horowitz, A. T., Fuks, Z. and Biran, S. (1988). Involvement of heparanase in tumor metastasis and angiogenesis. Is. J. Med., 24, 464-470.
13. Vlodavsky, I., Liu, G. M., and Gospodarowicz, D. (1980). Morphological appearance, growth behavior and migratory activity of human tumor cells maintained on extracellular matrix vs. plastic. Cell, 0.19, 607-616.
14. Gospodarowicz, D., Delgado, D., and Vlodavsky, I. (1980). Permissive effect of the extracellular matrix on cell proliferation in-vitro. Proc. Natl. Acad. Sci. USA., 77, 4094-4098.
15. Bashkin., P., Doctrow, S., Klagsbrun, M., Svahn, C. M., Folkman, J., and Vlodavsky, I. (1989). Basic fibroblast growth factor binds to subendothelial extracellular matrix and is released by heparitinase and heparin-like molecules. Biochemistry, 28, 1737-1743.
16. Parish, C. R., Coombe; D. R., Jakobsen, K. B., and Underwood, P. A. (1987). Evidence that sulphated polysaccharides inhibit tumor metastasis by blocking tumor cell-derived heparanase. Int. J. Cancer, 40, 511-517.
16a. Vlodavsky, I., Hua-Quan Miao., Benezra, M., Lider, O., Bar-Shavit, R., Schmidt, A., and, Peretz, T. (1997). Involvement of the extracellular matrix, heparan sulfate proteoglycans and heparan sulfate degrading enzymes in angiogenesis and metastasis. In: Tumor Angiogenesis. Eds. C. E. Lewis, R. Bicknell & N. Ferrara. Oxford University Press, Oxford UK, pp. 125-140.
17. Burgess, W. H., and Maciag, T. (1989). The heparin-binding (fibroblast) growth factor family of proteins. Annu. Rev. Biochem., 58, 575-606.
18. Folkman, J., and Klagsbrun, M. (1987). Angiogenic factors. Science, 235, 442-447.
19. Vlodavsky, I., Folkman, J., Sullivan, R., Fridman, R., Ishai-Michaelli, R., Sasse, J., and Klagsbrun, M. (1987). Endothelial cell-derived basic fibroblast growth factor: Synthesis and deposition into subendothelial extracellular matrix. Proc. Natl. Acad. Sci. USA, 84, 2292-2296.
20. Folkman, J., Klagsbrun, M., Sasse, J., Wadzinski, M., Ingber, D., and Vlodavsky, I. (1980). A heparin-binding angiogenic protein basic fibroblast growth factor—is stored within basement membrane. Am. J. Pathol., 130, 393-400.
21. Cardon-Cardo, C., Vlodavsky, I., Haimovitz-Friedman, A., Hicklin, D., and Fuks, Z. (1990). Expression of basic fibroblast growth factor in normal human tissues. Lab. Invest., 63, 832-840.
22. Ishai-Michaeli, R., Svahn, C.-M., Chajek-Shaul, T., Korner, G., Ekre, H.-P., and Vlodavsky, I. (1992). Importance of size and sulfation of heparin in release of basic fibroblast factor from the vascular endothelium and extracellular matrix. Biochemistry, 31, 2080-2088.
23. Ishai-Michaeli, R., Eldor, A., and Vlodavsky, I. (1990). Heparanase activity expressed by platelets, neutrophils and lymphoma cells releases active fibroblast growth factor from extracellular matrix. Cell Reg., 1, 833-842.
24. Vlodavsky, I., Bar-Shavit, R., Ishai-Michaeli, R., Bashkin, P., and Fuks, Z. (1991). Extracellular sequestration and release of fibroblast growth factor: a regulatory mechanism? Trends Biochem. Sci., 16, 268-271.
25. Vlodavsky, I., Bar-Shavit, R., Korner, G., and Fuks, Z. (1993). Extracellular matrix-bound growth factors, enzymes and plasma proteins. In Basement membranes: Cellular and molecular aspects (eds. D. H. Rohrbach and R. Timpl), pp 327-343. Academic press Inc., Orlando, Fl.
26. Yayon, A., Klagsbrun, M., Esko, J. D., Leder, P., and Ornitz, D. M. (1991). Cell surface, heparin-like molecules are required for binding of basic fibroblast growth factor to its high affinity receptor. Cell, 64, 841-848.
27. Spivak-Kroizman, T., Lemmon, M. A., Dikic, I., Ladbury, J. E., Pinchasi, D., Huang, J., Jaye, M., Crumley, G., Schlessinger, J., and Lax, I. (1994). Heparin-induced oligomerization of FGF molecules is responsible for FGF receptor dimerization, activation, and cell proliferation. Cell, 79, 1015-1024.
28. Ornitz, D. M., Herr, A. B., Nilsson, M., West, a., J., Svahn, C.-M., and Waksman, G. (1995). FGF binding and FGF receptor activation by synthetic heparan-derived di- and trisaccharides. Science, 268-432-436.
29. Gitay-Goren, H., Soker, S., Vlodavsky, I., and Neufeld, G. (1992). Cell surface associated heparin-like molecules are required for the binding of vascular endothelial; growth factor (VEGF) to its cell surface receptors. J. Biol. Chem., 267, 6093-6098.
30. Liderl O., Baharav, E., Mekori, Y., Miller, T. Naparstek, Y., Vlodavsky, I., and Cohen, I. R. (1-989). Suppression of experimental autoimmune diseases and prolongation of allograft survival by treatment of animals with heparinoid inhibitors of T lymphocyte heparanase. J. Clin. Invest., 83, 752-756.
31. Lider, O., Cahalon, L., Gilat, D., Hershkovitz, R., Siegel, D., Margalit, R., Shoseyov, O., and Cohn, I. R. (1995). A disaccharide that inhibits tumor necrosis factor α is formed from the extracellular matrix by the enzyme heparanase. Proc. Natl. Acad. Sci. USA., 92, 5037-5041.
31a. Rapraeger, A., Krufka, A., and Olwin, B. R. (1991). Requirement of heparan sulfate for bFGF-mediated fibroblast growth and myoblast differentiation. Science, 252, 1705-1708.
32. Eisenberg, S., Sehayek, E., Olivecrona, T., and Vlodavsky, I. (1992). Lipoprotein lipase enhances binding of lipoproteins to heparan sulfate on cell surfaces and extracellular matrix. J. Clin. Invest., 90, 2013-2021.
33. Shieh, M-T., Wundunn, D., Montgomery, R. I., Esko, J. D., and Spear, P. G. J. (11992). Cell surface receptors for herpes simplex virus are heparan sulfate proteoglycans. J Cell Biol., 116, 1273-1281.
33a. Chen, Y., Maguire, T., Hileman, R. E., Fromm, J. R., Esko, J. D., Linhardt, R. J., and Marks, R. M. (1997). Dengue virus infectivity depends on envelope protein binding to target cell heparan sulfate. Nature Medicine 3, 866-871.
33b. Putnak, J. R., Kanesa-Thasan, N., and Innis, B. L. (1997). A putative cellular receptor for dengue viruses. Nature Medicine 3, 828-829.
34. Narindrasorasak, S., Lowery, D., Gonzalez-DeWhitt, P., Poorman, R. A., Greenberg, B., Kisilevsky, R. (1991). High affinity interactions between the Alzheimer's beta-amyloid precursor protein and the basement membrane form of theparan sulfate proteoglycan. J. Biol. Chem., 266, 12878-83.
35. Ross, R. (1993). The pathogenesis of atherosclerosis: a perspective for the 1990s. Nature (Lond.)., 362:801-809.
36. Zhong-Sheng, J., Walter, J., Brecht, R., Miranda, D., Mahmood Hussain, M., Innerarity, T. L. and Mahley, W. R. (1993). Role of heparan sulfate proteoglycans in the binding and uptake of apolipoprotein E-enriched remnant lipoproteins by cultured cells. J. Biol. Chem., 268, 10160-10167.
37. Ernst, S., Langer, R., Cooney, Ch. L., and Sasisekharan, R. (1995). Enzymatic degradation of glycosaminoglycans. Critical Reviews in Biochemistry and Molecular Biology, 30(5), 387-444.
38. Gospodarowicz, D., Mescher, A L., Birdwell, C R. (1977). Stimulation of corneal endothelial cell proliferation in vitro by fibroblast and epidermal growth factors. Exp Eye Res 25, 75-89.
39. Haimovitz-Friedman, A., Falcone, D. J., Eldor, A., Schirrmacher, V., Vlodavsky, I., and Fuks, Z. (1991) Activation of platelet heparitinase by tumor cell-derived factors. Blood, 78, 789-796.
39a. Savitsky, K., Platzer, M., Uziel, T., Gilad, S., Sartiel, A., Rosental, A., Elroy-Stein, O., Siloh, Y. and Rotman, G. (1997). Ataxia-telangiectasia: structural diversity of untranslated sequences suggests complex post-translational regulation of ATM gene expression. Nucleic Acids Res. 25(9), 1678-1684.
40. Bar-Ner, M., Eldor, A., Wasserman, L., Matzner Y., and Vlodavsky, I. (1987). Inhibition of heparanase mediated degradation of extracellular matrix heparan sulfate by modified and non-anticoagulant heparin species. Blood, 70, 551-557.
41. Goshen, R., Hochberg, A., Korner, G., Levi, E., Ishai-Michaeli, R., Elkin, M., de Grot, N., and Vlodavsky, I. (1996). Purification and characterization of placental heparanase and its expression by cultured cytotrophoblasts. Mol. Human Reprod., 2, 679-684.
42. Korb M., Ke Y. and Johnson L. F. (1993) Stimulation of gene expression by introns: conversion of an inhibitory intron to a stimulatory intron by alteration of the splice donor sequence. Nucleic Acids Res., 25; 21(25):5901-8.
43. Zheng B., Qiu X. Y., Tan M., Xing Y. N., Lo D., Xue J. L. and Qiu X. F. (1997) Increment of HFIX expression with endogenous intron 1 in vitro. Cell Res., 7(1):21-29.
44. Kurachi S., Hitomi Y., Furukawa M. and Kurachi K. (1995) Role of intron I in expression of the human factor IX gene. J. Biol. Chem. 10, 270(10):5276-5281.
45. Shekhar P. V. and Miller F. R. (1'994-5) Correlation of differences in: modulation of ras expression with metastatic competence of mouse mammary tumor subpopulations. Invasion Metastasis, 14(1-6):27-37.
46. Zhou G., Garofalo S., Mukhopadhyay K., Lefebvre V., Smith C. N., Eberspaecher H. and de Crombrugghe B. (1995) A 182 bp fragment of the mouse; pro alpha 1(II) collagen gene is sufficient to direct chondrocyte expression in transgenic mice. J. Cell Sci., 108 (Pt 12):3677-3 684;
47. Hormuzdi S. G., Penttinen R., Jaenisch R. and Bornstein P. (1998) A gene-targeting approach identifies a function for the first intron in expression of the alpha1(I) collagen gene. Mol. Cell, 18(6):3368-3375.
48. Kang Y. K., Lee C. S., Chung A. S. and Lee K. K. (1998) Prolactin-inducible enhancer activity of the first intron of the bovine beta-casein gene. Mol. Cells, 30; 8(3):259-265.
49. Chow Y. H.; O'Brodovich H., Plumb. J., Wen Y., Sohn K. J., Lu Z. Zhang F., Lukacs G. L., Tanswell A. K., Hui C. C.; Buchwald M. and Hu J. (1997) Development of an epithelium-specific expression cassette with human DNA regulatory elements for transgene expression in lung airways. Proc. Natl. Acad. Sci. USA, 23; 94(26):14695-14700.
50. Gottschalk U. and Chan S. (1998) Somatic gene therapy. Present situation and future perspective. Arzneimittelforschung, 48(11):1111-1120.
51. Ye S., Cole-Strauss A. C., Frank B. and Kmiec E. B. (1998) Targeted gene correction: a new strategy for molecular medicine. Mol. Med. Today, 4(10):431-437.
52. Lai L., and Lien Y. (1999) Homologous recombination based gene therapy. Exp. Nephrol., 7(1):11-14.
53. Yazaki N., Fujita H., Ohta M., Kawasaki T. and Itoh N. (1993) The structure and expression of the FGF receptor-1 mRNA isoforms in rat tissues. Biochim. Biophys. Acta., 20; 1172(1-2):37-42.
54. Le Fur N., Kelsall. S. R., Silvers W. K. and Mintz B. (1997) Selective increase in specific alternative splice variants of tyrosinase in murine melanomas: a projected basis for immunotherapy. Proc. Natl. Acad. Sci. USA, 13; 94(10):5332-5337.
55. Miyake H., Okamoto I., Hara I., Gohji K., Yamanaka K., Arakawa S., Kamidono S. and, Saya H. (1998) Highly specific and sensitive-detection of malignancy in urine samples from patients with urothelial cancer by CD44v8-10/CD44v10 competitive RT-PCR. Int. J Cancer, 18; 79(6):560-564.
56. Guriec N., Marcellin L., Gairard B., Calderoli H., Wilk A., Renaud R., Bergerat J. P. and Oberling F. (1996); CD44 exon 6 expression as a possible early prognostic factor in primary node negative breast carcinoma. Clin. Exp. Metastasis, 14(5):434-439.
57. Gewirtz A. M., Sokol D. L. and Ratajczak M. Z. (1998) Nucleic acid therapeutics: state of the art and future prospects. Blood, 1; 92(3):712-736.
58. Hida K., Shindoh M., Yasuda M., Hanzawa M., Funaoka K., Kohgo T., Ameniiya A., Totsuka. Y., Yoshida K. and Fujinaga K (1997) Antisense E1AF transfection restrains oral cancer invasion by reducing matrix metalloproteinase activities. Am. J. Pathol. 150(6):2125-2132.
59. Shastry B. S. (1998) Gene disruption in mice models of development and disease. Mol. Cell. Biochem. 1998 April; 181(1-2):163-179.
60. Carpelitier A. F., Rosenfeld M. R., Delattre J. Y., Whalen R. G., Posner J. B. and Dalmau J. (1998) DNA vaccination with HuD inhibits growth of a neuroblastoma in mice. Clin. Cancer Res., 4(11):2819-2824.
61. Lai W. C. and Bennett M. (1998) DNA vaccines. Crit. Rev. Immunol., 18(5):449-484.
62. Welch P. J., Barber J. R., and Wong-Staal F. (1998) Expression of ribozymes in gene transfer systems to modulate target RNA levels. Curr. Opin. Biotechnol., 9(5):486-496.
63. Durand P., Lehn P., Callebaunt I., Fabrega S., Henrissat B. and Momon J. P. (1997) Active-site motifs of lysosomal acid hydrolyses: invariant features of clan GH-A glycosyl hydrolases deduced from hydrophobic cluster analysis. Glycobiology, 7(2):277-284.
64. Thuong and Helene (1993) Sequence specific recognition and modification of double helical DNA by oligonucleotides Angev. Chem. Int. Ed. Engl. 32:666
65. Dash P., Lotan I., Knapp M., Kandel E. R. and Goelet P. (1987) Selective elimination of mRNAs in vivo: complementary oligodeoxynucleotides promote RNA degradation by an RNase H-like activity. Proc. Natl. Acad. Sci. USA, 84:7896.
66. Chiang M. Y., Chan H., Zounes M. A., Freier S. M., Lima W. F. and Bennett C. F. (1991). Antisense oligonucleotides inhibit intercellular adhesion molecule 1 expression by two distinct mechanisms. J., Biol. Chem. 266:18162-71.
67. Paterson Paterson B. M, Roberts B. E and Kuff E L. (1977) Structural gene identification and mapping by DNA-mRNA hybrid-arrested cell-free translation. Proc. Natl. Acad. Sci. USA, 74:4370.
68. Cohen (1992) Oligonucleotide therapeutics. Trends in Biotechnology, 10:87.
69. Szczylik et al (1991) Selective inhibition of leukemia cell proliferation by BCR-ABL antisense oligodeoxynucleotides. Science 253:562.
70. Calabretta et al. (1991) Normal and leukemic hematopoietic cell manifest differential sensitivity to inhibitory effects of c-myc antisense oligodeoxynucleotides: an in vitro study relevant to bone marrow purging. Proc. Natl. Acad. Sci. USA 88:2351.
71. Heikhila et al. (1987) A c-myc antisense oligodeoxynucleotide inhibits entry into S phase but not progress from G(0) to G(1). Nature, 328:445.
72. Reed et al. (1990) Antisense mediated inhibition of BCL2 prooncogene expression and leukemic cell growth and survival comparison of phosphodiester and phosphorothioate oligodeoxynucleotides. Cancer Res. 50:6565.
73. Burch and Mahan (1991) Oligodeoxynucleotides antissense to the interleukin I receptor m RNA block the effects of interleukin I in cultured murine and human fibroblasts and in mice. J. Clin. Invest. 88:1190.
74. Agrawal (1992) Antisense oligonucleotides as antiviral agents TIBTECH 10:152.
75. Uhlmann et al. (1990); Chem. Rev. 90:544.
76. Cook (1991) Medicinal chemistry of antisense oligonucleotides—future opportunities. Anti-Cancer Drug Design 6:585.
77. Biotechnology research news (1993) Can DNA mimics improve on the real thing? Science 262:1647.

1. A method of inducing or accelerating a healing process of a wound, the method comprising administering to the wound a therapeutically effective amount of a recombinant isolated and purified heparanase, said heparanase having or being activatable to have an endo-β-glucuronidase catalytic activity as an active form of a heparanase protein as set forth by SEQ ID NO:10, so as to induce or accelerate the healing process of the wound.

2. The method of claim 1, wherein said wound is selected from the group consisting of an ulcer, a burn, laceration, a surgical incision, necrosis and a pressure wound.

3. The method of claim 2, wherein said ulcer is a diabetic ulcer.

4. The method of claim 1, wherein said heparanase is contained in a pharmaceutical composition adapted for topical application.

5. The method of claim 4, wherein said pharmaceutical composition is selected from the group consisting of an aqueous solution, a gel, a cream, a paste, a lotion, a spray, a suspension, a powder, a dispersion, a salve and an ointment.

6. The method of claim 4, wherein said pharmaceutical composition includes a solid support.

7. The method of claim 0.4, wherein said heparanase has a concentration in a range of from about 0.005 microgram per 1 cm² to about 50 microgram per 1 cm² of wound area.

8. The method of claim 7, wherein said heparanase has a concentration in a range of from about 0.5 microgram per 1 cm² to about 5 microgram per 1 cm² of wound area.

9. The method of claim 1, wherein-said heparanase is present in a concentration in a range of from about 10 micrograms to about 150 micrograms per dose.


SEQUENCE LISTING (47 sequences)

SEQ ID NO: 1 - DNA, 27 bp, Artificial sequence
  Synthetic oligonucleotide
ccatcctaatacgactcactatagggc

SEQ ID NO: 2 - DNA, 24 bp, Artificial sequence
  Synthetic oligonucleotide
gtagtgatgccatgtaactgaatc

SEQ ID NO: 3 - DNA, 23 bp, Artificial sequence
  Synthetic oligonucleotide
actcactatagggctcgagcggc

SEQ ID NO: 4 - DNA, 22 bp, Artificial sequence
  Synthetic oligonucleotide
gcatcttagccgtctttcttcg

SEQ ID NO: 5 - DNA, 15 bp, Artificial sequence
  Synthetic oligonucleotide
ttttttttttttttt

SEQ ID NO: 6 - DNA, 23 bp, Artificial sequence
  Synthetic oligonucleotide
ttcgatcccaagaaggaatcaac

SEQ ID NO: 7 - DNA, 24 bp, Artificial sequence
  Synthetic oligonucleotide
gtagtgatgccatgtaactgaatc

SEQ ID NO: 8 - protein, 9 aa, Artificial sequence
  Peptide derived from tryptic digestion of human heparenase
YGPDVGQPR

SEQ ID NO: 9 - DNA, 1721 bp, Homo sapiens
ctagagctttcgactctccgctgcgcggcagctggcggggggagcagccaggtgagcccaagatgctgctgcgctcgaagcctgcgctgccgccgccgctgatgctgctgctcctggggccgctgggtcccctctcccctggcgccctgccccgacctgcgcaagcacaggacgtcgtggacctggacttcttcacccaggagccgctgcacctggtgagcccctcgttcctgtccgtcaccattgacgccaacctggccacggacccgcggttcctcatcctcctgggttctccaaagcttcgtaccttggccagaggcttgtctcctgcgtacctgaggtttggtggcaccaagacagacttcctaattttcgatcccaagaaggaatcaacctttgaagagagaagttactggcaatctcaagtcaaccaggatatttgcaaatatggatccatccctcctgatgtggaggagaagttacggttggaatggccctaccaggagcaattgctactccgagaacactaccagaaaaagttcaagaacagcacctactcaagaagctctgtagatgtgctatacacttttgcaaactgctcaggactggacttgatctttggcctaaatgcgttattaagaacagcagatttgcagtggaacagttctaatgctcagttgctcctggactactgctcttccaaggggtataacatttcttgggaactaggcaatgaacctaacagtttccttaagaaggctgatattttcatcaatgggtcgcagttaggagaagattatattcaattgcataaacttctaagaaagtccaccttcaaaaatgcaaaactctatggtcctgatgttggtcagcctcgaagaaagacggctaagatgctgaagagcttcctgaaggctggtggagaagtgattgattcagttacatggcatcactactatttgaatggacggactgctaccagggaagattttctaaaccctgatgtattggacatttttatttcatctgtgcaaaaagttttccaggtggttgagagcaccaggcctggcaagaaggtctggttaggagaaacaagctctgcatatggaggcggagcgcccttgctatccgacacctttgcagctggctttatgtggctggataaattgggcctgtcagcccgaatgggaatagaagtggtgatgaggcaagtattctttggagcaggaaactaccatttagtggatgaaaacttcgatcctttacctgattattggctatctcttctgttcaagaaattggtgggcaccaaggtgttaatggcaagcgtgcaaggttcaaagagaaggaagcttcgagtataccttcattgcacaaacactgacaatccaaggtataaagaaggagatttaactctgtatgccataaacctccataacgtcaccaagtacttgcggttaccctatcctttttctaacaagcaagtggataaataccttctaagacctttgggacctcatggattactttccaaatctgtccaactcaatggtctaactctaaagatggtggatgatcaaaccttgccacctttaatggaaaaacctctccggccaggaagttcactgggcttgccagctttctcatatagtttttttgtgataagaaatgccaaagttgctgcttgcatctgaaaataaaatatactagtcctgacactg

SEQ ID NO: 10 - protein, 543 aa, Homo sapiens
MLLRSKPALPPPLMLLLLGPLGPLSPGALPRPAQAQDVVDLDFFTQEPLHLVSPSFLSVTIDANLATDPRFLILLGSPKLRTLARGLSPAYLRFGGTKTDFLIFDPKKESTFEERSYWQSQVNQDICKYGSIPPDVEEKLRLEWPYQEQLLLREHYQKKFKNSTYSRSSVDVLYTFANCSGLDLIFGLNALLRTADLQWNSSNAQLLLDYCSSKGYNISWELGNEPNSFLKKADIFINGSQLGEDYIQLHKLLRKSTFKNAKLYGPDVGQPRRKTAKMLKSFLKAGGEVIDSVTWHHYYLNGRTATREDFLNPDVLDIFISSVQKVFQVVESTRPGKKVWLGETSSAYGGGAPLLSDTFAAGFMWLDKLGLSARMGIEVVMRQVFFGAGNYHLVDENFDPLPDYWLSLLFKKLVGTKVLMASVQGSKRRKLRVYLHCTNTDNPRYKEGDLTLYAINLHNVTKYLRLPYPFSNKQVDKYLLRPLGPHGLLSKSVQLNGLTLKMVDDQTLPPLMEKPLRPGSSLGLPAFSYSFFVIRNAKVAACI

SEQ ID NO: 11 - DNA, 1721 bp, Homo sapiens; identical to SEQ ID NO: 9
  CDS (63)..(1691)

SEQ ID NO: 12 - DNA, 824 bp, Mus musculus
ctggcaagaaggtctggttgggagagacgagctcagcttacggtggcggtgcacccttgctgtccaacacctttgcagctggctttatgtggctggataaattgggcctgtcagcccagatgggcatagaagtcgtgatgaggcaggtgttcttcggagcaggcaactaccacttagtggatgaaaactttgagcctttacctgattactggctctctcttctgttcaagaaactggtaggtcccagggtgttactgtcaagagtgaaaggcccagacaggagcaaactccgagtgtatctccactgcactaacgtctatcacccacgatatcaggaaggagatctaactctgtatgtcctgaacctccataatgtcaccaagcacttgaaggtaccgcctccgttgttcaggaaaccagtggatacgtaccttctgaagccttcggggccggatggattactttccaaatctgtccaactgaacggtcaaattctgaagatggtggatgagcagaccctgccagctttgacagaaaaacctctccccgcaggaagtgcactaagcctgcctgccttttcctatggtttttttgtcataagaaatgccaaaatcgctgcttgtatatgaaaataaaaggcatacggtacccctgagacaaaagccgaggggggtgttattcataaaacaaaaccctagtttaggaggccacctccttgccgagttccagagcttcgggagggtggggtacacttcagtattacattcagtgtggtgttctctctaagaagaatactgcaggtggtgacagttaatagcactgtg

SEQ ID NO: 13 - DNA, 1899 bp, Homo sapiens
gggaaagcgagcaaggaagtaggagagagccgggcaggcggggcggggttggattgggagcagtgggagggatgcagaagaggagtgggagggatggagggcgcagtgggaggggtgaggaggcgtaacggggcggaggaaaggagaaaagggcgctggggctcggcgggaggaagtgctagagctctcgactctccgctgcgcggcagctggcggggggagcagccaggtgagcccaagatgctgctgcgctcgaagcctgcgctgccgccgccgctgatgctgctgctcctggggccgctgggtcccctctcccctggcgccctgccccgacctgcgcaagcacaggacgtcgtggacctggacttcttcacccaggagccgctgcacctggtgagcccctcgttcctgtccgtcaccattgacgccaacctggccacggacccgcggttcctcatcctcctgggttctccaaagcttcgtaccttggccagaggcttgtctcctgcgtacctgaggtttggtggcaccaagacagacttcctaattttcgatcccaagaaggaatcaacctttgaagagagaagttactggcaatctcaagtcaaccaggatatttgcaaatatggatccatccctcctgatgtggaggagaagttacggttggaatggccctaccaggagcaattgctactccgagaacactaccagaaaaagttcaagaacagcacctactcaagaagctctgtagatgtgctatacacttttgcaaactgctcaggactggacttgatctttggcctaaatgcgttattaagaacagcagatttgcagtggaacagttctaatgctcagttgctcctggactactgctcttccaaggggtataacatttcttgggaactaggcaatgaacctaacagtttccttaagaaggctgatattttcatcaatgggtcgcagttaggagaagattatattcaattgcataaacttctaagaaagtccaccttcaaaaatgcaaaactctatggtcctgatgttggtcagcctcgaagaaagacggctaagatgctgaagagcttcctgaaggctggtggagaagtgattgattcagttacatggcatcactactatttgaatggacggactgctaccagggaagattttctaaaccctgatgtattggacatttttatttcatctgtgcaaaaagttttccaggtggttgagagcaccaggcctggcaagaaggtctggttaggagaaacaagctctgcatatggaggcggagcgcccttgctatccgacacctttgcagctggctttatgtggctggataaattgggcctgtcagcccgaatgggaatagaagtggtgatgaggcaagtattctttggagcaggaaactaccatttagtggatgaaaacttcgatcctttacctgattattggctatctcttctgttcaagaaattggtgggcaccaaggtgttaatggcaagcgtgcaaggttcaaagagaaggaagcttcgagtataccttcattgcacaaacactgacaatccaaggtataaagaaggagatttaactctgtatgccataaacctccataacgtcaccaagtacttgcggttaccctatcctttttctaacaagcaagtggataaataccttctaagacctttgggacctcatggattactttccaaatctgtccaactcaatggtctaactctaaagatggtggatgatcaaaccttgccacctttaatggaaaaacctctccggccaggaagttcactgggcttgccagctttctcatatagtttttttgtgataagaaatgccaaagttgctgcttgcatctgaaaataaaatatactagtcctgacactg

SEQ ID NO: 14 - protein, 592 aa, Homo sapiens
MEGAVGGVRRRNGAEERRKGRWGSAGGSARALDSPLRGSWRGEQPGEPKMLLRSKPALPPPLMLLLLGPLGPLSPGALPRPAQAQDVVDLDFFTQEPLHLVSPSFLSVTIDANLATDPRFLILLGSPKLRTLARGLSPAYLRFGGTKTDFLIFDPKKESTFEERSYWQSQVNQDICKYGSIPPDVEEKLRLEWPYQEQLLLREHYQKKFKNSTYSRSSVDVLYTFANCSGLDLIFGLNALLRTADLQWNSSNAQLLLDYCSSKGYNISWELGNEPNSFLKKADIFINGSQLGEDYIQLHKLLRKSTFKNAKLYGPDVGQPRRKTAKMLKSFLKAGGEVIDSVTWHHYYLNGRTATREDFLNPDVLDIFISSVQKVFQVVESTRPGKKVWLGETSSAYGGGAPLLSDTFAAGFMWLDKLGLSARMGIEVVMRQVFFGAGNYHLVDENFDPLPDYWLSLLFKKLVGTKVLMASVQGSKRRKLRVYLHCTNTDNPRYKEGDLTLYAINLHNVTKYLRLPYPFSNKQVDKYLLRPLGPHGLLSKSVQLNGLTLKMVDDQTLPPLMEKPLRPGSSLGLPAFSYSFFVIRNAKVAACI

SEQ ID NO: 15 - DNA, 1899 bp, Homo sapiens; identical to SEQ ID NO: 13
  CDS (94)..(1869)

SEQ ID NO: 16 - DNA, 594 bp, Homo sapiens
attactatagggcacgcgtggtcgacggcccgggctggtattgtcttaatgagaagttgataaagaattttgggtggttgatctctttccagctgcagtttagcgtatgctgaggccagattttttcaggcaaaagtaaaatacctgagaaactgcctggccagaggacaatcagattttggctggctcaagtgacaagcaagtgtttataagctagatgggagaggaagggatgaatactccattggaggctttactcgagggtcagagggatacccggcgccatcagaatgggatctgggagtcggaaacgctgggttcccacgagagcgcgcagaacacgtgcgtcaggaagcctggtccgggatgcccagcgctgctccccgggcgctcctccccgggcgctcctccccaggcctcccgggcgcttggatcccggccatctccgcacccttcaagtgggtgtgggtgatttcgtaagtgaacgtgaccgccaccggggggaaagcgagcaaggaagtaggagagagccgggcaggcggggcggggttggattgggagcagtgggagggatgcagaagaggagtgggaggg

SEQ ID NO: 17 - DNA, 21 bp, Artificial sequence
  Synthetic oligonucleotide
ccccaggagcagcagcatcag

SEQ ID NO: 18 - protein, 21 aa, Artificial sequence
  Synthetic oligonucleotide
AGGCTTCGAGCGCAGCAGCAT

SEQ ID NO: 19 - DNA, 22 bp, Artificial sequence
  Synthetic oligonucleotide
gtaatacgactcactatagggc

SEQ ID NO: 20 - DNA, 19 bp, Artificial sequence
  Synthetic oligonucleotide
actatagggcacgcgtggt

SEQ ID NO: 21 - DNA, 21 bp, Artificial sequence
  Synthetic oligonucleotide
cttgggctcacctggctgctc

SEQ ID NO: 22 - DNA, 23 bp, Artificial sequence
  Synthetic oligonucleotide
agctctgtagatgtgctatacac

SEQ ID NO: 23 - DNA, 22 bp, Artificial sequence
  Synthetic oligonucleotide
gcatcttagccgtctttcttcg

SEQ ID NO: 24 - DNA, 23 bp, Artificial sequence
  Synthetic oligonucleotide
gagcagccaggtgagcccaagat

SEQ ID NO: 25 - DNA, 23 bp, Artificial sequence
  Synthetic oligonucleotide
ttcgatcccaagaaggaatcaac

SEQ ID NO: 26 - DNA, 23 bp, Artificial sequence
  Synthetic oligonucleotide
agctctgtagatgtgctatacac

SEQ ID NO: 27 - DNA, 24 bp, Artificial sequence
  Synthetic oligonucleotide
tcagatgcaagcagcaactttggc

SEQ ID NO: 28 - DNA, 22 bp, Artificial sequence
  Synthetic oligonucleotide
gcatcttagccgtctttcttcg

SEQ ID NO: 29 - DNA, 24 bp, Artificial sequence
  Synthetic oligonucleotide
gtagtgatgccatgtaactgaatc

SEQ ID NO: 30 - DNA, 22 bp, Artificial sequence
  Synthetic oligonucleotide
aggcaccctagagatgttccag

SEQ ID NO: 31 - DNA, 24 bp, Artificial sequence
  Synthetic oligonucleotide
gaagatttctgtttccatgacgtg

SEQ ID NO: 32 - DNA, 25 bp, Artificial sequence
  Synthetic oligonucleotide
ccacactgaatgtaatactgaagtg

SEQ ID NO: 33 - DNA, 22 bp, Artificial sequence
  Synthetic oligonucleotide
cgaagctctggaactcggcaag

SEQ ID NO: 34 - DNA, 22 bp, Artificial sequence
  Synthetic oligonucleotide
gccagctgcaaaggtgttggac

SEQ ID NO: 35 - DNA, 23 bp, Artificial sequence
  Synthetic oligonucleotide
aacacctgcctcatcacgacttc

SEQ ID NO: 36 - DNA, 22 bp, Artificial sequence
  Synthetic oligonucleotide
gccaggctggcgtcgatggtga

SEQ ID NO: 37 - DNA, 22 bp, Artificial sequence
  Synthetic oligonucleotide
gtcgatggtgatggacaggaac

SEQ ID NO: 38 - DNA, 22 bp, Artificial sequence
  Synthetic oligonucleotide
gtaatacgactcactatagggc

SEQ ID NO: 39 - DNA, 19 bp, Artificial sequence
  Synthetic oligonucleotide
actatagggcacgcgtggt

SEQ ID NO: 40 - DNA, 27 bp, Artificial sequence
  Synthetic oligonucleotide
ccatcctaatacgactcactatagggc

SEQ ID NO: 41 - DNA, 23 bp, Artificial sequence
  Synthetic oligonucleotide
actcactatagggctcgagcggc

SEQ ID NO: 42 - DNA, 44848 bp, Homo sapiens
ggatcttggctcactgcaatctctgcctcccatgcaattcttatgcatcagcctcctgagtagcttggattataggtctgcgccaccactcctggctacaccatgttgcccaggctggtcttgaactcttgggctctagtgatccacccgccttggcctcccaaagtgctgggattacaggtgtgagccatcacacccggccccccgtttccatattagtaactcacatgtagaccacaaggatgcactatttagaaaacttgcaatggtccacttttcaaatcacccaaacatgttaaagaaattggtatgactgggcatggcacagtggctcatgcctgcaatcctagcattttgtgaggctgagacgggcagatcacgaggtcaggagattgagaccatcctgacagacatggtgaaatcccatctctactaaaaatacaaaacaattagccgggggtgatggcaggcccctgtagtcccagctactcgggaggctgaggcaggagaatggcgtgaatccaggaggcagagcttgcagtgagccgagatggtgccactgcactccagcctgggcgacagagcgagactccgtctcaaaaaaaaaaaaaaagaaagaaattggtatgactgttgactcacaacaggagtcaggggcatggggtggggtgtaagattaatgtcatgacaaatgtggaaaagaaacttctgtttttccaactccacgtctgctaccatattattacactcttctggtagtgtggtgtttatgtgtgaattttttttcatatgtatacagtaattgtaggatatgaacctgattctagttgcaaaactcactatgagcttagcttttaagttgcttaagaataggtagatctatgcaaataatgataattattattattattttaagagagggtctcactttgtcacccaggctggagtgcagtggtgtgattaagggtcactgcaacctccacctcccaggctcaaataaacctcccacctcagcctccccagtagctggaaccacaggcacgggccaccacgcctggctaattttttgtattttttgtagagatggggtttcatcatgttgcccaggctgttcttgaattcctcggctcaagcaatcctcccaccttggcctcccaaaatgctggcatcacaggcatgatggcatcactggcatcacataccatgcctggcctgatttatgcaaattagatatgcatttcaaaataatctatttttatttgttgccttattggtggtacaatctcaagtggaaaaatctaagggttttggtgttatttgcttactcaaccaatatttattagactcttactaagcaccaacatgatcacatgcctgagctatggctagcatagcgtgtgagacaaacttaatctctgttttggtggagcatataatctagtagatgaagccaatgttgagcaacatcacaatactaacaaattgaggatgctacgagagtgtctaacaaattgaggatgctacgagagtgtctaacaaattgaggatgctatgagagtgtgtcatggagagctgcctggagattgagagaaagcttccttgagggaagttacatttcagctgaaacacactgccatctgctcgaggttttgtaactgcattcacatcccgattctgacacttcacatcccgattctgacacttcacccagttactgtctcagagcttgggtccgcatgtgtaaaacaaggacagtatgcacttggcagggttgtgagaagggaagagaacacaagtaaagcacctgtatcaggcatacagtaggcactaagcgtgcgatgcttgctatgattatacatcagtgtaagcatcaaggaaaagctgaagaaaagtctgaccaacagcgaaagataaatgcgcagaggagaaatttggcaaaggctccaaattcaggggcagtccgtactctacactttgtatgggggcttcaggtcctgagttccagacattggagcaactaaccctttaagattgctaaatattgtcttaatgagaagttgataaagaattttgggtggttgatctctttccagctgcagtttagcgtatgctgaggccagattttttcaagcaaaagtaaaatacctgagaaactgcctggccagaggacaatcagattttggctggctcaagtgacaagcaagtgtttataagctagatgggagaggaagggatgaatactccattggaggttttactcgagggtcagagggatacccggcgccatcagaatgggatctgggagtcggaaacgctgggttcccacgagagcgcgcagaacacgtgcgtcaggaagcctggtccgggatgcccagcgctgctccccgggcgctcctccccgggcgctcctccccaggcctcccgggcgcttggatcccggccatctccgcacccttcaagtgggtgtgggtgatttcgtaagtgaacgtgaccgccaccgaggggaaagcgagcaaggaagtaggagagagccgggcaggcggggcggggttggattgggagcagtgggagggatgcagaagaggagtgggagggatggagggcgcagtgggaggggtgaggaggcgtaacggggcggaggaaaggagaaaagggcgctggggctcggcgggaggaagtgctagagctctcgactctccgctgcgcggcagctggcggggggagcagccaggtgagcccaagatgctgctgcgctcgaagcctgcgctgccgccgccgctgatgctgctgctcctggggccgctgggtcccctctcccctggcgccctgccccgacctgcgcaagcacaggacgtcgtggacctggacttcttcacccaggagccgctgcacctggtgagcccctcgttcctgtccgtcaccattgacgccaacctggccacggacccgcggttcctcatcctcctggggtaagcgccagcctcctggtcctgtcccctttcctgtcctcctgacacctatgtctgccccgccagcggctctccttcttttgcgcggaaacaacttcacaccggaacctccccgcctgtctctccccaccccacttcccgcctctcattctccctctccctcccttactctcagaccccaaaccgctttttggggggtatcatttaaaaaatagatttaggggttacaagtgcagttctgttccatgggtatattgcattgtggtggcatctgggctcttagtgtaactgtcacccgaatgttgtacattgtatctaataggtaatttctcatccctcatccctctcccaccctcccaccttttggagtctccagtgtctactattccactaagtccatgtgtacacattgtttagcgcccactctaaatgagcctttttgtttcattcattctgtaagtgttgaataggcaccacctaaggtcaggtataagtggaaatttgaaaaagaaactgcccacttgccccagtacttccctagccaagaggagggaaaccaggcaggtgcacctgaaggcctgtgagtgcttgatttgctgtgcagtgtaggacaagtaagattgtgcatagccttctgtatttaagactgtgttaggaagatttctctttcttttcttttctttttcttttttcttttcttttttttttttaggcagatgaaaagggcgtcacagaacaggaataaaaatctaaatattcaataaatgagacctaggagactactgcagtgacttacaaagtcctaataaaaagatgtctctccaaaatggggctgcaaaatgtggtgctgccttatcagctctaagttttttccttacctgagaaagaaggaacctgatgcaggttcagggctcctgccccatgaatgcaggctgactccaagatggggagctacagggacaatcccaggtcttctaggcctcttatttaggccctgggagcctccagagatggccacatcttgaccagcccagatagagggaaagatcaccattatctcacctctgtgtcaaatacctagatgctgtcctccctgagcccacactatagttgccagcgctaatttaatgggtagtgtactggttaagagatggacagaccatcctggcttgactctcagctctggcaaagatgagtgacttggtttttccatatctcttggccacaccaaccttgatttcttcagctgtagaatggaatttctcaagcttgcctcaaggattattgcccgaggatttgatgatatggtaagagcttctcagtgtttgacccatagtaagtgtttgacgtttcaaacgaattgtttctttctaggacatggtgagcatttggtagccattcaccggttttctgtttctttggatcatagttaacctctccttttccttctggcactacaattttctggtggggaagaatccttactttctgcccttccccttaaggataggaagctgatactaggcagcaactagttgggggataggaagattgttccagagaaatgctgaaccatagggctccagatcacaggaccccagtcttagcttgctggggtgtggggtgggggggggcggttactgaacatgggtatgaagtagatgtccatttactgaaatgtgaggacctgaggcctcttctattgctgtagccagcatattccccaacctctccccaagaaaggacagatgggggttcccccctggagtaacaggtccaaaagaaaaaacatacagtgggacttccaggatctgggcctgatcacccagcagtcaagctccccgcaattgactaacacccccctaacacgtagaaattccaatctgcaatttagtgaggatgatacctttattcttcttaaatacatctcttcatttcccagagcacccttttttcccctcctctgcacctttttgttaaagactggagtataatgaaataccaagagagcataacatgtgatacataaaactttttttctggtttacaaaacagttcattcttgtccatacgtgcttctctccaaggctggctgctgtctgttccagcccgcttcgcttggagaggccatctgccatacctgctccccagacgcatcgacaagcacacccagagtgttatctgctaagacctaaaagagggaggaaccccctctcctcatctaagacctagcttctaaattagagtgtgagggtccatctccccaggaggggcacagggcccaaacagcccagccatctcagaagacaacactaagctttgtaggggtccacagtagaggagagtaagacgcctgttgtttaatttattacagttcctcaaaagtgaagatgtgtgggcgggatggcaagagctgagcagacgaaagctgaaggaataaggaaagagaggaggacacaaacagctgacacttcctcagttcttgtcatttgcctggccctgttctaagcaccttctaggtattaatccatttagtcttggctacaacactgtgagtaactagttttgtcacccccattttaaaaatgaagaaagtgaggctcagggaggttaagtaacttggccacagtttgaaactagactctgatcacatgagataatagtgcccataaaaagggaaagcagattatattttttaaaggaaagagagtaggatatggtagaaaaagattgtttggaaaggaattgagagattgatataatgaaaagaagcattcacatgagagtaacagtatcagggcccaaaccttcatctaaggtacttcaaagaggcctaagcaaacttagtcactggcgtggttctagtctccatgatggcaaatacattgtgtacagcccaactccacacaaaacttaaataccaatgatagagcaatctaaaatttgaaagaaaaaatctttcaatttgtcgtcttcccagagggacttaatcaagaaaccaatcaaaatacttcctaagcctaactgtgtgcagaactccaaagagagcccagccctaaatcaacactgtccaatggaaatataatataatgtgggcctcatatgcaaggtcatatgtaattttaaattttctagtagccatattaaaaaggtaaaaagaaacaagtgaaattaattttaataattttatttagttcaatagatccaaaatgttttctcagcatgtaatcaatataaaaatattaatgaggtatttattattccttttctcaaaccaagtctattctataatctggcgtgtattatttacagcacttctcagactatatttctttctttcttttttttttccgagacaattttgctcttgtcacccaagctagagtacaatggcgttacctcggctcactgcaacctccgcctcccgggttcaagttattctcctgcctcagtctcccaagtagctgggactagaggcatgcaccaccacgcctggctaattgtgtatttttagtagagacagggtttcaccatgttggccaggctaatctcaaactcctgagctcaggtgatatgcccacctcggcctcccaaagtgttgggattacaggcgtgagccactgcacccggcctcagattaactatatttcaagcgttcagtagccacatgtagctagtgctatggtagtggacagtacagatctgcatttcaattaagacacgtatacaagcatagttcactaatgcacggtaaaaaaaagtatagtgctgagtcggtggtagaaatcctaaatactgcagagcaaaagtggtacgaacagcaatctcagtgataatgcaaccatgcttgcttttcattgcaatttgcttattttccttcagcaaagttcatccatttttgccaattcaataaatatttactgataaaaactttcaatattagattcttgcatcttcatagacagagttgcttttcacatttagaaaattacttatcaatgttaaacacacgttttgataaccagtgttggaaagaggtgcagactccccatgtgcctattgatggcagaaatattcacagccaaagggaaacaaagggctggggacaatcacacacctcatgtctcctaactcctgggaagtgctgtccctctgattgagctcttattattgccttccccactaaccctgtccactgtgccctggagccctttgcagggttacctgctctgtcctcctcacagaatatctcctctacctccttgtccaagctacaacttggctattctctgatgacactgtcttccctgtagcccttttgagtaatggctgcatattctcccatagtccagttcttttcctgttctccagtctggcttctggatgacagcccactagtttgaactccatactgctatagttcaagtcccttttgacttgttaccttgggcaaattacctccttttgttcaggttccttgtttgtaaaatgacgataataatgccatttgcttcagtgggttattttgaaattgagtgaaagaaggcgggtagcttccctacacgctcagtgtagactagcctgatgtgcattacgggtgatgccatgactcagtgtgttttcctcatctccacatctggctctcatccagtgctcctgcttacggcactctgtccccctcttacttactcccccttattaactgaagactggcactgatctcacagtttcctctccacttcctagtctcaccatcatcctagatgacttcaagtcacctagataaactgtctcagtttcttcactcacatttttttataacagataatgttacactcaagttgtaacagaaccagcttatccagctcatgaaatgtatgcatttcatctcaactctgtattcagtgacatcctgtgggtatctggaaatcagccatggtgagaatatttaccatggaaattggcaaatactaaaaagcagagcacctttttttctgagagccagaccatagctcttctactccatagcacccatcataacaatttttaaatacctccactgaacagcttcttcctctctctacttcttccatatctgatttgagcttcttaatttatcatgtgaaccactcttgtaataataaccccaaatccctgttccattgttcttcctgctaaaatactaaacctggtttagtccaaccatattttctctctttggaatctacagggtggcccaaaaacctggaaatggaaaaatattacttattaattttaatgtatattaataagccattttaatgcttcatttccagtctcagtggccaccctgtatagctgggctattgagctcttgcgggaggagggagtggacagtctcccagccacacagactgatgttgcaccaaacattttttagcttccagacttccctggcccttagtgttacccttaactctccatttctctgcctttcacattctctactttttaaaaatctctgactccaccttcaccttatcattcttagcacatgaccatacttctgcttcccaaagaaaatgagcaattacttccttttccttttcctcctgtcatcaaatctgcagacatgtcatgcctaagtccagctttcctcctttctctgatctcagtctgcttcttccatttctgccctgaatcccgtcccctccccaacccccaaggacttcgctctatcagtcacctcttccctctcctgtatcttcaactcctcccattttactggcttcttcctcaagcctttccccaagcctttcccatctcaattacctcctcgcacatgcctctgcagaaaccaccccgtttcttccctcccctcggcagcctgttcttcctgttctgccctcatgatggcaccatcattgtgtcactaaaatcaatctctccgacatcatcaatggccttcctttgttgggaaacctaataaacactttatcttatttggtctttgttatgggttgaatgaggttaccccgaaatccatattagaagtcctaacccccagtacctcagaatgtgactttatttgggaatagggtcattgcagacgttattagttaggatgaggtcatactggaatgtgatgggctgcttatctaatatgactgatgtccttataacaaggagaaatttggagacagacacgcacatagggagaataccatgtgatgacaggagttatggagttggagtcaaaaagctatgggaacttaggagaaagacctggaacaaatcctttcctgcgcctagagagggagtatggccctgccactaccttgaattcaacgtttcggcttttcaaaactgtaagacaatacatttctgttgttcaaaccaattagtttgcagtactctgcgactgcagccctaacaaactaatacagtctcttggaggcatttggcaaggttgacaatggaagcactttcttacccctttaggtctgtcgcctttcttgttggggggtgttttctaacaattcctctccatctctctctctctagtttgtcttaaacattggtgttcttcagacttctgacctaggccttcttttcacttcacatattcccctgggtggtctcacccacttccagaaattacttaaattactgctcatgcagtactgtgctggaaactgtttaacaactggctctctgggaagaggggagactggttgatggtttttgctgatttctgtggtgtaaatactccctccatggccaattccaaactgccaacagtttaacaactggctcacaaattttctccaaatttaacatttggctttcacaggccaacaacgtggtacagccaactccagcacacctctgcttttgtgtcagagagaagtaacttatttttgtacaaaaggtaaaataaaaacacctgcaggccccctttttttccttaacaaactgctctagaaatagaatagctgaagcttcttttatgcattcatctgttatttccatgtcactgtggtggtgggattatttttcctttatttttcttgtatatggttgaaatactgtacctttgatcagttttagttttatggcatgttttgcacccatattaaatctagtttttgtcagagggcgtcaatattattttctcaaaacaagaaaatatttcattgcaaaggagacaaacaaaaaggtccttaataccaaaactttgaaatgtgatttcttgtacttggcagtgtccaagtggtaaacccaaacagtattgggttttcattttgttcaggaaagtctttgtctggcagcgacttacccttacatcaggcgggccttgctcattcattcacttaagtatttattaaacaccagcggtgtgccaagtacttatctaggtatcgggtagattctgataagtcagtcaggtccctgctctcagggagcttgcagcagagatgggggctgcaatagagagtaagccaaggaaatgaaaaaggaagttgatttcagagagtgatgaatgctatgaagaaaatgaaggcagcgcagtgtgatggagagtgacccaaggtggtacagtttgtacctctaaggaccagactgtgacccaggtcactcacagatgcccgtcatgtgatgccacagcaacttttccaggtgctcgtttcctcccacttcccagtctcttgcccagccgcgactgcttacaaatacagctagaggaatctaaatgaggttcctctatcatcaaacccaatcaaaatgccaaggaacagaatcagtgcctggctgaaggcagtggaacagggccagcctggagtggttctctctgaggaagttcctcatcttggttttagggccataccttgtgacctgtgagctaggggttgccagtccctgacatttctactgaggactcgcctgtctatattcccggcctgtatgtgtctcctgagttccagacacacagggcgaagcgcctgatggatggaagtatgttttttggtgttccattggtatctcaaattctacaaaacttagtgccccttctcctccctgttcctccccatcttcagtctatcacctgttcctcatccagcaaatgatattaccatcttccaaggagcttcccaggagtaatccttgactcctcctcaacatccaattaataatcaaatctaggccaggtacaatagctcacgcctataatcccagcactttgggaggctgaggcaggtggatcatttgaggccaggagttcaagaccagcctggccaacaaggtgaaacctgtctcatttaaaaaaagttattttaaaaactcaaatctattatttctacctctaagtgtgtcttgaatttatccatctctctccatctctgagctgttaccttacctcagtccatcacgttttgtctacgttaacatgaccagagtcttgttcttagtctggtgaggtcactccagctgcttcagatccttccatggctcaccgttgccctcatataaagttggcactcctggacatgtggcttacggggccctccgtgatgtggccctatttgcttctccattctgttctctcccagcctctctgcccccatctctaggcaccaaccacacccttctgctcgtcaatggtgccagcttctcttctatctctggtctttggacagacttttcccttcacctggaatgctttcttcaatcctaccccactctctttaatctagataaggtttattctttttgaatgtctagcagtgaaaccatttcccctgaaaaaccttctctaaccaaccccctaccctcagcccaaggtctagattaggagtccctctgaatgtttccatagcatttttaaagaattgcctatttacttgttcgtatctatcactaaactacaaattgtatgagaacagccactatctctgcctggttcaccattcatctccagcaactagcataatgcctggcagagtcagcctgcaacaaatatttgttgaataaattaacagatggctttatctccttaagtaaatcttgcttttttcacctattaaaacagacgcacaggccaggtgtggtggcccatgcctgtaatcccagcactttggcaggctgaggtgggcggatcacctgaggtcaggagttcaagaccagcctggccaacatggtgaaaccccatctctaataaaaatacaaaaattagctgggcatggtggtgggtgcgtatagtcccagctactagggaggctgaggcaagagaatcgcttgaacccaggaggcagaggtggcagtgagccgagatcatgccactgtactccagcctggatgacagagaccctgtctcaaaacacacacacacacacacacacacacacacacacacacacacacacacacaccaagttgtataatttaaaatataacgtgcttgttatggaacacttgtaaaatacaggaaagtaatgaaaaagtctaccatctagctcaccacataatgaccattgctatcatcctggcataattctctcctgtatataaatatatattcttttattgttaaaattacactatgagtactatttatttattttactgtggcaaaatgcgcaaaacataaaatcttgccattttaaggtatgcagtttggtgcattcaccacactcacattgttgtgcaaatatcaccactatctatctcagaacttcttcgtcttcccaaactgaaactctgtacccattaaacaatagtgcatcctctgttttcccctccctacaatttatttttatttgggtttgtaccaaactgaaaatagctgcttcttccttacttagttcagattagcatttccatttatttagccgtggttttgaggatgccatgacagatgccatccttcctagagctctttggggctgtcaggtatttcagtcagggtgaattcgggttgataacattttaaaatctcactttattctgaggttcctagtgtcagagcccaccgtatttttagggactcccaagttacaaacaaaaatatggtgaggaggaatcactgaagttttaacacaagagacttacattttgttcaatttctatcttttagtttatttcctaagcataaagaaatactttgaaaattttacatagcattatacatatttaattaagcatgagcacatcttaaaactttaaattttagatcagatctttaattcctaggatattaagaggtactggcaatttggccaggtgtggtggttcacgcctataatcccaacactttgggagggtgaagtgggcgaattgctagagcccaggaggtggaggctgcaatggcctgagatcacgccatcgtactccagcctggatgatgagaatgaaatcctgtctcaaaaaaaaaaaaaaaaaaaaaaagaagaagaagaagtattggcaatcagtgctccaggaataatttcctgacttgaaataaacctacatgtagacaaactaattaggccattccaagagttgctagcattggtttaatatgttttcagagcattccaggaagcagtgtggccagcattgcatgtttgatacttcagaaatgtatgacaggtgtttctcttacccaggtcttctgttttcttagttttgctcatgtaaatatttatgaacatcctcatctttttgagggaagggattatagatcattctaattccattttctagcatttggtaccattctaagcacatgataggcacccatttggagcatttttggcttgacagaatatgcatttagaattgttcaaattagaggtgtcagtgatgggaattagaatactatataattctaagtcatttgacttaaatacaaaagaatgattttccttggtggggaatggtgaagggaggcaggagttaagaagaggagaagagatcctaagtcatttataaacttctctggaaagacaggtgtgtgaagactttttaaaaagtcattcaccaaattgtgtgtgtgtgtgtgtgtgtgttttaaatagactttattttttagagcagttttaggttcacagcaaaattgaatgcaaggacagagatttcccataaaccccctgcccacacacatgcatagcctccctcattatcaacatccccaccagagaggtgtttgttctagttgatgaacctacactgacacatcattatcacccaaagtccatagttcacggcagggttcactgtcggtgtacattctatgggtttgagcaaatgtataatgacatgtatccaccattatagtaacatacagagtattttcagtgccctgcaaatcccctgttctccacctattcatccctccctctctgcatttccacccccagcccctggtaaccgctgatctttttactgtcccatagtttcggacgatctatttttcagacagacacagagctgtctttcccttagtttctattctatcatttctttctccccatccatcataaaaggctatgagttttttttaagtgttgaacaccatcctacttgtcaagttaaaacataagctcctggctgggtacagtggctcatgcctgtaatctcagcattttgggaggctgtggcagaagcatcacttgaagccagaagtttgagaccagcctgggcaacatagcaagaccccatccctccacacacaaacacacacacacacacacacacacacacacacacacacacacacacacaaaaacaagctcttgccagaattagagctacaaattgccctcaggttcctagaagatcagtccttcaattagattcagattgagatgcttcctcttttaaacaatgattccctttctatcatgcccaataagaaaacaaataaaaattaaacaatactgcctgtaatctcagctacccaggaggcagaagcagaactgcttcaacccggcaagcagaagttgcagtgaagtgagatcgcgccactgcactccagcctgggaaacagagcaagattctgtctcaaaaacaaaacaatgtgatttcctcctctaagtcctgcacagggaaatgttaagaaataggtccaccaggaaagaaggaagtaagaatgtttgactagattgtcttggaaaaaatagttatactttcttgcttgtcttcctaacagttctccaaagcttcgtaccttggccagaggcttgtctcctgcgtacctgaggtttggtggcaccaagacagacttcctaattttcgatcccaagaaggaatcaacctttgaagagagaagttactggcaatctcaagtcaaccagggtgaaaatttttaaagattcactctatattttaattaacgtcagtccgtcatgagaatgctttgagaaaactgttatttctcacacctaacaattaatgagattaacttcctctcccctcatctgacctgtggaggaatctgaacaagaggaggaggcagtgggcaggtttccttatcatgatgtttgtcatgttcagtgtgaggcctcacaaaaaaaaaaaaaaaaaaaaaaggcgtcctggatataactgagagctcattgtacagtaaatattaataaaacagtgattgtagctgaaggatagaactgcttggagggagcaagtgggtagaatcgcgtcaaactaaagagcatttctagccaaagacacaatgatagattgaaggatatttattctaaatatagaatatgggtgaacgagatctgtggacttctgggctccaacgttagattctgattttagcaagcttgtcaggggattctgatattgaaaggctgtggccttcacctgagaaacctgccctagggggccatgaaaatttgtcctgtctttcagaagtgctatcagacatcaaatggaagttaaatcgtatcttaacaattactaggatgggcgcagtgactcacacctgtaatcccaacactttgggaggctgaggcaggaggatcacttgagcccaggagttcgggaccagcctgggcaacatagagagacgttgtctctattttttaataatttaaagagaaaaaaatactgaaaatattgtatacaccactgaattataataatgtgtatataatgtatatattcattatgaggaatatttgattatttcatatattatatcttttccttctgtttattttatccagttatgaagtatttagaacaattcatcagtaattggggctaaattgacagaatagtaatcagagaaaatagaaaaagacagatgggttatctttgaataccaggttggagttgtttatgggtttgttttttgttttgggggcgtttttttagacagagtcccactctgttgcccaggctggagtgcagtggcacaagcatggcccactgcatccttgacctcttgggctcaagcaatcttcccaccttagcctcctgagtagctgggaccacaggtgcatgtcaccacacccagctaatttttttattttttgtagagacagtctttctatgttatccaggctgatctcaaactcctgcactcaagtgatccccctgccttggcgtcccaaagtattgggattataggcatagccaccacacccaacctagtttctatttagacttggccctttcccaccagtcatttgtgtccaaaagatctcataaatgtagacaggaaactgtcctttgctcatcagttttcttcatcctgtgtctagggggatggtcggtgggggaaactggggttatgcaagttcctctgaaacatcctctgtgagcccagggatggatgaggcaccagccgccagcgagtcagtgtgcagctttccagaaaggaagtcatcagccagtcagccggccctggcagccagcacccggcaaccctgctgtcttgtgataaagaaatggtctgcctgacaggatggtgtggatttttcttttttcttttttttttttttgagacagggtctggctctgtcgcccaggctggagtgcaatggcgggatcttggctcactgcagcctctgcctcccaggctcaaggcatcctcccacctcggtctcccgagtagctgggaccacaggcacacaccaccacgcccaactaagttttcgtatttttagtagaggcagggttttactatgttgtccaggctagtctcaaactcctgagctcaagctatccatctgccttggcctcccaaagagctggaattacaagcgtgagccactgtgcctgaccagggtggattttttcaagtgcacatgttgtggtcccagaagctctgatggtaccaaattccaagcgaaaaaaagtcaatggttcccacccatcctacctcccatgatggcaagaggaaatcaccacactgcagatacagtccatgtaaaacaaattgctatggattttgaaagtgaaccttaagagaactgcactatgttttcttcattagagttctctggtaatttccagctttttttttttttttttttagacagtgtctcgctttgtcgcccagtgtcacccaggctggagtgcagtgacgtgatctcggctcactgcaacctccgcctcgtgggttgaagtgattctcctgcctcagcctcctgagtagctgtattttagtagagacgaggtttcaccatttggccaggctggtctcgaactcctgacctcaagtgattcgcccatctcagcctcccaaagtgctgggattacaggtgtgagccactgcacccggccagtaatttcaagcttctgaggagccctttgaattgttaaataacttgtagctatgtccaacatatccatgttcagtgtatgttcgatatttcttaggaaacctgcccttggttgttttctttgtggtaattcatgagccggcaaatttgacatgtgttacagaatataccttttctctgctctcctacctcataaccagaacttaattatcctgctttagtcacataaatagctaactaaataaatatatgagatttcagtctgctcactgtgaaaatagaccttctaaatgatctcttccacttgcagatatttgcaaatatggatccatccctcctgatgtggaggagaagttacggttggaatggccctaccaggagcaattgctactccgagaacactaccagaaaaagttcaagaacagcacctactcaagtaagaaatgaaaggcaccctagagatgttccagccccaaagatatttgaataggttggactcgggcaccaatctagcaagtcctacggaagttgtataaagctgaaaatactgaagcatttcccaaatgggaaatcctaaactcaaaacttgctttttggtttttttgtttgtttgttttttcttcatctgacattgcttagtagtcacagaatgaaagataaatcaatcattcatgatctaacaatgaccttcagtgctctaaaaaactacggagtcaaggaaaacatgaatatattcctcatgtaaaattaaaatacagacatataaagggcaaaacatgaacatcattcataccttgaggtccgtccccctcccagaaataacccccagtatgccttggtttagagcattaagcaggagggccctgagtcactccagacagtcttgaccaccaagcagcattctctttttgtttcctctgtggcttttgcaaacacagggctagctcagctacccattagtatgttttcagtcactaaaacagtcttccagtcttcaaattaggatgacattgtcacatggggctttaaagcaagtgaaacaaggaaccccctttttttttttttttgagatggaatctcactcttgtcgcccagcctggagtgcaatggcgcaatcttggctcactgcaacctccacctcccaggttcaagagattctcctgccttagcctcctattcattatgaggaatatttgattattcagttcctgtagggtaaagatattacccccgatcatattattgattattgagtagctgagattacaggtgcctgccaccacgaccggctaattttttgtattttttagtagagacagggtttcaccatgttggccaggctccaggctcgtctcgaactcctgacctcaggtgatccacccacctcagcctcccaaagttctgggattacaggcgtgagccaccactcctggccacaatccttttttaactatgaaatatatttttatctgaagtttgatgtttatacccaactgagggatgatgttcccatatctcagttaaagaaataacctgctcagatacttcaagctcttcttttgacttttgaaaataaatgatcttgaagttactatactttgtttgggttagttaacattatttaaagtatattattttaattaattatctttgtaagattttactgtatactacctggagttcaatgtatcagatggatttcaaatttatgtacattttttatgtatatggtacagaaaaaaatgtgatccataagaaatcagaaaatagcgcatatgctaatagctaatgttgtcctctaaaaaacttatttttgcatttttaagagggggatatactctgacactttaataagtgtaattaattattgactggaatttggcatgaggcagggccatttcagatcccattaaaggaatgacacataccagagaaccacagaagtaaggccacatttgtaataaatcattatagctctgctaggagaagacccagttgtattaggtaattaatggatttgctcttaaaacacatgtcccggaagatataggtgagtcttggggggccgcattaaacattataccaatgtatcttacatttctaagaaagttttactactttacaggatctttctgttaccaaaatggaaggtttccaactccaggacttggctttcatagttcctacaccaggggaaatgccttcctttgctaactatgcaaccaggttagttagtgtaagtccagccaccctgttggcaatgctaaaaggtacaacaaacacagaattttatttgcatttgtaaacatttgatttctggctcgaaattttcagttttcatgggcacgtcatggaaacagaaatcttctgtgtttagtttgggcacctactcattgtagtgacaaatatttcagaagccaataggggattccacaaattgttctgaacctgtggctgagactggtaatggctgagtgacatggggacataccacaaaagaagaggtagcaaaaggctgctgagataaggacatgttcattgcttagctagtggcctgcacccttaaaacacatgtcccaggctgggtgctgtggctcacgcctgtaatcccagcactttgggaggctgaggcgggtggattacctgaggtcaggagttcgagaccaacctggccaacatagtgaaacctcatttctactaaaaatacaaaaattagccaggcatggtggcgggcgcctgtagtcccagctactcaggaggcaggcaggagaattacttgaatctgggaggcagaggttgtggtgagccgagattgcgccaccgcacgctagcctgggcgacaaagtgagactctgtctcaaaaaaacaaaaacaaaaaacaaacaaacaaaaaacaacaacaacaaaaaaacgggtatcccagaagatacaggtaagttttctaacacaggtcctcttgtatggtgcgttccacttaagtagaagatgacaaaaacatttgtcatgagaatatagactcacattttaaacctgtttgagcaggaaaaggaagcaatgttacagatgtaattctgggtgtgactgcagaaaggatgactcccttattaaagtagtcatcctgagtgagctaactctttgtacttcctcttctcctcctgttcccctcatcaccccattcttccgttgcctacacccaggcccacattggatgctgacatagacttacatggtacagtccaagggaaagatctgccatttttttcaatgtgtcatcttggttatcttcattccaaggatctctccactctttatacagtaagagatgagagtctggaaaggattgggaataagataatgaattgtaagttttaaattgttcttcgtattttggggaaggagtaggctaggtggtccttctgttttttttttgtttttttttttaaagtagatgtggccagacgtggtggctcacgcctgtaatcccagcactttgagaggctgaggcaggtggatcacttgatgtcaggagttcaagaccagcctggccaacacagtgaaaccccgtctttactaaaaatacaaaaactagccgggcttggtggcgtccacctgtagtcccagctactgcagaggtggaggcaggagaatcacttgaacccgggaggtggaggttgcagtgagccaagatcatgccattgtactccagcctgggcgacagaacaatactctgtctcaaaaaaaaagagaaaagaaaagaaaaaaagaatggatttgaactcagtcgtcaatagcctctattccaggagatgttacagttgattatgttatagggggtgtataatagaatttcgagctatgtaaattccaagtgcatttggaagaatgaagaaatggaggaagggtaaagtatgagtgcaagcattccaggttttttgaaaatgctataatctttgttcagggctagtacaaagtgctatttagctgtaagggttttttgtgatttacagacagttttcacatgtgtcatttcaaccttggttttatggcgaaggcatgtgatggtgcttgtcccaggactttagatccatatctgaggttcctgtcgggcaaagatattacccctgatcatattatagtctataagtgggagagttgtgcctggagctcaagtcttatgatttctgatccagggcacttcctacaacatgattttgcaatataaaagcctataatgtgtgactaaagcaggtcactcaccccttgtaacagactctagtaatggtactgccaccaaacggctgcgtgatattgggcaaagacttaccttatttgaatctcagtttcctcctagaaaaatgagggtggaggttaagcataggctgatgatcctaaagcctccatactgccctaaactgtggctctaagatccagtagaatgctgggtcacaggactctagggagcttttcaaacccaaatgtctgtcattccttgatggtaggcagcagtttatggaagtgggcgacacagcaaatatcaaaatacctaaagcagcttgcaagagttgtttctgcctagtggtctttatagttaatattaaatagttaattttttttttttttgagacagagtcttgctctgttacccaggctgcagtgcagtggcacaatctcggctcactgcaacctccacctcccgggtttgagcaattctgtctcagcctcccaagtagctgggactacaggtgcatgccactgcacccagctaatttttgtatttttagtagagacggggtttcaccatattgggcaggctggtctcgaactcttgacctcaggtgatccacctgcctcagcctcccaaagtgctgggattacaggcatgagccactgcacccagcttaaatagctaatatttaatattattctatagttattcaagtaattcaggccaaagacttagaaacaaaacaaaaagccacttttaaggagaaagggtgtaagtttgccagatagatagagatctttcttttttaactacaagagttcaggaatgaattactctttaacaaacgactatagatatacatgaaaattggaaggacttattatgcatatgataatcaatttaaagacaacacttaaaattatattgttgccactctcaaaaagtggtaatagaacagctaatggtttaaaaagcagagtacagaagttcccaaacttatggcaccttaatatcgcagaaaactttttaaagcatgcctaggccacaaaaaatacctgtattttgattattaaattgtaaggtctacacaacctaatagtaataggtccaatagtaatgctgtccaatagatgttgatgtttttttccttgcaaacttaaaagatcctacagtgcctctgtaaatagcactgcctggttagagttgaatttcagataaataatttttttcatgttaattatttttcttttctttacttttttttttgtttttttgtttttttgtttttttttttgagacagggtctcattctgttgcccaggctgctgtgcaatggcatgatcatggctcactgcagccttgacctccctgggctcaggtgatcctcccacctcagcctcccaagtagctagctgggactacaggtgcttaccatcatgcccggctaatttttgtgttttttgtagagatgtggttttgccatgttgcccaggctggtcttgaactcctgggctcaagtgatccgcccgcctcggcctcccaaagtgctaggatgacaggcatgagccactgcacctggcccctgggcgaagtatttcttaatggttacataggacatacactaaacattatttattgtctatatgaagttcaagtttaactaggtgccctgcacttttagttgctaaatcctgtagctgtacccatgcattcactggtgctccccagcttgccttgcacagagtttggaaaccatagtcctataactctaggccaattttttaatgtaaaatttgattcattttaaattaataaataataacaggaatttttttaaaaattgttttaaatataattaaaattatcaaaatattttttaactgaacttgtgactagagatatttagattatgaagagtggggtttatgctaactaatgacagtctggctatgcatgtggagcactgagctataaattgtggcttccccaattctcctgatgtcacttgaacaaaacctaagtgtcagaccagagcttctggtatcttccatgggatttcattcaacagctggagcaaatgaagtcagattgattttttttaatttgtccaattttgttgtctcaaaaacataattataatcatttattagaactagaatttcttcagtttaacaacagaaatagttattcattatgaaaagcgaatctggaggccttcattgtggtgccaatctaaccattaaattgtgacgtttttcttttaggaagctctgtagatgtgctatacacttttgcaaactgctcaggactggacttgatctttggcctaaatgcgttattaagaacagcagatttgcagtggaacagttctaatgctcagttgctcctggactactgctcttccaaggggtataacatttcttgggaactaggcaatggtgagtaccccagggaacaattcattaataaggagattccccactagcattatttcttttcttttctttttcttttctttttttttttttttgagacagagtctcgcactgctgcccaggctggagtgcagtggcgccacctcggctcacttgaagctctgcctcccaaaacgccattctcctgcctcagcctcccgagtagctgggactacaggcacccgccaccgcgcccggctaatttttttttttttttttttttttttttttttgcatttttagtagagacggggtttcaccgtgttagccaggatggtcttgatctcctgacctcgtgatctgccctcctcggcctcccaaagtgctgggattacaggcgtgagccaccaggcccggctagcattatttcttatgacactttttttttttttttgagacggagtctcgctctgtcgcccaggctggagtgcagtggcgccatctcggctcactgcaagctccacctcccaggttcacgccattctcctgcctcagcctcccgagtagctgggactacacgcacccgccaccacgcccggctaatttttttgtatttttagtagagacggggtttcaccgtgttagccaggatggtctctatatcctgaccccatgatctgcccgcctcggcctcccaaagtggtgggattacaggcgtgagccactgcgcccggccaacactctttttattattagcaaatatacttctgcctgggcacattcttgcaagtgctcaacaatgcaacttttggaagtgcatgtggcagaaactcctgctgtatttattccagaacctattattgctaatcccagtttatgttacatttgaagtgagaaccagttggagccagcaacgttcccagctccaaagttcccttgagattttcagaatcacttaaccctattatgcttggcaacctggactcagcaaaactgggaagtcagcagtttgttttattcatcccttcctttctcagtttctcaaatgtgtcagttaatctcagtaaccccattgcaaccttcattacctgcccaagcggtctagaacttgccagtatagaatcctacgtgggtcaagctcctgactgtctccttcttcactctttttttgcaaagaacttgtaaattttaactataagtattcatgattcgccacatttattcaaaacatagagtgctttttccacatatcagccaatggaaataaggattaaatgggaaatgaaatgtagtaataggataagcacaagtcttcttcctgctcaaacttttttttttttttttttcagacaagatcttgctctgttacccaggctggagtgcagtggcgtgttcatagctcaatgtaacctccaactcctgggctcatgcaatctctcacacctcagccccctgattagctaggactacactatgcctagccaattttttttcttttgtctggttgtgttgcccaggctgtctcgatctcctggcctcaagtaatcctcctgcctcggccttctaaagtgctgggattataggcatgagccactgtgcccggtctcaaacctttttttccaaagtaaatgaagttattagatatggaatatagtctagttcccagatatccatatccattggtttattaccctcattattaacttcaaattgtttaatagaccctcatatctcagttatacagttaaaatttttgttttgtttttctggagtatcttatttataactatgagttttactttacttatttattttattttttgagacagacgcttgctctgtcactcaggctggagtgcggttgcgtgatcatggctcactatggcctcgaccttctgggctcaagtgatcctctccctcagcctcccaagctgagactacaggcatgcaccaccacatctagctaatttttttttttccccatggaacaaggctttactatgttacccagagtggtctcaaactcctggcctcaggggatcctcctgtctcagcctaccaaaatgctgggattacaggcatgagccatagcgccagacctggttttacttttcttgactttgaattacaagtttttgtaatttggaaaatgttttgttgcttttaaatactgctgtatgtttgcttttaaatacaacatttctcgatatatattttgagaattgctgtctttcagaacctaacagtttccttaagaaggctgatattttcatcaatgggtcgcagttaggagaagattttattcaattgcataaacttctaagaaagtccaccttcaaaaatgcaaaactctatggtcctgatgttggtcagcctcgaagaaagacggctaagatgctgaagaggtaggaactagaggatgcagaatcactttacttttcttctttttccttttgagacagagtctcactctgtcagccagactggagtgcagtggtacaatcatggctcactgcaacttcgacctcccaggctcaagcaatcctcccatctcagtcccacaaatagctgggactacaggtgcacatcaccacacctggctactttaaaaaaatttttttgtagagatggggtctccctgtgttgcccaggctggtctcttgaattcctgtgctcaagccatccttccacctcagcctcccagagtgccaggattacaggcatgagccaccacacccagccaccacttttcttaaaaaaaaaaaaagattctctctggtagacaatcctcaatagtccacatgttattaaacaatctgctgcctgaatacatgatttaccaaaaaaaggaaattttgacgggttcagaatatcaagggatctgaggcaaatgtcacctatgataaaatttgctatcaaaattaggaagtttgtgtttacctgatcctaaagcagtaaccagcccatttctagggaataaaactctcatgcgtatattgtgcatatatatgtattatatgactgagtgataataaaattttttttctagcttcctgaaggctggtggagaagtgattgattcagttacatggcatcagtaagtatgtctcctattcttaatactaggaaagtaaggctagctttatttattacctagtattcaaaaagttagttcatttaactgccaattgactgcagttcaaataagaaacaaatagtgtctcaagtagcactgtactccaattttaatattaataaaaaaaattttaagttattttaaataatgtagtggtttctataaagatcactttatacagaagaacagtgccaattaacccatggaacatataagtagctaaaaccaattgcttgccaaagaaccagtaacccaggagtacatgtccttgccactgtgttttttcaagacagagtaactgatttctagttacttgcatagaatggactcctcctcataactcccttccatcttggtctttccctagtagaacttctacctttttttagtaacaggtgagtgggagaggtaagaaggagaataaggtcagcaattaacctaaaagcagaaagtaaaatttgttattttttttctgaatattttctgtgtaatttagctactatttgaatggacggactgctaccagggaagattttctaaaccctgatgtattggacatttttatttcatctgtgcaaaaagttttccaggtaatagtctttttaaactttttaatgtaaaaccagaatccttattttatagtctagctagttctaaattctataggtatgtatatttacatgtttttctaattttagagaacaagcactatgacttatccactgttagttttccccttagcattgggtcttaccccatgtacgtgattagaaatttgaaatatttccaatagcctttagtagaattaactcacatagatgataagaatgggttggttcacttcatgttccttccacagcctactatttcaataaaagaaagtttcccaagacctaaatgactatgaacatattttataactatataggaggggtgggtctaggaatacaaagttttgaatgctgttaatcttcaacaccacagttgaaaccacaggtcagcttttttgcaattaccatggatacttttctgttctataggtggttgagagcaccaggcctggcaagaaggtctggttaggagaaacaagctctgcatatggaggcggagcgcccttgctatccgacacctttgcagctggctttatgtgagtgaagcagcgctggccttaggggtcagagtgcagctcttctccatccttctattctgctgaaatagctccccagccaaaaagcagatcaaagaccgtttcagtggctgagccccaaaattcatgccagattttgcaagaaaatgatttactaaagcttgagggacatctttaacaagtgttccaaattaatcactataaggatgaattgtttcagaaattttggcctttaattatggcccataaatatgtcaagtagtccttactctaaagaagtacactgtaaaagaatgcatatagccggatatggtagttccctgtaatcccaatactttgggaggccaaggtgggaggattgcttgagcccaggagtttgaggctgcagtgagttatgatggtgccactgcactctagactgggcaacagagtgagactgtctttttttttcccctctgtcacccagactggagggcagtggcacgatctcacctcactgcaacctctgcctcccggattgaagcgattctcctgcctcagcgtcctgagtagctgggactacaggagtatcaccgcactgggctaatttttgtatttttagtagagacggggttttgacatgttgcccaggctggtctgaaacccatgagctcaagtgatctgcctacctcagccttccaaaatgctgggattacggacatgagctaccacgcccggccacaccctgtctcttaaaaaaaaaaaaaatgcaagttagagcatattacagctttgtctctcaggaggatacttagtgtatgtagctataattcatagattcccaagaagtttagagcctaaagtatgaggtcccaccagaggggctatcattaaatttaaagatttgttaaatcatctcattgtccaacaccacaaacttgattgctttaaaatactggtttagttacatttagtaactctattagtgcttttaatctatactgctatatcctcacattgagattttttttcttttctcttccatcttcattcttttttctctcatcctcattcttataagcctagaatacatcacaaatcctttatgcccatggaagcaagaggaataaagaatggagatgtttgttttgccattaactaaagatctggggtgtcggggagaagggggatagagaaggagaagtgggaagaggtgtccataatagcttaggtgcaattctgcttattttacattttacccccgctgactgccactttttcttcagccctcacacattgtttgtgcagggacctcataggaccaggaattgtctatagaggtgggaatttgtctcaccctgaaagggatacctctagcatggtaatagtcttctaggatttgttatcatatggaaagatgtaaagggagggattctgctgctgctgctgctgctgcatgcagttgccatttcatttaaatgacttatttataattgatgacacttttctggcttcctgttaattcctccctcaaagatcaataaaccagaaccaggcatggtggcatgcacttgtggtcctgtaaccacccaacaggttcaccttgcctgctgtctagatagagccaattatcaagacaggggaattgcaaaggagaaagagtaatttatgcagagccagctgtgcaggagaccagagttttattattactcaaatcagtctccccgaacattcgaggatcagagcttttaaggataatttggccggtaggggcttaggaagtggagagtgctggttggtcaggttggagatggaatcacagggagtggaagtgaggttttcttgctgtcttctgttcctggatgggatggcagaactggttgggccagattaccggtctgggtggtctcaaatgatccacccagttcagggtctgcaagatatctcaagcactgatcttaggttttacaacagtgatgttatccccaggaacaatttggggaggttcagactcttggagccagaggctgcattatccctaaaccgtaatctctaatgttgtagctaatttgttagtcctgcaaaggtagacttgtccccaggcaagaagggggtcttttcagaaaagggctattatcatttttgtttcagagtcaaaccatgaactgaatttcttcccaaagttagttcagcctacacccaggaatgaagaaggacagcttaaaggttagaagcaagatggagtcaatgaggtctgatctctttcactgtcataatttcctcagttataatttttgcaaaggcggtttcagtcccagctacttgggaggctgagacaggaggattaatggagcccaggagtttgaggttgcagagagctatgatcacgccactgcactccagcctgggtgacagagtgagaccctgtctctaaataaataaataagtaaataaataaatacataaataaaatcaagatggtgtgcaattagaattgagcgattttgtttccaaacctcaagaaagcttggtcttgctctgtcccaggtggctggataaattgggcctgtcagcccgaatgggaatagaagtggtgatgaggcaagtattctttggagcaggaaactaccatttagtggatgaaaacttcgatcctttacctgtaagtgaccattattttcctaattctagtggagtagattaaagtcaactcaggacctctggtgttaacctcctatgaacagtcagtcctctcagtaactagccaaatcatgagatgatgaattagaaggagccttagatagcatccaatctaacatttttttgtgtgtttgaagagaagaaatcaagagctaggaataactttttaaaggtaagccatttgcagtatagtgtggattttgtttaaaaggggataatttgaaattttatgactcattatacaagacaaaataagttggattttcaaatgttttacaaagtaaatcaaagttataattgcctacagtacgcaaagcttcaaaacattttttatgttatgaaattgtaatttatttaaccttaaaatgagccagtaccatgtgtttgcttaaaaatctcatgctaagaatttactatgttgttaataatcttcaagatatttatgaataaagtcttatttctaatccttcctccaactgtatctggtgctaaatcaggaaatgtttcttcccaaaaagcctcgtggaagatctgtatgtctaaatatatgtcagggataatacagatgtagccctgcgaagcatgaccttgatttttatagtctaaaatgtcatttgcagatatctattttctaagaataattcctaaaagaattatttgaatgttgtaggaaagctaagaaattttgcaaagagcgtacgtgaaaatataagctaggcttttgtggtttgtggatagacttcccaacaaaattgctttttatctatagtgatccaagcttgtggaacatattagtcatctttttttagaaaattcttagaaaagtgatcttgcaaaaatggaatttatctttccccaagtatattctgtcatgtatagagttaaactaagcatagtaatttcaccagacaaacattcaaaatctactcctgacctttttatctcatccaaattttcccagggcccagacataaacctttgccttacgaactctttgtatatgcactaaatatgcttctccttcaaggttctcagtcagctagaaaaatgtgcaagagtaaatggtacccttctcacttgtagatccaagagaattagacttaaactcactctacatgtctgtgactttattttatttgcatgacagtcctgtgaggtggcaaggcaggtatcttggatccattttttagataaggaagttcaaattgagaagaggttgcatgatttacaggaagccatactgtagtcctatgttactcttaaaaatcccattcaaatcctgcttctgaggcctgcatactttctaccctaccagtcattgacccatgcttatgtctcctttgaaaacattgattccactcttgtctccagtgaaaaagtggaatttaagcagagaaacaaaagccatttgtcttgttaagtctactttccctctactttcaagaaggaaagttggggtatgtgttgaatggtgatttatttatttatttattattttaaaaattgatacaaggtcttactgtattgtgcaggctggtctcaaactcctgggctcaagtgatcatcccacctcagcctcccagtgttgggattacagcatgaaccattgtgcccaccaccgatccgcagttttttaagaaaaacttttactatagaaaattttaatcatatacaaaatacagaggaaagtatatgaacccactttaggagactagaatatgccaccccaaaatatgccactttggcataaggattatttcgagctaaaggcaactgggaagaaacacatagaagaaaagttctctgtccttctccatttgcctaaaagcaggacatgaatcttaaaagtccccctccttccctttctaccaggaaaaacaagagttaatcactgaagataacttcagacccttatcagtgtagagatggcactagaagaatctatattacatactcatttattttccttcccacaacttgccaccccagagactaaaaatccttttcctttgtcatgtctcttgtccaaaaatttgctctataagctggagttctaagccacctctttgagaattacttgttccctggtattttctgttaacatacatgtattaatatacatgttaacaagcttctgtttgtttttctcctgttttctgtcttgttacagaggtccatcccaactaagaactaaagagtaggaggaaaatataatttcctcctgcatactttgatcttgtttaatccgtaacccttcccacttttcacctcctacctattagattactttgaagcaaatttcagatatattactttatctataaatatttcagtatgtgctaggtgtggtggctcacacctgtaatcccaacactttgggaagctgaggcaggaggatcacttgagcccaggagttcaagaccagctacggcaacaaaaaatcaaaaacttatctgggcatggtggcacatgcctgtggtcccagctacatgagaggctgaggcaggaggatcgctttagcccaggaggttgaggctgcagtaagctgcattcacaccactgcactccagcctgggtgacagagtaagaccatgtctcaaaaaaatacatattttagtatgtatcctttttgtaaaaacacaatacttttatcatactttaaataataacaataattccttagtatcaccaaatattttgtcagtgtctcacattttccttattgtctaaaatattgttgatagttattcaaatcagaatccaaacaaggtccatatattacatttggttgacaagtctcttaagtttgttcatctttaagttcttcctccctctctttcatctcttgtaatttattaatgtgaaaaaacaggtaatttgttctatagtatttcctacattatagagtttgctacatttattccctatgatatcatttagcatgttcctctgtcccctgtgtttcctgtaaactggtagttatacctagaagcttgagtttattcaggtttttaattgtattttttttgcaagaattctttattatctgcttctggaagcacagaatgtctggttgtgtctggttttgatcttgacagctactgatgaccattgcctaatccattactttattggggtggggggaataaggttttaaaataaattttttttaaagatttttttaactgttattttgagacagtgtctcatttcgtttcccaggctggagtgcagtggcacaatcacggctcactgcagccttgacctcctgggatcaggtgatcttctcacctcagcctcctgggtacctggaactacaggtgcacaccaccacacctggctaattttttgtattttgtgtacagaaggggtttcatcatgtttcccagactggtcttgaactcctgggttcaagtgatctacccacttcagcttcccaaaatcctgggattacactttggccaccgtgcctggcctaaatgaaattatttgtctctaaacagacagaagttttactttaaaaatttgtctttgtgtgtacatgtgtttgtgtatgtgtgtgtgtctaaaagtttggctttgagctttgctttgaattcttggatgaacaataaccaagaatacttaaactctgatcattcttgacagatatcccctacaggctatggccttttgaattgtgtcctccagtgataaaaagcagcaagcacgatactgctctcagattcatggtggtcacatgtgaggtgaaaaaaaaaaaaaagatgaatcctatttaaatgcccccaggataacagtgatactctttgtaggataactatttgcttgccactggtttcattaaataaggacataagtaaagatctatttttgtctctttctccccaaccaccacaactaggattattggctatctcttctgttcaagaaattggtgggcaccaaggtgttaatggcaagcgtgcaaggttcaaagagaaggaagcttcgagtataccttcattgcacaaacactgacaagtaagtatgaaacacaccctttaccaatcatcaagttttagtgggtaagcctgtaactttactcaaacaccctgttgcatgtgtctatacattgcataagtataggcagttgcaatttagtaaagttttatacaacgattttattttattttatttttagaagaaaaatgctacttttgttgttgttgttttttgagacggggcctcgctcgtcacccaggctggagtgcagtggtgcaatctcagctcactgcaacctccgcctcccgggttcaagtgattcttgaagaggagaacaataataacaacaatattattttcaaaagttgtgaccgcagtttctggagttgagaagacatcgagatttttgtagcctcatactcttgctttaggtagcaaaaaatgttcctaaatctcaggaatattctctagataggtttcaatctatcattcctgataagatgatgctgaaatactaattctagccaaaaaagaccagctaccatttccgattgttggggactgggaactctggatagtgaggaccccagtaggaagtagcgaggggaatggtttgaatggataaattcataaaaaatgtcagtagatttaattttcttatacatttcagtctttttataaggctaggaaaagcccctgtttttatggtttataatttgaattcacatgaacccacaaaatttgccttttaccttcctatgtctgaaaatggatagtctggctggcctcttaacaacccagctggcagagctgtgaggatctcagtgtgctctagcccagacattggtagcatgaacggcaacatttttaattgtgttttcaaaataggagcacactagcggtctaaaacgatcataaaagaaggatactaagagggcccactgtcattatggatcctaatacttaggatgcattatggattgtcattatggatactaatacttaggatcacatttgtaattgagtttttaattgcttaaattagatacatatttctattaagttaacctctttgcttttagtccaaggtataaagaaggagatttaactctgtatgccataaacctccataatgtcaccaagtacttgcggttaccctatcctttttctaacaagcaagtggataaataccttctaagacctttgggacctcatggattactttccaagtaagtaattttccttgttcattccaaactttcaataaatttattggtgtttatcagaatagagagtttggacagggagcaaaagacaaagtcaactatatcaagttctaataattcttaatattcaggaaatttatgtatgaatacttactaatatgagtataactcatcctaagagtctaaagcaaaaggatgtgaacacaaactagcagttatcttagagaataagtttgcatttcaaaataacttgacatatcaagatccactcaacgcatttaaattatttactctaaaaagacataattcttggtaacacattcactaaagcaaaatatacctttatataattgctatcaaaggtatgtgggttggtataaaatatcataccatgtgagatcagtgtgattcctttacagcattaatttttattggttagagtaagaaaaagaatagctagagtatatttcttaagtagattctcatacactttggtttcaaaaaccaattattgactacatcttataaaagcctgtattcaatggagtgccaaaaaatgactatgagtcttaaagagttaggcatataaatattttaaggtttctgttcaatgtatgttggaaggagttcctttctcatgactattctcatattggagcataaaaagagtttacaggcttggcgcagtggctcatgcctgtaatcccaatactttgggaagctgaagcaggcagatcacttcagcccaggagtttgagaccagcctgggcaatatggcaaaactctctctacaaaatataccaaaattagccaggcgtggtggtgcatgcctgtagtcccagctacttgggaagctgaggtgggaggattgcttgagcccaggggggtcatggctgcagtgagctgtgatggtgcctctgtcacccagcctgggtgacagagtgagaccctgtctcaaaaaaataaataaataaaaattaagagtttacaaaattctcaccatctcctcccatctttgcaaatgccacataagtgatgtgttccaggactattagcctcggaacctgaggcagtacagtaagcacgctttctccaaagtcctgtcccccacagacaaacattatttacactgggtactgctcttttattttttcccctctatgctttattttactataactataatcatataacatgtaataggaaaaaggcagggtcgggggagagatccagaagtcttcccaagagcctttccaacatagcctctgtagacattttttctttcttctttttttttttttttttttttctgagacagagtctcactctgttgtccaggctagagtgcagtggcgtgatctaggctcactgcaacctccgcctcctgggttcaagcaattctcccacctcagcctccctagtagctgggattagaggcatgcatcaccacgcctggctaatttttgtatttttagtagagatgaggtttcaccatgtgggccaggctggtcttgaactcctgacctcaagtgatccacctgccttagcctcccaaagtgctaggattacacgagtgagccaccgtgccctgcccctattacattctgatcacacatttcatgttttataattggaaaactggtgaaattatagacaatgttttgttcccctaaattctctttgatgagtatatattacttacactcttctgtctttaaaattttgcaaaatagtatcctagataagtttatgagtgcacagtctgtacgcttactcatattaatgacctcggagagttaaacaacagtcacctttaaaaattattactatcattatcattatttttgaggcgggggtctcattctgtctcccaggctggagagtagtggtgcggtcacagctcactgcagccaccgctacctgggctcaagtgatccttcctcctcagccttctgagtagctgagaccacaggcttatgctaccacacctggctaattttttaactttttgtagagacgatgtctcattatgttgcccaggctggtctcaaactcctaagctcaagtgatcttcctcagcctcccaaagtgctgggattacaggcatgaaaaactgcacccagccctaaaaattattagggtcctgcatagtaagactttaataaatatttaaatgaacatctggtttttttaaaaaaaaaatagagacaaggtctcactatattgcccaagctggtctcgaactcctggactcacgcaatcctgctgccttagccgcccaaagtgctgggattacaggcatgacccacctcatctgggctgagtgaacatatttttaacataaaggccgtattttatatttatctcatacattttgcccagcatccccatttccgccgaatctgttgcttgctaattccttccagcttcatttcatctgaaatttgacaaacatcttctatttctttgtcgtcatgttattgacttcagaatataaaataaaacactatacccaaattaaaccccaccctcattgcccagcctgatgtgaaaataatcagcatacattaagcttacccttgatatatgtgtagcatcttttagataaatatacagctgattaagcaatatagcctgatggtataatatcttgcccatgtacctcatcttatctccagcaggattaattcacagtgatcagatttacctttaaactttgtagcaaaatatcctctccaaaagcatatctaaaacttttgtgtgtactcttgcaagtttcttaatttcatgcagaacaggctcttaccactgttagctggagatattttcaagacctatttttgtttgtggtttcctgatgatggtcatggcatttcccccttcactccatctaaaaattgaggtgatacaggcttttaaacaaaaccaactcatatagactgagtacaactgcaatgcaggcatgctaacctctgctacaatcatgggcgtgctattgatatgtcttaagttacagaacacagggctgagcgtctcattaggtcaaaatgtaaaccagtttttctgctcactgatgcttaatgaggacagggtgtgagagatttctttaaggaaaacaaatatataataatgctacatggaaaaatatctaacattagagaattaagtaaataaactaatatactcacaccatggaatcttgtgcagacattaaaattatgtagtggatggatgtttaatggtgtgagaaaaagttaggatgtgctggggtggggggaagaatcaagttttaagaaaatacagtatacccatacttaagtaaaaaaaaaaaaaaaggtatgtacagtcatgtgttgcttaatgatggggatacattccgagaaatgtgtcgataggtgatttcatccttgtgtgaacatcatagagtgaacttacacaaacctagatggtctagcctactatgtatctaggctatatgactagcctgttgctcctaggctacaaacctgtaaagcatgttactgtagcgaatatacaaatacttaacacaatggcaagctatcattgtgttaagtagttgtgtatctaaacatatctaaaacatagaaaactaatgtgttgtgctacaatgttacaatgactatgacattgctaggcaataggaattataattttatccttttatggaaccacacttatatatgcggtccatggtggaccaaaacatccttatgtggcatatgactgtatacatgtacacaaaaaatagatgaaagaatgaatatacatcaaaatatttaaaatggttataatgacttaggttacttttatttatcttagtaataataatgatgatagataatacttttatagtgtttactatataaaagacactgttataagtgttctacatactttacatgtattacctaaatgatataaatataactctgacagtaactaatcttatacgttctcttttcttttttttttttttctttttttagacagaatcttgctctaccaggctggagtgcagggtgcaatctcggctcactgcaacctccgcctcccaggttcaaacgattctcatgtctcagcctcctgagtagctgggactacaggcacacaccaccatgcccggctaatttttgtatttttgggtagagatggagttttgccatgttggccaggctgatcttgaactcctggcctcaagtgatctgcctgcctcagcctcccaaagtgctgggattacaggtgtgaaccactgtgctcggcctaatcttacaagttttcaatatttaaagagtgctaactttgttgacaatataaaacatatttgagaaaaagagatataagcatcttatttagaattatgaaaatatcaatagacctacagccgactaaagcttttcttcataagctcttgcctatattgattcgctcctgtgaatatgcattaatttgatttaaataataagtatgtataagaaataacacttttccttaatttttaagaacgttcaacagtttttaatttgaattccaatagtgaaatacatagaaaatataaaattttctgtagtttagccaaattgtttttgtttcaccacagcattctaccaaaatttcttaataacagtaagaaaatgaatgcatacctcctgcagggagaggggagttaggcagtttatgggcatagttacaagtgagaaatttcattggctaccatttacgctaaattcataaaaactgcattcaattctatatatctattttctttacataaaaaaggtttcaattattggccattaaataaaatagccaccattccagaagttgtgtcatgtttatcctttttataccaccatcatattgcctattatatagattgtgtgtgttccattttctgtaatgggccagacagtaagtatttctggctttggagtccatatggtctctatcataactactcatctctgccattgtagcttaaagattatctaggtcaaatgcctaagtgatatagtgttgaaatacaagttatataatataggctgccacaaaaaaaaatttatttggtctaaaaaagatttcatgacttttgtagcagcatgggtggggcatgcaccacttggttaactcggtgtatctttctcctttgcagatctgtccaactcaatggtctaactctaaagatggtggatgatcaaaccttgccacctttaatggaaaaacctctccggccaggaagttcactgggcttgccagctttctcatatagtttttttgtgataagaaatgccaaagttgctgcttgcatctgaaaataaaatatactagtcctgacactgaatttttcaagtatactaagagtaaagcaactcaagttataggaaaggaagcagataccttgcaaagcaactagtgggtgcttgagagacactgggacactgtcagtgctagatttagcacagtattttgatctcgctaggtagaacactgctaataataatagctaataataccttgttccaaatactgcttagcattttgcatgttttacttttatctaaagttttgttttgttttattatttatttatttatttattttgagacagaatctctctctgtcacccaggctggagtgccatggtgcgatcttggctcactgcaactttaagcaattctcctgcctcagcttcctgagtagctgggattataggcgtgtgccaccacgcccagctactttctatattttttgtagagatggagtttcgccatattggccaagctggtctcgaactcctgtcctcgaactcctgtcctcaagtgatccacccgcctcagcctctcaaagtgctgggattacaggtgtgagccaccacacccagcagtgttttatttttgagacagggtatcattctgttgcccaggcttgagtgcagtggtgcaatcatagatcactgcagccttttaactcctgggctcaagtcatcctcctgcttagcctcccaagtagctaggaccacagacacatgccatcacacttggctatttttaaaaaattttttgtagagatggggtctcgctatgttacccaaactggtcctgaactcctggactcaattgatcctcccaccttggccttccaggtgctgggatttctttgggagtacagcatggtacagcaggagatcatttgatgttacctctgtgcagtgttgctagtcagcgaaagactataatacctgtggggacagcgattagccaccacaaccagtctttatttaaagttattaaaaatggctgggcgcagtggctcacacctgtaatcctagcactttgggaggccgaggcagatggatcacctgacgtgaggaatttgagaccagcctggccaacatggtgaaaccccatctctactaaaaaatacaaaaattagctgggtgtggtcctgtagtcccagctacttgggaggctggggcaggagaattacttgaacccaggaggcagaggttgcagtgagccgagattgtgccactgcactccagcctgggtgacagagagagattccatctcaaaaaaacaagttattaaaaatgtatatgaatgctcctaatatggtcaggaagcaaggaagcgaaggatatattatgagttttaagaaggtgcttagctgtatatttatctttcaaaatgtattagaagattttagaattctttccttcatgtgccatctctacaggcacccatcagaaaaagcatactgccgttaccgtgaaactggttgtaaaagagaaactatctatttgcaccttaaaagacagctagattttgctgattttcttctttcggttttctttgtcagcaataatatgtgagaggacagattgttagatatgatagtataaaaaatggttaatgacaattcagaggcgaggagattctgtaaacttaaaattactataaatgaaattgatttgtcaagaggataaattttagaaaacacccaataccttataactgtctgttaatgcttgctttttctctacctttcttccttgtttcagttgggaagcttttggctgcaagtaacagaaactcctaattcaaatggcttaagcaataaggaaatgtatattcccacataactagacgttcaaacaggccaggctccagcacttcagtacgtcaccagggatctgggttcttcccagctctctgctctgccatctttagcgctggcttcattctcagactctggtagcatgatggctgtagctgtttcatgggccccttcaaacctcatagcaaccagaggaagaaaatgagccattttttgagtctccttcatagacttgaataactctttttcagagcttctcacagcaaacctctcctcatgtctcctcatgtcttattgttcagaaatgggtaatgtggccatttcaccagtcactgccaacaacaacgaggttcctataattgtctctgagtaaccctttggaatggagagggtgttggtcagtctacaaactgaacactgcagttctgcgctttttaccagtgaaaaaatgtaattattttcccctcttaaggattaatattcttcaaatgtatgcctgttatggatatagtatctttaaaattttttattttaatagctttaggggtacacactttttgcttacaggggtgaattgtgtagtggtgaagactcggcttttaatgtacttgtcacctgagtgatgtacattgtacccaataggtaatttttcatccattaccctccttccgccctcttcccttctgagtctccaacatcccttataccactgtgtatgttcttgtgtacctacagctaagcttccacttataagtgagaacatgcagtatttggttttccattcctgagttacttcccttaggataacagcccccagttccgtccaagttgctgcaaaatacattattcttctttatggctgagtaatagtccatggtacatatataccacattttctttatccacttatcagttgatggacacttaggttaattccattcaatttcattcaatttaagtatatttgtaaggagctaaagctgaaaattaaattttagatctttcaatactcttaaattttatatgtaagtggtttttatattttcacatttgaaataaagtaatttttataaccttgatattgtatgactattcttttagtaatgtaaagcctacagactcctacatttggaaccactagtgtgttgtttcaccccttgttatactatcaggatcctcga

SEQ ID NO: 43 - DNA, 2396 bp, Mus musculus
tttctagttgcttttagccaatgtcggatcaggtttttcaagcgacaaagagatactgagatcctgggcagaggacatcctagctcggtcagatttgggcaggctcaagtgaccagtgtcttaaggcagaagggagtcggggtagggtctggctgaaccctcaaccggggcttttaactcagggtctagtcctggcgccaaatggatgggacctagaaaaggtgacagagtgcgcaggacaccaggaagctggtcccacccctgcgcggctcccgggcgctccctccccaggcctccgaggatcttggattctggccacctccgcaccctttggatgggtgtggatgatttcaaaagtggacgtgaccgcggcggaggggaaagccagcacggaaatgaaagagagcgaggaggggagggcggggaggggagggcgctagggagggactcccgggaggggtgggagggatggagcgctgtgggagggtactgagtcctggcgccagaggcgaagcaggaccggttgcagggggcttgagccagcgcgccggctgccccagctctcccggcagcgggcggtccagccaggtgggatgctgaggctgctgctgctgtggctctgggggccgctcggtgccctggcccagggcgcccccgcggggaccgcgccgaccgacgacgtggtagacttggagttttacaccaagcggccgctccgaagcgtgagtccctcgttcctgtccatcaccatcgacgccagcctggccaccgacccgcgcttcctcaccttcctgggctctccaaggctccgtgctctggctagaggcttatctcctgcatacttgagatttggcggcacaaagactgacttccttatttttgatccggacaaggaaccgacttccgaagaaagaagttactggaaatctcaagtcaaccatgatatttgcaggtctgagccggtctctgctgcggtgttgaggaaactccaggtggaatggcccttccaggagctgttgctgctccgagagcagtaccaaaaggagttcaagaacagcacctactcaagaagctcagtggacatgctctacagttttgccaagtgctcggggttagacctgatctttggtctaaatgcgttactacgaaccccagacttacggtggaacagctccaacgcccagcttctccttgactactgctcttccaagggttataacatctcctgggaactgggcaatgagcccaacagtttctggaagaaagctcacattctcatcgatgggttgcagttaggagaagactttgtggagttgcataaacttctacaaaggtcagctttccaaaatgcaaaactctatggtcctgacatcggtcagcctcgagggaagacagttaaactgctgaggagtttcctgaaggctggcggagaagtgatcgactctcttacatggcatcactattacttgaatggacgcatcgctaccaaagaagattttctgagctctgatgcgctggacacttttattctctctgtgcaaaaaattctgaaggtcactaaagagatcacacctggcaagaaggtctggttgggagagacgagctcagcttacggtggcggtgcacccttgctgtccaacacctttgcagctggctttatgtggctggataaattgggcctgtcagcccagatgggcatagaagtcgtgatgaggcaggtgttcttcggagcaggcaactaccacttagtggatgaaaactttgagcctttacctgattactggctctctcttctgttcaagaaactggtaggtcccagggtgttactgtcaagagtgaaaggcccagacaggagcaaactccgagtgtatctccactgcactaacgtctatcacccacgatatcaggaaggagatctaactctgtatgtcctgaacctccataatgtcaccaagcacttgaaggtaccgcctccgttgttcaggaaaccagtggatacgtaccttctgaagccttcggggccggatggattactttccaaatctgtccaactgaacggtcaaattctgaagatggtggatgagcagaccctgccagctttgacagaaaaacctctccccgcaggaagtgcactaagcctgcctgccttttcctatggtttttttgtcataagaaatgccaaaatcgctgcttgtatatgaaaataaaaggcatacggtacccctgagacaaaagccgaggggggtgttattcataaaacaaaaccctagtttaggaggccacctccttgccgagttccagagcttcgggagggtggggtacacttcagtattacattcagtgtggtgttctctctaagaagaatactgcaggtggtgacagttaatagcactgtg

SEQ ID NO: 44 - protein, 535 aa, Mus musculus
MLRLLLLWLWGPLGALAQGAPAGTAPTDDVVDLEFYTKRPLRSVSPSFLSITIDASLATDPRFLTFLGSPRLRALARGLSPAYLRFGGTKTDFLIFDPDKEPTSEERSYWKSQVNHDICRSEPVSAAVLRKLQVEWPFQELLLLREQYQKEFKNSTYSRSSVDMLYSFAKCSGLDLIFGLNALLRTPDLRWNSSNAQLLLDYCSSKGYNISWELGNEPNSFWKKAHILIDGLQLGEDFVELHKLLQRSAFQNAKLYGPDIGQPRGKTVKLLRSFLKAGGEVIDSLTWHHYYLNGRIATKEDFLSSDALDTFILSVQKILKVTKEITPGKKVWLGETSSAYGGGAPLLSNTFAAGFMWLDKLGLSAQMGIEVVMRQVFFGAGNYHLVDENFEPLPDYWLSLLFKKLVGPRVLLSRVKGPDRSKLRVYLHCTNVYHPRYQEGDLTLYVLNLHNVTKHLKVPPPLFRKPVDTYLLKPSGPDGLLSKSVQLNGQILKMVDEQTLPALTEKPLPAGSALSLPAFSYGFFVIRNAKIAACI

SEQ ID NO: 45 - DNA, 2396 bp, Mus musculus; identical to SEQ ID NO: 43
  CDS (594)..(2198)

SEQ ID NO: 46 - DNA, 385 bp, Rattus norvegicus
cggccgctgctgctgctgtggctctgggggcggctccgtgccctgacccaaggcactccggcggggaccgcgccgaccaaagacgtggtggacttggagttttacaccaagaggctattccaaagcgtgagtccctcgttcctgtccatcaccatcgacgccagtctggccaccgaccctcggttcctcaccttcctgagctctccacggcttcgagccctgtctagaggcttatctcctgcgtacttgagatttggcggcaccaagactgacttccttatttttgatcccaacaacgaacccacctctgaagaaagaagttactggcaatctcaagacaacaatgatatttgcgggtctgaccgggtctccgctgacgtgttga

SEQ ID NO: 47 - DNA, 541 bp, Rattus norvegicus
  misc_feature (507)..(507): Any nucleotide
aaatcaggacatatccttcacttatttgcctcttggtcatattggaggcatttgtattcatttttaataaccctcaaaatagtgcatgcaaagtgctaagcgtcatttgccacatggtgccattaactgtcaccacctgcagtggtctacttagagaacaccgcactggatgttaacactgaagcgcgtgccccgccctcccgaggctctggatccagcgttgaagcttgccccgccctcccgaggctctggatccagcactggagcatgccccgccctcccgaggctctggagcttgctaaggagtccgctccctaccgctggggttttgctttattcttatgaatgacacccctgaccgctttcgtctcaggggtactgtaatgccttttattttcatatacaagctgcgattttggcatttcttatgacaaaaaacccataggaaaaggcgggcacgcttagtgagcttcctgcggggagaggtttttctgttagagctggcanggtctgctcatcgaccatcttcaggcctcgtgcc